(19) United States
(12) Patent Application Publication
(10) Pub. No.: US 2024/0229023 A9
(43) Pub. Date: Jul. 11, 2024

(54) RNA-TRIGGERED PROTEIN CLEAVAGE AND APPLICATIONS BY THE CRISPR CAS7-11-CSX29 COMPLEX

(71) Applicants: Massachusetts Institute of Technology, Cambridge, MA (US); The University of Tokyo, Tokyo (JP)

(72) Inventors: Omar Abudayyeh, Cambridge, MA (US); Jonathan Gootenberg, Cambridge, MA (US); Hiroshi Nishimasu, Sibuya-Ku (JP); Kazuki Kato, Tokyo (JP); Cian Schmitt-Ulms, Cambridge, MA (US); Kaiyi Jiang, Cambridge, MA (US)

(21) Appl. No.: 18/234,690

(22) Filed: Aug. 16, 2023

Related U.S. Application Data
(60) Provisional application No. 63/421,689, filed on Nov. 2, 2022.
(60) Provisional application No. 63/398,329, filed on Aug. 16, 2022.

(51) Int. Cl. C12N 15/11; C12N 9/22; C12N 9/50; C12N 15/10

(52) U.S. Cl. CPC C12N 15/111; C12N 9/22; C12N 9/50; C12N 15/102; C12N 15/11; C12Y 304/22; C07K 2319/33; C07K 2319/50; C07K 2319/60; C07K 2319/61; C12N 2310/20

(57) ABSTRACT

Disclosed are methods of RNA-triggered protein cleavage by the CRISPR Cas7-11-Csx29 complex. A guide RNA specifically hybridizes to a RNA target, and Csx29 cleaves Csx30 when Cas7-11:Csx29 complex binds to the target RNA.

### RELATED APPLICATIONS

This application claims the benefit of priority to U.S. Provisional Patent Application Ser. No. 63/398,329, filed Aug. 16, 2022; and U.S. Provisional Patent Application Ser. No. 63/421,689, filed Nov. 2, 2022; the contents of each of which are hereby incorporated herein by reference in their entirety.

### GOVERNMENT SUPPORT

This invention was made with Government support under Grant No. HG011857 awarded by the National Institutes of Health (NIH). The Government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Nov. 28, 2023, is named MTV-20401_SL.xml and is 224,938 bytes in size.

### BACKGROUND

Prokaryotic CRISPR-Cas systems provide adaptive immunity against foreign nucleic acids, including phages and mobile genetic elements, via diverse mechanisms of programmed nucleic-acid cleavage. CRISPR-Cas systems are divided into two classes based on the number of components in the effector complexes responsible for defense via cleavage of invading nucleic acids programmed by a CRISPR RNA (crRNA) guide. However, the CRISPR-Cas system is not widely used to cleave proteins. This potential protease activity can be utilized for disease treatment and diagnosis. Accordingly, there is a great need to identify the protease activity of the CRISPR-Cas system.

### SUMMARY OF THE INVENTION

In one aspect the present disclosure provides a method of treating cancer. The method may comprise administering to a subject in need thereof an effective amount of a Cas7-11:Csx29 complex or a first nucleic acid encoding the Cas7-11:Csx29 complex. The method may further comprise administering an effective amount of a guide RNA that specifically hybridizes to a RNA target. The method may further comprise administering an effective amount of an apoptotic protein fused to a inhibitory peptide via a Csx30 linker or a second nucleic acid encoding the apoptotic protein fused to the inhibitory peptide via the Csx30 linker, the apoptotic activity of the apoptotic protein is inhibited by the inhibitory peptide and the apoptotic activity of the apoptotic protein is activated upon the cleavage of Csx30. In some embodiments, the cancer comprises cells comprising the target RNA; and Csx29 cleaves Csx30 when Cas7-11:Csx29 complex binds to the target RNA.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the apoptotic protein is caspase 2, caspase 8, caspase 9, caspase 10, caspase 11, caspase 12, caspase 3, caspase 6, or caspase 7. In some embodiments, the apoptotic protein is an immune activating cytokine. In some embodiments, the immune activating cytokine is a cytokine or a chemokine. In some embodiments, the immune activating cytokine is interleukin 12 (IL-12), interleukin 7 (IL-7), interleukin 15 (IL-15), interleukin 2 (IL-2), interleukin 18 (IL-18), interleukin 21 (IL-21), interleukin 23 (IL-23), interleukin 1 beta (IL-1β), interleukin 6 (IL-6), interleukin 8 (IL-8), CD40L, macrophage inflammatory protein 1 alpha (CCL3) (M1P-1α), macrophage inflammatory protein 1 beta (CCL4) (M1P-1β), interferon gamma (IFNγ), Interferon beta (IFNβ), tumor necrosis factor alpha (TNFα), interleukin-1 receptor antagonist (IL-1ra), or interleukin 10 (IL-10). In some embodiments, the inhibitory peptide inhibits the activity of the protein via steric hindrance.

In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46. In some embodiments, the cancer is hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, or superficial spreading melanoma.

In another aspect the present disclosure provides a method of identifying a cell type of a cell based on the presence of an RNA target in the cell. The method may comprise delivering into the cell a Cas7-11:Csx29 complex or a first nucleic acid encoding the Cas7-11:Csx29 complex. The method may further comprise delivering into the cell a guide RNA that specifically hybridizes to the RNA target. The method may further comprise delivering into the cell a fluorescent protein fused to an inhibitory peptide via a Csx30 linker or a second nucleic acid encoding the fluorescent protein fused to the inhibitory peptide via the Csx30 linker, the fluorescence of the fluorescent protein is inhibited by the inhibitory protein and the fluorescence of the fluorescent protein is activated upon the cleavage of Csx30. In some embodiments, the cell type is identified as comprising the target RNA, if Csx29 cleaves Csx30 when Cas7-11:Csx29 complex binds to the target RNA and fluorescence is detected.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorescent protein is a green fluorescent protein, mCherry protein, a yellow fluorescent protein, a citrine fluorescent protein, a blue fluorescent protein, a cyan fluorescent protein, or a red fluorescent protein. In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

In another aspect the present disclosure provides a method of identifying a cell type of a cell based on the presence of a RNA target in the cell. The method may comprise delivering into the cell a Cas7-11:Csx29 complex or a nucleic acid encoding the Cas7-11:Csx29 complex. The method may further comprise delivering into the cell a guide RNA that specifically hybridizes to the RNA target. The method may further comprise delivering into the cell a fluorophore attached to a quencher via a Csx30 linker, the fluorescence of the fluorophore is inhibited by the quencher and the fluorescence of the fluorophore is activated upon the cleavage of Csx30. In some embodiments, the cell type is identified as comprising the target RNA if Csx29 cleaves Csx30 when Cas7-11:Csx29 complex binds to the target RNA and fluorescence is detected.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorophore is 6-carboxyfluorescein (FAM) or tetrachlorofluorescein (TET). In some embodiments, the quencher is tetramethylrhodamine (TAMRA). In some embodiments, the Csx30 linker is a polypeptide comprising an amino acid sequence selected from the group consisting of SEQ ID NOs: 36-46. In some embodiments, the Cas7-11 comprises D429A/D654A mutations.

In another aspect the present disclosure provides a method of modifying a genomic sequence in a target cell based on the presence of an RNA target in the cell. The method may comprise delivering into the cell effective amounts of a) a Cas7-11:Csx29 complex or a first nucleic acid encoding the Cas7-11:Csx29 complex, b) a guide RNA that specifically hybridizes to the RNA target, and c) a gene editing enzyme attached to an inhibitory peptide via a Csx30 linker or a second nucleic acid encoding the gene editing enzyme fused to the inhibitory peptide via the Csx30 linker. The gene editing activity of the gene editing enzyme may be inhibited by the inhibitory peptide and the gene editing activity of the gene editing enzyme may be activated upon the cleavage of Csx30.

In some embodiments, the gene editing enzyme is an endonuclease. In some embodiments, the gene editing enzyme is a zinc finger nuclease (ZFN), a transcription activator-like effector nuclease (TALENs), a Meganuclease, or a Cas9. In some embodiments, the genomic sequence is modified by gene knockout, insertion, site-directed mutation, deletion, integration, or base editing. In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

In another aspect the present disclosure provides a method of selectively enriching gene-modified cells. The method may comprise delivering into a mixture of gene-modified cells and non-gene-modified cells effective amounts of: a) a Cas7-11:Csx29 complex or a first nucleic acid encoding the Cas7-11:Csx29 complex, b) a guide RNA that specifically hybridizes to a RNA target, and c) an apoptotic protein fused to an inhibitory peptide via a Csx30 linker or a second nucleic acid encoding the apoptotic protein fused to the inhibitory peptide via the Csx30 linker. The apoptotic activity of the apoptotic protein may be inhibited by the inhibitory peptide and the apoptotic activity of the apoptotic protein may be activated upon the cleavage of Csx30. The non-gene-modified cells may comprise the target RNA and the gene-modified cells lack the target RNA. The Csx29 cleaves Csx30 when Cas7-11:Csx29 complex binds to the target RNA, triggering apoptosis in non-gene-modified cells and enriching the gene-modified cells.

In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the apoptotic protein is caspase 2, caspase 8, caspase 9, caspase 10, caspase 11, caspase 12, caspase 3, caspase 6, or caspase 7. In some embodiments, the apoptotic protein is an immune activating cytokine. In some embodiments, the immune activating cytokine is a cytokine or a chemokine. In some embodiments, the immune activating cytokine is interleukin 12 (IL-12), interleukin 7 (IL-7), interleukin 15 (IL-15), interleukin 2 (IL-2), interleukin 18 (IL-18), interleukin 21 (IL-21), interleukin 23 (IL-23), interleukin 1 beta (IL-1β), interleukin 6 (IL-6), interleukin 8 (IL-8), CD40L, macrophage inflammatory protein 1 alpha (CCL3) (M1P-1α), macrophage inflammatory protein 1 beta (CCL4) (M1P-1β), interferon gamma (IFNγ), Interferon beta (IFNβ), tumor necrosis factor alpha (TNFα), interleukin-1 receptor antagonist (IL-1ra), or interleukin 10 (IL-10). In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

In another aspect the present disclosure provides a method of identifying a mutation in the transcriptome of a cell based on the presence of an RNA target in the cell. The method may comprise delivering into the cell effective amounts of: a) a Cas7-11:Csx29 complex or a first nucleic acid encoding the Cas7-11:Csx29 complex, b) a guide RNA that specifically hybridizes to the RNA target, and c) a fluorescent protein fused to an inhibitory peptide via a Csx30 linker or a second nucleic acid encoding the fluorescent protein fused to the inhibitory peptide via the Csx30 linker. The fluorescence of the fluorescent protein may be inhibited by the inhibitory protein and the fluorescence of the fluorescent protein may be activated upon the cleavage of Csx30. The RNA target may comprise the mutation, and the mutation may be identified, if Csx29 cleaves Csx30 when Cas7-11:Csx29 complex binds to the target RNA and fluorescence is detected.

In some embodiments, the mutation is a single-nucleotide polymorphism (SNP), a single-nucleotide variant (SNV), a single-nucleotide substitution, a point mutation, a single-nucleotide deletion, and a single-nucleotide insertion, an alternatively spliced region, a deletion, or a frameshift. In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorescent protein is a green fluorescent protein, mCherry protein, a yellow fluorescent protein, a citrine fluorescent protein, a blue fluorescent protein, a cyan fluorescent protein, or a red fluorescent protein. In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

In another aspect the present disclosure provides a method of identifying a mutation in the transcriptome of a cell based on the presence of an RNA target in the cell. The method may comprise delivering into the cell effective amounts of: a) a Cas7-11:Csx29 complex or a first nucleic acid encoding the Cas7-11:Csx29 complex, b) a guide RNA that specifically hybridizes to the RNA target, and c) a fluorophore attached to a quencher via a Csx30 linker. The fluorescence of the fluorophore may be inhibited by the quencher and the fluorescence of the fluorophore may be activated upon the cleavage of Csx30. The RNA target may comprise the mutation, and the mutation may be identified, if Csx29 cleaves Csx30 when Cas7-11:Csx29 complex binds to the target RNA and fluorescence is detected.

In some embodiments, the mutation is a single-nucleotide polymorphism (SNP), a single-nucleotide variant (SNV), a single-nucleotide substitution, a point mutation, a single-nucleotide deletion, and a single-nucleotide insertion, an alternatively spliced region, a deletion, or a frameshift. In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorophore is 6-carboxyfluorescein (FAM) or tetrachlorofluorescein (TET). In some embodiments, the quencher is tetramethylrhodamine (TAMRA). In some embodiments, the Csx30 linker is a polypeptide comprising an amino acid sequence selected from the group consisting of SEQ ID NOs: 36-46.

In some embodiments, the Cas7-11 comprises D429A/D654A mutations. In some embodiments, the first nucleic acid, the second nucleic acid, and/or the guide RNA is administered or delivered with lipid nanoparticles (LNPs). In some embodiments, the first nucleic acid, and/or the second nucleic acid is a DNA, RNA, or a coding RNA. In some embodiments, the coding RNA is an mRNA, a self-replicating RNA, a circular RNA, a viral RNA, or a replicon RNA. In some embodiments, the Cas7-11:Csx29 complex, and/or the protein is administered or delivered via extracellular Contractile Injection System (eCIS) or engineered virus-like particles (eVLPs). In some embodiments, the RNA target is SERPINA1 RNA, scgb1a1 RNA, ADAR1 mRNA, FOXM1 mRNA, or H2AFX mRNA.

### BRIEF DESCRIPTION OF THE DRAWINGS

FIGS. 1A-D show Cryo-EM structures of the Cas7-11-crRNA-Csx29 complexes with and without the target RNA. FIG. 1A shows domain structures of Cas7-11 and Csx29. FIG. 1B shows nucleotide sequences of the crRNA and its target RNA. Disordered nucleotides are indicated by dashed circles. PFS, protospacer flanking sequence. FIG. 1B discloses SEQ ID NOS 84-85, respectively, in order of appearance. FIGS. 1C-1) show overall structures of Cas7-1 i-crRNA-Csx29 (FIG. 1C) and Cas7-11-crRNA-Csx29-tgRNA (FIG. 1D). The bound zinc ions are shown as spheres. The disordered L1 and L2 linkers are not shown for clarity.

FIGS. 2A-2E show interaction between Cas7-11 and Csx29. FIG. 2A shows structure of Csx29 in the Cas7-11-crRNA-Csx29 complex. FIG. 2B shows interface between Cas7-11 and Csx29 in the Cas7-11-crRNA-Csx29 complex. The Cas11 and INS domains are omitted for clarity. FIG. 2C shows location of the Csx29 active site. The catalytic residue H615 of the Csx29 protease is shown. FIGS. 2D-2E show interfaces between Cas7-11 and Csx29 in Cas7-11-crRNA-Csx29 (FIG. 2D) and Cas7-11-crRNA-Csx29-tgRNA (FIG. 2E). Csx29 is shown as a surface representation, except for the AR, which is shown as a ribbon representation. The AR and APD are disordered in the Cas7-11-crRNA-Csx29-tgRNA structure in (FIG. 2E).

FIGS. 3A-3E show target RNA-triggered Csx30 cleavage by Csx29, FIG. 3A shows schematic of the RNA-triggered Csx30 cleavage by the Cas7-11-crRNA-Csx29 complex. TR, target RNA without a PFS; CTR, cognate target RNA with a non-matching PFS; NTR, non-cognate target RNA with a matching PFS. FIG. 3A discloses SEQ ID NO: 86. FIG. 3B shows RNA-triggered Csx30 cleavage by the Cas7-11-crRNA-Csx29 complex. The Cas7-11-crRNA-Csx29 complex was incubated with Csx30 at 37° C. for 10 min in the presence or absence of the target RNA (CTR). The wild-type (W) and catalytically inactivated (FIG. 3D) versions of Cas7-11 and Csx29 were used. FIG. 3C shows effects of the complementarity between the crRNA 5′ tag and tgRNA PFS on the Csx30 cleavage. The dCas7-11-crRNA-Csx29 complex was incubated with Csx30 at 37° C. for 5, 10, or 15 min in the presence of the target RNA (TR, CTR, or NTR). FIG. 3D shows proteolytic cleavage site in Csx30. The Csx30 site cleaved by Csx29 is indicated by a triangle. The Csx30 structure was predicted using AlphaFold2, and the Ca atoms of M427 and K428 at the cleavage site are indicated by spheres. FIG. 3D discloses SEQ 1D NO: 37. FIG. 3E shows Csx29-mediated cleavage of the Csx30 mutants. The dCas7-11-crRNA-Csx29 complex was incubated with the Csx30 mutants at 37° C. for 10 min in the presence or absence of the target RNA (CTR). In (FIG. 3B), (FIG. 3C), and (FIG. 3E), the proteins were analyzed by SDS-PAGE, and the gel was stained with CBB.

FIGS. 4A-4J show effects of Csx30 and Csx31 on bacterial cell growth. FIG. 4A shows schematic of bacterial growth assays for studying the Csx30 and Csx31 functions. FIG. 4B shows Growth curves and end-point analyses (FIG. 4C) of E. coli expressing either full-length Csx30, the N-terminal fragment (residues 1-427) of Csx30 (Csx30-1), or the C-terminal fragment (residues 428-565) of Csx30 (Csx30-2). FIGS. 4D-4E show growth curves (FIG. 4D) and end-point analyses (FIG. 4E) of E. coli expressing either Csx30-1, full-length Csx30 and Csx31, or Csx30-1 and Csx31. In FIGS. 4B-4E, growth was compared between induced and uninduced expression conditions. In FIGS. 4C and 4E, significance was calculated via two tailed Student's t test (**** p<0.0001; n.s., not significant), Data are shown as mean+s.e.m. (n=3). FIG. 4F shows heatmap comparing the survival percentages of bacteria expressing either Csx30-1, Csx30-2, full-length Csx30 and Csx3l, full-length Csx30 alone, Csx30-1 and Csx3l, or Csx30-2 and Csx3l, cultured at three different temperatures. Percent survival was calculated by the ratio of OD600 of the bacterial culture under the induced conditions over the OD600 for the non-induced conditions. Color scale shows percent survival from 0 to 100 percent. FIG. 4G shows confocal images of E. coli expressing either EGFP alone, EGFP-Csx30, or EGFP-Csx31 and unlabeled Csx30. White outlines indicate the shapes of individual E. coli cells, FIG. 4H shows schematic of the mammalian application of the Cas7-11-Csx29-Csx30 degron reporter system for RNA sensing in live cells. FIG. 4I shows citrine fluorescence of HEK293FT cells transfected with either the Gluc target or pUC19 control target in the presence of the Cas7-11-Csx29-Csx30 degron reporter. Significance was calculated via two tailed Student's t test (****, p<0.0001; n.s., not significant). Data are shown as mean+s.e.m. (n=3). FIG. 4Q shows RNA-triggered Csx30 reporter cleavage in HEK293FT cells. The N-terminally FLAG-tagged citrine-Csx30-degron reporter was transfected either with or without the Gluc target and with a targeting or non-targeting (NT) guide. Forty-eight hours post-transfection, total protein was extracted from the transfected HEK293FT cells and analyzed by western blot with an anti-FLAG antibody.

FIG. 5 shows potential mechanism of cell growth inhibition by the Cas7-11-Csx29 effector complex. The schematic presents a proposed mechanism of the RNA-triggered proteolytic activation of Csx30 by the Cas7-11-Csx29 complex, which induces cell growth inhibition as part of anti-viral immunity. The Csx30 NTD probably binds RpoE as an anti-sigma factor, and affects cell growth and viability through unknown mechanisms. Csx31 likely functions as an antitoxin, thereby protecting the cell from the toxic effect of the Csx30 NTD.

FIGS. 6A-6F show Cryo-EM analysis of the Cas7-11-crRNA-Csx29 complex. FIG. 6A shows single-particle cryo-EM image processing workflow. FIG. 6B shows representative micrograph at a magnification of ×105,000. FIG. 6C shows representative 2D averaged class images from the particles used for final reconstruction. Number of particles and resolution of reconstruction are indicated for each class. FIG. 6D shows Fourier shell correlation (FSC) curves. Map-to-map FSC curve was calculated between the two independently refined half-maps after masking (blue line), and the overall resolution was determined by gold standard FSC=0,143 criterion. Map-to-Model FSC was calculated between the refined atomic models and maps (red line). FIG. 6E shows directional FSC plots calculated in the 3DFSC server. FIG. 6F shows Euler angle distribution of particles in the final reconstruction.

FIGS. 7A-7F show Cryo-EM analysis of the Cas7-11-crRNA-Csx29-tgRNA complex. FIG. 7A shows single-particle cryo-EM image processing workflow. FIG. 7B shows representative micrograph at a magnification of ×105,000. FIG. 7C shows representative 2D averaged class images from the particles used for final reconstruction. Number of particles and resolution of reconstruction are indicated for each class. FIG. 7D shows FSC curves. Map-to-map FSC curve was calculated between the two independently refined half-maps after masking, and the overall resolution was determined by gold standard FSC=0.143 criterion. Map-to-Model FSC was calculated between the refined atomic models and maps. FIG. 7E shows directional FSC plots calculated in the 3DFSC server. FIG. 7F shows Euler angle distribution of particles in the final reconstruction.

FIGS. 8A-8D show Cryo-EM density maps. FIGS. 8A-8B show Cryo-EM density maps for Cas7-11-crRNA-Csx29 (FIG. 8A) and Cas7-11-crRNA-Csx29-tgRNA (FIG. 8B). FIGS. 8C-8) show Cryo-EM density maps for Cas7-11-crRNA-Csx29 (FIG. 8C) and Cas7-11-crRNA-Csx29-tgRNA (FIG. 8D).

FIGS. 9A-9C show structural comparison of the Cas7-11 complexes in different states. FIGS. 9A-9C show structures of Cas7-11-crRNA-tgRNA (PDB ID: 7WAH) (FIG. 9A), Cas7-11-crRNA-Csx29 (FIG. 9B), and Cas7-11-crRNA-Csx29-tgRNA (FIG. 9C). The bound zinc ions are shown. The disordered L1 and L2 linkers are not shown for clarity. The disordered regions (residues 1043-1126) in the INS domain are indicated by dashed circles in (FIG. 9B) and (FIG. 9C). The bound RNA molecules are shown on the right of the complexes.

FIGS. 10A-10C show RNA recognition by Cas7-11. FIG. 10A shows recognition of the crRNA 5′ end by the Cas7.1 domain. The density map is shown as a gray mesh. The possible location of U(-16) and the pre-crRNA processing site are indicated by a dashed circle and a triangle, respectively. FIGS. 10B-10C show recognition of the guide-target duplex by Cas7-11 (FIG. 10B) and Csm (PDB ID: 6IFY) (FIG. 10C). The catalytic residues (D429A/D654A of Cas7-11 and D33N of Csm) are depicted as space-filling models. The target RNA cleavage sites are indicated by triangles. The thumb-like β-hairpins are indicated by circles in the schematics.

FIG. 11 shows structural comparison between Csx29 and human separase. Overall structures of Csx29 and human separase (PDB ID: 7NJ1). The catalytic residues are depicted as space-filling models. Securin (separase inhibitor) is colored gray. The close-up views of the protease active sites are shown in insets.

FIGS. 12A-12D show interaction between Cas7-11 and Csx29. FIG. 12A shows interface between Cas7-11 and Csx29 in the Cas7-11-crRNA-Csx29 complex. Cas7-11 and Csx29 are shown as ribbon and surface representations, respectively. The INS and CTE domains of Cas7-11 are omitted for clarity. FIG. 12B-12D show structures of the Cas7.1-Cas7.4 domains in Cas7-11-crRNA-tgRNA (PDB ID: 7WAH) (FIG. 12B), Cas7-11-crRNA-Csx29 (FIG. 12C), and Cas7-11-crRNA-Csx29-tgRNA (FIG. 12D). The bound zinc ions are shown as spheres. The α-helical insertion in the Cas7.4 ZF motif is highlighted.

FIGS. 13A-13D show interface between Cas7-11 and Csx29. FIG. 13A shows interface between Cas7-11 Cas7.4 and Csx29 NTD. FIG. 13B shows interface between Cas7-11 Cas7.3/L2 and Csx29 NTD. FIG. 13C shows interface between Cas7-11 L2 and the Csx29 NTD/TPR. FIG. 13D shows interface between Cas7-11 Cas7.3 and Csx29 TPR1/2.

FIGS. 14A-14D show target RNA-induced conformational change in the Cas7-11-Csx29 complex. FIGS. 14A-14B show interfaces between Cas-11 and Csx29 in Cas74-crRNA-Csx29 (FIG. 14A) and Cas7-11-crRNA-Csx29-tgRNA (FIG. 14B). FIG. 14C shows recognition of the tgRNA non-matching PFS by Cas7-11. The density map for the RNA molecules is shown as a gray mesh. FIG. 14D shows superimposition of Cas7-11-crRNA-Csx29 and Cas7-11-crRNA-Csx29-tgRNA. A potential steric clash between the tgRNA non-matching PFS and Csx29 (TPR1 and AR2) is indicated by a dashed circle.

FIGS. 15A-15B show target RNA and Csx30 cleavage by the Cas7-11-Csx29 complex. FIG. 15A shows the Cas7-11-crRNA-Csx29 complex was incubated with a 5′-Cy5-labeled ssRNA target at 37° C. for 10 min, and then analyzed by 15% TBE-urea PAGE, The gels were visualized, using either Cy5 or SYBR Gold fluorescence. The wild-type (W) and catalytically inactivated (D) versions of Cas7-11 and Csx29 were used. FIG. 15B shows RNA-triggered Csx30 cleavage by the Cas7-11-crRNA-Csx29 complex. The Cas7-11-crRNA-Csx29 complex was incubated with Csx30 at 37° C. for 5 min in the presence of the target RNA (CTR). The wild-type (W) and catalytically inactivated (D) versions of Cas7-11 and Csx29 were used.

FIG. 16 shows N-terminal analysis of Csx30. Elution profiles for N-terminal seven residues in the ˜15 kDa Csx30 fragment (Csx30-2) were shown.

FIGS. 17A-17D show effects of Csx30 and Csx31 on bacterial cell growth. FIG. 17A shows growth curves of E. coli expressing the non-induced full-length Csx30, the N-terminal fragment (residues 1-427) of Csx30 (Csx30-1), or the C-terminal fragment (residues 428-565) of Csx30 (Csx30-2). These curves serve as non-induced controls for the curves in FIG. 4B. FIG. 17B shows effects of Csx30 and Csx3l on bacterial growth at a range of arabinose concentrations. End-point analysis of E. coli expressing arabinose-inducible full-length Csx30, the N-terminal fragment (residues 1-427) of Csx30 (Csx30-1), the C-terminal fragment (residues 428-565) of Csx30 (Csx30-2), or full-length and N- or C-terminal Csx30 fragments conjugated to Csx31, OD₆₀₀ values are shown for bacteria at concentrations ranging from 0 to 2% arabinose in the growth media, including the 1% value used for other experiments in the study. FIG. 17C shows electrostatic surface potential of the Csx30 and Csx31 structures predicted using AlphaFold2. The predicted structures suggested that Csx30 and Csx31 have negatively and positively charged surfaces, respectively. FIG. 171) shows growth curves of E coli expressing non-induced Csx30-1, full-length Csx30 and Csx3l, or Csx30-1 and Csx3l. These curves serve as non-induced controls for the curves in FIG. 4D.

FIGS. 18A-18E show interaction between Csx30, Csx3l, and RpoE. FIGS. 18A-18B show elution profiles of the Csx30-Csx3l-RpoE complex from a gel-filtration column. Csx30, Hiss-tagged Csx31 (“His₆” disclosed as SEQ ID NO: 83), and His₆-tagged RpoE (“His₆” disclosed as SEQ ID NO: 83) were co-expressed in E. coli, and purified by Ni-NTA and HiLoad 16/600 Superdex 200 columns. In (FIG. 18A), the Csx30-Csx3l-RpoE complex was loaded onto a Superdex 200 Increase column. In (FIG. 18B), the Csx30-Csx31-RpoE complex was incubated with the Cas7-11-crRNA-Csx29-tgRNA complex, and then loaded onto a Superdex 200 Increase column. The fractions indicated by orange lines were analyzed by SDS-PAGE, and the gels were stained with CBB. FIG. 18C shows predicted structure of the Csx30-Csx3I-RpoE complex. The structures of Csx30-Csx3l and Csx30-RpoE were predicted using AlphaFold2, and then they are superimposed based on the Csx30 NTDs. The Csx30 CTD in Csx30-RpoE is omitted for clarity. FIG. 18D shows structural comparison of D. ishimotonii RpoE (model) and E. coli RpoE (PDB ID: 6JBQ). FIG. 18E shows structural comparison of the Csx30 CTD (model) and CagX (PDB ID: 60EG).

FIG. 19 shows multiple sequence alignment of the N-terminal domain of the Csx30 orthologs. The figure was prepared using the Muscle5 program and ESpript3 (world wide web at espript.ibcp.fr/ESPript/ESPript). The cleavage site between M427 and K428 of D. ishimotonii Csx30 (WP_-124327587.1) is indicated by a triangle. FIG. 19 discloses SEQ ID NOS 87-104, respectively, in order of appearance.

FIG. 20 shows multiple sequence alignment of the C-terminal domain of the Csx30 orthologs. The figure was prepared using the Muscle5 program and ESpript3. Three families are represented by a single sequence and are not therefore aligned, FIG. 20 discloses SEQ ID NOS 105-122, respectively, in order of appearance.

FIG. 21 shows Western blot analysis of the mammalian citrine-Csx30-degron reporter. RNA-triggered reporter cleavage in mammalian cells. The FLAG-tagged citrine-Csx30-degron reporter was transfected either with or without the Gluc target and with a targeting or non-targeting (NT) guide. Forty-eight hours post-transfection, total protein was extracted from the transfected HEK293FT cells and analyzed by western blot with anti-FLAG and anti-ACTB (control) antibodies.

FIG. 22 shows Potential mechanism of cell growth inhibition by the Cas7-11-Csx29 effector complex, Schematic presentation of a proposed mechanism for the RNA-triggered proteolytic activation of Csx30 by the Cas7-11-Csx29 complex, which induces cell growth inhibition as part of anti-viral immunity. The Csx30 NTD probably binds RpoE as an anti-sigma factor, and affects cell growth and viability through unknown mechanisms. Csx31 likely functions as an antitoxin, thereby protecting the cell from the toxic effects of the Csx30 NTD.

### DETAILED DESCRIPTION OF THE INVENTION

Prokaryotic CRISPR-Cas systems provide adaptive immunity against foreign nucleic acids, including phages and mobile genetic elements, via diverse mechanisms of programmed nucleic-acid cleavage. CRISPR-Cas systems are divided into two classes based on the number of components in the effector complexes responsible for defense via cleavage of invading nucleic acids programmed by a CRISPR RNA (crRNA) guide. In Class 1 systems, which encompass types I, III, and IV, target nucleic acids are degraded by multi-protein effector complexes, whereas, in Class 2 systems, including types II, V, and VI, the effector complexes are formed by a single multidomain Cas protein (Cas9, Cas12, and Cas13, respectively). Beyond primary effector nuclease function, both Class 1 and Class 2 CRISPR-Cas systems deploy a wide-array of accessory proteins to enhance the antiviral activity of the primary effector nuclease, including secondary nuclease activation via cyclic oligoadenylate generation in type III-A/B/D systems and target RNA-dependent pore formation by Csx28 in type VI-B systems.

Unlike typical Class 1 effectors, the type III-E effector Cas7-11 (also known as gRAMP) is a single-protein, multidomain effector that consists of four Cas7 domains (Cas7.1-Cas7.4) and a Cas11 domain, and likely evolved from the more complex type III-D multi-subunit effectors via domain fusions, Cas7-11 associates with a crRNA and cleaves complementary single-stranded RNA (ssRNA) targets at two defined positions, using the Cas7.2 and Cas7.3 domains, respectively. Whereas the type VI effector Cas13 displays promiscuous RNase activity, Cas7-11 exhibits specific, guide RNA-dependent RNA cleavage activity in human cells, and has been used as a novel RNA-targeting tool with high specificity and low cell toxicity. The type III-E locus contains multiple conserved accessory proteins, including Csx29 (a caspase-like putative protease with fused TPR and CHAT domains), Csx30 and Csx31 (proteins with unknown functions), and RpoE (an alternative sigma factor). Cas7-11 forms a complex with Csx29, suggesting a potential mechanism of RNA-guided protease activity for antiviral immunity. The cryo-electron microscopy (cryo-EM) structure of Desulfonema ishimotonii Cas7-11 in complex with its cognate crRNA and target RNA (tgRNA) provides mechanistic insights into the pre-crRNA processing and tgRNA cleavage. However, how Cas7-11 cooperates with the other proteins encoded in the type III-E locus (Csx29, Csx30, Csx31, and RpoE), and how Cas7-11 binds to Csx29 and potentially activates its protease activity remain unknown.

The type III-E Cas7-11 effector nuclease associates with a CRISPR RNA (crRNA) and the putative caspase-like protease Csx29, and catalyzes crRNA-guided target RNA cleavage. Here, we report cryo-electron microscopy structures of the Cas7-11-crRNA-Csx29 complex with and without target RNA, and demonstrate that target RNA binding induces a conformational change in Csx29 and results in the protease activation. Biochemical analysis confirmed that Cas7-11-bound Csx29 cleaves Csx30 in a target RNA-dependent manner, Reconstitution of the system in bacteria uncovered Csx30-dependent cellular toxicity regulated by Csx31, and showed that Csx29-mediated cleavage produces toxic Csx30 fragments, promoting growth suppression. We find that Csx30 can bind both Csx31 and the associated sigma factor RpoE, suggesting that Csx30 inhibits RpoE and modulates cellular stress response towards infection. Thus, the RNA-guided nuclease and protease activities of the Cas7-11-Csx29 effector complex mediate protease-based programmed growth suppression in bacterial immunity. Furthermore, we engineered the Cas7-1-Csx29-Csx30 system for programmable RNA sensing in mammalian cells.

In this disclosure, we demonstrate that the type III-E Cas7-11-Csx29 effector complex is an RNA-activated nuclease-protease, in which Csx29 specifically cleaves another type III-E associated protein Csx30. A structural comparison of the Cas7-11-crRNA-Csx29 complexes with and without a target RNA revealed that target RNA-binding induces a structural change in Csx29, likely activating the Csx29 protease activity. Consistent with this structural finding, our biochemical analysis demonstrated that Csx29 is a target RNA-triggered protease that cleaves Csx30 at a unique site. The Cas7-11-Csx29 complex is activated when bound to a target RNA with a non-matching PFS, suggesting a potential mechanism for self-targeting avoidance in the natural host. Analysis of the effects of Csx30 and Csx31 on bacterial growth suggested that the Csx29-mediated Csx30 cleavage releases the N-terminal fragment of Csx30 in complex with Csx31, inhibiting host cell growth (FIG. 22). Furthermore, our biochemical and structural analyses indicated that Csx30, Csx31, and RpoE can form a ternary complex, in which Csx30 extensively interacts with RpoE, suggesting that Csx30 inhibition of RpoE activity is a potential mechanism of the observed cell growth arrest. It is also possible that Csx30 cleavage by Csx29 facilitates the dissociation of RpoE from Csx30, allowing RpoE to engage in a transcriptional response to viral infection. Taken together, these findings show that the type III-E Cas7-11-Csx29 effector complex is an RNA-triggered programmable nuclease-protease capable of cleaving ssRNA targets and the Csx30 protein, unleashing a downstream signaling cascade that affects cell growth, likely via transcriptional regulation. Leveraging the programmable nature of this system, we developed a molecular RNA sensor for transcripts in mammalian cells, demonstrating the potential of this system for sensing and therapeutic applications, analogous to recent mammalian RNA sensor systems developed.

Thus, in the type III-E CRISPR-Cas systems, the Cas7-11-Csx29 effector complex likely degrades ssRNA transcripts of phage genes and stimulates potentially toxic host cell stress responses through the Csx29-mediated Csx30 cleavage (FIG. 22).

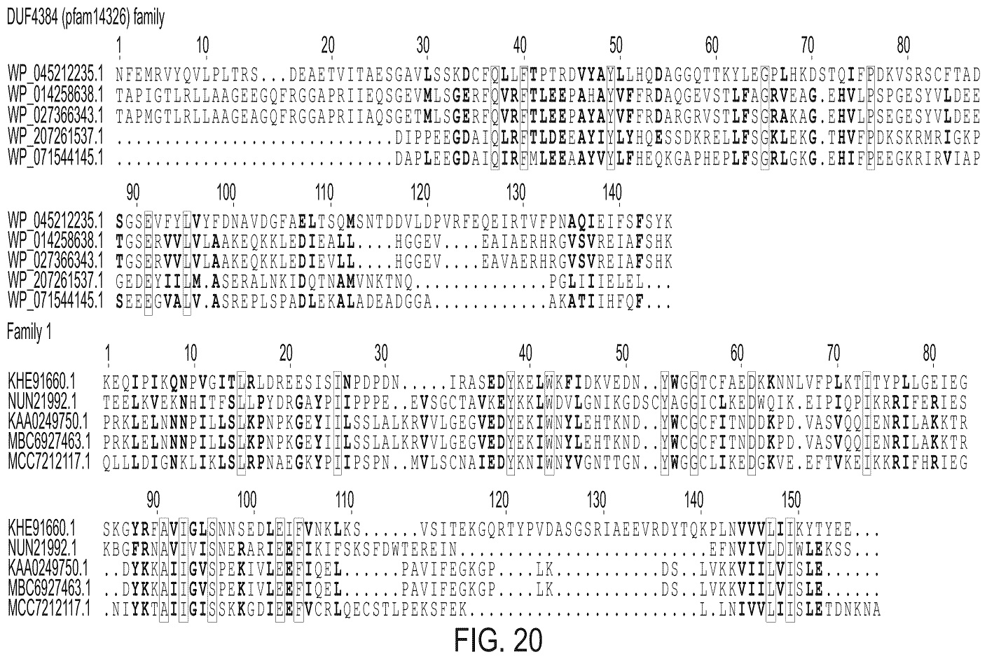

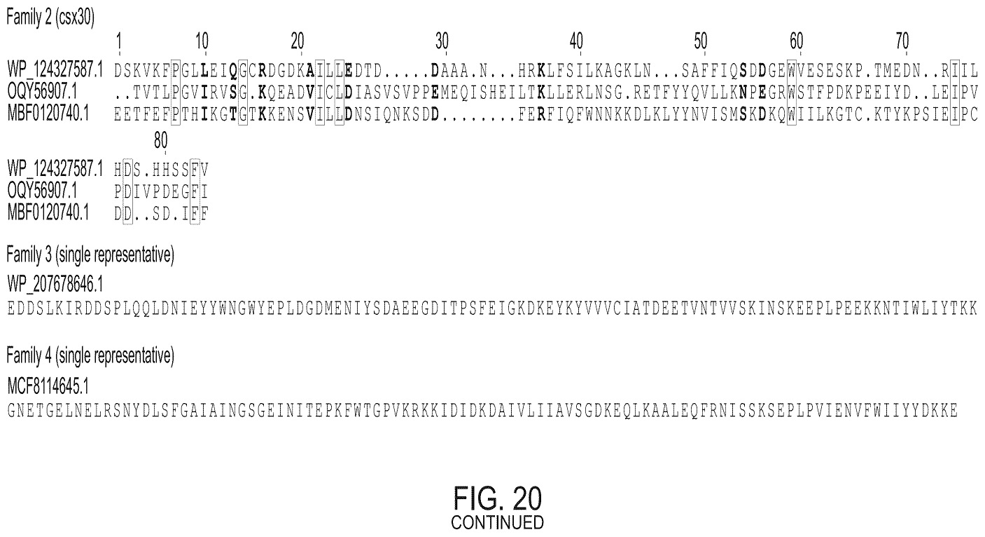

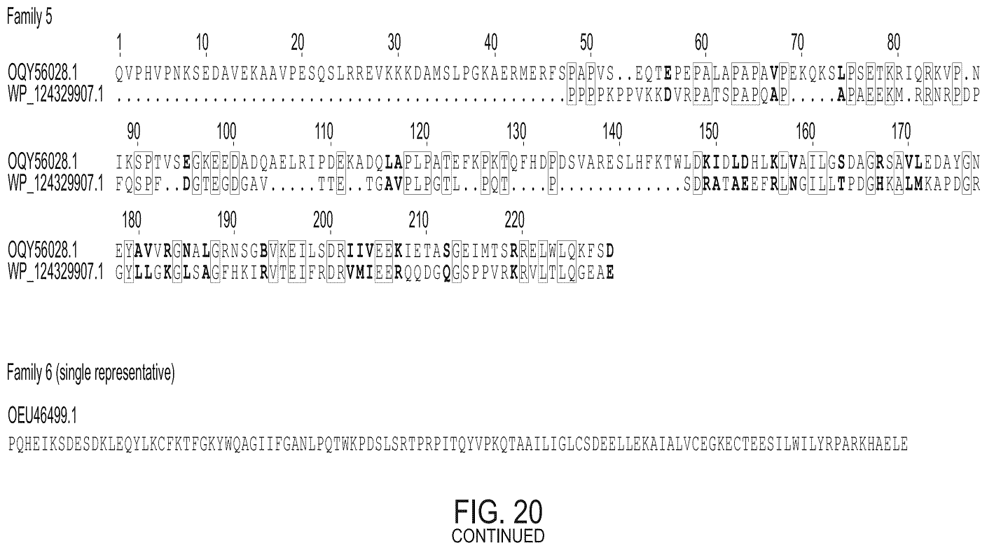

This type of programmed growth 30 suppression, through cell death or growth arrest, appears analogous to that caused by the bacterial membrane pore-forming toxins gasdermins, which are switched on via the release of auto-inhibitory peptides by associated proteases that become activated during phage infection. Moreover, given the high diversity of Csx30 CTDs (FIG. 20), further explorations of other subtype III-E systems might reveal additional functions associated with Cas7-11-mediated 35 target RNA recognition. Given our protein localization data and the unexpected structural similarity between the Csx30 CTD and pore-forming proteins in type IV secretion systems, Csx30 and Csx31 co-localize near the cell membrane or inner foci in cells, potentially with RpoE, modulating activity. Csx29-mediated proteolysis would liberate Csx30 NTD, Csx31, and RpoE into the cytoplasm, potentially restoring or further modulating RpoE activity and leading to cell death or growth arrest. This type of programmed growth suppression, through cell death or growth arrest, might be analogous to that caused by the bacterial membrane pore-forming toxins gasdermins that are activated via proteolytic cleavage and release of auto-inhibitory peptides by associated proteases activated during phage infection. Moreover, given the high diversity of Csx30 CTDs (FIG. 20), further exploration of other subtype III-E systems might reveal additional functions associated with Cas7-11-mediated target RNA recognition.

Among the CRISPR-Cas systems, a biological, if not mechanistic, analogy can be found in type VI systems, where the Cas13-crRNA effector complex recognizes complementary phage mRNAs and cleaves both phage (specifically and in cis) and host (indiscriminately and in trans) transcripts, stalling the cell growth, and with it, the infectious cycle. Similarly, in some type III systems, the CRISPR-Lon protease can be activated via cyclic oligoadenylates upon RNA recognition by type III effector complexes, and specifically cleaves the associated CRISPR-T protein, releasing a toxic fragment. Our characterization of the subtype III-E system highlighted the remarkable diversity of CRISPR-associated functions activated by programmable nucleic-acid recognition, thereby motivating continued exploration of CRISPR-associated proteins and their potential programmable functions that may have useful roles for biology applications. Our findings that the type III-E Cas7-11-Csx29 effector complex is a so far unique RNA-triggered nuclease-protease establish a new paradigm of prokaryotic signal transduction in viral immunity, and could pave the way for the development of new RNA/protein-targeting technologies, including in vitro diagnostics and cellular RNA sensing.

### Methods of Use

In one aspect the present disclosure provides methods of treating cancer. The method may comprise administering to a subject in need thereof an effective amount of a Cas7-11:Csx29 complex or a first nucleic acid encoding the Cas7-11:Csx29 complex. The method may further comprise administering an effective amount of a guide RNA that specifically hybridizes to a RNA target. The method may further comprise administering an effective amount of an apoptotic protein fused to a inhibitory peptide via a Csx30 linker or a second nucleic acid encoding the apoptotic protein fused to the inhibitory peptide via the Csx30 linker, the apoptotic activity of the apoptotic protein is inhibited by the inhibitory peptide and the apoptotic activity of the apoptotic protein is activated upon the cleavage of Csx30. In some embodiments, the cancer comprises cells comprising the target RNA; and Csx29 cleaves Csx30 when Cas7-11:Csx29 complex binds to the target RNA.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA).

CRISPR RNA or crRNA is a RNA transcript from the CRISPR locus. CRISPR-Cas (clustered, regularly interspaced short palindromic repeats—CRISPR associated systems) is an adaptive immune system found in bacteria and archaea to protect against mobile genetic elements, like viruses, plasmids, and transposons. The CRISPR locus contains a series of repeats interspaced with unique spacers. These unique spacers can be acquired from MGEs. Pre-crRNA is formed after the transcription of the CRISPR locus and before being processed by Cas proteins. Mature crRNA transcripts contain a partial conserved section of repeat and a sequence of spacer that is complementary to the target DNA. crRNA forms an effector complex with a single nuclease or multiple Cas proteins called a Cascade (CRISPR-associated complex for antiviral defense).

In some embodiments, the apoptotic protein is caspase 2, caspase 8, caspase 9, caspase 10, caspase 11, caspase 12, caspase 3, caspase 6, or caspase 7. In some embodiments, the apoptotic protein is an immune activating cytokine. The apoptotic proteins can initiate or amplify cell death signaling. In some embodiments, the immune activating cytokine is a cytokine or a chemokine. In some embodiments, the immune activating cytokine is interleukin 12 (IL-12), interleukin 7 (IL-7), interleukin 15 (IL-15), interleukin 2 (IL-2), interleukin 18 (IL-18), interleukin 21 (IL-21), interleukin 23 (IL-23), interleukin 1 beta (IL-1β), interleukin 6 (IL-6), interleukin 8 (IL-8), CD40L, macrophage inflammatory protein 1 alpha (CCL3) (M1P-1α), macrophage inflammatory protein 1 beta (CCL4) (M1P-1β), interferon gamma (IFNγ), Interferon beta (IFNβ), tumor necrosis factor alpha (TNFα), interleukin-1 receptor antagonist (IL-1ra), or interleukin 10 (IL-10).

Cytokines are a broad and loose category of small proteins (˜5-25 kDa) important in cell signaling. Due to their size, cytokines cannot cross the lipid bilayer of cells to enter the cytoplasm and therefore typically exert their functions by interacting with specific cytokine receptors on the target cell surface. Cytokines have been shown to be involved in autocrine, paracrine and endocrine signaling as immunomodulating agents. Cytokines include chemokines, interferons, interleukins, lymphokines, and tumour necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations.

In some embodiments, the inhibitory peptide inhibits the activity of the protein via steric hindrance.

In another aspect the present disclosure provides methods of identifying a cell type of a cell based on the presence of a RNA target in the cell. The method may comprise delivering into the cell a Cas7-11:Csx29 complex or a first nucleic acid encoding the Cas7-11:Csx29 complex. The method may further comprise delivering into the cell a guide RNA that specifically hybridizes to the RNA target. The method may further comprise delivering into the cell a fluorescent protein fused to a inhibitory peptide via a Csx30 linker or a second nucleic acid encoding the fluorescent protein fused to the inhibitory peptide via the Csx30 linker, the fluorescence of the fluorescent protein is inhibited by the inhibitory protein and the fluorescence of the fluorescent protein is activated upon the cleavage of Csx30. In some embodiments, the cell type is identified as comprising the target RNA, if Csx29 cleaves Csx30 when Cas7-11:Csx29 complex binds to the target RNA and fluorescence is detected.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorescent protein is a green fluorescent protein, mCherry protein, a yellow fluorescent protein, a citrine fluorescent protein, a blue fluorescent protein, a cyan fluorescent protein, or a red fluorescent protein.

Fluorescent proteins are members of a structurally homologous class of proteins that share the unique property of being self-sufficient to form a visible wavelength chromophore from a sequence of 3 amino acids within their own polypeptide sequence.

The green fluorescent protein (GFP) is a protein that exhibits bright green fluorescence when exposed to light in the blue to ultraviolet range. The label GFP traditionally refers to the protein first isolated from the jellyfish Aequorea victoria and is sometimes called avGFP. However, GFPs have been found in other organisms including corals, sea anemones, zoanthids, copepods and lancelets.

Yellow fluorescent protein (YFP) is a genetic mutant of green fluorescent protein (GFP) originally derived from the jellyfish Aequorea victoria. Its excitation peak is 513 nm and its emission peak is 527 nm. Like the parent GFP, YFP is a useful tool in cell and molecular biology because the excitation and emission peaks of YFP are distinguishable from GFP which allows for the study of multiple processes/proteins within the same experiment.

Red fluorescent protein (RFP) is a fluorophore that fluoresces red-orange when excited. Several variants have been developed using directed mutagenesis. The original was isolated from Discosoma, and named DsRed. Others are available that fluoresce orange, red, and far-red.

In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

In another aspect the present disclosure provides methods of identifying a cell type of a cell based on the presence of a RNA target in the cell. The method may comprise delivering into the cell a Cas7-11:Csx29 complex or a nucleic acid encoding the Cas7-11:Csx29 complex. The method may further comprise delivering into the cell a guide RNA that specifically hybridizes to the RNA target. The method may further comprise delivering into the cell a fluorophore attached to a quencher via a Csx30 linker, the fluorescence of the fluorophore is inhibited by the quencher and the fluorescence of the fluorophore is activated upon the cleavage of Csx30. In some embodiments, the cell type is identified as comprising the target RNA, if Csx29 cleaves Csx30 when Cas7-11:Csx29 complex binds to the target RNA and fluorescence is detected.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorophore is 6-carboxyfluorescein (FAM) or tetrachlorofluorescein (TET).

A fluorophore (or fluorochrome, similarly to a chromophore) is a fluorescent chemical compound that can re-emit light upon light excitation. Fluorophores typically contain several combined aromatic groups, or planar or cyclic molecules with several 71 bonds.

In some embodiments, the quencher is tetramethylrhodamine (TAMRA). In some embodiments, the Csx30 linker is a polypeptide comprising an amino acid sequence selected from the group consisting of SEQ ID NOs: 36-46. In some embodiments, the Cas7-11 comprises D429A/D654A mutations.

In another aspect the present disclosure provides methods of treating a bacterial infection. The method may comprise administering to a subject in need thereof an effective amount of a Cas7-11:Csx29 complex or a first nucleic acid encoding the Cas7-11:Csx29 complex. The method may further comprise administering an effective amount of a guide RNA that specifically hybridizes to a RNA target. The method may further comprise administering an effective amount of a bacterial toxic protein fused to a degron via a Csx30 linker or a second nucleic acid encoding the bacterial toxic protein fused to the degron via the Csx30 linker. The toxic activity of the bacterial toxic protein is inhibited by the degron and the toxic activity of the bacterial toxic protein is activated upon the cleavage of Csx30. In some embodiments, the bacteria comprises the RNA target; and Csx29 cleaves Csx30 when Cas7-11:Csx29 complex binds to the target RNA. By fusing a bacterial toxic protein (such as the CcdB toxin) with a degron (such as an SsrA tag) with a Csx30 linker, we can engineer a protein that, in the absence of Csx29 activity, is degraded by the degron tag. In the presence of Csx29 activation (such as during target recognition by the Csx29-Cas7-11 complex), the protease will cleave apart the toxin from the degron, stabilizing the toxin and leading to cell death. This system provides a sensitive and retargetable antibiotic application.

In another aspect the present disclosure provides a method of modifying a genomic sequence in a target cell based on the presence of a RNA target in the cell. The method may comprise delivering into the cell effective amounts of a) a Cas7-11:Csx29 complex or a first nucleic acid encoding the Cas7-11:Csx29 complex, b) a guide RNA that specifically hybridizes to the RNA target, and c) a gene editing enzyme attached to an inhibitory peptide via a Csx30 linker or a second nucleic acid encoding the gene editing enzyme fused to the inhibitory peptide via the Csx30 linker. The gene editing activity of the gene editing enzyme may be inhibited by the inhibitory peptide and the gene editing activity of the gene editing enzyme may be activated upon the cleavage of Csx30.

In some embodiments, the gene editing enzyme is an endonuclease. In some embodiments, the gene editing enzyme is a zinc finger nuclease (ZFN), a transcription activator-like effector nuclease (TALENs), a Meganuclease, a Cas9, or cas19. Endonucleases are enzymes that cleave the phosphodiester bond within a polynucleotide chain (namely DNA or RNA). Some, such as deoxyribonuclease I, cut DNA relatively nonspecifically (without regard to sequence), while many, typically called restriction endonucleases or restriction enzymes, cleave only at very specific nucleotide sequences.

For example, a CRISPR-Cas9 or CRISPR-Cas12 nuclease is fused with a degron by Csx30 linker.

In some embodiments, the genomic sequence is modified by gene knockout, insertion, site-directed mutation, deletion, integration, or base editing. In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

In another aspect the present disclosure provides a method of selectively enriching gene-modified cells. The method may comprise delivering into a mixture of gene-modified cells and non-gene-modified cells effective amounts of: a) a Cas7-11:Csx29 complex or a first nucleic acid encoding the Cas7-11:Csx29 complex, b) a guide RNA that specifically hybridizes to a RNA target, and c) an apoptotic protein fused to an inhibitory peptide via a Csx30 linker or a second nucleic acid encoding the apoptotic protein fused to the inhibitory peptide via the Csx30 linker. The apoptotic activity of the apoptotic protein may be inhibited by the inhibitory peptide and the apoptotic activity of the apoptotic protein may be activated upon the cleavage of Csx30. The non-gene-modified cells may comprise the target RNA and the gene-modified cells lack the target RNA. The Csx29 cleaves Csx30 when Cas7-11:Csx29 complex binds to the target RNA, triggering apoptosis in non-gene-modified cells and enriching the gene-modified cells.

In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the apoptotic protein is caspase 2, caspase 8, caspase 9, caspase 10, caspase 11, caspase 12, caspase 3, caspase 6, or caspase 7. In some embodiments, the apoptotic protein is an immune activating cytokine. In some embodiments, the immune activating cytokine is a cytokine or a chemokine. In some embodiments, the immune activating cytokine is interleukin 12 (IL-12), interleukin 7 (IL-7), interleukin 15 (IL-15), interleukin 2 (IL-2), interleukin 18 (IL-18), interleukin 21 (IL-21), interleukin 23 (IL-23), interleukin 1 beta (IL-1β), interleukin 6 (IL-6), interleukin 8 (IL-8), CD40L, macrophage inflammatory protein 1 alpha (CCL3) (M1P-1α), macrophage inflammatory protein 1 beta (CCL4) (M1P-1β), interferon gamma (IFNγ), Interferon beta (IFNβ), tumor necrosis factor alpha (TNFα), interleukin-1 receptor antagonist (IL-1ra), or interleukin 10 (IL-10). In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

For example, RNA guides are designed against wild type genomic sequence's RNA product. Upon sensing the wild type of unedited cells, a cascade is fused to a degron by Csx30 linker and thus causing the unedited cells to commit apoptosis.

In another aspect the present disclosure provides a method of identifying a mutation in the transcriptome of a cell based on the presence of a RNA target in the cell. The method may comprise delivering into the cell effective amounts of: a) a Cas7-11:Csx29 complex or a first nucleic acid encoding the Cas7-11:Csx29 complex, b) a guide RNA that specifically hybridizes to the RNA target, and c) a fluorescent protein fused to an inhibitory peptide via a Csx30 linker or a second nucleic acid encoding the fluorescent protein fused to the inhibitory peptide via the Csx30 linker. The fluorescence of the fluorescent protein may be inhibited by the inhibitory protein and the fluorescence of the fluorescent protein may be activated upon the cleavage of Csx30. The RNA target may comprise the mutation, and the mutation may be identified, if Csx29 cleaves Csx30 when Cas7-11:Csx29 complex binds to the target RNA and fluorescence is detected.

In some embodiments, the mutation is a single-nucleotide polymorphism (SNP), a single-nucleotide variant (SNV), a single-nucleotide substitution, a point mutation, a single-nucleotide deletion, and a single-nucleotide insertion, an alternatively spliced region, a deletion, or a frameshift. A mutation is an alteration in the nucleic acid sequence of the genome of an organism, virus, or extrachromosomal DNA. Viral genomes contain either DNA or RNA. Mutations result from errors during DNA or viral replication, mitosis, or meiosis or other types of damage to DNA (such as pyrimidine dimers caused by exposure to ultraviolet radiation), which then may undergo error-prone repair (especially microhomology-mediated end joining), cause an error during other forms of repair, or cause an error during replication (translesion synthesis). Mutations may also result from insertion or deletion of segments of DNA due to mobile genetic elements.

In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorescent protein is a green fluorescent protein, mCherry protein, a yellow fluorescent protein, a citrine fluorescent protein, a blue fluorescent protein, a cyan fluorescent protein, or a red fluorescent protein. In some embodiments, the inhibitory peptide inhibits the activity of the protein via steric hindrance. In some embodiments, the inhibitory peptide inhibits the activity of the protein via degrading the protein. In some embodiments, the inhibitory peptide comprises a specific degradation signal, or a degron. In some embodiments, the specific degradation signal, or a degron is derived from dihydrofolate reductase (DHFR). In some embodiments, the Csx30 linker is a polypeptide comprising an amino acid sequence selected from the group consisting of SEQ ID NOs: 36-46.

For example, CRISPR/Cas7-11 guide against the WT RNA sequence triggers the cleavage of Csx30 and thus causing apoptosis in the case of caspase-degron fusion. This guide cannot hybridize with RNA including point mutations on the hybridization region and thus can be used for identification of point mutations.

In another aspect the present disclosure provides a method of identifying a mutation in the transcriptome of a cell based on the presence of a RNA target in the cell. The method may comprise delivering into the cell effective amounts of: a) a Cas7-11:Csx29 complex or a first nucleic acid encoding the Cas7-11:Csx29 complex, b) a guide RNA that specifically hybridizes to the RNA target, and c) a fluorophore attached to a quencher via a Csx30 linker. The fluorescence of the fluorophore may be inhibited by the quencher and the fluorescence of the fluorophore may be activated upon the cleavage of Csx30. The RNA target may comprise the mutation, and the mutation may be identified, if Csx29 cleaves Csx30 when Cas7-11:Csx29 complex binds to the target RNA and fluorescence is detected.

In some embodiments, the mutation is a single-nucleotide polymorphism (SNP), a single-nucleotide variant (SNV), a single-nucleotide substitution, a point mutation, a single-nucleotide deletion, and a single-nucleotide insertion, an alternatively spliced region, a deletion, or a frameshift. In some embodiments, the Cas7-11 is a polypeptide comprising an amino acid sequence selected from the group consisting of SEQ ID NOs: 1-34. In some embodiments, the Csx29 is a polypeptide comprising an amino acid sequence selected from the group consisting of SEQ ID NO: 35 and 57-69. In some embodiments, the guide RNA is a pre-crRNA. In some embodiments, the guide RNA is a mature crRNA. In some embodiments, the RNA target is a single-strand RNA (ssRNA). In some embodiments, the fluorophore is 6-carboxyfluorescein (FAM) or tetrachlorofluorescein (TET). In some embodiments, the quencher is tetramethylrhodamine (TAMRA). In some embodiments, the Csx30 linker is a polypeptide comprising an amino acid sequence selected from the group consisting of SEQ ID NOs: 36-46.

In some embodiments, the Cas7-11 comprises D429A/D654A mutations. In some embodiments, the first nucleic acid, the second nucleic acid, and/or the guide RNA is administered or delivered with lipid nanoparticles (LNPs). In some embodiments, the first nucleic acid, and/or the second nucleic acid is a DNA, RNA, or a coding RNA. In some embodiments, the coding RNA is an mRNA, a self-replicating RNA, a circular RNA, a viral RNA, or a replicon RNA. In some embodiments, the Cas7-11:Csx29 complex, and/or the protein is administered or delivered via extracellular Contractile Injection System (eCIS) or engineered virus-like particles (eVLPs). In some embodiments, the RNA target is SERPINA1 RNA, scgb1a1 RNA, ADAR1 mRNA, FOXM1 mRNA, or H2AFX mRNA.

RNA targets: In the case of liver cells, SERPINA1 RNA is used to distinguish them from other cells. scgb1a1 RNA can be used to distinguish lung cells from other cells. In cancer detection, solid tumor mRNA, like ADAR1 mRNA, FOXM1 mRNA and H2AFX mRNA, can be used to classify cancer cells.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

As used herein, the singular forms “a”, “an,” and “the” include both singular and plural referents unless the context clearly dictates otherwise.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given ligand) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

“Immunotherapy” is treatment that uses a subject's immune system to treat cancer and includes, for example, checkpoint inhibitors, cancer vaccines, cytokines, cell therapy, CAR-T cells, and dendritic cell therapy.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, “circular RNA” or “circRNA” means a circular polynucleotide construct that encodes a peptide or protein as defined herein. Preferably, such a circRNA is a single stranded RNA molecule.

The term “replicon RNA” will be recognized and understood by the person of ordinary skill in the art to refer to an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding a peptide or protein as defined herein). Alternatively, the replicase may be provided on an independent coding RNA construct or a coding DNA construct. Downstream of the replicase may be a sub-genomic promoter that controls replication of the replicon RNA.

The terms “RNA” and “mRNA” mean a ribonucleic acid molecule, i.e., a polymer consisting of ribonucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e., ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

### Examples of Cas Proteins

In certain example embodiments, the CRISPR effector protein is a Cas7-11 type III-D/III-E ortholog selected from Table 1.

TABLE 1
shows Cas7-11 type III-D/III-E orthologs.

| SEQ ID NO & | |
|---|---|
| PROTEIN | |
| ID/CONTIG | Sequence |
| SEQ ID NO: 1 | MHTILPIHLTFLEPYRLAEWHAKADRKKNKRYLRGMSFAQWHKDKDGIGKPYITGTLL |
| WP_007220849 | RSAVLNAAEELISLNQGMWAKEPCCNGKFETEKDKPAVLRKRPTIQWKTGRPAICDPEK |
| | QEKKDACPLCMLLGRFDKAGKRHRDNKYDKHDYDIHFDNLNLITDKKFSHPDDIASERI |
| | LNRVDYTTGKAHDYFKVWEVDDDQWWQFTGTITMHDDCSKAKGLLLASLCFVDKLC |
| | GALCRIEVTGNNSQDENKEYAHPDTGIITSLNLKYQNNSTIHQDAVPLSGSAHDNDEPPV |
| | HDNDSSLDNDTITLLSMKAKEIVGAFRESGKIEKARTLADVIRAMRLQKPDIWEKLPKGI |
| | NDKHHLWDREVNGKKLRNILEELWRLMNKRNAWRTFCEVLGNELYRCYKEKTGGIVL |
| | RFRTLGETEYYPEPEKTEPCLISDNSIPITPLGGVKEWIIIGRLKAETPFYFGVQSSFDSTQD |
| | DLDLVPDIVNTDEKLEANEQTSFRILMDKKGRYRIPRSLIRGVLRRDLRTAFGGSGCIVE |
| | LGRMIPCDCKVCAIMRKITVMDSRSENIELPDIRYRIRLNPYTATVDEGALFDMEIGPEGI |
| | TFPFVFRYRGEDALPRELWSVIRYWMDGMAWLGGSGSTGKGRFALIDIKVFEWDLCNE |
| | EGLKAYICSRGLRGIEKEVLLENKTIAEITNLFKTEEVKFFESYSKHIKQLCHECIINQISFL |
| | WGLRSYYEYLGPLWTEVKYEIKIASPLLSSDTISALLNKDNIDCIAYEKRKWENGGIKFV |
| | PTIKGETIRGIVRMAVGKRSGDLGMDDHEDCSCTLCTIFGNEHEAGKLRFEDLEVVEEK |
| | LPSEQNSDSNKIPFGPVQDGDGNREKECVTAVKSYKKKLIDHVAIDRFHGGAEDKMKF |
| | NTLPLAGSFEKPIILKGRFWIKKDIVKDYKKKIEDAMVDIRDGLYPIGGKTGIGYGWVTD |
| | LTILNPQSGFQIPVKKDISPEPGTYSTYPSHSTPSLNKGHIYYPHYFLAPANTVHREQEMI |
| | GHEQFHKEQKGELLVSGKIVCTLKTVTPLIIPDTENEDAFGLQNTYSGHKNYQFFHINDE |
| | IMVPGSEIRGMISSVYEAITNSCFRVYDETKYITRRLSPEKKDESNDKNKSQDDASQKIRK |
| | GLVKKTDEGFSIIEVERYSMKTKGGTKLVDKVYRLPLYDSEAVIASIQFEQYGEKNEKR |
| | NAKIRAAIKRNEVIAEVARKNLIFLRSLTPEELKKVLQGEILVKFSLKSGKNPNDYLAEL |
| | HENGTERGLIKFTGLNMVNIKNVNEEDKDFNDTWDWEKLNIFHNAHEKRNSLKQGYPR |
| | PVLKFIKDRVEYTIPKRCERIFCIPVKNTIEYKVSSKVCKQYKDVLSDYEKNFGHINKIFT |
| | TKIQKRELTDGDLVYFIPNEGADKTVQAIMPVPLSRITDSRTLGERLPHKNLLPCVHEVN |
| | EGLLSGILDSLDKKLLSIHPEGLCPTCRLFGTTYYKGRVRFGFANLMNKPKWLTERENG |
| | CGGYVTLPLLERPRLTWSVPSDKCDVPGRKFYIHHNGWQEVLRNNDITPKTENNRTVEP |
| | LAADNRFTFDVYFENLREWELGLLCYCLELEPGMGHKLGMGKPMGFGSVKIAIERLQT |
| | FTVHQDGINWKPSENEIGVYVQKGREKLVEWFTPSAPHKNMEWNGVKHIKDLRSLLSIP |
| | GDKPTVKYPTLNKDAEGAISDYTYERLSDTKLLPHDKRVEYLRTPWSPWNAFVKEAEY |
| | SPSEKSDEKGRETIRTKPKSLPSVKSIGKVKWFDEGKGFGILIMDDGKEVSISKNSIRGNIL |
| | LKKGQKVTFHIVQGLIPKAEDIEIAK |
| SEQ ID NO: 2 | MNITVELTFFEPYRLVEWFDWDARKKSHSAMRGQAFAQWTWKGKGRTAGKSFITGTL |
| KHE91659 | VRSAVIKAVEELLSLNNGKWEGVPCCNGSFQTDESKGKKPSFLRKRHTLQWQANNKNI |
| | CDKEEACPFCILLGRFDNAGKVHERNKDYDIHFSNFDLDHKQEKNDLRLVDIASGRILN |
| | RVDFDTGKAKDYFRTWEADYETYGTYTGRITLRNEHAKKLLLASLGFVDKLCGALCRI |
| | EVIKKSESPLPSDTKEQSYTKDDTVEVLSEDHNDELRKQAEVIVEAFKQNDKLEKIRILA |
| | DAIRTLRLHGEGVIEKDELPDGKEERDKGHHLWDIKVQGTALRTKLKELWQSNKDIGW |
| | RKFTEMLGSNLYLIYKKETGGVSTRFRILGDTEYYSKAHDSEGSDLFIPVTPPEGIETKEW |
| | IIVGRLKAATPFYFGVQQPSDSIPGKEKKSEDSLVINEHTSFNILLDKENRYRIPRSALRGA |
| | LRRDLRTAFGSGCNVSLGGQILCNCKVCIEMRRITLKDSVSDFSEPPEIRYRIAKNPGTAT |
| | VEDGSLFDIEVGPEGLTFPFVLRYRGHKFPEQLSSVIRYWEENDGKNGMAWLGGLDSTG |
| | KGRFALKDIKIFEWDLNQKINEYIKERGMRGKEKELLEMGESSLPDGLIPYKFFEERECL |
| | FPYKENLKPQWSEVQYTIEVGSPLLTADTISALTEPGNRDAIAYKKRVYNDGNNAIEPEP |
| | RFAVKSETHRGIFRTAVGRRTGDLGKEDHEDCTCDMCIIFGNEHESSKIRFEDLELINGN |
| | EFEKLEKHIDHVAIDRFTGGALDKAKFDTYPLAGSPKKPLKLKGRFWIKKGFSGDHKLLI |
| | TTALSDIRDGLYPLGSKGGVGYGWVAGISIDDNVPDDFKEMINKTEMPLPEEVEESNNG |
| | PINNDYVHPGHQSPKQDHKNKNIYYPHYFLDSGSKVYREKDIITHEEFTEELLSGKINCK |
| | LETLTPLIIPDTSDENGLKLQGNKPGHKNYKFFNINGELMIPGSELRGMLRTHFEALTKSC |
| | FAIFGEDSTLSWRMNADEKDYKIDSNSIRKMESQRNPKYRIPDELQKELRNSGNGLFNR |
| | LYTSERRFWSDVSNKFENSIDYKREILRCAGRPKNYKGGIIRQRKDSLMAEELKVHRLPL |
| | YDNFDIPDSAYKANDHCRKSATCSTSRGCRERFTCGIKVRDKNRVFLNAANNNRQYLN |
| | NIKKSNHDLYLQYLKGEKKIRFNSKVITGSERSPIDVIAELNERGRQTGFIKLSGLNNSNK |
| | SQGNTGTTFNSGWDRFELNILLDDLETRPSKSDYPRPRLLFTKDQYEYNITKRCERVFEI |
| | DKGNKTGYPVDDQIKKNYEDILDSYDGIKDQEVAERFDTFTRGSKLKVGDLVYFHIDG |
| | DNKIDSLIPVRISRKCASKTLGGKLDKALHPCTGLSDGLCPGCHLFGTTDYKGRVKFGFA |
| | KYENGPEWLITRGNNPERSLTLGVLESPRPAFSIPDDESEIPGRKFYLHHNGWRIIRQKQL |
| | EIRETVQPERNVTTEVMDKGNVFSFDVRFENLREWELGLLLQSLDPGKNIAHKLGKGKP |
| | YGFGSVKIKIDSLHTFKINSNNDKIKRVPQSDIREYINKGYQKLIEWSGNNSIQKGNVLPQ |
| | WHVIPHIDKLYKLLWVPFLNDSKLEPDVRYPVLNEESKGYIEGSDYTYKKLGDKDNLPY |
| | KTRVKGLTTPWSPWNPFQVIAEHEEQEVNVTGSRPSVTDKIERDGKMV |
| SEQ ID NO: 3 | MKITLRFLEPFRMLDWIRPEERISGNKAFQRGLTFARWHKSKADDKGKPFITGTLLRSAV |
| OQY58162 | IRAAEHLLVLSKGKVGEKACCPGKFLTETDTETNKAPTMFLRKRPTLKWTDRKGCDPD |
| | FPCPLCELLGPGAVGKKEGEAGINSYVNFGNLSFPGDTGYSNAREIAVRRVVNRVDYAS |
| | GKAHDFFRIFEVDHIAFPCFHGEIAFGENVSSQARNLLQDSLRFTDRLCGALCVIRYDGDI |
| | PKCGKTAPLPETESIQNAAEETARAIVRVFHGGRKDPEQAQIDKAEQIQLLSAAVRELGR |
| | DKKKVSALPLNHEGKEDHYLWDKKAGGETIRTILKAAAEKEAVANQWRQFCIELSEEL |
| | YKEAKKAHGGLEPARRIMGDAEFSDKSVPDTVSHSIGISVEKETIIMGTLKAETPFFFGIE |
| | SKEKKQTDLMLLLDGQNHYRIPRSALRGILRRDIRSVLGTGCNAEVGGRPCLCPVCRIM |
| | KNITVMDTRSSTDTLPEVRPRIRLNPFTGSVQEKALFNMEMGTEGIEFPFVLSYRGKKTL |
| | PKELRNVLNWWTEGKAFLGGAASTGKSIFQLSDIHAFSSDLSDETARESYLSNHGWRGI |
| | MENSIVHESPLEGGAGGCSFGLSDLPKLGWHAEDLKLSDIEKYKPFHRQKISVKITLNSP |
| | FLNGDPVRALTEDVADIVSFKKYTQGGEKIIYAYKSESFRGVVRTALGLRNQGNDDITG |
| | KKNVPLIALTHQDCECMLCRFFGSEYEAGRLYFEDLTFESEPEPRRFDHVAIDRFTGGAV |
| | NQKKFDDRSLVPGKEGFMTLIGCFWMRKDKELSRNEIEELGKAFADIRDGLYPLGAKGS |
| | MGYGQVAELSIVDDEDSDDENNPAKLLAESMKNASPSLGTPTSLKKKDAGLSLRFDEN |
| | ADYYPYYFLEPEKSVHRDPVPPGHEEAFRGGLLTGRITCRLTVRTPLIVPNTETDDAFNM |
| | KEKAGKKKDAYHKSYRFFTLNRVPMIPGSEIRGMISSVFEALSNSCFRIFDEKYRLSWRM |
| | DADVKELEQFKPGRVADDGKRIEEMKEIRYPFYDRTYPERNAQNGYFRWDARISLTDN |
| | SMRKMEKDGVPRNVIYKLNTLKNKAYKSEKSFLFDLKNKAGGVGRYKKLVLKHAEVR |
| | GGEIPYYSHPTPTDCKLLSLVGPNRQLCRQDTLVQYRIIKHRRGAKPEEDFMFVGTPSEN |
| | QKGHKENNDHGGGYLKISGPNKIEKENVLTSGVPSVPENMGAVVHNCPPRLVEVTVRC |
| | GRKQEEECKRKRLVPEYVCADPEKKVTYTMTKRCERIFLEKSRRIIPFTNDAVDKFEILV |
| | KEYRRNAEQQDTPEAFQTILPENGTVNPGDLLYFREEKGKAAEIVPVRISRKVDDRHIGK |
| | RIDPELRPCHGEWIEDGDLSKLDAYPAEKKLLTRHPKGLCPACRVFGTGSYKSRVRFGF |
| | AALKGTPKWLKEDPAEPSQGKGITLPLLERPRPTWAVLHNDKENSEIPGRKFYVHHNG |
| | WKGISEGIHPISGENIEPDENNRTVEVLDKGNRFVFELSFENLEPRELGLLIHSLQLEKGL |
| | AHKLGMAKSMGFGSVEIDVESVRVKHRSGEWDYKDGETVDGWIEEGKRGVAAKGKA |
| | NDLRKLLYLPGEKQNPHVHYPTLKKEKKGDPPGYEDLKKSFREKKLNRRKMLTTLWEP |
| | WHK |
| SEQ ID NO: 4 | MLKLKVKITYFQPFRVIPWIKEDDRNSDRNYLRGGTFARWHKDKKDDIHGKPYITGTLL |
| KPA14974 | RSALFTEIEKIKIHHSDFIHCCNAIDRTEGKHQPSFLRKRPVYTENKNIQACNKCPLCLIM |
| | GRGDDRGEDLKKKKHYNGKHYQNWTVHFSNFDTQATFYWKDIVQKRILNRVDQTCG |
| | KAKDFFKVCEVDHIACPTLNGIIRINDEKLSQEEISKIKQLIAVGLAQIESLAGGICRIDITN |
| | QNHDDLIKSFFETKPSKILQPNLKESGEERFELAKLELLAEYLTQSFDANQKEQQLRRLA |
| | DAIRDLRKYSPDYLKDLPKGKKGGRTSIWNKKVADDFTLRDCLKNQKIPNELWRQFCE |
| | GLGREVYKISKNISNRSDAKPRLLGETEYAGLPLRKEDEKEYSPTYQNQESLPKTKWIIS |
| | GELQAITPFYIGHVNKTSHTRSTIFLNMNGQFCIPRSTLRGALRRDLRLVFGDSCNTPVGS |
| | RVCYCQVCQIMRCIKFEDALSDVDSPPEVRHRIRLNCHTGVVEEGALFDMETGFQGMIF |
| | PFRLYYESKNEIMSQHLYEVLNNWTNGQAFFGGEAGTGFGRFKLLNNEVFLWEIDGEE |
| | EDYLQYLFSRGYKGIETDEIKKVADPIKWKTLFTKLEIPPEKIPLTQLNYTLTIDSPLISRD |
| | PIAAMLDNRNPDAVMVKKTILVYEQDSSTHKNVPKEVPKYFIKSETIRGLLRSIISRTEIK |
| | LEDGKKERIFNLDHEDCDCLQCRLFGNVHQQGILRFEDAEITNKNVSDCCIDHVAIDRFT |
| | GGGVEKMKFNDYPLSASPKNCLNLKGSIWITSALKDSEKEALSKALSELKYGYASLGGL |
| | SAIGYGRVKELTLEENDIIQLTEITESNLNSQSRLSLKPDVKKELSNNHFYYPHYFIKPAP |
| | KEVVRESRLISHVQGHDTEGEFLLTGKIKCRLQTLGPLFIANNDKGDDYFELQHNNPGH |
| | LNYAFFRINDHIAIPGASIRGMISSVFETLTHSCFRVMDDKKYLTRRVIPESETTQKRKSG |
| | RYQVEESDPDLFPGRVQKKGNKYKIEKMDEIVRLPIYDNFSLVERIREYHYSEECASYVP |
| | SVKKAIDYNRMLAQAADSNREFLYNHPEAKSILQGKKEVYYILHKQESKNRGKTKEINP |
| | NARYACLTDENTPGSRKGFIKFTGPDMVTVNKELKSKIAPIYDPEWEKDIPDWERSNQE |
| | SNHKYSFILHNEIEMRSSQKKKYPRPVFICKKNGVEYRMQKRCERIFDFTKEEEKDKEIVI |
| | PQKVVSQYNAILKDNKENTETIPGLFNSKMVNKELEDGDLVYFKYKEGKVTELTPVAIS |
| | RKTDNKPMGKRFPKISINGKMKPNDSLRSCSHTCTEDCDDCPNLCESVKDYFKPHPDGL |
| | CPACHLFGTTFYKSRLSFGLAWLENNAKWYISNDFQQKDSKKEKGGKLTLPLLERPRPT |
| | WSMPNNNAEVPGRKFYVHHPWSVENIKNNQGNQKDISLKPDSDAIKIKENNRTIEPLGK |
| | DNVFNFEISFNNLRDWELGLLLYAIELEDHLAHKLGMAKAFGMGSVKIEIKNLLIKGSIN |
| | DISKAELIKKGFKKLGIDSLEKDDLSEYLHIKQLREILWFSDKPVGTIEYPKLENKTNSRIP |
| | SYTDFVQEKDHETGFKNPKYQNLKSRLHILQNPWNAWWKNEE |
| SEQ ID NO: 5 | MTTTMKISIEFLEPFRMTKWQESTRRNKNNKEFVRGQAFARWHRNKKDNTKGRPYITG |
| WP_124327589 | TLLRSAVIRSAENLLTLSDGKISEKTCCPGKFDTEDKDRLLQLRQRSTLRWTDKNPCPDN |
| | AETYCPFCELLGRSGNDGKKAEKKDWRFRIHFGNLSLPGKPDFDGPKAIGSQRVLNRVD |
| | FKSGKAHDFFKAYEVDHTRFPRFEGEITIDNKVSAEARKLLCDSLKFTDRLCGALCVIRF |
| | DEYTPAADSGKQTENVQAEPNANLAEKTAEQIISILDDNKKTEYTRLLADAIRSLRRSSK |
| | LVAGLPKDHDGKDDHYLWDIGKKKKDENSVTIRQILTTSADTKELKNAGKWREFCEKL |
| | GEALYLKSKDMSGGLKITRRILGDAEFHGKPDRLEKSRSVSIGSVLKETVVCGELVAKTP |
| | FFFGAIDEDAKQTDLQVLLTPDNKYRLPRSAVRGILRRDLQTYFDSPCNAELGGRPCMC |
| | KTCRIMRGITVMDARSEYNAPPEIRHRTRINPFTGTVAEGALFNMEVAPEGIVFPFQLRY |
| | RGSEDGLPDALKTVLKWWAEGQAFMSGAASTGKGRFRMENAKYETLDLSDENQRND |
| | YLKNWGWRDEKGLEELKKRLNSGLPEPGNYRDPKWHEINVSIEMASPFINGDPIRAAV |
| | DKRGTDVVTFVKYKAEGEEAKPVCAYKAESFRGVIRSAVARIHMEDGVPLTELTHSDC |
| | ECLLCQIFGSEYEAGKIRFEDLVFESDPEPVTFDHVAIDRFTGGAADKKKFDDSPLPGSPA |
| | RPLMLKGSFWIRRDVLEDEEYCKALGKALADVNNGLYPLGGKSAIGYGQVKSLGIKGD |
| | DKRISRLMNPAFDETDVAVPEKPKTDAEVRIEAEKVYYPHYFVEPHKKVEREEKPCGHQ |
| | KFHEGRLTGKIRCKLITKTPLIVPDTSNDDFFRPADKEARKEKDEYHKSYAFFRLHKQIM |
| | IPGSELRGMVSSVYETVTNSCFRIFDETKRLSWRMDADHQNVLQDFLPGRVTADGKHIQ |
| | KFSETARVPFYDKTQKHFDILDEQEIAGEKPVRMWVKRFIKRLSLVDPAKHPQKKQDN |
| | KWKRRKEGIATFIEQKNGSYYFNVVTNNGCTSFHLWHKPDNFDQEKLEGIQNGEKLDC |
| | WVRDSRYQKAFQEIPENDPDGWECKEGYLHVVGPSKVEFSDKKGDVINNFQGTLPSVP |
| | NDWKTIRTNDFKNRKRKNEPVFCCEDDKGNYYTMAKYCETFFFDLKENEEYEIPEKARI |
| | KYKELLRVYNNNPQAVPESVFQSRVARENVEKLKSGDLVYFKHNEKYVEDIVPVRISRT |
| | VDDRMIGKRMSADLRPCHGDWVEDGDLSALNAYPEKRLLLRHPKGLCPACRLFGTGS |
| | YKGRVRFGFASLENDPEWLIPGKNPGDPFHGGPVMLSLLERPRPTWSIPGSDNKFKVPG |
| | RKFYVHHHAWKTIKDGNHPTTGKAIEQSPNNRTVEALAGGNSFSFEIAFENLKEWELGL |
| | LIHSLQLEKGLAHKLGMAKSMGFGSVEIDVESVRLRKDWKQWRNGNSEIPNWLGKGF |
| | AKLKEWFRDELDFIENLKKLLWFPEGDQAPRVCYPMLRKKDDPNGNSGYEELKDGEFK |
| | KEDRQKKLTTPWTPWA |
| SEQ ID NO: 6 | MSKTDDKIDIKLTFLEPYRMVNWLENGLRMTDPRYLRGLSFARWHRNKNGKAGRPYIT |
| KKO18793 | GTLLRSAVIRAAEELLSLNLGKWGKQLCCPGQFETEREMRKNKTFLRRRPTPAWSAETK |
| | KEICTTHGSACAFCLLLGRRLHGGKEDVNEDAPGSCRKPVGFGNLSLPFQPTKRQIQDV |
| | CKERVLNRVDFRTGKAQDYFRVFEIDHEDWGVYTGEITITEPRVQEMLEASLKFVDTLC |
| | GALCRIEIVGSADETKRTTSSKEGCPASTTTRDCSSSENDDTSPEDPVREDLKKIAHVIAN |
| | AFQNSGNREKVHALADAIRAMRLEESSIINTLPKGKSEKTTEQIEVNKHYLWDEIPVNDT |
| | SVRHILIEQWRRWQSKKDDPEWWKFCDFLGECLYKEYKKLTSGIQSRARVMGETEYYG |
| | ALGMPDKVIPLLKSDKTKEWILVGSLKAETPFFFGLETEQTEEVEHTSLRLVMDKKGRF |
| | RIPRSVLRGALRRDMRIAFDSGCDVKLGSPLPCDCSVCQVMRSITIKDSRSEAGKLPQIR |
| | HRIRLNPFSGTVDEGALFDIEVAPEGVIFPFVMRYRGEEFPPALLSVIRYWQDGKAWLGG |
| | EGATGKGRFALAKDLKMYEWKLEDKSLHAYIDTYGHRGNEHAIGTGQGIDGFRSGSLS |
| | DLLSDISKESFRDPLASYHNYLDKRWIKVGYQITIGAPLLSADPIGALLDPNNVDAIVFEK |
| | MKLDGDQVKYLPAIKGETIRGIVRTALGKRNNLLAKNDHDDCTCSLCAIFGNENETGKI |
| | RFEDLEVYDKDIAKKIDHVAIDRFTGGARDQMKFDTLPLIGSPERPLRLKGLFWMRRDV |
| | SPDEKARILLAFLEIREGLYPIGGKTGSGYGWVSDLEFDGDAPEAFKEMNSKRGKQASF |
| | KEKISFRYPSGAPKHIQNLKATSFYYPHYFLEPGSKVIREQKMIGHEQYYESYPSGASGE |
| | KLLSGRIICSMTTHTPLIVPDTGVIKDPENKHATYDFFQMNNAIMIPGSEIRGMISAVYEA |
| | MTNSCFRIFHEKQYLTRRISPEDKELREFIPGIVRIINGDVYIEKAEREYRLPLYDDVHIITN |
| | YEELEYEKYIKKNPGREQKIKNAHRFNKNIARIAESNRNYLCSLDRAVRREILSGRKKVN |
| | FRLVKVNDNKNPDKEAVELCKTGPLEGLVKFSGLNAVNISNLRPGTAEEGFDAKWDM |
| | WSLNIILNRMDVRNSQKKEYPRPALHFNHDGKEYTIPKRCERVFVRAEAGKRAETEGSY |
| | KVPRKVQEQYQNILRDYESNIGHIDNTFRTLIENCGLNNGSLVYFKPDNSRKEVVAITPV |
| | KISRKTDRLPQGDRFPHTSSDLRPCVRDCLDTEGDIRMLENSPFKRLFHIHPEGLCPACQL |
| | FGTTNYRGRVRFGFASLSDGPKWFRKDEGNETCHITLPLLERPRPTWSMPDDTSTIPGRK |
| | FYVHHMGYETVKKNQRTLVKTENNRTVKALDKENEFTFEVFFENLREWELGLLLHCLE |
| | LEPEMGHKLGMGKPLGFGSVKIRIDKLQKCVVNVKDGCVLWEPEEDKIQHYIAKGLGK |
| | LTTWFGKEWDRLEHIQGLRSLQRLLPL |
| SEQ ID NO: 7 | FESYARWCKSNSGLWKPYIPGTLLRSAVLESVEYLLALIGSKNKVEICPGLYTQSENNPD |
| RLC14096B | TKYLRRRPWYELHAQKEICKTRDTACPLCLLMRTKLDNDGDGETEKNVKFGNLYPTSP |
| | LEPLQKIRPRILNRMDPGTSKARDYFRVFEIENQLCSQFRGWIWLSGDLPNMELVKSLLA |
| | AGLSNVATLAGAVCRIRIVSTDNPSMKQDLTTQDLIDDFTNYYLKGDTPPANLAASGKG |
| | DAFPRFSPGSGDHPDTTGVSHADMASSHEGTALAKDIAEKCKDILSQISASEQLRRLADI |
| | MRDLRQDSNREIMYRQVAEENHEKASLLYKKTKKGDSIAALIAGKTEGMDAETWRRL |
| | CEFLGQTFYGEAKEAGLVETPVPRILGESERYSLQKKPTVRTDLAAELVPDIEFIIKGNLI |
| | AETPFFFGTDIATETHTDLPILLTSDRHFRIPRSVLRGILRRDLRLVTGSGCSVKLGRSEPC |
| | ACDVCQIMRSLTMRDCVSSCKVPPEIRHRIRLNPVTETVEEGALFDMEIGPQGISFPFVLR |
| | SRGVNSSFSTRLKNVLTWWSEGKIFMGGDKGTGKGRFTLAELEAYYFRLTTKRIGKNV |
| | WVIGNYLKSQGWRGAELETHFDSLKEWKSLSFSDSDVKVFTWHKITWKVSFEGPVLTN |
| | DPIAADIRNESDAVFYQKSVAGEKGPVYALKGEGLRGIVSSSLCKKKNLSSNLHEDCEC |
| | LRCKIFGSKHQEGNIRFEDMTVSQESEVREKLFDHVSIDRFTGGAANKLKFDDKPLVGN |
| | PLVFQGVFWVHQSIGNNEKTQEALSDAFKDVRDGLYPVGAKGSIGYGWIKGIEVVEGP |
| | DWLKDALSAEKTVEAGIASEESEYKLPDLPWISLLPKGRAIYNPHYFLGIPKVTPEREREP |
| | VGHDRFQTDLHTGRIICTLKTITPLIIPDTENDKAFEVENASADHERFKFMRMGSQAAIPG |
| | SAIRSMTSSVFEALTNSCFRVLDQKSHLSWRMEADDAGDYKPGRFEKKDDKAVIRKFK |
| | KKARFPFYAGPDTREAFTSDQIMGKEKVTLWVKDFEASLTVPDEIGWKKKRGYLKVTG |
| | PNKVEIDTENISENNPSPPDSWQDVRINDDGTIPDKKNRKFICQYGTTTYTVDKWCEAFF |
| | CDEEKDPYELAPDVERKYRLLMDSYHNNPQAPPQIFRSLPLFSETGPKKTLEHGDLVYF |
| | RLSEVNKQSQSKKQVRERVTDIVPVSISRIANNQPIGKHIAAAFRPCAYVCIEECEPCDAK |
| | TCPIPVYREGYPIKGLCRACHLFGTTGYKGRVRFSFAKLNGDAVWAKGAGGKDYFTLP |
| | LLEKPRPTWTMPNEGAKIPGRKFYVHHNEWKTVQEGKNPIDQKAIRPNPNNSSVEVLNL |
| | GNEFQFEVSFENLEEWELGLLLYCLELEPGLAHKLGRGKAFGFGSIEAEVSKIEMRIKSG |
| | TWKNETSGKEKFIQSGLSQVPSFFKQDEKQWNKVEQVKNIRKLLQLSWNKGNAVEPEV |
| | RYPALREKDDENKRPGYVELKDNGYDAGKKLVSPWAPWHPIKK |
| SEQ ID NO: 8 | MTKKPGTEDKATLWGKESASKSVKTILEESIQGFTVEQKRSFFANLADQLVSRAGEQGA |
| OGR07205 | KSVRSQGLIIGRKENYAKPSAQEPTRHHLYRQPSNASAFLATGWLIAETPFFIGSGTEGQ |
| | KQTDDQAESLHLRTLRDGHGRFRIPFTTIRGVMDKELRDILQAGCAKGRSLRAPCPCQV |
| | CTLMRRIQVRDAIAADILPPDLRMRTRIDPSHGTVAHLFSLEMAPQGLKLPFFLKLKGVE |
| | TIDPDKELLEILNDWSAGQCFLGGLWGTGKGRFRLDDLQWHRLELDNADYYTPLLQDR |
| | FFAGETISDLRQGLQSINIQPERIPAQTPSRNMPYCRVDCILEFKSPVLSGDPVAALFESDA |
| | PDNVAYKKPVVQYDETGRLRTTDPGPVEMLTCLKGEGVRGVVAYLAGKAYDQHDLS |
| | HDSCNCTFCQAFGNGQKAGSLRFDDFMPVQFESDQAGNFSWSPHTPHAMRSDRVALD |
| | VFGGAMPEAKFDDRPLAASPGKPLNFKSTIWYREDMGKEAGKALKRALIDLQNNMAAI |
| | GSGGGIGRGWVSRVCFEGDIPDFLEDFPEPITVTEPEQDSQLLKNQAVADETAVSACDTA |
| | DAPHPLAVTLEPGARYFPRVIIPRAPTVKRDECVTGQRYHTGRLSGKIFCELNTLGPLFVP |
| | DTDYSAGVPVPISDEQLAECQLQAVFENTSKFNEFFATYPEETVTKLKDLLCAADDKWI |
| | LAVKDITADLRQEIGEDTFQRIIRKAGHKTQRFHQINDEIGLPGASLRGMVLSNYQILTNS |
| | CYRNLKATEEITRRMPADEAKYRKAGRVTVSGDGAQKKYSIQEMEVLRLPIYDNMNTP |
| | DNMPDVAKQATTAKRCNNLMNEAAKTSRVELKARWREGQSKIKYQIIDALNKVDPIIQ |
| | VISSSKQINPNNGKTGWGYVKYTGANVFAKSLVAPIDCLRKKDAGHVCCQVNLNPAW |
| | EASNFDILINEKCPVERQSGPRPTLRCKGQDSAWYTLTKRSERIFTDKKPVPDPINIPPRE |
| | VKRYNELRDSYKKNTAHVPKPLQTFFNQESLANGDLVYFEVNQFGEASQLTPVSISRTT |
| | DLFPIGGRLPQGHKDLFPCTAMCLSECKNCVPASFCEFHSRSHEKLCPACSLAGTTGNRG |
| | RIKFSEAWLSGLPKWHSVSQDNVGRGLGVTMPRLERSRRTWHLPTKDAYLLGQSIYLN |
| | HPVPAILPSDQVPSENNQTVEPLGPKNIFSFQLAFDNLSIEELGLLLYSLELESGMAHRLG |
| | RGRALGMGSVQISVKDIQIRDNKSFLFSSNISKKSEWIQCGKDEFAQEAWFGESWDNID |
| | HIQRLRQALTIPVKGDVGCIRYPKLEAEGGMPDYIKLRKRLTPLCDREEPVRYRINPVQL |
| | ARMILPFVPWHGACPALLNEQVMIEAKRLTELXXXDRANWPC |
| SEQ ID NO: 9 | ASEDDDTPTLRKVLKDEINGQEDMWRKFCEALGNSLYDLSKKAKERKRTEALPRLLGE |
| RLC14096 | TEIYGLPMRENKEDEPLPSSLTYKFKWLIAGELRAETPFFFGTEVQEGQTSATILLNRDG |
| | YFRLPRSVIRGALRRDLRLVMGNDGCNMPIGGQMCECGVCRVMRHIVIEDGLSDCKIPP |
| | EVRHRIRLNCHTGTVEEGALFDMETGYQGMTFPFRLYCETENSDLDSYLWEVLNNWQ |
| | NGQSLFGGDTGTGFGRFELTEPKVFLWNFSKKEKHEAYLLNRGFKGQMPVQDVKTKSF |
| | KTKTWFQIHRELDISPKKLPWYSTDYRFNVTSPLISRDPIGAMLDPRNTDAIMVRKTVFC |
| | PDPNAKNRPAPATVYMIKGESIRGILRSIVVRNEELYDTDHEDCDCILCRLFGSIHQQGSL |
| | RFEDAEVQNSVSDKKMDHVAIDRFTGGGVDQMKFDDYPLPGCPAQPLILEGKFWVKD |
| | DIDDESKSALEKAFADFRDGLVSLGGLGAIGYGQIGDFELIGGSADWLNLPKPEENRTD |
| | VPCGDRSAQGPEIKISLDADKIYHPHFFLKPSDKNVYRERELVSHAKKKGPDGKSLFTGK |
| | ITCRLSTEGPVFIPDTDLGEDYFEMQASHKKHKNYGFFRINGNVAIPGSSIRGMISSVFEA |
| | LTNSCFRVFDQERYLSRSEKPDPTELTKYYPGKVKRDGNKFFILKMKDFFRLPLYDFDFE |
| | GEAESLRPNYDEDRNEEENKGKNKNTQKVKNAVEFNIKMAGFAKHNRDFLKKYKEQE |
| | IKDIFMGKKKVYFTAGKHKPNEAHDNDKIALLTKGSNKKAEKGYFKFTGPGMVNVKA |
| | GVEGEECDFHIDESDPDVYWNMSSILPHNQIKWRPSQKKEYPRPVLKCVKDGTEYVML |
| | KRSEHVFAEASSEDSYPVPGKVRKQFNSISRDNVQNTDHLSSMFQSRRLHDELSHGDLV |
| | YFRHDEKRKVTDIAYVRVSRTVDDRPMGKRFKNESLRPCNHVCVEGCDECPDRCKELE |
| | DYFSPHPEGLCPACHLFGTTDYKGRVSFGLGWHESNTPKWYMPEDNSQKGSHLTLPLL |
| | ERPRPTWSMPNKKSEIPGRKFYVHHPWSVDKIRNRQFDPAKEKQPDDVIKPNENNRTVE |
| | PLGKGNEFTFEVRFNNLREWELGLLLYSLELEDNMAHKLGMGKALGMGSARIKAEAIE |
| | LRCESAGQNAELKDKAAFVRKGFEFLEIDKPGENDPMNFDHIRQLRELLWELPENVSAN |
| | VRYPMLEKEDDGTPGYTDFIKQEEPSTGKRNPSYLSSEKRRNILQTPWKHWYLIPPFQAS |
| | AQSETVFEGTVKWFDDKKGFGFIKINDGGKDVFVHHSSIVGTGFKSLNEGDSVAFKMG |
| | VGPKGPCAEKVKKIGN |
| SEQ ID NO: 10 | MRRQRLLGDAEYYGGTGREQPASIVISTDSDPDHKVYEWIITGQLKAETGFFFGTKAGA |
| OPY65763 | GGHTDLSILLGKDGHYRVPRSVFRGALRRDLRVAFGAGCRVEVGRERPCECPVCKVMR |
| | QITVMDTISSYREAPEIRQRIRLNPYTGTVDKGALFDMEVGPEGIEFPFVLRFRGSKSFPSE |
| | LAAVIGSWTKGTAWLGGAAATGKGRFSLLGLSIHKWNLSTAEGRKSYLAAYGLRDAA |
| | DKTVKRLSIDKGGKGDVGLPAGLERDALPSSVREPLWKKLVCTVDFSSPLLLADPIAAL |
| | LGVEGDERIGFDNIAYEKRRYNGETNTTESIPAVKGETFRGIVRTALGKRHGNLTRDHE |
| | DCRCRLCAVFGKEQEAGKIRFEDLMPVGAWTRKHLDHVAIDRFHGGAEENMKFDTYA |
| | LAASPTNPLRMKGLIWVRSDLFETGHDGPTPPYVKDIIDALADVKRGLYPVGGKTGSGY |
| | GWIKDVTIDGLPQGLSLPPAEERVDGVNEVPPYNYSAPPDLPSAAEGEYFFPHVFIKPYD |
| | KVDRVSRLTGHDRFRQGRITGRITCTLKTLTPLIIPDSEGIQTDATGHKMCKFFSVAGKP |
| | MIPGSEIRGMISSVYEALTNSCFRVFDEEKYLTRRVQPKKGAKSSELVPGIIVWGQNGGL |
| | AVQQVKNAYRVPLYDDPAVTSAIPTEAQKNKERWESVPSVNLQGALDWNLTTANIAR |
| | DNRTFLNSRPEEKDAILSGTKPISFELEGTNPNDMLVRLVPDGVDGAHSGYLKFTGLNM |
| | VLKANKKTSRKLAPSEEDVRTLAILHNDFDSRRDWRRPPNSQRYFPRSVLRFSLERSTYT |
| | IPKRCERVFEGTCGEPYSVPSDVERQYNSIIDDISKNYGRISETYLTKTANRKLTVGDLVY |
| | FIADLDKNMATHILPVFISRISDEKPLGELLPFSGKLIPCEGEPPTILKKMAPSLLTEAWRT |
| | LISTHLEGFCPACRLFGTTSYKGRIRFGFAEHTGTPKWLREELDWARPFLTLPIQERPRPT |
| | WSVPDDKSEVPGRKFYLHHHGGNRIVESNLRNRPEVNQTKNNSSVEPISAGNTFTFDVC |
| | FENLEAWELGLLLYCLELSPKLAHKLGRAKAFGFGSVKIHVERIEERTTDGAYQDVTAV |
| | KKNGWITTGHDKLREWFHRDDWEDVDHIRNLRTVLRFPDADQEHDVRYPELKANNGV |
| | SGYVELRDKMTASERQESLRTPWYRWFPQNGTGGSGRHEQAATSQEQDTAKDESVLS |
| | ATQRRQAVIDVSDPDERLSGTVESFDRQKGDGYIGCGVRQFYVRLEDIRSRTALCEGQV |
| | VTFRARKEWEGHEAYDVEIDQ |
| SEQ ID NO: 11 | MLEKALADFRDGVVSLGGLGAIGYGRIGDFEVAEESGTWLKIPEKKLPEDSVQCGERYR |
| RLC02083 | FSSDPATRFEKEKIYYPHYFLKPSDEDVRRETRLVSHVYQEDTDGKTRLLTGTIRCRLTT |
| | EGPIFIPDTDDPKEDYFQMEIEGHKSYGFFRINEQVAIPGSSIRGMVSSVFEALTNSCFRVF |
| | DQKRYLSRSTKPDPRELEKYLPGKVKRIDNKWVLLELEDIFRLPLYDLKDVGPKSLDSA |
| | YGLEKFKNEKRFRLKKIENAVAFNKKMAGYAKHTREFLKNNYTETELGKILRGEMKV |
| | WFTIGHKPNSAHDNDKIALLTKKTNKRAKSGFIKFTGPSMVNIKADASNSDCEECRFDM |
| | KSEDKDGLIFHNAIECRPSQKKEYPRPVLKCVKEAVEYTMIKRCEQVFSEGKKPPRSYSI |
| | PDKSRRQYNGILKDNRDNTEHIPSFFRNRMKNKELSDEDLVYFRYKGKKVTNIAPVRVS |
| | R |
| SEQ ID NO: 12 | QYNLPLNPDAFPKFRWIVTGHLRAETPFFFGRGEIKDRTIEEETEQTSKTILLNKDNFFRL |
| RLC02082 | PRSVIRGALRRDLRLVIGNGCNTPVGGKFCECDVCRIMRHVVVEDTISSCRKPPEIRYNIR |
| | LNGHTRTVEEGALFDTETGYQGMRFPFRLCFETRASEFDPDTSEPIPKFDPYLSEVMKH |
| | WKAGQAVFGGDTGAGFGRFRLEGDIRFFSMDVAKKEEYDPYLLARGFKGMSSQEILEK |
| | IGSGRTYDWNSVPKIALNIPPNKLPWKEICYTIEVISPLISRDPIRAMMDPRNTDTIMIRKR |
| | VFVPDGKGGTLPEPESRYFIKSETLRGILRSLVGGNKTADGEYLCDLDHEDCDCVQCRL |
| | FGSIHQQGCLRFEDAEVWNSVRDKKMDHVAIDRFTGGALDQMKFDDYPLLGCPEYPVI |
| | LGGRFWIRDDISDKEKEIA |
| SEQ ID NO: 13 | MESIPVTLTFLEPYRVVEWYANEDRRSAERYLRGQSFARWHRKKNDKKGRPYITGTLL |
| OPY65764 | RSAAIRAAEELLSLSGGVWDGQHCCKGQFLSGGVKPEYMRKRPTYIWAEKEGACSAPD |
| | YCPFCIFLGDRDQAEKKAESQNGYPDKSYHIRFGNLSLPDPPPLLDLKEVAVERTLNRVD |
| | FQTAKAHDYFKVWEISHEDLGVYTGQIVIHYTGPWQEKVKSLLEGSLRFVDRLCGALC |
| | KIEMAPKPARPLPKSLSVDMTEHAKIIVTAFDDAKKAEKVRGLADAMRSMGSKGPTILD |
| | KLPAGHDDRDHHTWDVTIVDKTPLRTYLKGVLRADDAASWPALCKALGNALYDVSQ |
| | G |
| SEQ ID NO: 14 | EKQGFRDKGFNIVGSLKDAIGKEIGLREISLRPAKEETMPRWQCVEYTIIVNSPLHTADPI |
| RME63343 | EALLHSGNYDSVVYKKTVVRNGNIKQIPVFKGETIRGIVRTAFARILRTENVEFDEEHED |
| | CTCPLCQVFGNEHRAGRVRFEDLVIEGYTSEKKFDHVSIDRFTGGAAEKRKFDDLSLKG |
| | SPRRPIVLRGKVWIRNDMDSKGIEKLKQAFMDIRDGLYPLGSRGGIGYGWVTDLKIENT |
| | EVEEFRLDKVSTTEGSGPATEEFNFPSLPEIQLNKDAVYHPHYFIRPHEKVNREIRPVGHE |
| | RFHDDLLTGRIKCTLKTLTPLIIPDTEDPDAFGLQAEHKGHQNFRFFR |
| SEQ ID NO: 15 | MKSIPITLTFLEPYRILPWAEKGKRDKKEYLRGANYVRLHKDKNGKFKPYITGTLIRSAV |
| Ga0190306_10003932 | LSAIEMLLDITNGEWNGKECCLAKFHTEGEKPSFLRKKPIYIRAEKDEICTSRETACPLCL |
| | ILGRFDKAEKKEKDKEKFDVHFSNLNLYSSKEFSTIEELAPKRALNRIEQYTGKAQDYFT |
| | VYEALNKEFWTFKGRIRIKEDIYDKVTDLLFSALRCVEKIAGALCRIEIDKEPSQQKGFV |
| | KRQLSKQAKEDIEKIFQVVKDAQKLRLLSDCFRELTRMANKDELALPLGPEDDGHYLW |
| | DKIKVEGKTLRIFLRNCFSQYKDNWLCFCDEASKKGYQKYREKRHKLTDRELPTATPK |
| | HFAEKKDPQISPIYIDKDDKVYEWIIVGRLIAQTPFHFGDEEKAEGAILLTPDNRFRLPRT |
| | ALRGILRRDLKLAGASACEVEVGRSEPCPCDVCKIMRRVTLLDTVSEDLRDFLPELRKRI |
| | RINPQSGTVAEGALFDTEVGPEGLSFPFVLRYKCEKLPDSLTTVLCWWQEGLAFLSGES |
| | ATGKGRFRLEINGAFVWDLQKGLFNYIKNHGFRGEERLFLEGNEAELEKMGIQINTELL |
| | QPEMIKKEKNFTDFPYDLIKYQLNISSPLLLNDPIRAIALYEGEGKAPDAVFFKKYVFENG |
| | KIEEKPCFKAESIRGIFRTAVGRIKNVLTKNHEDCICVLCHLFGNVHETGRLKFEDLKIVS |
| | GQEEKFFDHVAIDRFLGGAKEKYKFDDKPIIGAPDTPIVLEGKIWVKKDINDEAKETLSQ |
| | AFSDINTGIYYLGANGSIGYGWIEEVKALKAPSWLKIKEKPNFEKDTSLNISAIMNEFKK |
| | DIQTLNLDKTYLPYGFLKLLEKVKRTSSPITHERFYENHLTGFIECSLKVLSPLIIPDTETPE |
| | KEENGHKYYHFLKIDNKPIIPGAEIRGAVSSIYEALTNSCFRVFGEKKVLSWRMEGKDA |
| | KEFMPGRVSKKKGKLYMVKMQALRLPVYDNPALANEIRSGSIYEKYKNSKVEIIFFQTV |
| | EGIRKFLRGNFNNVEWKKVLVTGIDPLAILPSQKIPGNDKWVKNLQSKISPVRGYFKFTG |
| | PNKIETKRREEEKDEKLRTKANKVSCLQKDKWYEAMHNHVEYKQDYTPPNSPKTEPLE |
| | RPRNIPCFVCSDKEKIYRMTKRCERVFVSLGENAPKYEIPISAIKRYEVILSAYRENWERN |
| | KTPELFRTRLPGDGRTLNEDDLVYFRADENEKVKDIIPVCISRIVDEVPLIKRLSQELWPC |
| | VLAECPLLGFECKKCELEGLPEKIWFRINKDGLCPACRLFGTQIYKSRVRFSFAYAKNW |
| | KFYDGYITLPRLESPRATWLILKEKDKHYIKYKVCGRKFYLHNSTYEDIINNSKKEKEKK |
| | TENNASFEVLKEGEFTFKVYFENLENWELGLLLLSLTGLGEAIKIGHAKPLGFGSVKIEA |
| | KKIYFREEAGKFHPCEKADEYLKKGLNKLTSWFGKNEINEHMRNLLLFMTYYQNLPKV |
| | KYPDFDGYAKWRCSYVEQDKVEYFQNRWIVAS |
| SEQ ID NO: 16 | MIINITVKFLGPFRMLEWTDPDNRNRKNREFMRGQAFARWHNSNPQKGSQPYITGTLV |
| Ga0193932_104825 | RSAVIRSAENLLMLSEGKVGKEKCCPGEFRTENRKKRDAMLHLRQRSTLQWKTDKPLC |
| | NGKSLCPICELLGRRIGKTDEVKKKGDFRIHFGNLTPLNRYDDPSDIGTQRTLNRVDYAT |
| | GKAHDFFKVWEIDHSLLSVFQGKISIADNIGDGATKLLEDSLRFTDRLCGAICVISYDCIE |
| | NSDGKENGKTGEAAHIMGESDAGKTDAENIANAIADMMGTAGEPEKLRILADAVRALR |
| | IGKNTVSQLPLDHEGKENHHLWDIGEGKSIRELLLEKAESLPSDQWRKFCEDVGEILYLK |
| | SKDPTGGLTVSQRILGDEAFWSKADRQLNPSAVSIPVTTETLICGKLISETPFFFGTEIEDA |
| | KHTNLKVLLDRQNRYRLPRSAIRGVLRRDLRTAFGGKGCNVELGGRPCLCDVCRIMRGI |
| | TIMDARSEYAEPPEIRHRIRLNPYTGTVAEGALFDMELGPQGLSFDFILRYRGKGKSIPKA |
| | LRNVLKWWTKGQAFLSGAASTGKGIFRLDDLKYISFDLSDKDKRKDYLDNYGWRNRIE |
| | ALSLEKMPLDRMNDYAEPLWQKVSVEIEIGSPFLNGDPIRALIEKDGSDIVSFRKYADDS |
| | GKEVYAYKAESFRGVVRAALARQHFDKEGKPLDKEGKPLLTLIHQDCECLICRLFGSEH |
| | ETGRLRFEDLLFDPQPEPMIFDHVAIDRFTGGAVDKKKFDDCSLPGTPGHPLTLKGCFWI |
| | RKELEKPDEDKSEREALSKALADIHNGLYPLGGKGAIGYGQVMNLKIKGAGDVIKAAL |
| | QSESSRMSASEPEHKKPDSGLKLSFDDKKAVYYPHYFLKPAAEEVNRKPIPTGHETLNS |
| | GLLTGKIRCRLTTRTPLIVPDTSNDDFFQTGVEGHESYAFFSVNGDIMLPGSEIRGMLSSV |
| | YEALTNSCFRVFDEGYRLSWRMEADRNVLMQFKPGRVTDNGLRIEEMKEYRYPFYDR |
| | DCSDKKSQEAYFDEWERSITLTDDSLEKMAERKGDISPKDLKVLKSLKGKNYKSTEGLL |
| | AAFKDKGGDTGGNILGLIFKYAERIGDVPRYEHPTDTDRMMLSLSEYNRNQKSDGKRA |
| | YKIIKPASKLGKGAYFMFAGTSVENKRICNPACTDKANKSVKGYLKISGPNKLEKYNISE |
| | PELDGVPEDRNCQIIHNRIYLRKIFVANAKKRKERDRLVGEFACYDPEKKVTYSMTKRC |
| | ERIFIKDRGRTLPITHEASELFEILVQEYRENAKRQDTPEVFQTLLPDNGRLNPGDLVYFR |
| | EEKGKTVEIIPVRISRKIDDSPIGKRLREDLRPCHGEWIEGDDLSQLSEYPEKKLFTRNTEG |
| | LCPACRLFGTGAYKGRLRFGFAKLENDPKWLMKNSDGPSHGGPLTLPLLERPRPTWSM |
| | PDDTLNRLKKDGKQEPKKQKGKKGPQVPGRKFYVHHDGWKEINCGCHPTTKENIVQN |
| | QNNRTVEPLDKGNTFSFEICFENLEPYELGLLLYTLELEKGLAHKLGMAKPMGFGSIDIE |
| | VENVSLRTDSGQWKDANEQISEWTDKGKKDAGKWFKTDWEAAEHIKNLKKLLFLPGE |
| | EQNPRVIYPALKQKDIPNSRLPGYEELKKNLNMEKRKEMLTTPWAPWHPIKK |
| SEQ ID NO: 17 | MTQITIQVTFFHPFRVVPWNHRDHRKTDRKYLRGGTFAKWHCTASEGKSGRPYITGTLL |
| Ga0190283_10011062 | RSALFAEIEKLIAFHDPFKCCRGKDKTENGNAKPLFLRRRPRADCDPCGTCPLCLLMGRS |
| | DTVRRDAKKQKKDWSVHFCNLREATERSFNWKETAIERIVNRVDPSSGKAKDYMRIW |
| | EIDPLVCSQFNGIITINLDTDNAGKVKLLMAAGLAQINILAGSICRADIISEDHDALIKQFM |
| | AIDVREPEVSTSFPLQDDELNNAPAGCGDDEISTDQPVGHNLVDRVRISKIAESIEDVESQ |
| | EQKAQQLRRMADAIRDLRRSKPDETTLDALPKGKTDKDNSVWDKPLKKDILPSPRMPA |
| | SEDDDTPTLRKVLKDEINGQEDMWRKFCEALGNSLYDLSKKAKERKRTEALPRLLGET |
| | EIYGLPMRENKEDEPLPSSLTYKFKWLIAGELRAETPFFFGTEVQEGQTSATILLNRDGYF |
| | RLPRSVIRGALRRDLRLVMGNDGCNMPIGGQMCECGVCRVMRHIVIEDGLSDCKIPPEV |
| | RHRIRLNCHTGTVEEGALFDMETGYQGMTFPFRLYCETENSDLDSYLWEVLNNWQNG |
| | QSLFGGDTGTGFGRFELTEPKVFLWNFSKKEKHEAYLLNRGFKGQMPVQDVKTKSFKT |
| | KTWFQIHRELDISPKKLPWYSTDYRFNVTSPLISRDPIGAMLDPRNTDAIMVRKTVFCPD |
| | PNAKNRPAPATVYMIKGESIRGILRSIVVRNEELYDTDHEDCDCILCRLFGSIHQQGSLRF |
| | EDAEVQNSVSDKKMDHVAIDRFTGGGVDQMKFDDYPLPGCPAQPLILEGKFWVKDDID |
| | DESKSALEKAFADFRDGLVSLGGLGAIGYGQIGDFELIGGSADWLNLPKPEENRTDVPC |
| | GDRSAQGPEIKISLDADKIYHPHFFLKPSDKNVYRERELVSHAKKKGPDGKSLFTGKITC |
| | RLSTEGPVFIPDTDLGEDYFEMQASHKKHKNYGFFRINGNVAIPGSSIRGMISSVFEALTN |
| | SCFRVFDQERYLSRSEKPDPTELTKYYPGKVKRDGNKFFILKMKDFFRLPLYDFDFEGE |
| | AESLRPNYDEDRNEEENKGKNKNTQKVKNAVEFNIKMAGFAKHNRDFLKKYKEQEIK |
| | DIFMGKKKVYFTAGKHKPNEAHDNDKIALLTKGSNKKAEKGYFKFTGPGMVNVKAGV |
| | EGEECDFHIDESDPDVYWNMSSILPHNQIKWRPSQKKEYPRPVLKCVKDGTEYVMLKR |
| | SEHVFAEASSEDSYPVPGKVRKQFNSISRDNVQNTDHLSSMFQSRRLHDELSHGDLVYF |
| | RHDEKRKVTDIAYVRVSRTVDDRPMGKRFKNESLRPCNHVCVEGCDECPDRCKELEDY |
| | FSPHPEGLCPACHLFGTTDYKGRVSFGLGWHESNTPKWYMPEDNSQKGSHLTLPLLERP |
| | RPTWSMPNKKSEIPGRKFYVHHPWSVDKIRNRQFDPAKEKQPDDVIKPNENNRTVEPLG |
| | KGNEFTFEVRFNNLREWELGLLLYSLELEDNMAHKLGMGKALGMGSARIKAEAIELRC |
| | ESAGQNAELKDKAAFVRKGFEFLEIDKPGENDPMNFDHIRQLRELLWFLPENVSANVRY |
| | PMLEKEDDGTPGYTDFIKQEEPSTGKRNPSYLSSEKRRNILQTPWKHWYLIPPFQASAQS |
| | ETVFEGTVKWFDDKKGFGFIKINDGGKDVFVHHSSIVGTGFKSLNEGDSVAFKMGVGP |
| | KGPCAEKVKKIGN |
| SEQ ID NO: 18 | MTKIPISLTFLEPFRLVDWVSESERDKSEFLRGLSFARWHRIKNQREDENQGRPYITGTLL |
| Ga0073580_1036305 | RSAVIKAAEELIFLNGGKWQSEECCNGQFKGSKAKYRKVECPRRRHRATLKWTDNTCS |
| | DYHNACPFCLLLGCLKPNSKENSDIHFSNLSLPNKQIFKNPPEIGIRRILNRVDFTTGKAQ |
| | DYFYVWEVEHSMCPKFQGTVKINEDMPKYNVVKDLLISSIQFVDKLCGALCVIEIGKTK |
| | NYICQSFSSNIPEEEIKKLAQEIRDILKGEDALDKMRVLADTVLQMRTKGPEIVNELPRGI |
| | EKKGGHWLWDKLRLRKKFKEIANNYKDSWQELCEKLGNELYISYKELTGGIAVKKRII |
| | GETEYRKIPEQEISFLPSKAGYSYEWIILGKLISENPFFFGKETKTEEQIDMQILLTKDGRY |
| | RLPRSVLRGALRRDLRLVIGSGCDVELGSKRPCPCPVCRIMRRVTLKDARSDYCKPPEV |
| | RKRIRINPLTGTVQKGALFTMEVAPEGISFPFQLRFRGEDKFHDALQNVLVWWKEGKLF |
| | LGGGASTGKGRFKLEIEHVLKWDLKNNFHSYLQYKGLRDKGDFNSIKEIEGLKVETEEF |
| | KVKKPFPWSCVEYTIFIESPFVSGDPVEAVLDSSNTDLVTFKKYKLEESKEVFAIKGESIR |
| | GVFRTAVGKNEGKLTTENEHEDCTCILCRLFGNEHETGKVRFEDLELINDSAPKRLDHV |
| | AIDRFTGGAKEQAKFDDSPLIGSPDSPLEFTGIVWVRDDIDEEEKKALKSAFLDIKSGYYP |
| | LGGKKGVGYGWVSNLKIESGPEWLRLEVQEKSSQENVLSPVILSEVMDIEFNPPKIDEN |
| | GVYFPYAFLRPLNEVKRTREPIGHNEWKKSLISGYLTCRLELLTPLIIPDTSEEVIKEKVN |
| | NGEHPVYKFFRLGGHLCIPAAEIRGMISSVYEALTNSCFRVFDEKRLISWRMTAEEAKRP |
| | DPKKSEEQNRMRFRPGRIIKKDKKFYAQEMLELRIPVYDNKDKRNEISQNDPTRPSEYN |
| | HPTEPERIFFSNAEKIRNFLKRNSNYLHGSTPLLFRQWSISNRYDKIALIGNKSQGHLKFT |
| | GPNKIEVSEGTKCPKYETIPGRDEWDKAVHNYVEPGKFVTVISRKKGQKPKAVQRRRN |
| | VPAFCCYDYNTNRCFVMNKRCERVFKVSRDKPKYEIPPDAIRRYEHVLRKYRENWERY |
| | DIPEVFRTRLPGDGETLNEGDLVYFRLDENNRVLDIIPVSISRISDTQYLGRRLPDHLRSC |
| | VRECLYEGWGDCKPCKLSLFPEKMWIRINPEGLCPACHLFGTQVYKGRVRFGFARAGS |
| | NWKFREEQLTLPRFETPRPTWVIPKRKDEYQIPGRKFYLHHNGWEEIYKKNKKNEIKKE |
| | KNNATFEVLKQGTFYFKVFFENLELWELGLLIFSAELGGEEFAHKLGHGKALGFGSVKI |
| | SVDKIILRRDPGQFEQRGQKFKRDAVDKGFCVLENRFGKTNFKIYLNNFLQLLYWPNNK |
| | KVKVRYPYLRQEDDPEKLPGYVELKKHQMLKDDNRYSLFARPRAVWLKWTEMVQRD |
| | KS |
| SEQ ID NO: 19 | MSVEEFYVRLTFLEPFRVVPWVRNGDERKGDRIYQRGGTYARWHKINDSHGQPYITGT |
| Iso3TCLC_1001005823 | MLRSAVLREIENTLTLHNTYGCCPGGTRTTEGKLEKPLYLRRRDGFEFENHAEKPCSEE |
| | DPCPLCLIQGRFDKLRRDEKKQFVRQGNISFCSVNFSNLNISSGIKSFSWEEIAVSRVVNR |
| | VDPNSGKAKDFFRVWEIDHKLCPNFLGKMSISLSEKLEDVKALLAVGLAQVNVLSGAL |
| | CRVDIIDPETQKDTVHQHLIQQFVTRIQDKEKGDAADIPAFTLPPAGLSPSSNEWNDTIKS |
| | LAEKIRKIKELEQGQKLRQMADVIRELRRKTPAYLDQLPAGKPEGRESIWEKTPTGETLT |
| | LRQLLKSANVPGESWRAFCEELGEQLYRLEKNLYSHARPLPRLLGETEFYGQPARKSDD |
| | PPMIRASYRAFPSYVWVLDGILRAETPFYFGTETSEGQTSQAIILCPDGSYRLPRSLLRGVI |
| | RRDLRAILGTGCNVSLGKVRPCSCPVCEIMRRITVQQGVSSYREPAEVRQRIRSNPHTGT |
| | VEEGALFDLETGPQGMTFPFRLYFRTRSPYIDRALWLTINHWQEGKAIFGGDIGVGMGR |
| | FRLENLQIRSADLVSRRDFSLYLRARGLKGLSREEVTRIGLNEEQWEAVMADDPGTHYN |
| | PFPWEKISYTLLIHSPLISNDPIAAMLDHDNKDAVMVQKTVLFVDESGNYSQMPHHFLK |
| | GSGIRGACRFLLGRKDAPNENGLTYFEADHEECDCLLCSLFGSKHYQGKLRFEDAELQD |
| | EVEAIKCDHVAIDRFHGGTVHRMKYDDYPLPGSPNRPLRIKGNIWVKRDLSDTEKEAV |
| | KDVLTELRDGLIPLGANGGAGYGRIQRLMIDDGPGWLALPERKEDERPQPSFSPVSLGP |
| | VHVNLKSGSDTADVYYYHPHYFLEPPSQTVSRELDIISHARTRDSGGEALLTGRILCRLIT |
| | RGPIFIPDTNNDNAFGLEGGIGHKNYRFFRINDELAIPGSELRGMVSSVYEALTNSCFRIM |
| | EEGRYLSRRMGADEFKDFHPGIVVDGAKIREMKRYRLPLYDTPDKTSRTKEMTCPELFT |
| | RKDGRPERAKKFNEEIAKVAVQNRAYLLSLDEKERREVLLGNREVTFDECPDDEYSDD |
| | EYSELKYAQKYKDFIAVLKKNGQKRGYIKFTGPNTANKKNEDAPDKNYRSDWDPFKL |
| | NILLESDPECRVSNIHCYPRPLLVCIKDKAEYRIHKRCEAIFCSIGSPSDLYDIPQKVSNQY |
| | RTILQDYNDNTGKIVEIFRTQIKHDQLTTGDLVYFKPAANGQVNAVIPVSISRKTDENPL |
| | AKRFKNDSLRPCAGLCVEDCNECPARCKKVADYFNPHPRGLCPACHLFGTTFYKGRVR |
| | FGFAWLTGEDGAPRWYKGPDPCDSGKGRPMTIPLLERPRPTWSIPDNSFDIPGRKFYVH |
| | HPYSVDGIDGETRTPNNRTIEPLAEGNEFVFDIDFENLRDWELGLLLYSLELEDSLAHKL |
| | GLGKPLGFGTVQINIRGISLKNGSKGWDTKTGDDKNQWIKKGFAHLGIDIKEANERPYI |
| | KQLRELLWVPTGDNLPHVRYPELESKTKDVPGYTSLLKEKDLADRVSLLKAPWKPWKP |
| | WSGTAPHPDKGTNRLRASIVERDRIQRKTDTAKPEKKEETKVGKSSSSDIEKRYVGTVK |
| | WFNDKKGYGFILYGTDEEIFVHRSGVADNSIPKEGQKVGFRIERGARGSHAVEVKAIE |
| SEQ ID NO: 20 | MPRFQLSLTFFDEPFRLIEWTDKSNRNSANTQWMRGQGFARWHKITLEKGFPFVTGTA |
| SESD01000293.1 | VRSKIIREVEALLSRNKGTWNGIPCCSGFFDTKGPSPTHLRYRPTLEWEYGKTVCTSEAD |
| | VCPLCLLLGRFDQAGKKSDTPCQSTDYHVHWENLSAGVAQYRLEDIAQKRTSNRVDFF |
| | SKKAHDHYGVWEVTAVKNLLGYIYISDAITESHQKTVISLLKAALSFTDTLCGANCKLE |
| | LSDEPVDSIHSNQSASNFNPHSGAAPSQCSQSMPPFNMDQETKELANTLCKAFTGNMRH |
| | LRTLADAVREMRRMSPGISSLPRGRLNKEGEITAHYLWDERIDEKTIRQVLEDTIELSPA |
| | RSIIYKNWISFCNQLGQKLYERAKDNDPILERKRPLGEAAFSKVPTSSHAPRHDMNSRVK |
| | GGFTREWIIVGTLRALTPFYMGTGSQAGKQTSMPTLQDSNDHFRLPRTALRGALRRDIN |
| | QASDGMGCVVELGPHNLCSCPVCQVLRQIRLLDTKSKFSMPPAIRQKICKNPVLSIVNEG |
| | SLFDVELGIEGETFPFVMRYRGGAKIPDTIITVLSWWKNERLFIGGESGTGRGRFVLECPR |
| | IFCWDVEKGQNDYIQYHGFRNKEDELLSVYSTVSGLAEKNDVNLNNARDFSFDKICWE |
| | VQFDGPVLTGDPLAALFHGNTDSVFYKKPILKSGEKEPSYQWAIKSDTVRGLIRSAFGK |
| | RDALLIKSHEDCDCLLCEAFGSKHHEGKLRFEDLTPKSDEIKTYRMDHVAIDRISGGAV |
| | DQCKYDDEPLVGTSKHPLVFKGMFWINRDSSVEMQRALIAAFKEIRDGLYPLGSNGGT |
| | GYGWISHLAITNGPDWLNLEEVPLPQPTADIPVEECTAEPYPKFQKPDLDQNAVYYPHY |
| | FLQPGKPAERERHPVSHDHIDDKLLTGRLVCTLTTKTPLIIPDTQTNTMLPPNDAPEGHK |
| | SFRFFRIDDEVLIPGSEIRGMVSTVFEALTGSCFRVINQKAHLSWRINADMAKHYRPGRII |
| | QNNEKMFIQPYKMFRLPFYAGFDPRNCLSEKQLLGIEPVKLWVKDFVASLVKPQTDIDI |
| | EWKEKIGFVRVTGPNKVEVDSSNTPDPSLPECESDWKDIHITEDGSTPSKNDRVYRCQL |
| | KGVTYTVAKWCEAFWVKDEGKKPITVNAEAINRYHLIMKSYQDNPQSPPIIFRSLPVLN |
| | YKQDQKIIGSMIFYRESAKSDKIVNEIIPVKISRTADTELLAKHLPNNDFLPCAATCLNEC |
| | DTCNAKTCKFLPLYREGYPVNGLCPSCHLFGTTGYQGRVRFGFAKMNGNAKFCQGGE |
| | RPEDRAVTLPLQERPKLTWVMPNENSTIPGRKFFLHHQGWKKIVDEGKNPINGDVIEPD |
| | ANNRTVEPLAAGNDFSFEVFFENLREWELGLLRYTLELESELAHKLGMGKAFGFGSVKI |
| | KIKSVDLRKQGEWEKATNTLVSEDKKSSWYNIHTVNNLRTALYYVEDDKIQVNYPKLK |
| | KDNESDNRPGYVEMKKTAFPVRDILTTPWWPWWPPTPPPMNQSGNQSYARSEEPARIT |
| | ESQPEVYKTGTVKFYKHDKKFGFITMDGRENIHFAGNQICRPETSLQSGDKVKFIEGENY |
| | KGPTALKVERLKG |
| SEQ ID NO: 21 | MRLKINIHFLEPFRLIEWHEQDRRNKGNSRWQRGQSFARWHRRKDNDQGRPYITGTLL |
| OBJA01001127 | RSVVIRAVEEELARPDTAWQSCGGLFITPDGQTKPQHLRHRATVRARQTAKDKCADRQ |
| | SACPFCLLLGRFDQVGKDGDKKGEGLRFDVRFSNLDLPKDFSPRDFDGPQEIGSRRTINR |
| | VDDETGKAHDFFSIWEVDAVREFQGEIVLAADLPSRDQVESLLHHALGFVDRLCGARC |
| | VISIADQKPAEREERTVAAGDEKATIADYDQVKGLPYTRLRPLADAVRNLRQLDLAELN |
| | KPDGKFLPPGRVNKDGRRVPHYVWDIPLGKGDTLRKRLEFLAASCEGDQAKWRNICES |
| | EGQALYEKSKKLKDSPAAPGRHLGAAEQVRPPQPPVSYSEESINSDLPLAEWIITGTLRA |
| | ETPFAIGMDAPIDDDQTSSRTLVDRDGRYRLPRSTLRGILRRDLSLASGDQGCQVRLGPE |
| | RPCTCPVCLILRQVVIADTVSETTVPADIRQRIRRNPITGTAADGGLFDTERGPKGAGFPF |
| | SLRYRGHAPMPKALRTVLQWWSAGKCFAGSDGGVGCGRFALDNLEVYRWDLGTFAF |
| | RQAYSENNGLRSPEEEFDLAVIHELAEGLAKEDGQKILKGTEPFTCWQERSWQFSFTGP |
| | LLQGDPLAALNSDTADIISFRRTVVDNGEVLREPVLRGEGLRGLLRTAVGRVAGDDLLT |
| | RSHQDCKCEICQLFGSEHRAGILRFEDLPPVSPTTVADKRLDHVAIDRFDQSVVEKYDDR |
| | PLVGSPKQPLVFKGCFWVQTSGMTHQLTELLAQAWRDIAAGHYPVGGKGGIGYGWIN |
| | SLVVDGEKITCRPDGDSISLTTVTGDIPPRPALTPPAGAIYYPHYFLPPNPEHKPKRSDKII |
| | GHHTFATDPDSFTGRITCKLEVVTPLIVPDTEGEQPKDQHKNFPFFKINDEIMLPGAPLW |
| | AAVSQVYEALTNSCFRVMKQKRFLSWRMEAEDYKDFYPGRVLDGGKQIKKMGDKAIR |
| | MPLYDDSTATGSIKDDQLISDCCPKSDEKLQKALATNQKIALAAKHNQEYLAQLSPDER |
| | EEALQGLKKVSFWTESLANNEAPPFLIAKLGEERGKPKRAGYLKITGPNNANIANTNNP |
| | DDGGYIPSWKDQFDYSFRLLGPPRCLPNTKGNREYPRPGFTCVIDGKEYSLTKRCERIFE |
| | DISGGENQVVRAVTERVREQYREILASYRANAAGIAEGFRTRMYDTEELRENDLVYFKT |
| | AKQADGKERVVAISPVCISREADDRPLGKRLPAGFQPCSHVCLEDCNTCSAKNCPVPLY |
| | REGWPVNGLCPACRLFGAQMYKGRVNFGFARLPDDKQPETKTLTLPLLERPRPTWVLP |
| | KSVKGSNTEDATIPGRKFYLRHDGWRIVMAGTNPITGESIEKTANNATVEAIMPGATFTF |
| | DIVCENLDQQELGLLLYSLELEEGMSHTLGRGKPLGFGNVRIKVEKIEKRLSDGSRREMI |
| | PPKGAGLFMTDKVQDALRGLTEGGDWHQRPHISGLRRLLTRYPEIKARYPKLSQGEDK |
| | EPGYIELKSQKDENGVPIYNPNRELRVSENGPLPWFLLAKK |
| SEQ ID NO: 22 | MSNQTRWIIEGTLELITPLHIGTGLDEKERDENKETRWLEAVALDHKGQPYIPGASLKGA |
| OBEQ011807420 | LRALAKRHDFRNLFDNKEVDGDFVRQAEFLSAWCVPDTDKGRLIQPRVAIDRVTGTAQ |
| | DKKLFQTRLITPGTRFAMKIVVQNAVENEIADLLGLLNLLPDDPQFSLGAYANQGQGRV |
| | QWFGKIQTRCFGINEAKAWYEEIRKDESKCWTAFAKPKNVSTPPTPAKEAQLTLPLNLA |
| | FHTPFLVKQAGIKADDADAVPRRTHDDKIVLPASSLRGRLRTQSERILRTLGCETPQGHT |
| | APAYRKGQPHDDLAVLLFGAAGWRGIVQTSDCIVEDKSIKTRRHEMLAIDRFTGGGKD |
| | GAKFNVDYVECPTLAGKLSLDLARLKNAKLKGGKDALLPALGLMTLMLRDLAEGDIPF |
| | GYGISKGYGQCRASSALGDWAELLKQHLGADSADTTVQALREYLGNPKGQELKLDPPS |
| | ADATQAGVPAQQNAAKTQAQGAQEKFHNPYHFIPLSKPDISQWPEPQKLTEKGHSHDR |
| | YASLSGRIVCRLTTQTPLFIGSEQTTPTNPQAPKSLHPFKLNNGLAIPATSLRGMISSLFES |
| | VSNSNFRVLDEKTYSMRKTMQQSLSAMGRIVRHDQKLYLLPLTLPTLPQGPHGVYDLG |
| | EKWSAVFDWQPPPLRIYFDPPPRRTYQSQQPCYMKLSTVKYSESNPNQIIAGENLGALRF |
| | PRGNQNTQFLIGQSNQDECPITQAEYAQKSEDERNEYTSGWVRTLVKPGRDLPRSVKHH |
| | VFLPDVFIDAPPPVNDLYPIPDSVIQRFHDLADQVLASMNLKPEEIVDSTNLLPYTPVGRR |
| | SDSDCRDTRLQAGDLIFFDIDPPLHPGEKSQITEISFSSIWRSGIGKDHLLTTPDLLTNFDV |
| | NLQPHGMPGRTQSLSPAELLFGLVGTQNDQATTAYAGKVRIGFGLPEEGHNPRLDARIT |
| | LKELSSPKPPSPALYFRKKSGKDEYVSKANLADKPEDYILRGRKMYLHAWRKNEQVVE |
| | LSDTGHDGGVRPPWVSKFDESADEGNKRRVSIEPIAKDESFYFEVDFHNLSRTELAQLC |
| | ATLYPNEKFEHRLGMGKPLGLGSIKITPLSLFLVNRSQRYATDGLDKPRYHAVWHTGTA |
| | SEPRWPDHLQREQQGIAFEGVSTAPTVMSLAAEAKVSDDVKRALELLGNPDEITVPVHY |
| | PQLHNGLMESKQFDWFVQNDKSGRDQPANNRQHLSSFTKDTEKLEPLIRIMRR |
| SEQ ID NO: 23 | MTTPSAPKSSLPALHWLIRAELEVLTPLHLGTGTDQRITPDAPDADPYWQADIALDADG |
| OVOO01000106 | RPYLPGASLKGALKALARRRQVDAPCLPLFGDLNRGGAPHPDCIPPRRTRAGLAEFRDA |
| | LQSHATQADGPDTAQPRIAIDRITGSVVDKKLFHTQTVPVGTRFSVEIILRRADQNLAAQ |
| | LVALLQHGPTDPDFRLGAHANLGFGRVGLYGNIDTRRFGPQQAQHWFAAAQTQADAR |
| | WTDFAEAVTLTAPAPAPAQPAPHRLALPLSLTFHTPFLVKQPEHKHRKPQDNAPDGTPR |
| | QRGDRALLPGASLRGRLRSQAERILRTLGCKVAQGHAVPPVKNNTCPDPATLLFGTAG |
| | WRGLLRTDDCTGTAPATLVDHDMLAIDRFTGGGKDGAKFKLRYAECPTLEGQLSLDLS |
| | RLRSARLDGANAADTPWIALGLLTLVLRDLAEGDIPFGHGSAKGYGRCRAQGLPDRWR |
| | QALEAHFGPNADARALAALRAWCRTHATAALDAPCSLAGSAPTPAAAAPSGQAAPAD |
| | AFHNPYHFIPFSQPDIDRWLSPDAHRKTGGHSRYRGLSGRLVCALTTVTPLFVGAAART |
| | PASDQHPKPVAGFALQNQPAIPATSLRGLLSSLFESISGSNLRVLHPTPYSIRKTTKEALSA |
| | IGRIVERNGELKLYPLTLPTIHQNADNAYPVPARWRKVFYWESPVPLRVYFGSRKQTYD |
| | SRQPHYLPIQELSYLPNDSDCIAPDQGDLRFPSRDRDRKFLIGQCPISRYDCPIPETDLPKL |
| | SPQERPRYTRGWVRSLWTSNREKELPHTVKHQLFIPDPVETPAADDLLPIPQGVLDTFHA |
| | LADLALAGQHWGKDETPADDQLLPFTPAGRQRHDADRPPRDADRQTRLQPGDLVCFD |
| | LGDDGAVSEISFSSIWREGLRLAGKPNLATTADLLAQVSPHLLPLGMPGRSARLSPVEQL |
| | FGVVEYRPPQTAKGTRKPTDAPAAYALAGKLQVGFGRPARPFEREPAVTLKELSTPKPP |
| | SPALYFRPKAGDGYVSKAKLASQPQDYAPAGRKHYLHALRRQGQVARLDNSGHVPSD |
| | GSGRPPWQSRFDGQEDSGNKRRVRVEPIPAGETFHFEIDFDNLSPTELEQLCATLLPHPA |
| | FEHRLGMGKPIGLGSVKLAVEGLLLVDRPRRYAEDEPNAPRHHRGWRANADAGWPDH |
| | LQGDSPAAPLEATEQPAALAERAMARVPADVRRALQLLGNPGAVAAPVHYPQVKDAQ |
| | IEEKHYLWFVANDDEKTAGGNRHLPRLHANSPGLPTLPRLVKREKDHSSNTGKPRRK |
| SEQ ID NO: 24 | MIPDLRSLVVHISFLTPYRQAPWFPPEKRRNNNRDWLRMQSYARWHKVAPEEGHPFITG |
| PDWI01005922.1 | TLLRSRVIRAVEEELCLANGIWRGVACCPGEFNSQAKKKPKHLRRRTTLQWYPEGAKS |
| | CSKQDGRENACPFCLLLDRFGGEKSEEGRKKNNDYDVHFSNLNPFYPGSSPKVWSGPEE |
| | IGRLRTLNRIDRLTTKAQDFFRIYEVDQVRDFFGTITLAGDLPRKVDVEFLLRRGLGFVST |
| | LCGAQCEIKVVDLKKKQNNKEDSILPVSEVPFFLEPEVLAKMCQDVFPSGKLRMLADVI |
| | LRLREEGPDNLTLPMGSQGLGGRLPHHLWDVPLVSKDRETQTLRSCLEKIAAQCKSEQT |
| | QFRLFCQKLGSSLFRINKGVYLAPNSKISPEPCLDPSKTIRTKGPVPGKQKHRFSLLPPFE |
| | WIITGTLKAQTPFFIPDEQGSHDHTSRKILLTRDFYYRLPRSLLRGIIRRDLHEATDKGGC |
| | RVELAPDVPCTCQVCRLLGRMLLADTTSTTKVAPDMRHRVGVDRSCGIVRDGALFDTE |
| | YGIEGVCFPLEIRYRGNKDLEGPIRQLLSWWQQGLLFLGGDFGIGKGRFRLENMKIHRW |
| | DLRDESARADYVQKCGLRRGVGDDTAINLEKDLSLNLPESGYPWKKHAWKLSFQVPLL |
| | TADPIMAQTRHEEDSVYFQKRIFTSDGRVVLVPALRGEGLRGLLRTAVSRAYGISLINDE |
| | HEDCDCPLCKIFGNEHHAGMLRFDDMVPVGTWNDKKIDHVSCSRFDASVVNKEDDRS |
| | LVGSPDSPLHFEGTFWLHRDFQNDVEIKTALQDFADGLYSIGGKGGIGYGWLFDMEIPR |
| | SLRKLNSGFREASSIQDALLDSAKEIPLSAPLTFTPVKGAVYNPYYYLPFPAEKPERCLVP |
| | PSHARLQSDRYTGCLTCELETVSPLLLPDTCREKDGNYKEYPSFRLNNTPMIPGAGLRA |
| | AVSQVYEVLTNSCIRIMDQGQTLSWRMSTSEHKDYQPGKITDNGRKIQPMGKQAIRLPL |
| | YDEVIHHVSTPGDTDDLEKLKAIVLELTRPWKELPEEQKKKRFEKCKNILDGRMLQQKE |
| | LRALENSGFAYWRDKTSLTFDSFLKDAIEQEYPRYSGDYQRIKALVVNITLPWKLLKKE |
| | ERHKRFDKCRRILKGQQPLTKDERKALEESGFANWHGRELLFDRFLKDENSCLIKAETT |
| | DRVIASVAKNNRDYLFEIKQQDFARYKRIIQGLERVPFSLRSLAKSKETSFQIACLGLRRG |
| | RFLRKGYLKISGPNNANVEISGGSHSNSGYSDIWDDPLDFSFRLSGKSELRPNTQKTREY |
| | PRPSFTCTVDGKQYTVNKRCERVFEDSAAPAIELPRMVREGYKGILTDYEQNAKHIPQG |
| | FQTRFSSYRELNDGDLVYYKTDSQGRVTDLAPVCLSRLADDRPLGKRLPEEYRPCAHV |
| | CLEECDPCTGKDCPVPIYREGYPARGFCPACQLFGTQMYKGRVRFSFGVPVNSTRSPQL |
| | KYVTLPSQERPRPTWVLPESCKGKEKDVPGRKFYLRHDGWREMWGDDDKPDSRPSSE |
| | ECQDIIEGIGPGEKFHFRVAFENLDKNELGRLLYSLELDAGMNHHLGRGKAFGFGQVKI |
| | RVTKLERRLEPGQWRSEKICTDLPVTSSELVISSLKKVEERRKLLRLVMTPYKGLTACYP |
| | GLERENGRPGYTDLKMLATYDPYRELVVQIGSNQPLRPWYEPGKSFKPSPGNDCTGRG |
| | GSVSKSLISEPKVVPAIAPFCEGVVKWFNSVKGFGFIETKEQRDIFVHFSAIRGEGYKILEP |
| | GEKVRFEIGEGRKGPQAINVIRIR |
| SEQ ID NO: 25 | MKMNKTWPFREHWEISGYLRTVTPLHIGSGRTVTRPELTVADRDELVDINAVVTDYTG |
| VAPF01001339.1 | KPYLPGSTIKGALHAWLQKRLKEESRTCLIQLFGQEEEAEEKKKNNHGGKAEFFDARVI |
| | FPHTGPGSLPYWDDCRQTYVAATVAIDNITRTARHRHLIHAEMVPPGVTFALTLAGPLD |
| | EEDIGLLLAALQGFNESPPALVIGAHTANGLGRFSWELSTVRRFGKEHLQGWLEAETRA |
| | MRTEAMQPLSREGVEDLLQDGIAQIDHNEDQVRLGLELCFDGPFLVNDPPTKKEQDDK |
| | KKRRSNTPNLRPLRDAIGRPCLPESSVRGALRAQAERIIRTMEGTCSEDNPAYKKEIHTD |
| | AEIEELSAVCRVFGAPGWKSLLEISDFEFVDGEDCDNIQEFVAIDRFTGGAKDKAKFNAE |
| | YIGSPRFTGTIALDKRRDLPDWGKGLLYLVLRDLAEGDITLGFGRSKGYGVCRAKIKNL |
| | DLLLPEKSVAALHKKFAISPADDKPATDQIAEDTTSGNLGISAGGPAQKTTESYEPPNPS |
| | GPGTFHNPYHFVPVVKPSAADRQHWLDKGILPSASENKTQHTHACYLDTTNGKKIYHG |
| | RIVCRLQAETPMFIGGRHRENTEPTEILPFTLGGKPAIPATSLRGMLSSIAEAASNSSLRVL |
| | EDKTLSYRKSMRANRNEDKPLSALGMIKKIETGDKVEYRLLPLTLPTLVKRGQYYILPE |
| | EYQTMFPDGRAKLKVYLNSNYTASDGTNQDFLKGKKSWRLPHGEIYYMKLCQDFSLQ |
| | NGQLTFDSQNQNMLHFPKNRNNFVVGQRSIDNTPPMTKAEWRTNHQTGVPGMLRIMQ |
| | ASGRNFPTGRYHEIFIPVPTKKDCKQLYPVDEKAVERFLDLAGEQTKSQQNEKNLKQYQ |
| | ILPYHPVGTKRNTDPETNDRYMDLNSGDIVYFRPDATGTKVEEISFSSIWRDRVEDDNH |
| | NRAGVHAFFGNIDKELLPFNPKRAEISPAELLFGFVEERERGKVDDGQAPAFAGKVRVS |
| | FGRLSSEKKPDTIFQDQVTLKALSSPKPPSPALYFTGNGNGSIAKPDLTLSRHSPQGRKFY |
| | LHAWQEENEIIKFLSNGKKTSPTVINGLYPWESKSNLSRQLPDKHAKLKSAITPIKKGTV |
| | FYFHVDFDNLSEWELGLLCYALQPGKEFRHKLGMGKPIGLGSIHIEPAGLYLVHRGNRY |
| | SLDGVPDNSRYNGGIWQSEDKRLQEWQELYPRESTAAASSAAASPADFALKFSGTMTP |
| | SVQQALKRLGDPDNVVAPVHYPQVEGADLEEETYKWFVANDVGSQTKVGNRTTCTEE |
| | AARKSMLPLAGRGPLPRLKRYKWCP |
| SEQ ID NO: 26 | MAAVQDRWTLMDQQGNELKRFRITAELETASSLHIGASETVEHDLIKNDDGTPVQINAL |
| DRKI01000155.1 | ITGAGGLPIIPGSTIKGRFLARLRERGVDSALLETLFGKGHDRETEDQGRGGRAEFHDAP |
| | LCHRLSGARHFPYWRPERQIWVKAQTAVDRHRGTALRRSLRYTEMVPPGVRFRLTITG |
| | CMTDAEADVLFALLEDLGDPRQACSFGGAGADGNGTMRLFGRPEVYCLDRSGILGWL |
| | ASFEKGGNGGMAMTAAALLQADTVQRRADKVRQAWQPPDVGPRLHVELRFSGPFLV |
| | NDPSRNTPDITQAPDMVPLVDEDGNPMLPASSFRGALRAQAERIIRTLGGRCCDTSSPCR |
| | PLGSSDKVGELCLACQVFGAPGWGTTLHIQGFTCTSVFRREQEQTFVAIDRFHGGCKEG |
| | ALYTIRHAESPRFEGHLVIDPRMPAWGRGLLALVFRDLREGDITFGLGAGKGYGVVDA |
| | AVVQDMAELEPYVEAFREQCRQHQGMADCHSAPSPQPLRDHDLAEIPPAEEAPGETFL |
| | NPYHFVPIREPDTGSWLARDELDSSCCHSHGFYRQQVDDRPLYHGRLTCLLETETPLFIG |
| | ATGDSSVPSRIENYRLGNRIAIPAASLRGMLSSLAEAASNSAMRVLHQGILSYRKKAKN |
| | ALREIGMIVLRDGKRFILPLVPLMEVTKLRHAYTDPAMKHFLDDKNSWSPRCNRVYYL |
| | GRDGNQIPAETRGAGMRPGILRLLGREGRHDALQNKKHEYFIEIPERYVDQDHCFDYR |
| | MFIRDRARNGTLVPISPVAWERYHCLAEERTLSQKNDPELREDKACASLKWLPFHPKGR |
| | VRERDPENDVCHLSLRHGDLVFYAEQNRVVSEISFSAIWRSRVETSDSYQAVTVDCFVP |
| | KELRPFNRDRRAISPAELLFGFVELDESEHSTEKSRYEQMAFAGKVRLSAGLPVEDVEDS |
| | ALLEPKPIVLKALSSPKPPSPPFYFVMRDGSGAYIAKKDLSPDRHRIKGRKHYLHGLRQR |
| | GNPDRVQSLDRYGHATETAANPPWETCHPEERPQIKVRVQPVRRKTKFFFHLDFSNLSR |
| | WELGLLCYVLRPTACFRHKLGMGKPLGLGSVRIDIASLQLIDRVRRYGTDDLTAGRYN |
| | MGGHFNASCLDLLPQQDSPAPDDSGAAPDPGTLRQDFVKTMDETVFRALDLLGNPAHV |
| | QRPVHYPQVREMDIEDQTFLWFVDNDKQWKDALQPLTSSSTQLPPLTRRNKR |
| SEQ ID NO: 27 | MTTVKEKSWAFTGLKRWKIITTLETQTPLHIGSGEVAEIEINDSQGDRRQVQANAIIRGK |
| DRNY01000543.1 | DDKPIIPGSTLKGKLRSHFESCLDHSKALERVFGKEYQSDEEQGRGGLAEFHDAVCSYV |
| | APGNSYYPNWNEARNTYIEASTTIDCHTGTAADATLHYNECVPPGTRFLVTVTGAMSD |
| | KDAALIVAALQAFGDETNPIHLGAEEANGKGRMGLFGNVEVSCLDHDDIIAWISQGSDA |
| | RMATDKFKPLGKEKVNDLAKNITTPTATGGAQRQHFGIELKFDGPFLVNDPSKYSKGD |
| | GDQPAVHQPLTDRSNNPILPARSFRGAIRAQAERIIRTMGGACCDTQSPCQNSGQLCIAC |
| | QMFGTTGWKTTLSISDFTYDGEYRPAKTQQFVAIDRFHGGGKDGALFSIKYFERPVLKG |
| | GISLKLRNQNADELSWRKGLLALLFRDLQEGDITFGFGANKGYGGVEEACITNADVIST |
| | ADIEAFRAKCHANHADSWCSPVSKPTNRDDKSSLPSINPATGAGHAFHNPYHFIPIKAPD |
| | TSTWLDKHKLATPGSPHSHAYYRSCSDDDKPLHHGRITCKLTAETPLFVGSGDAENQLT |
| | DSEAKLKEHYQLNNKLAIPATSLRGLISSLAEAASNSALRVLDNGVLSYRKPASRALRKI |
| | GILFKREEQWRLVQMEGNLANAIKLKSAYTNQKMMDFLANKQSWSPEHNVVYYLSAD |
| | FRPGDVPQETYLAGRICGILRILGGKDGDRKNELENKKHELFIRVDEQYVDTEINRFDYE |
| | EYVRQGGIPVSPSAVERYTELADQRSLSQKNSRDLKGDNNCCSDKWLPFHLKGAARYK |
| | KEKACLLPLREYDLVYFDSDGTQVTEISFSAIWRDRVADKVHAFFPEELRPFNQKRKWI |
| | SPAELLFGFVELNDNKDERDHAQAFTGKVRVAAGVLSPDDSIRQGDLQEHEPIMLKALS |
| | SPKLPSPALYFKQKSGDHRYIAKPDLKKASHQAQGRKIYLHALRDQKDDVQKLNTKGQ |
| | PANGNGAHLPWKTADEDERPQLKVRIRPLKPGTSFYFHLDYNNLTEWELGLLCYVLQP |
| | SETFRHKLGMGKPIGLGTVKIEIATLQTIDRQKRYREAGANEHRHNGSNWVNESLRDEL |
| | ERLPGTVELSPDRQPEAKLRPDELRQSFIATMDNDIYRAIELLGDPHNIKYPVHYPQVRN |
| | KSIEQENFKWFVANDSGSGDQRKGTGIDAKEEPMRSIDQISTTIPTLNRYEWNGD |
| SEQ ID NO: 28 | MARNNKQYHFIPRWEIKVNLTTRSFLHIGCDEFTDRPGLEIEQKDGSKVKAEINAFIKDS |
| DTXS01000070.1 | NGKPYLPGSTIKGNIRKWLETNKKADEETCKLFNTLLGFTVKMQDEGCGGSAEFHNAVI |
| | SSPLEDGNNFPYWDVDLQTSVETSTVIDRVTGTVVDGRLFSTEVVPPEVSFTLIITGAMT |
| | EQQVSLLTAVIKDGFAEDCPTPITIGADSGNGFGRFRFDSIHMKCLGTGEVLNWLEDGSQ |
| | DMAATAMRSLSPDDIEQHIIKGRNYLKSPSVSDTVTIEFGFAGPFLVNDPSRKKRKEDID |
| | HQPLRDSAGNARLPAKSIRGAMRSQAEKIIRTLGGWCCDPVNPCPSVFSVVEINDRLCLA |
| | CRVFGATGWKSRISIQKVEYKGTAESTRQETVQDFVAIDRFHGGGKETAKFDASFSWRP |
| | QYSILMHIPSDLEGWAKGLLALTFRDFKEGDIFLGYGRSKGYGRVDSDSVKPGIDTMLT |
| | ESNLELFRRKCDDNPGEYPCKTRQPPNLVQPVERNNLTEAADEGSFHNPYHFIPTPKPMI |
| | ESWLAKEDFDETMHDSHALYRDVDENEEPLYHGKISCTLTTETPVFVGGKHDPRNDTE |
| | PQQVDHYTENGEIAIPATTLRGLLSSLSEAASNSSMRVLDDGMMSYRQPVGSGSLSAIG |
| | MVVIRDGKKFIYPLALPIFGERDKLPQEYHIMFPYTQKAPLKVYLERAYLAGNMKSFLD |
| | KQNSWNLLNEKIFYLPVPEFSFSRVHTMGAENRDVLKISRRGNLILGARLPVNLCPRSKE |
| | KALPGDIPGILRILGKEGRDGEVPVGKKHELFIPVSDGFASNPRSFIDNLTSKELFKIPDEV |
| | VDRFEELADDRTTQQIKHPGNVKNNNQWLPFHLKGCTRNDGLTGKDEKRLRVQEGDL |
| | MYFRPSPQSPQVAEISFSAVWRGRVNKTVHNYFPPELVQFNKNREKISPAELLFGFVQQ |
| | DKHEKSLSFAGKVVLSSGKQLRETESVSRENEVTLKILASPKLPSPSLYFKRENYIEGGN |
| | YIAKNEMNNSSNIKPRGRKQYLHALSNSEDPKGVQKISRTGSVDDGGNYPWQSMNNDN |
| | IKQKVCIRPVSKDGCFTFEMEFENCTEWELGMLLYALRPSQQYRHKIGMGKSIGLGTVR |
| | IDINNLQFIHRKNRYNAGIIDVPRYNYEAGHDMDYFHNKFADTIMPEIKNSIELLGDPRN |
| | VRFPVHYPQVHGADIEDKTYQWFVANDSGTNNGQNGAAYKKNKAEESSLTELDEISNT |
| | IPGLERHEWLGR |
| SEQ ID NO: 29 | MALKTWTLNGEERWHISVVLETVTPLHIGSGEFCYRPELTNADQKPVDINACIKGANNL |
| JABFST010000317.1 | PIIPGSTVKGKFNAWLTARQVDTPLLEAIFGKGHNPDDDDQGSGGKVEFHDAWISTKIK |
| | DTSTWPYWQVATQTFIDAATAIDRHSRTALDASLHYTECVPPGVQFTLNITGVMQEHEA |
| | ALIIAALDRFDQHDDQPYFGAGDANGQGQLILVGHLAVKVMGKTEITEWLAHENNKAS |
| | DMAMSHARSLGAEDIAGLIKLGQTLLKPVPPTVSLGIQLQFAGPFLVNDPYAVKKLEAD |
| | PKTKIDHYPLLDNHKKPRLPSASIRGVLRSQAERIIRSLGVHCCDTRDPCPSLYKHQDLSQ |
| | LCLACQIFGAAGWKSVINISDFTCVDANELKTQEFIAIDRFHGGGKDGAKFNAKHSERP |
| | YFQGRITLSPRMANHQLDWGKGLLALVIRDLQEGDLSFGFGANKGYGALESVLITGIDQ |
| | LQTDAIEAFRRLCVTQAAPQAFITPTSAVVIGDKAPLVVTDKKLPDNSFHNPYHFIPINSP |
| | DTRHWLPTETDLAESHHSHAYYRQQPELFHGQLICRLYTETPTFIGASKKDDTLPAELD |
| | NYRLNGQLAIPATSLRGMISSLAEAASNSAMRVLDNGLLSYRKDASLALSKIGITFINRQ |
| | GQWQLIPMEKIKLKNAYSAENMRLFVEQSHSWSPDYNTVYYFSEKAGAFDVPQRTPKP |
| | GWQPGILRLLGKEGRSQELENKKHEWFIPVPENYIDKQLNAFKYQEYLKDNSSKAIDIPA |
| | PVLNRYNELAYQRTLSQKKDTELVADGDSPAWLPFHLKGQQRQPQMVGKHLVYTLPM |
| | TEYSLVYYAATNKVATEISYSSIWRGRVQDDADQAATVNHFIPDDLLPFNPKRTSLSPAE |
| | LLFGFTELDPDKHSNDPTRSFAGKVRIGAATLAAYPSNDSDLLAPEHITLKALSSPKLPSP |
| | ALYFRTLQGNNSNVYIPKHELNPNHHTAKGRKYYLHATRTPDQKRILKLSDQGHPPQN |
| | NAVKLPWLSHQETKNLQLKVKIKPIKPKQSFYFQVDFNNLTAWELGLLCYALRPTIDFR |
| | HRIGMGKPLGLGSVKIDILALQTLDRQKRYAQDSQDSARYNQHRWVNSSVTDMLAQA |
| | GYDVIEPTANPLVPKDLKTLFSQTMAANIDRALTLLGEPQHVKQPVHYPQVRDTAIQVR |
| | DTAIEEESYQWFVANDNLSDNSSAAKQTLHDITETSEGLPTLIRHQKKKETQP |
| SEQ ID NO: 30 | MSDTQKQAIHENKWHFRGIKRWEISAYLKTLSPLHIGDGGTIPVTIKDTQGKNREVEVN |
| PDPY01000001.1 | SVITGKAALPIIPGSTIKGRLRHYFSKHFSDKALLNKVFGEESDATDDDQGRGGLAEFHD |
| | AKWNPEKNRNLQGRYPYWNNTRKTYIEVSTAINRHTGAAKDKSLHHTECVPPGTVFEI |
| | KITGSMDDRCAALVVAALEAIQTTGSRIFLGAEDANGNGRIGLTGKITVKQMDQAHIIQ |
| | WLQKDSTTCVASFSNVKAENETQVKQMVQRHIAPKLNSVVSAAGPSYDITLHFDGPFV |
| | VNDSDKCKAEDTPDIYPLEEKNGVPAFPVRSFRGAIRSQAERIIRTIGGQCCDGSINNTCK |
| | NPKNLCIACEMFGSTGWKTSIEMDPFLCVDRELKPFIIQEFVAIDRFHGGGKDEAKFNAA |
| | HYQAPVFKGKVRVSQRVGNDISWRKGLLALIFRDLKEGDIYFGFGTNKGYGAVKKAEI |
| | NPDGNASDFSESDIEAFINKCREKKGLYNCNPIKKPGKTKVSKNLPPAIVPLDRTDSKFY |
| | NPYHFIPVKKPNTSSWAEKTAFGTADSPHSHGFYRKQTNEQQPLYSGRLICMLTSETPFF |
| | IGAQAESDPTENENQASLRHPYQLDGEPAIPSTSLRGLISTMTEAAANCAMRVLDSEIISY |
| | RKPMNPSHILSALGMVTKRGEDFWLIPLAMPALSLNDEEHNYKLDKRYRTMFPDGLAK |
| | LKVYLEKAYSNNVMKTFLNNENTWTLAQSKIHYLPLTPIQMQNGGINSYYNNLRTPSRS |
| | NNFLIGQTVAHGNGIPASGPGAGMVPGILRILGKEHRQNDLPQNKKHELFIPVPDAFVAD |
| | PKTFLDTATAFLIPRNVIDAFEKIAEKQTQSQKQDKLKHDEERLPFHLKGTRREQNHTLQ |
| | IKTGDLVYFRPNAKGDEVEEIAFSSIWRGKTSGTTADFFPDKELLPFNRNRSRVSPAELLF |
| | GFTENNPKEMKIDRGLAFAGKIRISAGTLSDKFSDTTESDLFEPETTLKALSSPKPPSPAL |
| | YFKEKKSGTQYIKKQDLNPGKHEIQGRKIYLHALRNENNQNVQRITSQGKFDNAANRT |
| | QPWVSQNEERNHLKTKCKPLKSGLNFFFHIDFNNLTQWELGLLCYALRPCETFRHKIGM |
| | GKPIGLGTVKIDIAVLQTIDRYARYTDTTQDSERYNQGAWISQELQNEIPNQYKGKGISN |
| | KKGMLSPEDCRKVEMETMDADIQRAIELLGDPGNVTSPVHYPQLDRKNIETKNYEWFK |
| | QNEIEQQVLKPITKNTTHLTPFARWEQG |
| SEQ ID NO: 31 | MNLPTWKLNNEKRWHISIVLTTATPLHIGSGEFCEHDDVKNNDGEPVKINACIKGSKGR |
| NZ_JMLA01000001.1 | PIIPGSTIKGKLYEWLKTRNTEENLLEKLFGKGHNSVSQDQGRGGKAEFHDAEIIEPLTGS |
| | QPWPYWREEHQAFIAASTAIDRHKQVALQQSLHYMETVPAGIRFKFTFTGVMRDEEAA |
| | LLIAALDSFDKNQNQPCFGVDRANAYGRMELHGHLHVKVMGATEISSWLNSFSENDKK |
| | MAMESARNLEQQEINTLIKQGNALFKASCDEVKLGLTLKFKGPFLVNDPYAVKILSSNE |
| | NAKTDHYPLLDKNRNPYLPVSSFRGVLRSQAERIIRTLGGKCCSTDDPCKPIFDKGDLSK |
| | LCLACQIFGASGWKTVINIHDFKAINKSKKTKQDFVAIDRFHGGGKDGAKFDATHFERP |
| | EFEGAISFSPRMANNDLDWGKGLLALVLRDMQEGDMTFGYGANKGYGGLESASITGIE |
| | QITSDIQAFRDKCVASPQTWLCDEAVKPANQQDKIPPAGIQVANSGFHNPYQFIPSKDPD |
| | TGHWLPVLGLNADSHHSHAFYRDQTDNGEKLYHGRLICCLNTETPIFIGADKKKDTEPA |
| | EINNYRLNGELAIPATSLRGMISSLAEAASNSAMRVLDNGLLSYRKTADDALRKVGMVI |
| | YVDNKSFIIKLNDAIKLKQTYTPGNMKDFIEKSNSWSPEHNTVYYLDNNQIPQESYMNG |
| | MKPGILRILGKEGREQELENKLHELFIPVPLEYVDTENNKEDYQAYKKAFLYRAIEIPEPV |
| | LKRYSELADQRTMSQKSNKELKKDDTCQSVGWLPFHLKGTKRQLDDKHKVGKLQIDE |
| | YDLIYYEASGKEVTEVAFSSIWRGRVETNSSQANKVYSFIPGELLPFNESRKKVSPAELLF |
| | GFTQINKDGSKADDKAQAFAGKVRISAGTISEYPESEANLLEQEVTLKALSTPKLPSPAL |
| | YFRTINGNGSAYISKQELEPSKHLAKGRKYYLHALRTGDNKVQKLGSQGETANGGDSK |
| | LPWVTHNPDERPQLKVKIKPIKAEFIFSLDENNLTEWELGLLCYALRPTDSFRHRIGMGK |
| | PLGLGSVKIDIMALQTINRQQRYAQDGLEENRFNRHNWVNPPHQPRLDKAGYSISLSST |
| | PLNPEILRATFTKTMNADIYRTLELLGNPQNVKRPVHYPQVENHNIEQENYKWFVAND |
| | QGSGKGRNKIDPAEKALKILTENSDCLPTLSRLDWRDE |
| SEQ ID NO: 32 | MNNKGSNMTDTVKSGRWIITGQFQLVTPMHIGTGLDEEMDKQSGESVDKKQNNSWIQ |
| NZ_FOGH01000010.1 | AIALDLNKKPYIPGASIKGALKALARRYYCASNLNIFGDTIDTKDGDNKRKSVTVAGQA |
| | EFLNAWYAADQEDKPFDTITRVAIDRVTGTAEDRKLFNTRRVNPGVCFNYKIIIQNACET |
| | EIQYLLDLLRKAAKDPSFSLGAGANQSQGKVRCLSSCVRYFGKQEMHDWFRAIQNGKQ |
| | EHWQIFAKPSNIKYADLERPDIIANSLNLPLTLDFHTPFLVKASKKKDEAKNEADAKPRT |
| | NHQGQVILPASSLRGRLRAQAEKILRTMGQDIPQGHAAPAYDGIAHRDLISLLFGTAGW |
| | KGIVCASDLIHSIPEYALQFNGVRETISDLSDTVKSCIIVDLVKTSTAAEKTEEQLHIRIVD |
| | SAGSLIVHKSENSSWANDTFRDASVKDNFKARLKEIADPQDLSDALRADIKKRAFQLAT |
| | LTRHEMVAIDRFTGGGKEGAKFNVDYIECPTLTGAIYLDLHRLKQAQLKNDEDALKPA |
| | LGLISLLLRDLAEGDIAFGFGANKGYGQCREHAVLDNWEERLKKIGAGLTIDGALQALR |
| | DTVALEPPAEFPPEIEKTTDDNQPEAPDFNLKPASNGFHNPYHFIPLNNPKIGDWPEAKA |
| | ETLKANREGHDQYHTGKFSGRIVCSLTTQTPLFIGAETKPSTSDREPSEARPFKLNGKHAI |
| | PATSLRGMLSSLFESVSNSNFRVLHPEHYSVRKSLDDYVALSAMGRIVDDQGELKLQPL |
| | TLPTLFGNRNNVPAKWEKIFGTPSEDDFLRIYFDDIPSKFSSNKRYFYNCKATELKDFIKS |
| | DKYFIGKRTPTVFPKSSTEKSHLESLEFIDVEKFKKAVENLEITPGNNPYIHGWVRNLKD |
| | EFREDIPDNVKHHVFLPDTTKRVSPLEIPPHVKKRFHELADLALAGLHLKQGETIASPYKI |
| | LPYTPIGRNKLENHIHRVPNDLTCYMTRLKKGDLVFFDVDNDGQITEISFSSIWRAGIGT |
| | KNKLQTTADLLSQRDPNLVQLGMGVRTKNTDRFKLSPAERLFGVVEHRDDDNTTVEN |
| | VNQPNDKAQAFAFAGKVRIGFGLPDKKTTVNGVSPVTLKELSSPKPPSPAFYLKRKNND |
| | DFVSKKVAAECSETMTLRGRKCYLHAWREQNGNVMKLDAIGVNSGGSTCKPPWKTH |
| | KPAANDQKEFEEDKNKFITSRQVKIAPISENTPFYFEIDENNLDATELAQLCATLQPAPKF |
| | EHRLGMGKPLGLGSVKIEPVGLFLINRHQRYTTDSTNCDRYHYAWLKGEHAAWDWPE |
| | YFRQNVVTADCTQTFNDTFDKLVQNGLAGTDADIKHALQLLGDPQYIGVPVHYPIAGN |
| | STLENKHFEWFGNNDKASVLRQKAQANSKNHHYQPKQQATPEEPQYLHTITKDSKQIS |
| | LLKKNKIEDIENRDQQKHRYSNHRR |
| SEQ ID NO: 33 | MFPKGRQMRRQRLLGDAEYYGGTGREQPASIVISTDSDPDHKVYEWIITGQLKAETGFF |
| MVRP01000104.1 | FGTKAGAGGHTDLSILLGKDGHYRVPRSVFRGALRRDLRVAFGAGCRVEVGRERPCEC |
| | PVCKVMRQITVMDTISSYREAPEIRQRIRLNPYTGTVDKGALFDMEVGPEGIEFPFVLRF |
| | RGSKSFPSELAAVIGSWTKGTAWLGGAAATGKGRFSLLGLSIHKWNLSTAEGRKSYLA |
| | AYGLRDAADKTVKRLSIDKGGKGDVGLPAGLERDALPSSVREPLWKKLVCTVDFSSPL |
| | LLADPIAALLGVEGDERIGFDNIAYEKRRYNGETNTTESIPAVKGETFRGIVRTALGKRH |
| | GNLTRDHEDCRCRLCAVFGKEQEAGKIRFEDLMPVGAWTRKHLDHVAIDRFHGGAEE |
| | NMKFDTYALAASPTNPLRMKGLIWVRSDLFETGHDGPTPPYVKDIIDALADVKRGLYP |
| | VGGKTGSGYGWIKDVTIDGLPQGLSLPPAEERVDGVNEVPPYNYSAPPDLPSAAEGEYF |
| | FPHVFIKPYDKVDRVSRLTGHDRFRQGRITGRITCTLKTLTPLIIPDSEGIQTDATGHKMC |
| | KFFSVAGKPMIPGSEIRGMISSVYEALTNSCFRVFDEEKYLTRRVQPKKGAKSSELVPGII |
| | VWGQNGGLAVQQVKNAYRVPLYDDPAVTSAIPTEAQKNKERWESVPSVNLQGALDW |
| | NLTTANIARDNRTFLNSRPEEKDAILSGTKPISFELEGTNPNDMLVRLVPDGVDGAHSGY |
| | LKFTGLNMVLKANKKTSRKLAPSEEDVRTLAILHNDFDSRRDWRRPPNSQRYFPRSVLR |
| | FSLERSTYTIPKRCERVFEGTCGEPYSVPSDVERQYNSIIDDISKNYGRISETYLTKTANRK |
| | LTVGDLVYFIADLDKNMATHILPVFISRISDEKPLGELLPFSGKLIPCEGEPPTILKKMAPS |
| | LLTEAWRTLISTHLEGFCPACRLFGTTSYKGRIRFGFAEHTGTPKWLREELDWARPFLTL |
| | PIQERPRPTWSVPDDKSEVPGRKFYLHHHGGNRIVESNLRNRPEVNQTKNNSSVEPISAG |
| | NTFTFDVCFENLEAWELGLLLYCLELSPKLAHKLGRAKAFGFGSVKIHVERIEERTTDG |
| | AYQDVTAVKKNGWITTGHDKLREWFHRDDWEDVDHIRNLRTVLRFPDADQEHDVRY |
| | PELKANNGVSGYVELRDKMTASERQESLRTPWYRWFPQNGTGGSGRHEQAATSQEQD |
| | TAKDESVLSATQRRQAVIDVSDPDERLSGTVESFDRQKGDGYIGCGVRQFYVRLEDIRS |
| | RTALCEGQVVTFRARKEWEGHEAYDVEIDQ |
| SEQ ID NO: 34 | MTTTMKISIEFLEPFRMTKWQESTRRNKNNKEFVRGQAFARWHRNKKDNTKGRPYITG |
| WP_124327589 | TLLRSAVIRSAENLLTLSDGKISEKTCCPGKFDTEDKDRLLQLRQRSTLRWTDKNPCPDN |
| MUTANT | AETYCPFCELLGRSGNDGKKAEKKDWRFRIHFGNLSLPGKPDFDGPKAIGSQRVLNRVD |
| | FKSGKAHDFFKAYEVDHTRFPRFEGEITIDNKVSAEARKLLCDSLKFTDRLCGALCVIRF |
| | DEYTPAADSGKQTENVQAEPNANLAEKTAEQIISILDDNKKTEYTRLLADAIRSLRRSSK |
| | LVAGLPKDHDGKDDHYLWDIGKKKKDENSVTIRQILTTSADTKELKNAGKWREFCEKL |
| | GEALYLKSKDMSGGLKITRRILGDAEFHGKPDRLEKSRSVSIGSVLKETVVCGELVAKTP |
| | FFFGAIDEDAKQTALQVLLTPDNKYRLPRSAVRGILRRDLQTYFDSPCNAELGGRPCMC |
| | KTCRIMRGITVMDARSEYNAPPEIRHRTRINPFTGTVAEGALFNMEVAPEGIVFPFQLRY |
| | RGSEDGLPDALKTVLKWWAEGQAFMSGAASTGKGRFRMENAKYETLDLSDENQRND |
| | YLKNWGWRDEKGLEELKKRLNSGLPEPGNYRDPKWHEINVSIEMASPFINGDPIRAAV |
| | DKRGTAVVTFVKYKAEGEEAKPVCAYKAESFRGVIRSAVARIHMEDGVPLTELTHSDC |
| | ECLLCQIFGSEYEAGKIRFEDLVFESDPEPVTFDHVAIDRFTGGAADKKKFDDSPLPGSPA |
| | RPLMLKGSFWIRRDVLEDEEYCKALGKALADVNNGLYPLGGKSAIGYGQVKSLGIKGD |
| | DKRISRLMNPAFDETDVAVPEKPKTDAEVRIEAEKVYYPHYFVEPHKKVEREEKPCGHQ |
| | KFHEGRLTGKIRCKLITKTPLIVPDTSNDDFFRPADKEARKEKDEYHKSYAFFRLHKQIM |
| | IPGSELRGMVSSVYETVTNSCFRIFDETKRLSWRMDADHQNVLQDFLPGRVTADGKHIQ |
| | KFSETARVPFYDKTQKHFDILDEQEIAGEKPVRMWVKRFIKRLSLVDPAKHPQKKQDN |
| | KWKRRKEGIATFIEQKNGSYYFNVVTNNGCTSFHLWHKPDNFDQEKLEGIQNGEKLDC |
| | WVRDSRYQKAFQEIPENDPDGWECKEGYLHVVGPSKVEFSDKKGDVINNFQGTLPSVP |
| | NDWKTIRTNDFKNRKRKNEPVFCCEDDKGNYYTMAKYCETFFFDLKENEEYEIPEKARI |
| | KYKELLRVYNNNPQAVPESVFQSRVARENVEKLKSGDLVYFKHNEKYVEDIVPVRISRT |
| | VDDRMIGKRMSADLRPCHGDWVEDGDLSALNAYPEKRLLLRHPKGLCPACRLFGTGS |
| | YKGRVRFGFASLENDPEWLIPGKNPGDPFHGGPVMLSLLERPRPTWSIPGSDNKFKVPG |
| | RKFYVHHHAWKTIKDGNHPTTGKAIEQSPNNRTVEALAGGNSFSFEIAFENLKEWELGL |
| | LIHSLQLEKGLAHKLGMAKSMGFGSVEIDVESVRLRKDWKQWRNGNSEIPNWLGKGF |
| | AKLKEWFRDELDFIENLKKLLWFPEGDQAPRVCYPMLRKKDDPNGNSGYEELKDGEFK |
| | KEDRQKKLTTPWTPWA |

### Examples of Csx29

The tgRNA dissociates from the effector complex after the Cas7-11-mediated cleavage and that the Csx29 protease is only active as long as a target RNA is bound to the Cas7-11-Csx29 complex. In certain example embodiments, the Csx29 has a sequence listed in Table 2. In certain example embodiments, the nucleic acid encoding Csx29 has a sequence listed in Table 6.

TABLE 2
shows examples of Csx29 protein sequences.

| SEQ ID NO & | |
|---|---|
| PROTEIN | |
| ID/CONTIG | Sequence |
| SEQUENCE ID | MSNPIRDIQDRLKTAKFDNKDDMMNLASSLYKYEKQLMDSSEATLCQQGLSNRPNSFS |
| NO: 35 | QLSQFRDSDIQSKAGGQTGKFWQNEYEACKNFQTHKERRETLEQIIRFLQNGAEEKDAD |
| CSX29 | DLLLKTLARAYFHRGLLYRPKGFSVPARKVEAMKKAIAYCEIILDKNEEESEALRIWLY |
| | AAMELRRCGEEYPENFAEKLFYLANDGFISELYDIRLFLEYTEREEDNNFLDMILQENQD |
| | RERLFELCLYKARACFHLNQLNDVRIYGESAIDNAPGAFADPFWDELVEFIRMLRNKKS |
| | ELWKEIAIKAWDKCREKEMKVGNNIYLSWYWARQRELYDLAFMAQDGIEKKTRIADS |
| | LKSRTTLRIQELNELRKDAHRKQNRRLEDKLDRIIEQENEARDGAYLRRNPPCFTGGKR |
| | EEIPFARLPQNWIAVHFYLNELESHEGGKGGHALIYDPQKAEKDQWQDKSFDYKELHR |
| | KFLEWQENYILNEEGSADFLVTLCREIEKAMPFLFKSEVIPEDRPVLWIPHGFLHRLPLHA |
| | AMKSGNNSNIEIFWERHASRYLPAWHLFDPAPYSREESSTLLKNFEEYDFQNLENGEIEV |
| | YAPSSPKKVKEAIRENPAILLLLCHGEADMTNPFRSCLKLKNKDMTIFDLLTVEDVRLSG |
| | SRILLGACESDMVPPLEFSVDEHLSVSGAFLSHKAGEIVAGLWTVDSEKVDECYSYLVE |
| | EKDFLRNLQEWQMAETENFRSENDSSLFYKIAPFRIIGFPAE |
| SEQUENCE ID | MRYSSRTNCEAIDNLAEALQDQENMPEIARRVLEFEAENAKPENALCQHGLPHTKKAA |
| NO: 57 | SQIAGVRDKHSEFYDNALLDLVEEWLKTYEEAKKLTHRERRQEMEDKIRVLQPVLQAK |
| CSX29 | GKDADPRFLSLLARIYLYRGMLFRPKGFTTPARKIEALKKAVQLSEKAVEKEKDNPNFL |
| | RTWAQAALELEAIPETSFKVSSGLLKDAAVCINRDGIHSLNDLQVILEYAESEGKTSFLQ |
| | HVLVEKRYWKRPFDLFLLKARAAFALNRMDDVRYFLKSAMDKTPKALSSPFWDHLVD |
| | FLKKLRTKEGSDLWKEMAVAAHRLCREKEVKIANNIYLYRHWARQKSLYNMAFLAQN |
| | DLKEKAKIADSLKSRPVLRYQALREMKEHQNIAKLLEQDDQERDGGYHKQQVEMDER |
| | TGKRLSEKMEKAGVSYENLPVPWISVHFYLNESENSEDEGSKGYALIFDALTQSWKERR |
| | FDYAKLHRKFMTWQEAYISAKKSSFAKDSLVELCREIGNTMPFLFDTACIRDGAPVLWI |
| | PHGFLHRLPLHAAIRDEATNEIFLENHASRYLPAWSILNSASARRGKDSYMIKRFRAEDY |
| | EKEPFSELEDMEWDNEEHEKLATPDDLKHFMAKNPGVFAVLCHGHGDILNPLKSWLEL |
| | EGGGVSVLDILRYEKANLSGTRVLLGACEADMAPPVEYAIDEHVSLSAAFLSHKAQEVI |
| | AGLWEINIGEADECYAEILDCSDLSTELKDWQCDWVEKWRDDVEASGDNSTFYHITPF |
| | RIMGFPLKLKENNESEAKQ |
| SEQUENCE ID | MEHKTMTEPAGQNPSATDNDFEKFIIDTGCVFFATPQEDPKYQNNKVEWHQGLCRFAQ |
| NO: 58 | NDSPPTVIGSAIFFLQKLQEPGLFSGLPVSPELCSKISKDKNEIVAYHQQCILRLCEELLVK |
| CSX29 | GREAKEHRERRQAFDQAIKFLLVLKKGTSSDTPSPNGHIHFQDQVSILLAEAYYLRGKII |
| | RPKGFSVPAKKIETLEVAEKILVDLVARDTTGKARRLRAMVHIDLAALRDPADDSGNLQ |
| | DYRQALEQAVSSIGDTKTCGRDEIVIILARAEDNAGWTGSDGLSARLEELVNNGAAGPL |
| | DQARAYLLLGQNNLAVTQTEKAITRMAATDNPTPFSHEDWRLLVRLLRDLKHQNTAGI |
| | DKLILDTWRKVHQIERQTKNGMHVRWYWSRQRDLYDLAFHAAGNDARLKAQIADSL |
| | KARPALHLGQAADLGLAVEQMEAGLLDRYMPGKMLEQTTDMAAPAAPGSAGWPELP |
| | RPWIAVHFYLSNGFGHPEGKQQGHALIQDSSKGDGKDTWSERTFDYFPIWAAFMTWQE |
| | NYQRLKKEAAPDLERLCQVMGRQMPFLFAPEDLPLERPVVFVPHDFLHRLPLHAALIDN |
| | GEESGIPAQSHPITYLPGWWMVTSQAANPNETASKNTPSPVAPVALVHWDNSEDIHDII |
| | KQANGTVVVNASRSDWLKLKHNAVGLKVLYCHGQAGYTNPFASSLKLDGGGLYLKD |
| | VVKGPPLVGRFILAACESDLVLPASTTLDEYFSFSTGLLQKGAAEILGTLWEVNETDALS |
| | LIETVLRAPASGNLSFVLRDWLRDNLRSLTTELFYDIAAFRALGGPYPVDTKEEHR |
| SEQUENCE ID | MNTVELLQEEERLTLDLVFLPPGSKNKEQKKNALVDLLLKIVEHGELTRKYSALLTLSR |
| NO: 59 | GALRGEVHFGEKLLPSPEACANLAKPEEIKKMIRQHFQYRLDLLEAIVKKAADNTYSHA |
| CSX29 | RRRKALRIAIKELEQICEEALDELCFKARLLLAEALFERGRIVRPKGFSEPGKKKELFQKA |
| | INCIEGNCSEEALRLRARIYLQWYRFFHDEPPCDLDDIFTKALAVTDDKMLKTELLLLCG |
| | ERKEPDPYTDDLRALLNDQNVSPLSRARAAVLLEDWERCNVEIYEAIEDLGKTDFFQQD |
| | WELVVTLLKKNYNQFHGWSRACTRLWEITVEKESKDAGHGCVLRWYWSRQRDVYNL |
| | AFAAFEECEDKARVVDSLKNRPAHHFSQLEQLAQSSDIIKQWIESEEIINQDSFAHSLRRH |
| | EKGAKSHSGGSLRIFPCLPKGWIAVHFFLASWPEPKGYALIHNADTNTWEQRDFKYEQL |
| | WATYIAWQEVSLHNKIRESALLLKSLCETLGKEMRWLFDEFLFPKERRRVLFVPHDFLH |
| | RLPLHMAIDIESQTVFAAKQPVCYLPAYHLQNNITENKKTSIYALVNLRENKQQKKDEEI |
| | FAEKVEKMGAIVRRPALESDLLNLNPVPEKLVLYCHGIGHSANPFASKLCLGDTGVSYR |
| | DILALNRSLAGCRVLLFACETDLVPAQTSSIDEHLSISNALLQKGAFEVLGSLWALPGKTI |
| | YGITKTFIDNDDTSAVLHSSLKRLFEHYEKKNEKTRAQLLYNWASLRVLAPAREFS |
| SEQUENCE ID | MEEKHFLYNLYEKVKNYGDKAVFSGIKPSPGVCSKFKGLLNKPEATLTETFIKEVFKDE |
| NO: 60 | LRLEPNKAKARARTYDNLIRLLSYWQETPLLALLLARLCYERALLIMQKAYGKSKKKES |
| CSX29 | LLKQALNILDKILKHSDYPEALELKALVYLELKYMDVSPSDFSDILRPAFEKKKDCDAKI |
| | TLALAEAGNGEALTCLSSKTIPSNYNYLDRTRIAILENHRSLAKKWLNEALSKEVPFVFS |
| | SPWWDELIEVLNKLPPNLKFTFSTKAFEKIYTLERSFKIHNLHLLWYWSKLKDIYEMAFI |
| | ESISQKEYLKALFIADALKGRVMIKWHLMEKVLGEEFSDILEKEILGRLGYFVKSLEKKQ |
| | KPSSTTWSWPNLNEYFFDFIPPDFAVVHLFFTEKQFENQGYAFILQKDNNVELKSFNIEKI |
| | WNYFLQFKNAYLFADKYPVSTASFSSAVKSLLEILGEELSFLFDKIVCKYVLFIPYGFLH |
| | QLPLHAMKHEEKGYFFEKYLTAYFPAWSFVYTISPEENASKQIVMLKYFDRHKFSKLKN |
| | AFRSFSIKDPASKEDFLNLTSPLNTLVIVSHGEANLVNSFESTLKLNPPLTLKEILEHKNN |
| | AFRGSKVLLIGCETDLEVPPKKIVDEYISLSTIFLLKGAKEVIGTLWEVYADTAEEAFLKL |
| | LHTNGKESLEKYQQYLLQVLSEGEIQIEEYIPLRIHLHPKSYL |
| SEQUENCE ID | MNEEQTNRWPDLFDKFEDIIMEVDKKCDIEERTQFLRERKEFNAETLTSLEKADDWIRF |
| NO: 61 | GVILKVLSRESENSPVPVFTFRPSQNHCENLKKNSDKIDKAIAELNCKRAEKLLGHARTG |
| CSX29 | KVRKYTDRHRLVESAITLVWEQFEKRDNGQFKWLHIHVKTEKQACNFIAKCYLLRSKL |
| | ALPKGSSIPEKKLEALDNAWEWAKRGSPETDDLKMEVALQKHRWDPNLGKRWFQKQL |
| | NAFLDSNKLDLSNPLHWAVNDIVGDKALVSEEYDLEMLNDASVRNLTKNWEWKDKS |
| | GIPLYQARAAFRTHASDLDRRLINAVKKLKWLPLSHHLWEDTVALIKNVSEDDSENGK |
| | WEMAAILAWAICQNAEARIKLSVQLRWYWSRARELYDLAFQAALKRKRPFLLVRITDS |
| | EKSRPTIKMQAAEKSFANAAAFQTYLEAETLFATGNFNAGLKELNSVPIEKMRTRSVRA |
| | VPEGWAAVHFNIIDKNESHALIVENRECHSIRIDLPDVWDAFQKWNTERRDLKLIKKSET |
| | SLEILCEKSGIMLEPILNQIKSENILFIPYGFLHLVPLHASKIKKPDETYTYLFQEKQCLFLP |
| | SWSLAPVEKENIHTGEHDLLLLAKMRGKDIQNIMDREDWCNEKNAENIENTADDFFNC |
| | LFDTLERFRKPPHLLVLYCHGQGDFVNPYCSKFIMEGRPLTHQDIVQDLQDLPVLQGTK |
| | VILTACETDLVSRHFGLIDEHLSLATAFLCKGASQVIASLFTCTTDISCEIIVHAKDNPEKS |
| | LGQILQEKQNQWAANEALYRLSVFRVMGFPGSARAMEEEISP |
| SEQUENCE ID | MLLDSNALKEFLKKFKTFSQQSRKEQAKLLAKWEFFCNDTKFQAHPFGIEPEEELCKKV |
| NO: 62 | VKYSKDISLKSEAYLFLAREFIDNCKSQPNLLHREKRKYLEEAIRVLLEVIPEAETQKINL |
| CSX29 | LNEIYFTIAKAYLLRSQIFRPKGMTVPEKKKEALKKALEWVKRIDENNLEEAYLLKSEIY |
| | LELERIDERLADDAKETFEHGLNCKDCKAEPHIIAQIAVRWAELKNDINTKNILQTKVLE |
| | QSDVSHLEKAKAAFLLGQQNKVEQYLKRLSNELRNRYCLFSNPLWDGTVKFLKQLKDS |
| | NMDIWKDVSIKIWEVCEEKVRKASALHMRWYWSRQRDLYDLAFLAEEDPYKKAKIAD |
| | SLKSRPSQKYKVWEKEARKYLEQEEAALGKRYIKEIEYDETPLPKLVPFTSLPAPWIAIH |
| | FYLNHLEKKGYALIYDAKKQKWEQPLSFEFYPIFEAFEVWQTNYFERKIGAAKFLEGLC |
| | KEIGNQMSFLFELPSERPVLFITHDFIHRLPLHAAIKNRKLFLDNNPSMYLPAWGFITRND |
| | AENPRGRILLQNFKKYDFPKLKGMFGDQNPPPATRDHLKAMNEPPELLVILCHGTSDIV |
| | NPFSAKLHLAEGGITHREILRSELYINNSIVVLGACDTDLVPPITTSLDEHLSLNTAFLTKG |
| | ARAVVGTLWKIKAEEMETFILRLDNSSARSSNIVYIIQELQKEASKKWKQNKKPEILYES |
| | MCFKAIGYPLLRNTP |
| SEQUENCE ID | MENKAYSDALNLILEVDSKPDLIEGRQLLSQKRHVFEEALKTLKKAHSWSQLGALLCFL |
| NO: 63 | HEAYDYSLAPIEMPAREKALCANLDKYSDQIEEECTRINWQRAQGLRKTATDFSKEIKH |
| CSX29 | TSRERLLERAIRLAWSRFSPEGEWPYPAVAAKAEVCEFISRCYLERSKLALPKGSSIPEKK |
| | LEALNKAWHWAKKATATLNFCQMEIALERDRWEEDLPESWLETLLKEFLSSQKLDFKN |
| | PSHWVIAHRARSLRLGDSTYDKELLAIDPSKFEERKELLWLPLFQSYAALRLNNSEVPRF |
| | LERAINKLSRVPFSDPLWDATVSLVEEVAKEGKDKWEPVATKLWEICKEKEEQVRLSIQ |
| | LRWYWAQHQKLYALAFRAAIRQENCRLAAEIADSLKSRPTIKMMAIEKSMRSDEDREM |
| | SALQVEVDAVFAAGGFSHHYDNLLERVRELSKHKTPNQRRPIEDIPAGWAAVHFFLLSD |
| | EEGYALICKNGEFEKSPPLSLSRLWITYQAWEKARQTDPPDSYSLVEATERVCEALGDA |
| | FPFLFEIQENIIFIPHGFLHLLPLHAAKDDGRYLFLDKTCLYLPAWSLAPMGNKDSVSAQ |
| | DMLFVNWEDTALREQLLKHKWFVEIDSATGRDLIDNLNKSACPPGLLVIICHGQGDLTN |
| | PYNSRLLLAEGGITHRELLKSLPSVGGSRVILAACETDFAPSGSGILDEHLSVSAAFLQKA |
| | AGEVGGTLFAAMAEECSEFVLAAKANPEKPLYEVLQKKQNEWANKININRLVPFRIMG |
| | FPQNK |
| SEQUENCE ID | MNQNIDRAVGAILAIETATPLTESSTLAQRERHQKLLHDETKKIEQAFIALAQPPQCRAV |
| NO: 64 | EIAALSRFLQMTPLAVGPLRKRVICRAEPLKDDAHEQEIASHFNGLLLRLAKGLLASALN |
| CSX29 | PAGIPWRRRVLWLEKAAHIAHRFDKEPLADDKERTEAAGVLARCCLHLALAHLPKGKD |
| | KSAMAERQEDLLQSLMWAQKAIVLAGQDKLSGEEYKLLKALVLIELDNLSPGRFQQQL |
| | NYVLYDLAVIWLERDTATKPFHPQELFVLWRYLATDFEPDLNMLLFKGSNTSERTAAV |
| | QQASPEAERFRPLLPLIHAWSAWKLDPPNNKIAEVILQAVNNLDEHQVYEQVWKWTVD |
| | FLQELRNTGAVDWQLPAIAAWELCNKKEKELPFGFQIRQYWSRLDSLYRLAFDGALEL |
| | KDCMTAARIVDSLKSRTPLTWRDMDTLFAKLPKEKADQLREAFYSMEVQARMGFYAE |
| | AKEDANKLKKLLAAQVRKIRDIESVPAGWTVVHFHLREDQDLGYALACRLTADGMSY |
| | WTNHIFPVAGIRRAYDCWLEAYHGMEPGAREKSGYQLVELSEIMGKDLDFLFELAGED |
| | GARGLLFVPHGFSHLLPLHAAKKDGSYLFEKIPSLTLPAWEFAPDVDQIPVSDGQDFCFI |
| | SQRANEQDLVGNIERSHTWNGVCNKNAAWTNVLNTNKEWSKAPPRWLVFWCHGQAD |
| | PHVAFRSKLLLGTLGVSLFEIQEAALSLTGTKVVLAVCESDLAPPEEYEKTDDHLSLAAP |
| | FLLKGARQVLAAIWEGAQLDLLKAMKEMLSNQDKHSWEILRELQSCWMRQPGAIFND |
| | EYIRLYYAASFRILGFPEVATTNMATATAQEEIA |
| SEQUENCE ID | MTSLELIKKSYERGLSHSQVLSITLRDDNKWQQRLKNRNSAFREVLTSYVKSVQETAEII |
| NO: 65 | NYVGSALLFLNDDQDEYASSFDGVGFSAEKCSALAGAEDVILRFHLDQQIKLNNQILYN |
| CSX29 | IQEKGRLSHTIRRSALDQTIKNLLLMRQPPLCDLVDRKMQAQALSLSYLMRSRIIRQKGF |
| | SVPAKKIEGFQQALEALAFGYSKYPGDIEYLRIKSLILLEQDKIKATDSSGLEQCLKSYFG |
| | KLGIAGPDKADYPLILWYARKTNCHAYLDHILADGEPIEKLESAILLNCSSPEITGYANET |
| | ITDLSEKPFSHDDWKTIVAILKAHPGLALKDITIALWDAARQRESITTSNCHLRWYWSQQ |
| | QDIYEMAFHAADEASKKAEIADSLKGRPVLKRQAVEELARNDKSLKKYIDDQDAGWM |
| | GYIPKFKPAPSTSPPKKLKKTDNISQKKIFIATPQPWIAVHFFITTDAIGKKKGYAMVHDS |
| | QKNDQNNCWITHGPFNLDIVWSQYMIWQESYHRLGACGGDKNDSAPYMKKLCESIGK |
| | ELSFLFVLPREQPVVFIPNGFLHLVPIHMAIDVANSEKNPHQIWAYKRKFTYLPAWSLIGS |
| | DHGSASPYAASVQILKYFEEGEYNYNKLRRRNLLMNDQASSSDILALQNTSSHLFLLCH |
| | GTANPNRPFDSGLKLKNGRLTIREILSMPRIPGMAILLGACETDMVGATASPLDEHISVST |
| | SFLERGANEVIGGLFELRKKYTEDVALAIHDEMNNKYLYQIFTLFLSKKIDQYIKDKKCV |
| | SFYEMAAFRPLGLQSTVKSTPLENSVLKTQS |
| SEQUENCE ID | MTSSRTNCSFIDRIEKALQKEDLESTLPELALRLIEFETANAEPENALCQRGISNANNAAV |
| NO: 66 | RIAKALGEKSALADMAEVRIKDYEVRKPRLTHRQRRQYLEDTIRILQPEEEKSKESGML |
| CSX29 | ASLARVYLYRGVLYRPKGRITPARKTEAVRKAVRLSEKAIQNLSDKSGKAVFVWRTWA |
| | EAALELERAGDYSAPLETLEAAALQINADGITSLTDILILLRYAERSKKNAFKGKLTDLL |
| | DKKEHWWGHTSDIYLLKARIAFLFGHSDKEVWKYLKNALDHVPDAFSNPFWDDLVDF |
| | VKKLRDEESDMWKKTAIRAHGECRKKEAEIASGVVLRWYWSRQKDLYDLAFLAADH |
| | AEKKAEIADSLKSRPVLRYQTLRELKDIGTIGEILDREDEARDGRYLKTKPEPKEKEIVKE |
| | IKKKQAVPFKDMPEPWIAIHFYLNDFEEKGYALIFDATSRDDDGWKECRFDYRELHRKF |
| | MAWQELYFSGSEDSAADALVLLCREIGRAMPFLFDGTLPENSRVLWIPHGFLHRLPLHA |
| | AIRADENDTLFLEKHISRYLPAWNMLTSDSVKDNEASEDKGGFHMIKRLRPEDSDNYFK |
| | LNKRKWKNKEDEGIYRAREEDLKASMEKNPQALTLICHGHGDILNPLKSWLELEDSGM |
| | TVLDILKSEAKLSGTRVLLGACESDMAPPTEHTIDEHLSLCTVFLSHNAREIVAGLWEIQ |
| | TNMVDGCYNQILDSNDISEALKQWQEDQMKKRWKKKQDHTIFYLIAPFRVMGFPKRV |
| | SSEAN |
| SEQUENCE ID | MNNTEENIDRIQEPTREDIDRKEAERLLDEAFNPRTKPVDRKKIINSALKILIGLYKEKKD |
| NO: 67 | DLTSASFISIARAYYLVSITILPKGTTIPEKKKEALRKGIEFIDRAINKFNGSILDSQRAFRIK |
| CSX29 | SVLSIEFNRIDREKCDNIKLKNLLNEAVDKGCTDFDTYEWDIQIAIRLCELGVDMEGHFD |
| | NLIKSNKANDLQKAKAYYFIKKDDHKAKEHMDKCTASLKYTPCSHRLWDETVGFIERL |
| | KGDSSTLWRDFAIKTYRSCRVQEKETGTLRLRWYWSRHRVLYDMAFLAVKEQADDEE |
| | PDVNVKQAKIKKLAEISDSLKSRFSLRLSDMEKMPKSDDESNHEFKKFLDKCVTAYQDG |
| | YVINRSEDKEGQGENKSTTSKQPEPRPQAKLLELTQVPEGWVVVHFYLNKLEGMGNAI |
| | VFDKCANSWQYKEFQYKELFEVFLTWQANYNLYKENAAEHLVTLCKKIGETMPFLFC |
| | DNFIPNGKDVLFVPHDFLHRLPLHGSIENKTNGKLFLENHSCCYLPAWSFASEKEASTSD |
| | EYVLLKNFDQGHFETLQNNQIWGTQSVKDGASSDDLENIRNNPRLLTILCHGEANMSNP |
| | FRSMLKLANGGITYLEILNSVKGLKGSQVILGACETDLVPPLSDVMDEHYSVATALLLIG |
| | AAGVVGTMWKVRSNKTKSLIEWKLENIEYKLNEWQKETGGAAYKDHPPTFYRSIAFRS |
| | IGFPL |
| SEQUENCE ID | MKNRVQIEAIIRNLQGAARDSKTNKLSENIIAYDEYRKIHKSASLYQFGIIPAKESSSVLA |
| NO: 68 | ENETNHVACENAIFEMAEKNIENFSSEDIHKKRKETIESALRLLMGLYKDRHEKLQPRTF |
| CSX29 | VLIAKAYLLRSLITRPKGITIPEKKKEALKKGIGFVESAIKKIQSSENILSHSSDIDLLEKAW |
| | RIKSQLYLEYYRVNKDECDKNTLKEVLENSLISGCDKFDKNIEDVQIAIRYCELESSREY |
| | LEQIISSHLEGIEFEKARAYKLLELENENEDEIRKSMKVVIEEYLSGFSDPLWEDAVEFIN |
| | KLKSDNKNCWKELSLDMYKVCREQEAETASLHLRWYWSRQRRLYDLAFIAADKEEEK |
| | AKIADSLKSRLSLRWSALEETGKKSKNKREKEEISRILEAEAVAMLGGYIKGARKILKKR |
| | RRPLPDEQRSIPKDWIVIHFYVNQLENKCYALIYNKDENTWKCEFVKEYQRLFHVFLTW |
| | QTNYNRCKERAADSLVQLCKEIGNAMPFLFDECIIPQDKNVLFIPHDFLHRLPLHGAIHE |
| | KNNGVFLENHPCCYLPAWSFAAKENNAVVQGSILLKNFPEYSYEELVSNSTLWTSPVK |
| | DPASPDDLKTIIASPEMLVILCHGEADAVNPFNARLKLTGNGISHLEILQSTKMILKGSKII |
| | LGACETDLVPPLSDIMDEHLSITTAFLTNDAREILGTMYEALDVRISSIIQKIYRQEHYSS |
| | MMKQLWEWQKVGVENYRENGDTPAFYNTVVFRVIGLSI |
| SEQUENCE ID | MNDTLLRHLGLDIEKIAEEMQLLSADIEGNKEALVKTLVRYDEAKRIAKNAALWQFGL |
| NO: 69 | RPNQILFSVIDQTRQNQTMKEQAVRAVATQYLETFKQSREDGRDKCLTHNDQRELLES |
| CSX29 | ALKILVNFEKEMDGKIEPATCALIARTYLLRSAIMLPKGFTVPEKKKEALRKGSEYIRTID |
| | DLTEEALRVRGSLLLEQRHIDILEKNRESNGDNQTLIKELREALENGCDKENNTIEDVRIA |
| | LCYIELTDDKTDLLQKIIDSQLDFPGIELYRLKAYFLKGDYAAISDEALKEELSGIRENHP |
| | VWNEAMIFIKQLKDAQADCWRKLALAAYQVCRTRESETSSLHLRWYWSGYRLLYDLA |
| | FIAEDDLHRKAEIADSLKSRVSLHAKALDEIIKNDKEREEYYNAHAVAYAGGYVKGAG |
| | RIHTGRKEKDCDTNNVFKALPKDVAIVAFYLNYCEKNKDSRGRGYALIAENGTWNIKE |
| | FPFDSLYKAYLTWQTNYARHKESASPSLVELCEEIGRAMPFLFEITKKRIVFVPHDFLHR |
| | LPLHGAIKREWPKVLLEEYSCLYLPAWSLLHADTTKSSQTARKRMLIECFHEYDYHELQ |
| | TKINAQIKESKGVVWEKREKAKPKDLLQIPEAPEILMILSHGRADMTNPYYARLKLEGG |
| | DVSALEIMKAKTGTMSIKGSNVIMGCCETDLLPVLSTPIDEHVSPATALYTRGANFVVG |
| | TMWEINPIDIERHFIELLTKNDNSMLEGVGNWQREGLSDDKWKKHKESRFFYAIIGFRV |
| | LGIFT |

TABLE 6
shows examples of Csx29 DNA sequences.

| SEQ ID NO | Sequence |
|---|---|
| SEQUENCE ID | ATGAGATATTCATCCCGGACAAATTGCGAAGCAATTGACAACTTGGCAGAAGCTCT |
| NO: 70 | TCAGGATCAGGAGAATATGCCGGAGATTGCCAGACGGGTTCTTGAATTTGAGGCTG |
| | AGAATGCCAAACCGGAGAATGCCCTTTGTCAGCACGGACTGCCTCATACAAAGAAA |
| | GCGGCAAGTCAGATCGCCGGTGTTCGTGACAAACACTCAGAATTCTATGACAACGC |
| | GTTGCTTGATCTGGTCGAAGAGTGGCTGAAAACCTATGAGGAAGCGAAAAAGCTGA |
| | CCCACAGGGAACGACGTCAGGAGATGGAGGATAAAATACGCGTCCTTCAGCCAGTT |
| | CTCCAGGCAAAGGGGAAAGATGCTGACCCGCGCTTCTTGTCCCTGCTTGCCCGCATC |
| | TATCTTTACAGAGGAATGCTTTTCAGGCCCAAGGGATTTACCACACCTGCAAGAAAG |
| | ATTGAGGCTTTGAAAAAAGCGGTGCAGTTGTCTGAAAAAGCCGTGGAAAAAGAAAA |
| | AGATAATCCGAACTTTCTGAGAACATGGGCGCAGGCCGCGTTGGAGCTTGAAGCAA |
| | TTCCGGAAACATCTTTCAAAGTCTCCTCTGGTCTTTTGAAAGACGCGGCTGTCTGTAT |
| | AAACAGGGACGGAATTCATAGTCTGAATGACCTTCAGGTTATCCTTGAATATGCTGA |
| | AAGTGAAGGAAAGACTTCTTTTCTTCAACATGTTCTGGTTGAAAAACGTTATTGGAA |
| | ACGTCCCTTTGATCTGTTTCTTCTTAAAGCCAGGGCGGCTTTTGCCCTGAACCGGATG |
| | GATGATGTCAGATATTTTCTCAAATCGGCAATGGACAAAACGCCGAAAGCACTGTC |
| | CAGTCCTTTCTGGGATCATCTCGTTGATTTTCTGAAAAAGCTCAGGACAAAGGAAGG |
| | CTCGGATTTGTGGAAAGAGATGGCTGTGGCCGCGCATCGTCTATGTCGGGAAAAAG |
| | AGGTGAAGATCGCCAACAACATCTATCTGTACCGGCACTGGGCCAGACAAAAGTCG |
| | CTGTATAATATGGCTTTTCTCGCTCAGAACGATCTAAAGGAAAAAGCAAAAATAGC |
| | GGATTCGCTCAAATCCAGGCCGGTCCTCAGATATCAGGCATTGCGTGAGATGAAGG |
| | AGCATCAAAACATCGCCAAGCTTCTTGAGCAGGATGACCAGGAAAGGGACGGAGG |
| | CTATCATAAGCAACAGGTGGAAATGGATGAGCGAACCGGGAAAAGACTATCTGAA |
| | AAGATGGAAAAAGCCGGGGTGTCTTATGAGAATCTGCCGGTTCCTTGGATTTCAGTC |
| | CATTTCTATCTCAATGAATCAGAAAACTCTGAGGATGAAGGTAGCAAAGGATATGC |
| | GCTGATCTTTGACGCATTAACCCAATCGTGGAAGGAGCGGCGTTTCGATTATGCCAA |
| | ACTTCACCGGAAATTTATGACTTGGCAGGAGGCTTATATTTCTGCAAAAAAATCGTC |
| | TTTTGCGAAGGATTCTCTGGTGGAACTTTGCCGGGAGATCGGCAATACGATGCCGTT |
| | TCTCTTTGACACGGCGTGTATCCGGGATGGTGCTCCGGTGCTTTGGATACCTCATGG |
| | TTTTTTACACCGGCTTCCGCTCCATGCGGCCATTCGTGATGAAGCTACCAACGAAAT |
| | TTTTTTGGAAAACCATGCTTCCAGATATCTGCCGGCATGGAGCATTCTGAACTCAGC |
| | CTCCGCCAGAAGAGGAAAGGATTCTTATATGATCAAAAGGTTTCGTGCGGAAGACT |
| | ATGAAAAGGAGCCTTTTTCAGAACTGGAGGACATGGAATGGGATAATGAAGAGCAT |
| | GAAAAGCTCGCAACCCCTGATGATTTAAAACATTTTATGGCTAAAAACCCTGGGGT |
| | GTTCGCAGTTCTCTGTCACGGTCACGGTGACATTCTGAATCCTCTCAAGTCATGGTT |
| | GGAACTTGAAGGAGGCGGTGTCAGTGTACTTGATATTCTCAGATATGAAAAAGCGA |
| | ACCTTTCAGGAACCCGAGTCCTGTTAGGCGCATGCGAGGCGGACATGGCCCCGCCG |
| | GTGGAATATGCGATAGATGAGCATGTTTCATTGAGCGCTGCATTCCTGTCACATAAG |
| | GCTCAGGAAGTAATTGCAGGATTATGGGAGATAAATATCGGTGAGGCAGACGAGTG |
| | TTACGCCGAGATACTTGATTGCAGTGATCTTTCGACAGAATTGAAAGACTGGCAGTG |
| | TGACTGGGTTGAAAAATGGAGAGATGATGTTGAAGCCAGTGGAGATAATTCTACAT |
| | TCTATCATATTACCCCCTTCCGCATCATGGGCTTCCCCCTCAAACTGAAGGAAAACA |
| | ACGAAAGCGAGGCAAAACAATGA |
| SEQUENCE ID | ATGGAGCATAAAACCATGACCGAGCCTGCCGGGCAAAACCCATCGGCCACCGATAA |
| NO: 71 | TGATTTCGAAAAATTCATTATCGATACCGGCTGCGTTTTTTTCGCCACCCCTCAAGA |
| | AGACCCTAAATATCAGAATAATAAGGTTGAGTGGCACCAGGGGCTTTGCCGTTTTGC |
| | TCAAAATGACTCCCCGCCAACAGTAATTGGCTCAGCTATATTCTTCCTTCAAAAGCT |
| | CCAAGAGCCGGGGCTCTTTTCCGGTTTGCCCGTATCACCAGAATTATGTTCGAAAAT |
| | TTCGAAGGATAAGAACGAAATCGTTGCCTACCACCAGCAATGCATTTTGAGGCTTTG |
| | CGAAGAGTTGCTTGTAAAGGGCAGGGAAGCTAAAGAGCACCGCGAAAGAAGACAG |
| | GCATTCGACCAAGCAATAAAATTTTTGCTTGTCCTTAAAAAGGGTACCTCAAGCGAT |
| | ACCCCTTCCCCAAACGGCCATATTCATTTTCAGGACCAGGTTTCGATCCTTCTGGCA |
| | GAGGCATATTACCTGCGAGGCAAAATCATCCGACCCAAGGGTTTCTCCGTACCGGC |
| | CAAAAAGATCGAAACCCTTGAGGTGGCAGAAAAAATTCTTGTTGATCTCGTCGCTC |
| | GTGACACCACCGGCAAGGCTAGACGCTTGAGGGCAATGGTTCACATTGACCTGGCA |
| | GCTTTGCGCGACCCCGCTGATGACAGCGGTAACTTGCAGGACTATCGGCAGGCACT |
| | CGAACAGGCCGTTTCCTCCATCGGTGACACGAAAACGTGCGGTCGGGATGAAATCG |
| | TGATTATCCTGGCAAGGGCCGAGGATAATGCCGGGTGGACAGGAAGCGATGGGCTG |
| | AGCGCCCGGCTTGAAGAACTTGTGAACAACGGAGCAGCCGGACCATTGGACCAGGC |
| | CCGCGCTTACCTTTTGCTGGGACAGAACAACCTGGCGGTGACGCAGACGGAAAAAG |
| | CCATAACCCGAATGGCTGCCACCGACAACCCAACGCCCTTCAGCCATGAGGACTGG |
| | CGGTTGCTGGTTCGGCTGTTGCGTGACCTGAAACACCAAAATACAGCGGGTATTGAC |
| | AAACTCATTCTCGACACCTGGAGAAAAGTCCATCAGATCGAACGACAGACCAAAAA |
| | CGGTATGCATGTGCGCTGGTACTGGTCCCGCCAGCGGGATTTGTACGACCTGGCCTT |
| | TCACGCCGCCGGGAATGACGCCAGGCTGAAAGCGCAAATCGCCGACTCGCTCAAGG |
| | CCCGGCCCGCCCTGCACCTGGGCCAGGCCGCCGATCTTGGTCTTGCCGTGGAACAGA |
| | TGGAAGCGGGGCTTCTTGATCGCTACATGCCGGGAAAAATGCTCGAACAAACCACC |
| | GACATGGCCGCGCCGGCCGCGCCCGGTTCGGCTGGCTGGCCCGAACTGCCAAGGCC |
| | ATGGATTGCGGTACATTTTTATCTGAGCAACGGCTTCGGCCACCCCGAAGGAAAGC |
| | AGCAGGGCCACGCCCTGATTCAGGACAGCAGTAAAGGGGACGGGAAAGATACCTG |
| | GTCGGAAAGAACCTTTGACTATTTCCCCATCTGGGCTGCCTTTATGACCTGGCAGGA |
| | AAATTATCAGCGGCTGAAAAAGGAGGCGGCCCCGGATCTGGAAAGGCTGTGCCAGG |
| | TTATGGGCCGGCAGATGCCATTCCTCTTCGCCCCGGAAGACTTGCCACTTGAACGAC |
| | CGGTAGTGTTTGTTCCCCACGATTTCCTGCATCGCCTGCCCCTGCATGCTGCCCTCAT |
| | CGACAATGGCGAAGAAAGCGGGATTCCAGCGCAATCTCATCCGATCACCTACCTGC |
| | CCGGATGGTGGATGGTAACGAGTCAAGCGGCAAACCCCAACGAAACAGCATCTAAA |
| | AATACACCGTCGCCCGTGGCCCCAGTGGCCTTGGTTCATTGGGATAATTCAGAAGAC |
| | ATCCATGATATTATTAAACAGGCTAACGGCACTGTGGTCGTCAATGCCAGTCGATCC |
| | GATTGGCTGAAGCTGAAACATAATGCAGTGGGACTTAAGGTGCTCTATTGCCATGG |
| | CCAGGCTGGTTATACCAACCCTTTCGCCTCCAGCCTCAAATTGGACGGAGGCGGTCT |
| | GTACTTGAAGGATGTTGTTAAAGGGCCGCCTCTGGTCGGCCGCTTTATCCTTGCTGC |
| | CTGCGAGAGCGATCTGGTTCTGCCTGCCTCTACCACCCTGGATGAATATTTTTCCTTT |
| | TCTACCGGTTTATTGCAAAAAGGGGCCGCCGAAATTCTCGGCACTTTATGGGAAGTA |
| | AACGAGACCGATGCCCTCAGCCTGATCGAGACAGTCTTGCGGGCACCTGCTTCAGG |
| | CAACTTGTCTTTTGTGCTCAGGGACTGGCTCCGGGACAACCTCCGCTCTCTAACAAC |
| | AGAACTATTCTACGATATTGCCGCTTTTCGCGCGTTAGGCGGTCCATATCCAGTTGA |
| | TACAAAGGAAGAGCACCGATGA |
| SEQUENCE ID | ATGAATACAGTCGAATTACTTCAGGAGGAAGAACGCTTGACCCTGGATTTGGTCTTT |
| NO: 72 | TTGCCACCAGGTAGTAAGAATAAAGAGCAAAAAAAGAATGCTTTGGTAGACCTTTT |
| | GTTGAAAATAGTGGAGCATGGGGAATTAACCCGTAAATACTCGGCACTGCTGACCC |
| | TCTCTAGAGGGGCTTTACGGGGAGAAGTGCATTTCGGAGAAAAGCTTCTTCCATCTC |
| | CAGAGGCATGTGCAAACCTGGCCAAGCCGGAAGAGATAAAGAAGATGATAAGACA |
| | GCATTTCCAGTATAGGCTGGATTTGTTAGAAGCTATTGTAAAAAAGGCTGCTGATAA |
| | TACCTACTCTCACGCTCGCCGAAGAAAAGCGTTGCGAATTGCGATAAAAGAGCTGG |
| | AGCAAATATGTGAAGAAGCTCTGGATGAACTGTGTTTCAAGGCTAGATTATTGTTGG |
| | CCGAGGCGTTGTTTGAAAGGGGGCGGATTGTCAGACCTAAAGGGTTCTCTGAACCT |
| | GGAAAAAAGAAAGAGCTATTTCAAAAAGCTATTAACTGTATAGAAGGAAACTGTTC |
| | TGAAGAGGCCTTGAGACTAAGAGCCCGGATCTATCTTCAATGGTACCGCTTTTTTCA |
| | TGATGAACCACCTTGTGACCTGGATGATATTTTCACAAAAGCTCTTGCTGTAACTGA |
| | TGATAAAATGCTGAAAACTGAGCTTTTGTTATTGTGCGGGGAGCGCAAGGAGCCTG |
| | ATCCATATACAGATGACTTAAGAGCCTTGTTGAACGACCAAAATGTTAGTCCTCTCT |
| | CTAGGGCAAGAGCTGCGGTTCTTCTTGAAGATTGGGAACGATGTAATGTAGAGATA |
| | TATGAGGCCATTGAAGATCTTGGTAAAACCGATTTCTTTCAACAAGACTGGGAATTG |
| | GTCGTGACCTTGCTGAAGAAAAATTACAATCAGTTTCATGGGTGGTCCCGGGCATGT |
| | ACAAGGTTGTGGGAAATTACCGTTGAAAAGGAGAGCAAAGATGCCGGTCATGGCTG |
| | TGTTCTTCGTTGGTATTGGTCGAGGCAAAGAGACGTGTATAATCTTGCGTTTGCAGC |
| | TTTCGAAGAATGCGAAGATAAGGCCCGCGTTGTTGACTCTCTGAAAAACAGACCGG |
| | CCCATCATTTTTCTCAGTTGGAACAATTGGCTCAAAGTAGTGATATAATCAAACAAT |
| | GGATCGAAAGTGAAGAAATAATTAATCAGGATAGTTTTGCTCATTCTTTAAGGCGCC |
| | ATGAAAAGGGTGCGAAAAGTCACAGCGGTGGTTCTTTGCGTATATTCCCCTGCCTCC |
| | CAAAAGGATGGATTGCGGTCCATTTTTTTCTTGCTTCCTGGCCTGAGCCTAAAGGCT |
| | ATGCACTGATTCACAACGCCGATACCAATACATGGGAGCAAAGGGATTTTAAATAC |
| | GAACAATTGTGGGCGACCTATATCGCTTGGCAGGAGGTCTCCCTGCATAATAAAATC |
| | AGGGAATCTGCACTCCTGTTAAAGAGCCTTTGTGAAACATTGGGTAAGGAGATGCG |
| | GTGGTTATTTGATGAGTTTTTATTTCCAAAGGAGCGAAGGAGAGTGCTTTTTGTCCC |
| | CCATGATTTTCTCCATCGTCTCCCTCTGCACATGGCGATTGATATTGAATCTCAAACA |
| | GTTTTTGCCGCAAAACAGCCTGTTTGCTATCTCCCTGCCTATCATCTGCAAAATAATA |
| | TTACAGAGAATAAAAAGACAAGTATTTATGCGCTTGTTAATCTTAGAGAAAATAAG |
| | CAGCAAAAAAAAGATGAAGAAATATTTGCTGAAAAAGTAGAGAAGATGGGCGCTA |
| | TAGTGAGACGACCCGCACTGGAGAGTGATCTTTTAAATCTGAACCCGGTACCAGAA |
| | AAACTTGTTCTGTATTGTCATGGAATTGGACATTCAGCCAATCCTTTTGCATCTAAAC |
| | TATGCCTTGGTGACACTGGGGTGTCATATCGGGATATTCTTGCTTTGAACCGTTCTCT |
| | TGCGGGGTGCAGGGTTTTGCTTTTTGCCTGTGAAACAGATCTTGTTCCTGCTCAGAC |
| | ATCCAGTATTGACGAACATCTTTCCATTTCAAATGCCTTGTTGCAAAAAGGAGCTTT |
| | TGAAGTACTGGGGAGCCTTTGGGCCCTTCCAGGTAAAACGATTTATGGAATTACCAA |
| | AACCTTTATCGACAATGATGATACCTCCGCTGTGCTCCATAGTTCATTAAAAAGATT |
| | ATTTGAGCATTACGAGAAGAAAAATGAAAAAACTCGTGCACAGCTTCTCTATAATT |
| | GGGCGTCTTTACGTGTTCTCGCTCCTGCCAGGGAATTTAGTTGA |
| SEQUENCE ID | ATGGAAGAGAAACATTTTCTTTATAATCTTTATGAAAAGGTAAAAAATTATGGTGAT |
| NO: 73 | AAAGCTGTTTTTTCAGGTATTAAACCTTCTCCTGGAGTTTGCTCTAAGTTTAAAGGGC |
| | TTCTCAATAAGCCAGAAGCAACGTTAACGGAAACATTTATAAAAGAAGTATTTAAA |
| | GATGAACTTCGTTTAGAACCAAATAAAGCAAAAGCAAGAGCTAGGACTTATGATAA |
| | CCTTATCCGCTTATTATCCTACTGGCAGGAAACTCCTTTACTTGCACTTTTACTCGCA |
| | AGGCTATGTTATGAAAGAGCACTTCTTATTATGCAAAAGGCCTATGGTAAATCAAA |
| | AAAGAAAGAAAGTCTTTTAAAACAGGCTCTGAATATCCTTGATAAAATTTTGAAGC |
| | ACTCAGATTATCCAGAAGCATTAGAGCTTAAAGCCCTTGTATATCTTGAGCTAAAGT |
| | ATATGGATGTTTCTCCATCCGATTTTTCAGACATATTACGACCTGCTTTTGAGAAAA |
| | AGAAGGACTGCGATGCTAAAATAACGCTTGCTCTAGCAGAAGCCGGAAATGGAGAA |
| | GCGCTAACGTGTTTAAGTAGTAAAACCATCCCTTCTAATTACAATTATCTAGACAGA |
| | ACACGCATTGCTATACTCGAAAATCACCGTTCATTAGCAAAGAAATGGTTAAATGA |
| | AGCTCTTTCTAAAGAAGTCCCTTTTGTTTTTTCCTCTCCTTGGTGGGATGAACTTATA |
| | GAGGTGTTAAATAAACTTCCACCAAACCTAAAATTTACTTTCTCTACAAAAGCATTT |
| | GAAAAAATCTACACCTTAGAAAGAAGTTTCAAAATACACAATCTTCACCTCTTGTGG |
| | TATTGGTCAAAGTTAAAAGACATTTATGAGATGGCATTTATTGAATCAATTAGCCAA |
| | AAAGAATACTTAAAGGCACTCTTTATTGCTGATGCTCTAAAAGGAAGGGTAATGAT |
| | AAAGTGGCACCTTATGGAAAAAGTGTTAGGAGAAGAATTCTCTGATATCCTAGAAA |
| | AGGAGATCCTAGGTAGGCTTGGATATTTTGTTAAGAGTTTAGAAAAAAAACAGAAG |
| | CCATCTTCTACAACATGGAGTTGGCCTAATTTAAACGAGTACTTTTTTGACTTTATTC |
| | CTCCAGACTTTGCGGTAGTTCATCTCTTCTTCACTGAAAAACAATTTGAAAATCAAG |
| | GTTATGCCTTTATTTTGCAAAAAGATAATAATGTTGAGTTAAAATCTTTTAATATAG |
| | AAAAAATTTGGAATTACTTTTTGCAATTTAAAAATGCATATCTTTTTGCTGATAAAT |
| | ACCCTGTGTCAACTGCTAGCTTTTCAAGCGCTGTTAAATCTCTTTTAGAAATTTTGGG |
| | CGAAGAATTATCTTTTCTTTTTGATAAAATCGTTTGCAAATATGTCTTGTTTATACCA |
| | TATGGTTTTTTGCATCAACTTCCATTACATGCAATGAAACATGAAGAAAAAGGCTAT |
| | TTCTTTGAAAAGTATCTTACAGCTTACTTTCCTGCTTGGAGTTTTGTTTATACTATTT |
| | CACCTGAAGAGAATGCATCAAAACAAATTGTAATGCTGAAATATTTTGATAGGCATA |
| | AATTTTCTAAACTAAAGAACGCTTTTCGTTCTTTTTCTATTAAAGACCCTGCTTCAAA |
| | AGAAGACTTTTTAAATCTAACGTCTCCTTTAAATACTCTTGTTATTGTTTCTCATGGT |
| | GAGGCAAACTTGGTTAATTCCTTTGAATCAACTTTAAAATTGAACCCTCCCTTAACT |
| | TTAAAAGAAATCTTAGAACATAAAAACAATGCCTTTAGGGGCTCAAAGGTTCTTTTA |
| | ATCGGATGTGAAACAGATCTGGAGGTGCCACCTAAAAAAATAGTAGATGAATATAT |
| | TTCACTATCAACGATATTCTTGCTTAAAGGAGCAAAAGAAGTAATCGGAACGCTTTG |
| | GGAGGTTTACGCCGATACAGCAGAAGAAGCTTTCTTAAAGCTTCTTCATACAAATGG |
| | TAAAGAAAGCCTAGAGAAATATCAACAATACCTTTTGCAAGTTCTTTCTGAAGGAG |
| | AAATTCAAATTGAAGAATATATACCTCTTCGCATTCATTTACACCCAAAAAGTTATT |
| | TGTAG |
| SEQUENCE ID | ATGAATGAAGAACAAACAAACAGATGGCCGGACCTTTTCGACAAATTTGAAGATAT |
| NO: 74 | TATCATGGAGGTCGATAAAAAATGTGATATTGAAGAACGGACCCAATTTCTCAGAG |
| | AGCGCAAGGAGTTCAACGCTGAAACCCTTACCAGCTTGGAGAAAGCCGATGACTGG |
| | ATCCGTTTTGGCGTGATTCTCAAAGTCCTGTCAAGAGAATCTGAGAACTCCCCTGTG |
| | CCGGTGTTTACATTCCGGCCCTCACAGAACCATTGCGAGAATCTGAAAAAGAATAG |
| | CGATAAGATTGATAAAGCCATTGCGGAACTTAACTGTAAGCGTGCTGAGAAATTAT |
| | TGGGTCATGCCCGGACCGGAAAGGTCAGAAAATATACGGACAGGCATCGCTTGGTG |
| | GAATCAGCCATCACGCTGGTATGGGAACAGTTTGAAAAACGTGATAATGGCCAGTT |
| | CAAATGGCTTCATATCCATGTTAAAACTGAAAAACAGGCCTGTAATTTTATCGCAAA |
| | ATGTTATCTCCTCAGATCCAAGCTTGCGCTTCCCAAAGGCTCAAGTATTCCTGAGAA |
| | AAAACTGGAAGCCTTGGACAATGCGTGGGAATGGGCCAAAAGAGGATCACCTGAA |
| | ACAGATGATCTGAAGATGGAAGTTGCTCTCCAAAAGCACCGGTGGGATCCCAATCT |
| | TGGGAAGAGGTGGTTTCAAAAACAACTCAATGCTTTTCTTGATAGCAATAAACTTGA |
| | TCTTTCCAATCCCCTGCACTGGGCAGTGAACGACATTGTGGGGGACAAGGCCCTGGT |
| | TTCCGAAGAATACGATCTTGAGATGTTGAATGACGCGAGCGTCCGTAATCTTACGAA |
| | GAATTGGGAATGGAAAGACAAATCCGGCATCCCATTGTATCAGGCCCGGGCCGCAT |
| | TTCGCACCCACGCATCAGATTTGGACAGGAGACTCATAAATGCTGTAAAAAAACTG |
| | AAATGGCTTCCGCTTTCCCATCATCTTTGGGAAGATACTGTCGCCCTGATCAAAAAC |
| | GTCTCGGAAGATGACAGCTTCAATGGAAAATGGGAAATGGCAGCGATCCTGGCATG |
| | GGCAATATGTCAGAATGCCGAAGCTCGCATCAAACTGAGTGTGCAGTTGCGATGGT |
| | ACTGGTCCAGAGCCAGGGAACTTTACGACTTGGCTTTTCAGGCCGCGCTGAAAAGG |
| | AAAAGACCTTTTTTGCTGGTCAGGATCACCGATTCTGAGAAAAGCCGGCCCACCATC |
| | AAGATGCAGGCCGCGGAAAAATCGTTTGCGAATGCTGCCGCGTTTCAGACATACCT |
| | TGAAGCGGAAACCCTGTTTGCGACGGGCAATTTTAACGCGGGCTTGAAAGAACTAA |
| | ATTCTGTCCCCATTGAAAAAATGAGAACCCGAAGTGTTCGGGCGGTGCCGGAAGGA |
| | TGGGCAGCGGTTCACTTTAACATCATTGATAAAAATGAGAGTCACGCATTAATTGTT |
| | GAAAACAGAGAATGCCATTCGATTCGCATTGATCTCCCGGATGTGTGGGATGCTTTT |
| | CAGAAATGGAACACGGAGCGTCGTGATCTTAAACTGATAAAAAAATCCGAAACGTC |
| | ATTGGAAATCCTTTGTGAGAAATCCGGCATCATGCTTGAGCCGATACTGAATCAGAT |
| | CAAATCGGAGAATATCCTGTTCATCCCTTATGGTTTTCTTCACCTCGTGCCGCTCCAT |
| | GCGTCAAAGATAAAGAAACCGGATGAAACATACACATATCTGTTTCAGGAAAAACA |
| | ATGCCTGTTTTTGCCTTCCTGGTCATTGGCTCCGGTGGAAAAGGAGAATATTCATAC |
| | AGGTGAACATGATCTTCTGCTGCTGGCAAAGATGAGAGGAAAGGACATCCAGAATA |
| | TCATGGATCGAGAAGATTGGTGTAATGAGAAGAACGCAGAGAACATTGAGAACACC |
| | GCGGATGACTTCTTCAACTGTCTTTTTGATACACTGGAGAGATTCAGGAAACCGCCT |
| | CATCTCCTTGTTCTTTATTGTCACGGGCAGGGAGACTTTGTGAATCCCTATTGCTCAA |
| | AATTCATAATGGAGGGGAGACCCCTGACTCATCAGGATATTGTTCAGGATCTGCAA |
| | GACCTGCCGGTTTTACAAGGCACGAAGGTCATCCTCACTGCCTGCGAAACAGACCTT |
| | GTCAGTCGTCATTTTGGTCTGATAGATGAACATCTCTCACTGGCCACTGCTTTTCTGT |
| | GCAAAGGGGCCAGCCAAGTCATTGCCTCACTCTTCACATGTACGACGGATATCTCAT |
| | GTGAGATCATTGTCCATGCAAAAGATAACCCGGAAAAGTCTCTGGGACAGATCCTT |
| | CAGGAAAAACAGAACCAGTGGGCGGCCAACGAAGCATTATACAGACTGTCGGTCTT |
| | CCGGGTGATGGGCTTCCCCGGTTCGGCCCGGGCGATGGAAGAGGAGATATCGCCAT |
| | GA |
| SEQUENCE ID | ATGTTGCTTGACTCAAATGCGCTAAAAGAATTTTTAAAAAAGTTCAAAACATTTTCT |
| NO: 75 | CAGCAAAGTCGAAAAGAGCAAGCCAAACTGCTAGCTAAGTGGGAATTCTTTTGCAA |
| | TGATACCAAGTTCCAGGCCCACCCTTTTGGAATTGAGCCAGAAGAAGAGCTATGTA |
| | AGAAGGTAGTGAAGTATTCCAAAGACATTTCCCTAAAGAGTGAGGCTTATCTCTTTT |
| | TGGCCAGGGAATTTATAGATAATTGTAAATCTCAACCAAACCTCCTTCACAGGGAA |
| | AAACGTAAGTATCTTGAAGAAGCAATTCGTGTTTTACTGGAAGTCATTCCAGAAGCT |
| | GAAACACAAAAAATTAATTTATTAAATGAGATTTACTTCACCATAGCCAAGGCCTAT |
| | CTGCTTCGCAGTCAGATTTTCCGTCCAAAAGGAATGACCGTTCCAGAAAAGAAAAA |
| | AGAGGCCTTAAAAAAGGCTTTGGAATGGGTCAAAAGGATTGACTTTAATAATTTAG |
| | AAGAAGCATATCTGCTTAAGTCTGAGATATACTTGGAACTAGAGAGAATCGATGAA |
| | AGGTTGGCAGACGATGCTAAAGAAACATTTGAACATGGTCTTAATTGTAAGGATTG |
| | TAAGGCAGAGCCACATATAATTGCCCAAATTGCTGTGCGCTGGGCAGAATTAAAGA |
| | ATGATATTAACACTAAAAATATCCTACAAACAAAAGTTCTGGAGCAAAGCGATGTG |
| | AGCCACCTTGAAAAGGCAAAGGCAGCTTTTCTTTTGGGTCAGCAAAATAAGGTTGA |
| | GCAATATCTAAAAAGGCTTTCTAATGAACTTAGAAATCGTTATTGTCTTTTTAGTAA |
| | CCCTTTATGGGATGGTACTGTAAAATTTTTAAAACAGCTAAAAGACAGTAATATGGA |
| | TATCTGGAAAGACGTTTCAATTAAAATCTGGGAAGTCTGTGAAGAAAAAGTGCGTA |
| | AGGCAAGTGCGCTTCATATGCGCTGGTATTGGTCTCGCCAAAGGGACTTATATGACC |
| | TTGCTTTTCTAGCAGAAGAAGACCCATACAAAAAAGCAAAAATTGCAGATTCCCTA |
| | AAAAGTCGCCCTTCTCAAAAGTATAAAGTATGGGAAAAGGAAGCAAGAAAGTATCT |
| | AGAACAAGAAGAAGCCGCATTGGGCAAAAGATATATCAAAGAAATTGAATATGAT |
| | GAAACTCCTTTACCAAAACTAGTCCCGTTTACATCCCTTCCTGCTCCATGGATAGCC |
| | ATCCACTTTTATCTAAATCACTTGGAGAAAAAAGGATATGCTTTAATTTATGATGCA |
| | AAAAAACAAAAGTGGGAACAGCCGCTTTCATTTGAATTTTACCCTATTTTTGAGGCA |
| | TTTGAGGTTTGGCAAACAAATTATTTTGAAAGGAAGATAGGAGCTGCCAAATTTTTG |
| | GAAGGCTTGTGCAAAGAAATTGGGAACCAGATGTCCTTTCTTTTTGAATTACCCTCA |
| | GAGAGGCCAGTACTGTTTATCACCCATGACTTTATTCATCGATTACCACTGCATGCG |
| | GCCATAAAGAATAGGAAATTATTTTTAGACAACAATCCAAGCATGTATTTACCCGCG |
| | TGGGGATTTATAACAAGGAATGACGCTGAAAACCCAAGAGGAAGGATTCTTTTACA |
| | GAATTTTAAAAAATATGATTTCCCAAAGTTAAAAGGCATGTTTGGGGATCAGAACC |
| | CACCGCCAGCCACCCGTGACCATCTAAAAGCCATGAACGAGCCGCCTGAATTATTG |
| | GTAATTCTTTGTCATGGGACATCAGATATAGTGAATCCCTTTAGTGCAAAACTTCAT |
| | TTAGCAGAAGGAGGAATAACACATCGTGAGATTTTGCGCTCAGAACTTTACATCAA |
| | CAATAGCATCGTTGTTTTAGGGGCCTGTGATACAGATTTAGTCCCTCCCATTACAAC |
| | GAGTTTAGATGAGCATCTTTCTTTAAATACAGCATTTTTAACAAAAGGTGCCCGCGC |
| | AGTGGTCGGAACATTATGGAAAATAAAGGCTGAGGAAATGGAAACATTCATTTTAC |
| | GACTGGATAATTCATCTGCCAGAAGCTCAAATATAGTATATATAATTCAGGAACTGC |
| | AAAAAGAAGCAAGTAAAAAATGGAAACAAAACAAAAAGCCAGAAATCTTATATGA |
| | GAGTATGTGCTTCAAAGCAATAGGATATCCTTTATTAAGGAATACTCCATGA |
| | ATGGAGAATAAAGCTTACAGTGATGCACTAAATCTGATCCTGGAGGTTGATTCAAA |
| SEQUENCE ID | ACCTGATTTGATAGAAGGCCGCCAACTGCTGTCACAAAAAAGGCATGTCTTTGAAG |
| NO: 76 | AAGCCTTGAAAACCCTAAAGAAGGCTCATTCCTGGTCTCAGCTAGGTGCCTTACTCT |
| | GCTTTTTACATGAAGCGTATGATTATTCCCTTGCTCCTATTGAGATGCCGGCACGAG |
| | AAAAGGCTTTATGTGCCAATTTAGATAAATACAGCGACCAAATAGAGGAAGAATGT |
| | ACAAGGATCAATTGGCAACGGGCCCAGGGGCTACGGAAGACCGCGACAGATTTCTC |
| | CAAAGAAATAAAGCATACGAGCCGGGAGCGACTTCTTGAACGGGCTATTCGCCTTG |
| | CCTGGAGCCGTTTTTCTCCCGAAGGAGAATGGCCTTATCCGGCGGTTGCCGCAAAGG |
| | CTGAGGTGTGTGAATTCATCTCCCGTTGCTACCTGGAACGTTCCAAGCTGGCGCTAC |
| | CAAAGGGAAGTTCCATTCCGGAGAAGAAGCTTGAGGCTCTGAATAAGGCTTGGCAT |
| | TGGGCTAAAAAGGCTACCGCCACCTTGAATTTTTGTCAGATGGAAATAGCCTTAGAA |
| | CGTGATCGCTGGGAAGAAGACCTGCCTGAAAGCTGGTTAGAAACGCTCTTGAAAGA |
| | GTTTTTAAGCTCGCAAAAGCTTGATTTTAAGAACCCCTCACATTGGGTAATTGCCCA |
| | CCGAGCACGTTCCCTAAGATTGGGCGACTCCACCTATGATAAAGAGTTACTTGCTAT |
| | TGACCCTTCTAAATTTGAAGAACGCAAAGAATTATTATGGCTTCCCCTTTTCCAATCT |
| | TATGCCGCCCTGCGTTTAAATAACTCTGAAGTGCCGCGCTTCCTTGAAAGGGCAATC |
| | AATAAGCTGAGCAGGGTGCCTTTCTCTGATCCCCTCTGGGATGCTACGGTATCGCTG |
| | GTAGAGGAGGTTGCAAAAGAGGGAAAGGACAAATGGGAGCCCGTAGCTACCAAAC |
| | TCTGGGAGATCTGTAAAGAAAAGGAGGAGCAAGTCAGGTTAAGCATTCAACTTCGC |
| | TGGTATTGGGCACAGCACCAGAAACTCTATGCCCTGGCCTTCCGCGCTGCCATTCGC |
| | CAAGAAAATTGCCGGCTCGCTGCTGAAATAGCAGATTCTCTCAAGAGCAGACCAAC |
| | CATCAAGATGATGGCAATAGAAAAGTCCATGCGCAGCGATGAGGATCGGGAAATGT |
| | CCGCCCTGCAGGTCGAAGTCGATGCTGTCTTTGCCGCAGGGGGGTTCAGTCATCATT |
| | ACGATAACTTACTTGAAAGGGTGCGAGAACTTTCCAAGCATAAGACTCCAAACCAG |
| | AGGCGGCCGATAGAGGATATCCCGGCAGGCTGGGCTGCCGTCCATTTTTTCCTCTTA |
| | AGTGATGAAGAAGGCTATGCCCTCATTTGTAAAAACGGTGAATTTGAGAAAAGTCC |
| | TCCCTTAAGTCTCTCAAGATTGTGGATTACCTACCAAGCCTGGGAAAAGGCCAGACA |
| | GACTGATCCGCCCGATAGTTACTCATTAGTAGAAGCGACGGAAAGGGTGTGTGAGG |
| | CACTTGGAGACGCCTTTCCTTTCTTATTTGAAATCCAGGAAAATATTATTTTTATCCC |
| | TCACGGCTTTTTACATCTGCTCCCCCTCCATGCGGCGAAAGATGATGGAAGATACCT |
| | CTTTTTGGATAAAACATGTCTCTATCTGCCTGCGTGGTCACTCGCCCCTATGGGAAA |
| | CAAAGATTCTGTATCCGCCCAAGATATGCTCTTCGTAAACTGGGAAGATACTGCTCT |
| | TCGGGAGCAGCTATTGAAGCATAAGTGGTTTGTCGAGATAGATAGTGCGACAGGAA |
| | GGGATCTGATTGATAACTTAAATAAGTCTGCATGCCCACCGGGATTGTTGGTGATCA |
| | TCTGCCATGGCCAGGGGGATCTTACTAATCCTTACAATTCCCGACTGTTGCTGGCCG |
| | AGGGAGGAATAACCCACCGGGAACTTCTTAAATCTCTTCCATCAGTTGGCGGTAGC |
| | AGGGTCATCTTAGCCGCCTGTGAAACAGACTTCGCCCCGTCCGGCTCAGGCATACTC |
| | GATGAGCATCTCTCTGTATCTGCGGCTTTTTTGCAAAAGGCAGCAGGTGAGGTAGGA |
| | GGTACATTATTTGCTGCAATGGCCGAGGAATGTAGCGAATTCGTCCTGGCGGCTAAA |
| | GCTAACCCAGAGAAACCTCTTTACGAAGTGTTGCAAAAGAAGCAAAACGAATGGGC |
| | CAATAAGATAAATATTAACCGGCTAGTACCCTTCAGGATCATGGGATTTCCCCAGAA |
| | TAAGTAG |
| SEQUENCE ID | ATGAATCAAAATATCGATCGTGCGGTTGGTGCAATTCTAGCGATTGAAACAGCGAC |
| NO: 77 | ACCCCTTACCGAATCTTCAACACTCGCGCAACGTGAAAGGCATCAGAAGCTGCTGC |
| | ATGATGAAACCAAAAAGATTGAGCAAGCCTTCATAGCCCTGGCGCAGCCTCCCCAA |
| | TGCCGCGCGGTTGAGATAGCAGCCCTCAGCCGCTTTCTCCAGATGACCCCCCTAGCG |
| | GTTGGCCCGCTCCGCAAACGGGTTATCTGCCGGGCCGAGCCTCTGAAGGACGATGC |
| | GCACGAACAAGAGATCGCCAGCCATTTTAATGGACTTTTGCTCAGGCTGGCCAAGG |
| | GGCTCTTGGCCAGCGCACTAAATCCTGCGGGCATTCCTTGGCGGCGAAGGGTTCTGT |
| | GGCTTGAGAAGGCTGCCCATATCGCCCACAGGTTCGACAAGGAGCCCTTAGCCGAT |
| | GACAAGGAAAGAACCGAGGCAGCTGGCGTCCTGGCCCGTTGCTGCCTGCATCTGGC |
| | CCTTGCCCATTTGCCCAAGGGGAAAGATAAATCCGCCATGGCCGAACGGCAGGAAG |
| | ACCTTTTGCAGTCCCTGATGTGGGCGCAAAAAGCAATCGTCCTGGCAGGCCAGGAC |
| | AAGCTTTCCGGCGAAGAGTATAAACTGCTCAAAGCCCTTGTCCTTATCGAGCTCGAC |
| | AATCTTTCTCCAGGCAGGTTCCAACAACAACTCAATTATGTTCTTTATGACCTGGCT |
| | GTAATTTGGCTTGAACGCGATACTGCAACCAAACCTTTTCATCCACAGGAACTCTTT |
| | GTCTTATGGCGATATTTAGCAACTGATTTCGAACCAGATTTAAATATGTTGCTTTTCA |
| | AAGGATCCAATACTTCCGAGAGGACGGCAGCCGTGCAACAGGCCTCACCGGAAGCG |
| | GAGCGTTTCCGGCCGCTGCTCCCCTTGATTCACGCCTGGTCAGCCTGGAAACTTGAC |
| | CCTCCGAACAACAAGATTGCGGAAGTAATACTGCAAGCAGTCAACAATCTTGACGA |
| | ACATCAGGTCTATGAACAGGTATGGAAATGGACCGTGGATTTTCTCCAGGAACTCC |
| | GCAATACCGGCGCGGTTGATTGGCAATTACCGGCGATAGCGGCCTGGGAGCTTTGC |
| | AACAAAAAAGAAAAGGAACTTCCTTTCGGTTTTCAAATCCGCCAGTATTGGTCGCG |
| | GCTTGATTCCCTGTATCGTCTCGCTTTTGATGGAGCTCTGGAACTTAAAGATTGCATG |
| | ACCGCTGCGCGGATCGTCGATTCCCTCAAGTCCCGAACCCCCCTTACCTGGCGCGAC |
| | ATGGATACCCTTTTCGCCAAACTGCCGAAAGAAAAGGCCGATCAACTCCGAGAGGC |
| | CTTTTACTCCATGGAGGTCCAGGCCCGGATGGGTTTCTATGCGGAAGCCAAGGAAG |
| | ACGCGAATAAGCTCAAAAAACTGCTGGCCGCCCAGGTCCGCAAAATTCGGGATATC |
| | GAATCCGTGCCGGCCGGCTGGACTGTCGTACACTTCCACCTGCGCGAGGACCAGGA |
| | CCTTGGTTATGCCCTGGCATGCCGTTTGACGGCAGACGGCATGTCTTACTGGACTAA |
| | TCACATTTTCCCGGTTGCCGGAATCCGCCGAGCTTATGACTGTTGGCTTGAGGCGTA |
| | CCACGGCATGGAGCCTGGAGCAAGGGAGAAAAGCGGATATCAGCTTGTCGAACTGA |
| | GTGAAATCATGGGCAAAGACCTGGATTTTCTCTTTGAGCTTGCCGGGGAAGATGGG |
| | GCCAGAGGGCTCCTCTTTGTTCCTCATGGTTTTTCCCATCTCCTGCCGCTGCATGCCG |
| | CGAAAAAGGACGGCAGCTACCTTTTTGAAAAAATACCATCTCTTACCTTGCCGGCCT |
| | GGGAGTTTGCCCCCGATGTTGACCAGATCCCGGTATCGGATGGCCAAGATTTTTGTT |
| | TTATCTCGCAAAGGGCAAATGAACAGGATTTGGTCGGAAATATAGAACGTTCCCAT |
| | ACTTGGAACGGAGTGTGTAATAAAAACGCCGCATGGACAAATGTGCTTAACACAAA |
| | TAAAGAATGGAGTAAGGCACCGCCGCGTTGGCTGGTGTTCTGGTGCCATGGCCAGG |
| | CTGACCCCCATGTCGCGTTTCGTTCAAAACTTCTGCTCGGCACCCTTGGCGTCAGCCT |
| | CTTCGAGATCCAGGAGGCTGCCTTGAGTCTCACCGGCACCAAGGTAGTCCTGGCTGT |
| | TTGCGAGAGCGATCTTGCGCCCCCGGAAGAATATGAAAAAACCGATGACCATCTCT |
| | CTCTGGCTGCCCCTTTTCTGCTCAAGGGAGCCCGCCAGGTCTTGGCCGCAATCTGGG |
| | AAGGCGCTCAGCTTGATCTGCTGAAAGCCATGAAAGAAATGCTCAGCAACCAAGAC |
| | AAACATTCCTGGGAAATCCTCCGAGAACTGCAAAGCTGTTGGATGCGCCAACCCGG |
| | TGCCATTTTTAATGATGAGTACATCCGCCTTTATTATGCCGCCTCTTTCCGGATACTG |
| | GGTTTCCCGGAAGTTGCGACTACAAATATGGCGACTGCAACCGCCCAGGAGGAAAT |
| | AGCATGA |
| SEQUENCE ID | ATGACAAGCCTGGAATTGATCAAAAAATCGTATGAAAGAGGTTTATCACATAGTCA |
| NO: 78 | GGTTCTCTCGATTACCCTAAGAGATGATAACAAATGGCAGCAACGCCTGAAAAATC |
| | GAAATTCGGCATTCCGTGAAGTCCTGACTTCGTATGTCAAATCCGTTCAAGAAACTG |
| | CTGAAATCATAAACTATGTGGGTTCTGCTCTTCTTTTTTTAAATGATGATCAGGATGA |
| | ATATGCAAGTTCATTTGATGGGGTTGGATTTTCTGCTGAAAAATGTAGCGCTCTGGC |
| | TGGAGCAGAAGATGTGATATTGCGTTTTCATCTTGATCAGCAAATCAAATTAAACAA |
| | TCAAATTCTTTATAATATCCAGGAAAAAGGTCGGTTGTCTCATACAATCCGTCGATC |
| | AGCCCTGGATCAAACCATCAAAAACCTGTTGTTGATGCGTCAACCGCCGTTATGCGA |
| | CCTTGTTGATCGTAAGATGCAGGCACAGGCGCTTTCACTATCATATCTGATGCGCAG |
| | TCGAATCATTCGTCAGAAAGGGTTTAGCGTACCGGCAAAAAAAATTGAGGGTTTTC |
| | AGCAGGCGCTTGAGGCTTTAGCGTTTGGGTATAGTAAATATCCCGGTGATATAGAAT |
| | ACCTCCGCATAAAAAGTCTGATATTATTGGAACAGGATAAAATAAAAGCCACAGAC |
| | AGTTCTGGACTGGAACAATGCCTTAAATCTTATTTCGGTAAATTGGGAATCGCTGGA |
| | CCTGACAAGGCGGATTATCCATTGATTCTATGGTATGCCCGAAAGACAAATTGTCAC |
| | GCCTATCTGGACCACATTTTGGCAGATGGGGAACCCATTGAAAAGTTGGAATCGGC |
| | CATTCTCTTAAATTGTTCTTCACCTGAAATTACAGGCTATGCAAACGAAACAATTAC |
| | CGACTTGTCTGAAAAGCCTTTTTCTCACGACGACTGGAAAACAATCGTTGCTATTCT |
| | CAAAGCACACCCCGGTCTCGCTCTGAAAGATATCACGATAGCTCTCTGGGATGCTGC |
| | ACGACAGCGAGAATCCATCACTACCAGCAACTGCCATTTGCGCTGGTACTGGTCCCA |
| | GCAGCAGGATATTTATGAAATGGCATTTCATGCCGCTGACGAAGCATCAAAAAAAG |
| | CTGAAATCGCTGATTCACTGAAAGGCCGACCGGTGTTGAAACGTCAAGCTGTTGAA |
| | GAACTGGCCCGAAATGATAAATCCCTGAAAAAATATATTGATGATCAAGACGCAGG |
| | CTGGATGGGATACATCCCTAAATTCAAACCTGCACCTTCTACCAGTCCACCGAAAAA |
| | ACTCAAAAAAACAGACAACATAAGCCAGAAAAAAATATTCATTGCAACGCCGCAAC |
| | CCTGGATAGCCGTTCATTTTTTCATAACGACTGACGCAATAGGTAAAAAAAAAGGTT |
| | ATGCGATGGTTCATGATTCTCAGAAAAATGATCAGAATAATTGCTGGATAACACAT |
| | GGGCCATTTAATCTCGATATCGTCTGGAGTCAGTATATGATATGGCAAGAGTCATAT |
| | CACCGTCTGGGAGCATGTGGTGGTGATAAAAACGACTCAGCACCGTACATGAAAAA |
| | ATTGTGCGAATCTATAGGAAAAGAACTATCTTTTCTTTTTGTTCTTCCGAGAGAACA |
| | GCCAGTAGTTTTTATTCCGAATGGTTTTTTGCATCTGGTACCAATTCATATGGCGATA |
| | GATGTTGCAAACTCTGAAAAAAATCCGCACCAGATATGGGCTTATAAGAGAAAATT |
| | CACATACCTCCCTGCATGGTCATTGATAGGTAGTGACCATGGATCAGCATCGCCATA |
| | CGCCGCAAGTGTACAGATTTTAAAATATTTCGAGGAAGGGGAATACAACTATAATA |
| | AACTGAGAAGAAGAAATTTGCTTATGAATGATCAGGCTTCTTCATCAGATATTCTTG |
| | CGTTACAGAATACATCATCCCATCTTTTTCTTCTCTGTCATGGAACAGCCAACCCAA |
| | ATCGTCCTTTCGATTCCGGTCTGAAACTAAAAAATGGCAGACTAACCATCCGCGAAA |
| | TACTTTCCATGCCGCGAATACCTGGTATGGCTATTCTGCTGGGAGCATGTGAAACCG |
| | ACATGGTTGGTGCAACTGCTTCGCCTTTGGACGAACACATATCGGTTTCTACATCAT |
| | TTTTGGAAAGAGGCGCCAATGAAGTTATCGGTGGATTGTTTGAGCTTCGGAAAAAG |
| | TATACAGAAGATGTTGCCCTTGCTATCCATGATGAGATGAATAATAAATATCTTTAC |
| | CAAATTTTTACGCTTTTTTTAAGCAAAAAAATCGATCAGTACATCAAGGATAAGAAA |
| | TGTGTTTCTTTTTATGAAATGGCCGCTTTCCGTCCGCTTGGATTACAATCAACAGTAA |
| | AAAGTACACCACTGGAAAATAGCGTGTTAAAGACGCAATCGTAA |
| SEQUENCE ID | ATGACATCATCCCGGACGAATTGCAGTTTCATTGACAGGATTGAGAAGGCTCTGCA |
| NO: 79 | AAAAGAGGATTTGGAAAGCACTTTGCCCGAACTGGCGCTGCGACTTATCGAATTCG |
| | AGACAGCAAACGCCGAACCGGAAAACGCTCTCTGTCAGAGAGGAATTTCCAATGCA |
| | AACAATGCCGCCGTCCGGATCGCCAAAGCCCTGGGGGAAAAGTCCGCACTGGCAGA |
| | CATGGCTGAGGTGCGGATAAAAGATTACGAAGTCCGAAAGCCGCGATTAACGCATC |
| | GTCAGAGGCGCCAGTATCTCGAAGACACCATCCGCATCCTTCAGCCTGAGGAGGAA |
| | AAGAGCAAGGAATCTGGCATGCTTGCCAGTCTGGCCCGTGTTTATCTTTACAGGGGA |
| | GTGCTTTACAGACCAAAGGGACGGATCACGCCAGCGAGAAAAACAGAGGCCGTCA |
| | GGAAAGCGGTCCGTCTGTCGGAAAAGGCCATCCAAAACCTGTCAGACAAATCCGGA |
| | AAAGCTGTTTTCGTTTGGAGAACATGGGCCGAGGCCGCGCTTGAACTGGAACGGGC |
| | TGGAGACTATTCAGCGCCTTTGGAAACACTCGAAGCAGCGGCCTTGCAGATCAATG |
| | CCGACGGAATCACGAGTCTGACTGATATCCTGATCCTGCTGAGATATGCGGAACGT |
| | AGCAAGAAGAATGCTTTTAAAGGCAAGCTCACCGACCTGCTGGATAAAAAAGAACA |
| | CTGGTGGGGGCATACCTCTGATATATACCTCCTGAAAGCCAGAATCGCTTTTCTGTT |
| | CGGGCATAGCGACAAAGAAGTGTGGAAATATCTGAAAAACGCACTCGACCATGTCC |
| | CGGATGCTTTTTCCAATCCCTTCTGGGACGATCTTGTGGATTTTGTGAAGAAGCTCA |
| | GAGACGAGGAGTCGGATATGTGGAAAAAAACAGCGATTCGCGCACATGGCGAATG |
| | TCGGAAAAAGGAGGCGGAGATCGCCAGCGGCGTTGTCCTGCGCTGGTACTGGTCAA |
| | GACAGAAAGACCTCTATGATCTGGCCTTTCTTGCCGCGGACCATGCCGAGAAAAAA |
| | GCAGAAATAGCGGATTCTCTCAAGTCCAGACCGGTACTCCGATACCAGACTCTGAG |
| | GGAACTTAAAGACATCGGAACGATAGGTGAGATTCTCGACCGGGAGGACGAGGCA |
| | CGGGACGGGCGATATCTGAAGACAAAGCCGGAACCTAAGGAAAAGGAGATAGTGA |
| | AAGAAATAAAGAAGAAACAGGCGGTGCCTTTCAAAGATATGCCCGAACCATGGATT |
| | GCCATCCATTTCTACCTGAATGATTTCGAAGAAAAAGGATATGCTCTGATTTTTGAT |
| | GCCACATCCCGGGATGATGATGGATGGAAAGAATGCAGATTTGACTACCGCGAGCT |
| | TCATCGGAAGTTTATGGCGTGGCAGGAACTGTATTTTTCGGGCAGTGAAGATTCCGC |
| | TGCGGATGCGCTTGTGCTTCTGTGCCGCGAGATCGGCAGGGCCATGCCTTTTCTTTTT |
| | GACGGCACACTTCCGGAAAACAGCAGGGTTCTTTGGATACCCCACGGCTTTCTGCAT |
| | CGGCTTCCCCTCCATGCCGCTATTCGCGCCGACGAGAATGATACGCTCTTTCTGGAA |
| | AAGCATATCTCCAGATATCTTCCCGCGTGGAATATGCTGACATCGGACAGTGTCAAA |
| | GACAATGAGGCTTCCGAAGACAAGGGCGGTTTCCACATGATAAAAAGACTCCGGCC |
| | CGAAGACTCGGACAATTATTTCAAACTGAACAAAAGAAAATGGAAGAATAAGGAA |
| | GATGAGGGAATATATCGGGCCAGAGAAGAAGATCTGAAGGCATCTATGGAAAAAA |
| | ATCCCCAAGCCCTGACGCTTATTTGTCACGGCCACGGCGATATCCTGAATCCTTTGA |
| | AATCCTGGCTGGAACTGGAGGATTCCGGGATGACGGTTCTTGATATTCTCAAATCCG |
| | AAGCAAAATTATCAGGAACCAGAGTTTTGCTGGGAGCCTGCGAATCGGACATGGCC |
| | CCGCCCACGGAACACACCATTGACGAGCATCTGTCTCTCTGCACCGTATTTCTCTCC |
| | CATAATGCCCGGGAGATTGTTGCGGGGCTGTGGGAAATTCAGACCAATATGGTTGA |
| | CGGATGTTATAATCAGATACTCGACAGCAACGATATTTCAGAGGCTTTGAAACAAT |
| | GGCAGGAAGATCAGATGAAGAAAAGATGGAAGAAAAAACAGGATCACACCATTTT |
| | TTATCTTATTGCCCCCTTCCGTGTCATGGGCTTCCCTAAACGGGTCAGTAGTGAAGCT |
| | AATTGA |
| SEQUENCE ID | ATGAACAATACAGAAGAAAACATTGACCGTATCCAGGAACCGACCAGAGAAGACA |
| NO: 80 | TTGATAGAAAAGAAGCAGAACGGCTTCTTGATGAGGCTTTTAATCCAAGGACCAAA |
| | CCCGTCGATAGGAAGAAGATAATTAATTCTGCCCTGAAGATACTCATCGGTCTTTAT |
| | AAAGAGAAAAAAGACGATTTGACTTCCGCTTCTTTTATCTCCATTGCACGGGCATAT |
| | TACCTCGTAAGCATTACAATCCTTCCCAAAGGCACTACTATCCCGGAGAAGAAGAA |
| | AGAGGCGCTGAGAAAAGGAATTGAATTTATTGATCGCGCAATTAATAAATTCAATG |
| | GATCTATCCTCGATTCACAACGTGCATTCAGGATCAAGAGTGTTCTGTCCATAGAAT |
| | TTAATCGTATTGACAGGGAGAAATGTGACAATATAAAGCTAAAGAATCTACTTAAT |
| | GAAGCAGTTGATAAAGGATGTACAGATTTTGATACATATGAATGGGACATACAAAT |
| | TGCCATCCGCCTATGTGAATTGGGAGTAGATATGGAAGGTCATTTTGATAATCTTAT |
| | TAAATCGAATAAGGCAAATGATCTCCAAAAGGCAAAGGCATACTATTTTATAAAAA |
| | AGGATGATCACAAAGCAAAAGAACATATGGATAAATGTACAGCATCACTGAAATAT |
| | ACGCCTTGTTCTCATCGTCTCTGGGATGAAACGGTAGGTTTTATTGAAAGGTTAAAA |
| | GGTGATAGTTCTACATTGTGGAGGGATTTTGCAATAAAAACTTATAGGTCTTGCAGG |
| | GTGCAGGAGAAAGAAACGGGTACCCTTCGCCTGAGATGGTACTGGTCACGACACCG |
| | GGTATTGTATGATATGGCCTTCCTTGCCGTTAAAGAACAAGCTGATGATGAAGAACC |
| | AGATGTAAACGTTAAACAGGCTAAAATAAAGAAACTGGCAGAAATAAGTGATTCAC |
| | TGAAGAGCCGTTTCTCACTCCGTCTGTCTGATATGGAAAAAATGCCGAAATCAGATG |
| | ATGAGTCAAATCATGAGTTTAAGAAGTTCCTTGATAAATGTGTAACAGCATATCAGG |
| | ATGGTTACGTAATTAATAGATCTGAAGACAAAGAAGGTCAAGGAGAGAATAAAAG |
| | CACAACTTCTAAACAGCCAGAGCCGCGTCCGCAGGCAAAACTGTTGGAGTTGACAC |
| | AGGTACCGGAAGGCTGGGTGGTCGTCCACTTTTACCTGAATAAACTTGAAGGAATG |
| | GGAAACGCCATTGTTTTTGACAAATGTGCAAACTCTTGGCAATACAAAGAGTTTCAG |
| | TATAAGGAGCTCTTTGAAGTATTTTTGACCTGGCAGGCAAATTATAACCTTTACAAG |
| | GAGAACGCGGCAGAACATCTTGTAACGCTGTGCAAAAAAATTGGCGAGACAATGCC |
| | TTTTCTTTTTTGTGACAATTTTATTCCTAATGGTAAAGACGTACTTTTTGTCCCCCAC |
| | GATTTCTTACACAGGCTGCCTCTGCATGGCTCAATAGAAAATAAGACGAACGGAAA |
| | ATTATTCCTGGAGAACCATTCTTGTTGCTATCTCCCAGCATGGTCATTTGCTTCAGAA |
| | AAAGAGGCTTCAACTTCTGACGAATATGTTTTACTGAAGAATTTTGATCAAGGTCAT |
| | TTTGAAACTTTGCAAAACAACCAAATTTGGGGGACGCAGTCAGTCAAGGACGGCGC |
| | GAGTTCTGATGACTTGGAGAATATTAGGAATAATCCTAGATTGTTAACCATCCTCTG |
| | CCATGGCGAGGCGAATATGTCAAACCCGTTCAGGTCCATGCTCAAACTGGCAAACG |
| | GCGGTATAACGTACCTCGAAATACTAAATTCTGTTAAAGGTTTAAAAGGCAGCCAG |
| | GTAATCCTGGGCGCATGCGAGACAGACCTTGTTCCACCACTATCTGATGTAATGGAT |
| | GAGCATTATTCTGTTGCAACGGCATTACTTTTAATAGGTGCTGCTGGAGTTGTCGGG |
| | ACTATGTGGAAAGTTCGTTCAAATAAAACTAAAAGCCTCATTGAGTGGAAGCTCGA |
| | AAATATAGAGTATAAGTTAAACGAATGGCAAAAAGAAACTGGCGGGGCAGCCTAT |
| | AAAGATCATCCTCCTACGTTTTATAGATCTATTGCTTTTCGTAGTATAGGATTCCCTT |
| | TATGA |
| SEQUENCE ID | ATGAAAAATAGAGTACAGATTGAAGCAATCATAAGAAATCTTCAAGGCGCTGCAAG |
| NO: 81 | AGACTCTAAGACGAACAAACTATCAGAGAACATCATTGCTTATGACGAATACAGGA |
| | AGATCCATAAGAGCGCTTCTTTGTACCAATTTGGCATAATCCCCGCGAAAGAATCAT |
| | CATCGGTGCTTGCAGAGAATGAAACTAATCATGTCGCTTGTGAAAACGCTATTTTCG |
| | AAATGGCAGAAAAGAATATAGAGAATTTTTCCTCCGAAGATATACATAAGAAACGC |
| | AAAGAAACGATTGAATCTGCCTTGAGACTACTTATGGGTCTTTATAAGGATAGACAT |
| | GAAAAACTTCAGCCGAGAACCTTTGTCCTCATCGCAAAGGCATACCTTCTGAGAAG |
| | CCTTATTACTCGTCCAAAAGGTATAACGATACCCGAAAAGAAAAAAGAGGCGCTGA |
| | AAAAAGGGATTGGCTTTGTTGAAAGTGCCATTAAAAAAATCCAATCCTCCGAAAAT |
| | ATTTTATCTCATTCTTCTGATATAGATTTGCTTGAGAAGGCATGGAGGATCAAAAGT |
| | CAGTTGTATCTTGAGTATTATCGGGTTAACAAGGATGAATGTGACAAAAATACATTA |
| | AAAGAAGTTCTCGAAAATTCCCTAATATCGGGATGTGATAAATTTGACAAAAATAT |
| | CGAAGACGTACAGATTGCTATCCGCTACTGTGAATTAGAGAGTAGTAGAGAATATT |
| | TGGAACAAATTATTTCCTCTCACCTGGAAGGTATAGAATTTGAGAAGGCCAGGGCA |
| | TACAAACTCCTTGAACTTGAAAATGAAAATGAAGATGAAATAAGAAAAAGCATGAA |
| | GGTTGTTATTGAAGAGTATTTATCGGGTTTTTCTGACCCATTATGGGAAGATGCAGT |
| | TGAGTTTATCAATAAACTTAAATCCGACAATAAGAATTGCTGGAAGGAACTATCGTT |
| | AGATATGTATAAGGTTTGCCGAGAACAAGAGGCGGAAACTGCGTCTCTCCATTTGC |
| | GTTGGTACTGGTCAAGACAGAGAAGGCTTTACGACCTGGCATTCATTGCAGCAGAT |
| | AAAGAAGAAGAAAAGGCAAAAATTGCCGATTCATTGAAAAGTCGCCTCTCGCTTCG |
| | TTGGTCAGCATTAGAAGAGACGGGTAAAAAATCAAAAAATAAACGGGAAAAAGAA |
| | GAAATAAGCAGAATCCTTGAAGCCGAAGCAGTAGCGATGCTTGGAGGATATATCAA |
| | GGGTGCACGGAAGATCTTAAAAAAGAGGAGAAGACCTTTACCTGATGAGCAACGTT |
| | CCATACCAAAAGACTGGATAGTCATCCATTTTTATGTAAATCAATTAGAAAACAAGT |
| | GCTATGCCCTTATTTATAACAAGGATGAAAATACCTGGAAGTGTGAATTTGTTAAAG |
| | AATACCAAAGATTGTTTCATGTCTTTTTGACTTGGCAGACAAACTATAACCGATGTA |
| | AAGAGAGGGCTGCGGATTCCCTTGTGCAACTCTGCAAAGAGATTGGGAATGCCATG |
| | CCCTTTCTCTTTGATGAATGTATTATTCCACAAGATAAAAATGTTTTATTCATTCCCC |
| | ACGATTTCCTGCATCGATTGCCGCTTCATGGGGCAATACATGAAAAAAACAACGGT |
| | GTATTTTTAGAAAATCATCCATGCTGTTACCTTCCTGCGTGGTCATTTGCTGCGAAAG |
| | AAAATAATGCGGTAGTACAGGGAAGCATCTTGCTCAAGAATTTCCCTGAATATTCAT |
| | ATGAGGAGTTAGTTTCCAATTCAACACTATGGACTTCTCCGGTAAAAGACCCGGCGA |
| | GTCCTGATGACCTCAAAACAATTATCGCTTCACCGGAAATGCTTGTTATTCTTTGTCA |
| | TGGAGAAGCAGATGCTGTAAACCCTTTCAATGCCAGGCTTAAGTTAACAGGAAACG |
| | GCATATCGCATCTCGAGATATTACAAAGTACAAAGATGATTTTAAAAGGCAGTAAA |
| | ATAATCCTGGGCGCTTGTGAAACAGACCTCGTACCGCCACTATCAGATATTATGGAT |
| | GAACATCTGTCTATTACTACAGCATTTCTTACAAACGACGCCAGGGAGATTTTGGGA |
| | ACGATGTACGAGGCACTTGATGTACGCATATCAAGCATCATTCAAAAGATATATAG |
| | GCAAGAACATTATAGTAGTATGATGAAGCAATTGTGGGAATGGCAAAAAGTTGGGG |
| | TAGAAAATTATCGTGAAAATGGTGATACACCAGCATTTTATAACACCGTTGTTTTTC |
| | GTGTTATTGGATTATCAATATGA |
| SEQUENCE ID | ATGAATGATACCTTACTAAGACACCTGGGTTTAGACATTGAAAAAATTGCCGAAGA |
| NO: 82 | GATGCAGTTGCTTTCCGCTGATATTGAAGGAAACAAAGAGGCTTTGGTTAAGACGCT |
| | TGTCAGATATGACGAGGCAAAAAGGATCGCCAAAAATGCCGCGCTCTGGCAGTTTG |
| | GTTTGAGGCCGAACCAAATACTATTCAGTGTGATAGACCAAACACGTCAGAATCAA |
| | ACCATGAAAGAGCAGGCGGTGCGGGCGGTCGCGACACAGTATCTCGAGACATTTAA |
| | ACAGTCAAGAGAAGACGGCAGGGATAAATGCCTTACCCACAATGACCAGAGAGAA |
| | CTCCTGGAATCAGCGCTGAAGATTCTCGTTAACTTTGAGAAAGAGATGGACGGGAA |
| | AATTGAACCAGCAACATGCGCGCTCATTGCGAGAACATATCTGCTCAGAAGCGCTA |
| | TTATGCTGCCTAAAGGTTTTACGGTGCCGGAAAAGAAAAAGGAGGCATTGCGAAAG |
| | GGTAGCGAATATATACGCACGATTGACGATTTAACGGAAGAAGCATTGCGCGTTCG |
| | CGGCAGTCTTCTTCTGGAACAGAGGCATATTGATATTCTGGAAAAAAATCGTGAATC |
| | CAACGGCGATAATCAAACTCTTATAAAAGAGCTGCGCGAAGCCCTTGAAAACGGCT |
| | GCGACAAATTTAATAACACAATTGAAGACGTGAGGATTGCCCTCTGTTACATCGAAT |
| | TAACGGATGACAAGACAGATCTCTTGCAAAAGATAATAGACTCTCAACTCGATTTCC |
| | CGGGGATCGAACTCTACCGGCTCAAGGCCTACTTTTTGAAAGGTGATTATGCCGCTA |
| | TCAGTGATGAGGCTTTAAAAGAAGAGCTTAGCGGTATCCGTTTTAACCATCCGGTCT |
| | GGAATGAGGCAATGATCTTTATAAAACAGCTTAAGGATGCACAGGCAGATTGCTGG |
| | AGAAAATTAGCGTTAGCTGCTTATCAGGTATGCAGAACGCGAGAAAGTGAGACGTC |
| | CTCACTTCACCTTCGCTGGTACTGGTCTGGATACCGGCTGCTGTATGATCTTGCTTTT |
| | ATTGCAGAAGACGACCTCCACAGAAAGGCGGAAATTGCCGATTCGCTAAAAAGCCG |
| | GGTTTCCCTTCATGCAAAGGCGCTGGATGAAATTATTAAAAACGACAAGGAGAGAG |
| | AAGAATACTATAATGCCCATGCAGTTGCTTATGCTGGTGGGTACGTGAAAGGGGCG |
| | GGAAGAATTCATACGGGAAGGAAAGAAAAGGACTGCGACACAAACAATGTCTTCA |
| | AAGCACTTCCAAAGGATGTTGCAATTGTTGCCTTTTATCTGAATTACTGTGAAAAGA |
| | ACAAGGACTCACGGGGAAGGGGCTACGCCCTGATTGCGGAAAACGGCACATGGAA |
| | TATAAAGGAATTTCCCTTTGACAGCCTTTACAAGGCATATTTGACGTGGCAGACAAA |
| | TTATGCACGGCACAAGGAGTCTGCGTCACCGTCTCTGGTCGAATTGTGTGAAGAGAT |
| | AGGCAGGGCAATGCCCTTTCTTTTTGAAATAACGAAGAAAAGAATTGTCTTTGTGCC |
| | ACATGATTTTTTGCATCGATTGCCGCTGCATGGGGCAATAAAAAGAGAATGGCCGA |
| | AAGTCTTATTGGAGGAATACTCTTGCTTGTACTTGCCGGCCTGGTCATTGTTACACG |
| | CCGATACAACAAAATCCTCACAGACGGCCAGGAAAAGGATGCTCATAGAATGTTTT |
| | CATGAATATGATTATCATGAATTACAGACAAAGATAAATGCACAGATAAAAGAGAG |
| | TAAGGGTGTTGTATGGGAAAAGCGAGAGAAGGCAAAGCCAAAAGACCTTTTGCAG |
| | ATTCCTGAAGCGCCTGAAATCCTCATGATATTATCGCATGGAAGGGCCGATATGACA |
| | AACCCCTATTATGCAAGGCTTAAGCTGGAAGGTGGAGATGTATCTGCTTTGGAAATC |
| | ATGAAAGCCAAAACCGGAACCATGAGTATCAAGGGCAGCAACGTAATCATGGGTTG |
| | CTGTGAAACTGATCTGTTGCCAGTATTATCAACACCCATTGATGAACATGTGTCGCC |
| | AGCGACAGCATTATACACCAGGGGGGCAAATTTTGTAGTTGGAACCATGTGGGAAA |
| | TAAACCCCATAGATATAGAAAGGCATTTCATTGAACTATTAACGAAAAATGATAAT |
| | AGTATGTTGGAAGGTGTTGGAAATTGGCAAAGAGAAGGGTTGTCAGATGATAAATG |
| | GAAGAAGCACAAAGAATCGAGATTTTTCTATGCTATTATTGGATTCAGAGTATTAGG |
| | TATCTTTACATGA |

### Examples of Csx30 Linkers

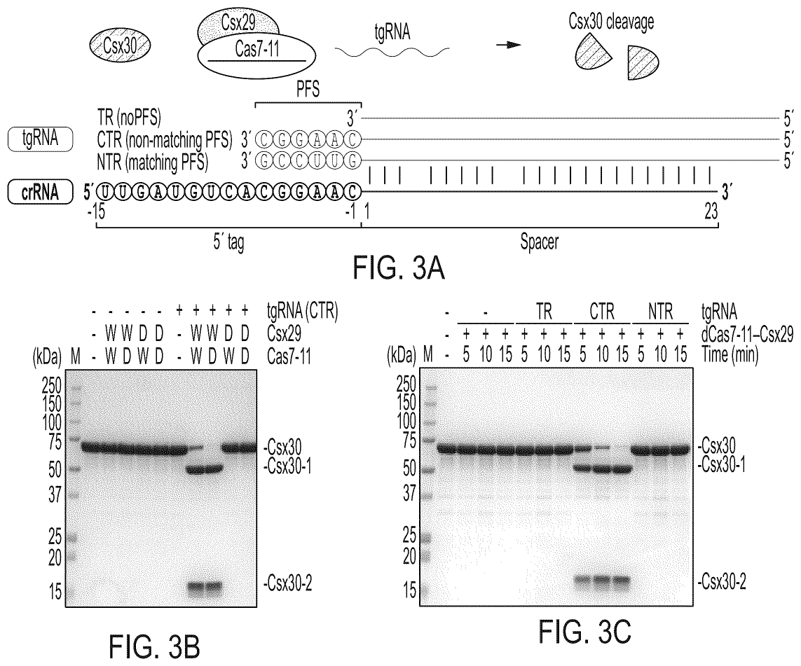

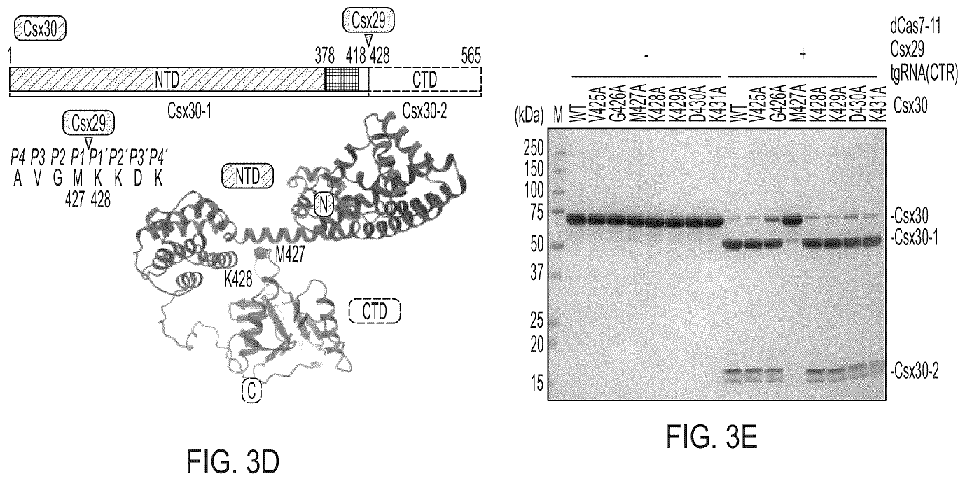

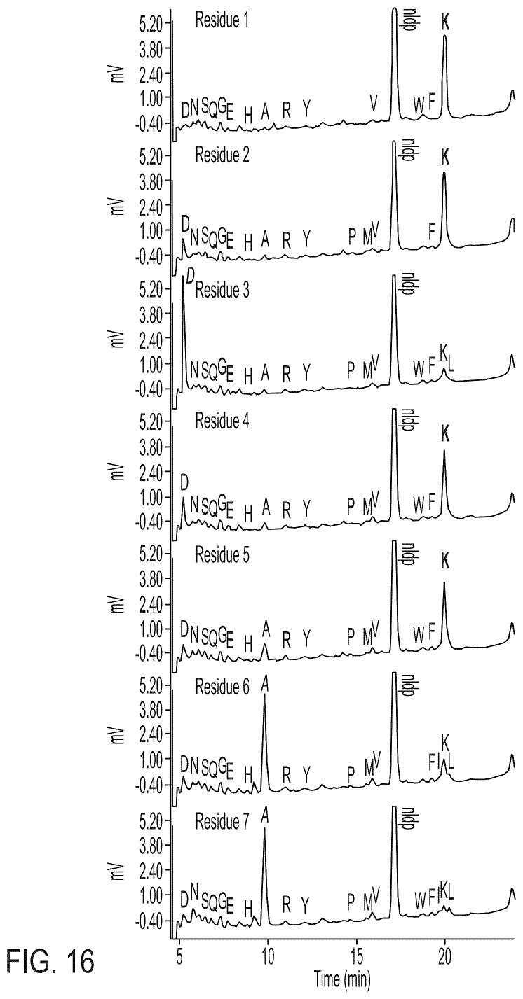

N-terminal analysis of the Csx30-2 fragment showed that it begins with K428 (FIG. 16), indicating that Csx30 is cleaved by Csx29 between M427 and K428 (FIG. 3D). A structural prediction using AlphaFold2 indicated that Csx30 consists of an N-terminal domain (NTD) and a C-terminal domain (CTD), which are connected by a linker region. The NTD (residues 1-377) contains two α-helical subdomains, whereas the CTD (residues 418-565) comprises a core β-barrel with flanking α helices (FIG. 3D). The cleavage site between M427 and K428 is located at a 0-hairpin in the Csx30 CTD (FIG. 3D). We examined the in vitro Csx29-mediated cleavage of eight Csx30 mutants, in which residues V425-K431 were individually replaced with an alanine. G416A and M427A mutations slightly and substantially reduced the Csx30 cleavage, respectively, whereas the other mutations had almost no effect (FIG. 3E). Thus, Csx29 seems to primarily recognize M427 at the P1 site within the AVGMIKKDK (SEQ ID NO: 37) sequence in Csx30 and cleaves Csx30 between M427 (P1) and K428 (P1′). Together, these results demonstrated that the Cas7-11-Csx29 complex catalyzes target RNA-triggered Csx30 proteolytic cleavage. In certain example embodiments, the Csx30 has a sequence listed in Table 3. In certain example embodiments, the nucleic acid encoding Csx30 has a sequence listed in Table 5.

TABLE 3
shows examples of Csx30 protein sequences.

| SEQ ID NO & | |
|---|---|
| PROTEIN | |
| ID/CONTIG | Sequence |
| SEQUENCE ID | MNTTTYNTTTDALLEWGKVYFQKEDFSEFLDNLEAYISDAGDSLKDELESGVEKLVLGI |
| NO: 36 | KSAEAVIFGEAVIGTTPENEAWYDAEESFLTLDCAVWLSQALDRVVRRQDASLADSLIA |
| CSX30 | RLDEAINRVAEKLYADNLSPLRFSSLNEIRRSALEATDEKYHYLFPWHGAACDVDENIL |
| | LILTEEYHLIGADKAGANLSEELRGDLPFIFAELERDEVLRAYVEKENALSLALENTMRE |
| | HWAFGLLEAARDEGYNHPYPADVGMRIHQVARAVFSQTNLSPAERLAVAIAGACFTPE |
| | ISEDRRLEILLDCEERVCEIEAPTGDDTSVRVIKDLKALADHRVRHEIPAESLVSLWFEQI |
| | EAAGTDFDTKTPMDELVLRMLSDNVITLSVDRKAASQTETDDVKPQKGKIIPFPVPDIAN |
| | DEVEYQKAVGMKKDKKAANDSKVKFPGLLEIQGCRDGDKAILLEDTDDAAANHRKLF |
| | SILKAGKLNSAFFIQSDDGEWVESESKPTMEDNRIILHDSHHSSFVWILDTGSMQLRQSV |
| | KCVKDALNKKTGSAKKLKPKTMIVWVTIPQEG |
| SEQUENCE ID | MQLTDKSRNELFSALLEWGKSHMLSPEIVQDISEIEENCEPFIKSDEFTNFLLNSVEKIRR |
| NO: 38 | QVEFAISLFTNIELVTATDEDSLWHDAEVSFIAFDRVNYLMEVLQFLIVPRKKEIAEKAM |
| CSX30 | KALDTIFEKTIGWLEESEFSPLRLVVLNESRRYHLEQIPEDERYRFPWYELYSDYSENTLE |
| | IIIENFDTFLSGKWEKLTREIPQEYLHEISLELKRDKLLLSRIKQEASYHKRLLAAVSKPS |
| | SLKLWRLGDEAALDYFLPGGVKKSGAVRVSLKLIEDAKIAFEADEICWKFLAAFCGPNLD |
| | DKQRLSLLDKVEERIKKIDIHKISENENILKTLKSWFEGNCDNAKLIKISFDTWIKMMEEK |
| | AINMHAVEFDESPEKLWNAIMELQKGRMGASNSVEKYINDFVNGCRQVWQIFKSTVDE |
| | PSVLYGARAIAASSEKSVQPRKLELNNNPILLSLKPNPKGEYIILSSLALKRVVLGEGVED |
| | YEKIWNYLEHTKNDYWCGCFITNDDKPDVASVQQIENRILAKKTRDYKKAIIGVSPEKI |
| | VLEEFIQELPAVIFEGKGPLKDSLVKKVIILVISLE |
| SEQUENCE ID | MYEHDYIGAILEWGKTKILSPHIIKYREDIEEYCKPLLESNEDELCDLLLRAIGELKDQVE |
| NO: 39 | SAISTFGDIKLVTSTEYEELWHKAGAEFCTFDKINYLMESVHYLICIRDKRKYNEILQELD |
| CSX30 | NVFSRITLWIEEGDFSPLRFVVLNEIRCESLLQIPEDERYRFPWYEAYSDYADDTVGIIIE |
| | NFNLFLSGNWDKLITEIPREHLIEVSFELKRDRQLFSVIELKAALHKNLLETMSKPTSLKL |
| | LAMWDDARLDYYLPEKVVEVGPGKVRDEVLKDVTSSREDTLFGRFLNAFCGPGMDDRQ |
| | RLNRLSKVEEEIKEVDISKASSPTKEVLSTLKLWFEGKCEDGKLTKISFETWLGKMERKA |
| | NEIDTESFNAHLETLLRGVPGLTKFLEEDVAKEPILEGSLCTIDLDDQQASTQKEQIPIKQ |
| | NPVGITLRLDREESISIMPDPDNIRASEDYKELWKFIDKVEDWYWGGTCFAEDKKNNLV |
| | FPLKTITYPLLGEIEGSKGYRFAVIGLSNNSEDLEIFVNKLKSVSITEKGQRTYPVDASGS |
| | RIAEEVRDYTQKPLNVVVLIIKYTYEE |
| SEQUENCE ID | MITTELEFSQTFDLLLKYGQISLETDFTDYRDELATEIRSTVAEKKDNDSIIQDISNLEDA |
| NO: 40 | INAACEFWKKIHHLVLSRDDESWSEAQDTFRIFDWSNYFSQAVFELKSDILNFHPELSLLI |
| CSX30 | LKSDELVGKVNDILIDDDTISMLRMVPLNSFRQWKISLLPENCRYMYPWYETYSELPDT |
| | FLASLAENWKNIGNGDVSQLETPENLSIEMIFDDLKADKILFQSIESNHHETILLKNALKS |
| | MYPHRLWNLSEDASFDNPLLEGVYEKGLIRLAIRILDEKVTPDEIIGQIFWVAFCGAGLS |
| | DKQRLENFKWVMPHIEKKCYVSENSIYLSILGKLQNWVAGESKGFEFANTVYKSWNEHLF |
| | KIANQHLLADVETDESEEQLINRINQIKAKIQIKDAIEDFKKIWESICTKLADIRSKENPF |
| | TVRCAGYAGVKEKEYTITYGKIPARLSIESIGGKIQLPNLPADLKVLEEVLLEEVLKDITE |
| | PYYSFGFSWNVDGEMKPIEEHNHDEDFNREIDDQFNDKKIQEILLVLEFDEKELKDTMK |
| | TFTEWIANPDVIKPPELKNVIVLYYSTS |
| SEQUENCE ID | MNQEVTTNKPTLKPGTLFSLPFPGPQMPSTYLALAQQDHRFECARASGGEEDYTGDHDIF |
| NO: 41 | WSGLIIEVWNTIYLTSELITAQGCALRPIPSDILEDCLEIRSQITEDQPEYPNDPNSSFDE |
| CSX30 | HTFQLIEKGANDALAKAIAGNSVTPLRIVPVWKKLGDLHEVITDKMKASILRTKLTLIYEN |
| | ALPPPQCLIWDSTDSFRIAVLAPDSNTNTVFTVWKKQMNSGWKAVVQVPYDIMKLALV |
| | TPEGSAILEILPSSNMQSAASLAERLFVARRESLAGLVFTSEPQLKTPHPYGVLEELATAN |
| | HSGLRALMLGLKNHIGFAVAAGLCCQNLPGILQLELFQLAEDMTKKSDSGKEGQEIGAL |
| | ADHLKIWQTSKSEPDWICKRAFKLWDNELVKNFAQAKAMKPSEDITHAFERYADWLT |
| | TWVPVLCEIEKGKTWTAIMAATIKENLSNVITGRLPQPQAAHATNVGRQPQDPSSANIL |
| | EAAITSIPDNWNPFDTAGTLRPLDARSRTAIKLLLENWKKKDQWFALLIIGEDNIHIVGPS |
| | NHCQTSPTAWEQNWISVFVILGHLEASVKKALDAVENIASGQPSNEDSPVLPTSPAEVA |
| | VIHWSINQ |
| SEQUENCE ID | MREEYYNSELTMTPLLKEMGRALLKTPNFVAGIQSMVAGFQEYSDGETIVLAEMSELLE |
| NO: 42 | AAREARVFLGGIQLNPPKNRKVQDEDELFLQAQEAFQALDSVLFCKTALEHLKKLDIW |
| CSX30 | KNTEVLSRLEEWQEDIAAWIGGEGGFSPLRLIVFQQIRRSILESFPEEVHYLFPWYEAFSE |
| | EEEGALFLLITNYDKIADPKKHSLLPEELRLKANAYYLELAMDKELRRDVAKAYKIHQA |
| | LAKAINESFAIRLFGILEEASMATPYPDDLLEYGFGSIAAQIIKGAKNIYWNRREQIGMAL |
| | LIGFCCEGLSDAVRFKLFSWVEDQMSKVDLGTSVNGSTGEKALSRLNTWVTSKVDKWL |
| | YSSEYENGLYALWFDEMRKNKNKVATSDEVSEEELCRLAEELIESYDKFWVIIQLLVGI |
| | QIIYKAASLALLSKVMKPLSLPVSMGPGAAPPPKFPTPYNLIGVPWVRREGIETFFEKSLR |
| | DIGDDAAKAYHAYEFSQERNHSYCVLYLGSEGEILDRRGPAPLPRRGKSIALELPENTAA |
| | VLFGIGPRTSLEAEWRILETPDLPVGEKSEVYWVLFAPPC |
| SEQUENCE ID | MNDKMIEKMITNRGKKVRPFHEAYEPEPGVIFLPPRQTEDWKSSYLIIEDVEDFYMCVR |
| NO: 43 | LSVREPAAAGIYDMIIEDMIIETWNALSIPKVMQIKGYRQISDKVTEAVLRVRRAWWDD |
| CSX30 | ELPDVSYPVGGDIFEVPDRLLFYESELQANEHLQRNLIEGTESLSRVKLWTRIGHKVGLT |
| | DYFEELQPIHLHGNFSAYIHHRYLFFLWEARHKTPPPEASVRETGSGLFEILSWKELKKE |
| | NIWEALVTIPEGVGSAVLKIGETECFLELLPSEDEPVSADSP |
| SEQUENCE ID | MESMRQYTTIFDTLIEIGRIKSINKGWLPEIEKYEKHILESAFRKNEEKAKQEMNHGINLL |
| NO: 44 | KEEVNKAKKSLFQDDIVFSDVELMEDTVEYCLNIFDEVVFAKIGIDEEAKKLENLKNIFN |
| CSX30 | VASQSIDKIFEGLFDWFYEEFEPYRLVLYNELRREYVRKIPEDLKHFFPWYNLYIEEDEN |
| | VFLKLALAGVGKQGKVKITPEFALAYEAIKRDKVLKAYVLQEYKDFKLFYDVIEENPYL |
| | ILFHEICEIGDFIELPEVIEQKGFFYVVDKTFNRLKERIRPIDALVLAVCCGPMEDERRIE |
| | WLKGLLDLPEQILKEGKHAREIAQFKETKRGVEHFIEKLFDVWVKECEKTAKELELEKKE |
| | ETYYQLFLSDVENLFSSISEKIKPYEPKPLEPILWLFIIAITKKLQRFSIRSDLMKEEAEE |
| | AVSSHFEIKTSLTVPLTLVPEARALKKRNVIKGDKRELEKALKRLRKFYYLVVAEKQGEAF |
| | LIEGPKERRSFPLIIEKISLPKDATFHVFLAYEKESLNCLLDISKQKEIETALKDILYIKV |
| | HLVD |
| SEQUENCE ID | MDMPPFYDLQFSPTMERLIGHARAEILDYPNHQTGGLPDVDQAILANDLVGFQKVLRQ |
| NO: 45 | ALSFLRSPATFDLAPGMGPSLAPVFNLLDNAVILEKYLIDRASLMGDKAVMQVLAIGEM |
| CSX30 | ATTKQAVPFHRFIPVNAWRRQFRDHLPEDVSCFFPWYDFWANEDELLLEEFVYLLPILQ |
| | QGDFSSFASQDAPRLRRLFAEIQRDKPFIAHLNKRHLLLQKTVQAVGKNTALRLFFLAD |
| | GCEPGVYLPELVLEKGLHTVAVAAMAQAPTSEEKRHELLFLAAFAGPELEDEERLAIFR |
| | KTVAFIGGAHALQEGGLMDKLRQWAKNTLDDQELSTSVYRRWRDLLNGTAPDIVTPF |
| | AYQDGQFSQVVGNLHTASVLEEKTVVPASDVQPGLWASLAAPAKKFWDNFTEIIGQFR |
| | ESLVLEPELSRAMSEEGGARERKPVEIPGLRLRQELMPEDGQFLTTESAREIRELLGDKIL |
| | YFITLAVDSATGQISATPPARLTRSSRIKDDIHGKPFVVGIGADKEGLEAAIDELTSPEGI |
| | RDQSKLDHILWIAITINDQILRDRHAP |
| SEQUENCE ID | MLNDYEERQTEGFLYSPALDLLLTWGSGSLFQGTEDEGEFVQGIEEEWHSLMEESDVKS |
| NO: 46 | ELHSGLEALTQDINDALDFLCHAIPESDRETALAQTALEILDHGVRLFQTTRNFSEQNHF |
| CSX30 | PEGFQVRAEALFRNITVDRAPSYIRLIPLNYWRQFMRGNISEHSFHLFPWYDEWADMPSE |
| | TIEILIENWNEISHGNFECSEMDTETLKVLFAELSNDRELLNYIQEEARFGHILPRAIGKS |
| | LVLRLLMIRNEEVGKHAAPQKVREKGFVACACHIIQKIKKIFRSEEERLEGIFLSGFCGPH |
| | LRENQRTELFTQVEHELKTLDVSLRPGSLLEELCQWSQGKVADEVFSRRVFEHWNNELS |
| | LSAKAVTERVPEDAAIFLRAVNELAFAKLETETVAEKIEWGIGRLFDINKLAVNELAFAK |
| | LETETVAEKIGRGIGCLPDKVINLIPSLIVRLKNPGYAGNGDDDDGNENGSVTLPRMINV |
| | CGHREGNNVNLLEASETIPDEIEKLLRQETGEVPKKLIPVMNSRKKLFYQVLAESSDDK |
| | WVFCFDKPKKTRAARIEVAETVYDRFVLLLDPGIKGLKHSSEVMMSVLNSVKAEDRQIS |
| | PYTVIIDLEIRDSEGGSK |

TABLE 5
shows examples of Csx30 DNA sequences.

| SEQ ID NO | Sequence |
|---|---|
| SEQUENCE ID | atgaatacaacaacatacaacaccacaacagacgcgcttttggaatggggcaaggtatattttcagaaagaggatttttctgaattccttgata |
| NO: 47 | atctcgaagcgtatatttcagatgccggggacagtctgaaagatgaacttgagagcggggggaaaagcttgttttgggcataaaatcggcg |
| | gaagcagttattttcggagaggctgtgatcgggactacgccggaaaatgaagcatggtacgatgccgaagaatcctttctgactttggattgt |
| | gccgtatggctgtcccaagcgcttgatcgcgttgttcgcaggcaggatgcgtcattggccgacagcctgatcgcccgcttggacgaagcca |
| | taaaccgcgtcgccgaaaaactgtatgcggacaatttatctccgcttcgtttttcatcgctcaatgaaatccgtcgaagcgctctggaggcaac |
| | ggatgagaaatatcattatctgtttccctggcacggggccgcatgtgatgttgatgaaaacattctgctgattctgacggaggaatatcatctg |
| | atcggggcagataaagccggagccaatctttcagaggaactgcggggcgacctgccttttatttttgccgagttggagcgggatgaggtgc |
| | tgcgggcatatgttgaaaaggagaacgccctttctcttgctttggaaaataccatgcgggaacactgggcattcggcctgctggaagcggc |
| | ccgtgatgaagggtacaaccatccctatcccgcagatgtcgggatgaggattcatcaggttgcccgtgccgtattttcccagacaaacctctc |
| | tccggcggaacgccttgctgttgccatagccggggcctgttttacgcctgagatcagcgaagaccggcggcttgagattctgctggactgt |
| | gaggagcgggtgtgtgaaatcgaagccccgaccggagacgatacatccgtccgcgttattaaagacttgaaagcgctggccgatcaccgt |
| | gtccgccatgaaatcccggcagaaagtcttgtcagtctgtggtttgagcagattgaggcggcggggacggattttgatacaaagacaccga |
| | tggatgaattggtgttgcgaatgctttccgataacgtcatcactctgtctgttgaccgaaaagcggcttctcagacagaaacagatgatgtgaa |
| | accgcagaaaggaaaaatcataccctttcctgttcctgatattgccaacgatgaagttgaataccaaaaagctgtgggaatgaagaaggata |
| | aaaaggcggctaatgacagcaaagtcaaatttcccggcctgcttgaaatccagggttgtcgtgacggggacaaggctattttgctggaaga |
| | cacagacgatgcggcagctaatcaccggaaactgttttccattctgaaagcaggtaaattgaattcagcttttttcattcaaagcgatgatgga |
| | gaatgggttgagtcggaatccaaaccgacgatggaagataaccgtatcatattacatgacagccatcattccagctttgtatggatattggata |
| | ccggttccatgcagctcagacagagtgtaaagtgtgtcaaagacgcattgaataagaagacagggagtgcgaagaaactgaaaccgaaa |
| | acaatgatcgtttgggtcacgattccgcaggagggatga |
| SEQUENCE ID | atgcaattaacagacaaaagtagaaacgaattgtttagcgctctccttgaatggggtaagtcacacatgctttcgccagaaattgttcaagata |
| NO: 48 | tctctgagatcgaggagaactgcgagccttttataaaaagtgatgaatttacaaactttttattaaattcagtggaaaaaatcaggcggcaagta |
| | gaatttgcaatttccttgtttaccaatatagagctagttactgcaactgatgaagattctctatggcatgatgcagaggtgtcttttattgccttcga |
| | tcgagtaaactatttgatggaggtattacagtttttaattgtaccccgcaaaaaggaaattgctgaaaaagctatgaaggcgttagataccatat |
| | ttgaaaaaacaataggctggcttgaagaaagtgagttttcaccattaaggttagtagtacttaatgaatcccgccgttatcatcttgaacaaata |
| | ccagaagatgaacgatacaggttcccttggtatgaattatattcggattatagtgaaaataccctggaaataattattgaaaacttcgacacttttt |
| | tgtcgggaaaatgggaaaaattaacgagggagataccccaagagtacctgcatgaaatatcgcttgaattaaaaagagataaactattacttt |
| | ctcgaataaaacaagaggcatcttatcataaaagattactcgctgcggtatcaaaaccctcatctcttaagttatggcgattgggagatgaagc |
| | ggcattggattactttcttcctggtggggtaaaaaaatctggtgcagtgcgagttagtttaaaattgatagaagatgcaaagattgcttttgagg |
| | cagatgagatttgttggaaattccttgctgctttttgtgggccaaatttggatgataaacaacgcctctccctgcttgataaagtggaggaaagg |
| | ataaagaaaattgatatacataaaatttcagaaaatgaaaatatcctgaaaacactaaaatcttggtttgagggaaattgcgataatgcaaaact |
| | tattaagatttcttttgacacttggattaaaatgatggaagaaaaagcaatcaacatgcatgctgtagagtttgacgaatcgccagaaaagttgt |
| | ggaatgccataatggaacttcaaaaagggagaatgggcgcttcaaattctgttgaaaaatatataaacgattttgttaatgggtgccgacaagt |
| | atggcaaattttcaaatcgaccgttgatgaaccatcggtattgtatggtgctcgggctattgctgcatcatctgaaaagtctgtgcaaccaaga |
| | aagttagaactcaataataatccaattcttctttcgttgaagccgaaccctaaaggagaatatattattttatcatcgctggcattaaaaagggta |
| | gttcttggtgaaggtgttgaggattatgagaagatatggaactatcttgagcataccaaaaatgattactggtgcggctgttttattacaaatgat |
| | gacaaacctgatgtagcttctgtacaacaaatagagaatcgaattttggcaaaaaaaacgagagattataaaaaagcaattattggtgtatctc |
| | ctgaaaaaatcgttttagaagaattcattcaggaattgccggctgtgatatttgaaggaaaaggcccgttaaaggattctttagttaagaaggtt |
| | atcatattggttatttcacttgaatga |
| SEQUENCE ID | atgtatgaacacgattatataggagcaatacttgaatggggcaagacgaagattttatccccacacattattaaatatcgcgaagatatagaag |
| NO: 49 | aatattgcaaaccattattagaatcaaacgaagatgaattatgtgatcttctcttaagagcaattggtgaacttaaagaccaggtggaatctgca |
| | atctctacatttggcgatattaagttggtgacatctactgaatacgaagagttatggcataaagcgggagcagagttttgtacttttgataagatt |
| | aattatctcatggaatctgttcattacttgatatgtattcgtgataagagaaagtataatgaaattttgcaagaactggataatgttttttcacgtatc |
| | actttatggatagaagaaggtgatttttctcctctcaggtttgtcgtcttaaatgaaatacgttgcgaatctttgttacaaataccggaagacgaac |
| | ggtatcggtttccatggtatgaagcttattctgattatgctgatgatacagttggaataattatcgagaattttaatctttttctatcaggcaactggg |
| | ataagttaataacagaaatacccagggaacatcttattgaggtatcttttgagctaaaacgtgaccgtcagcttttttctgtaatagaacttaaag |
| | cggccttgcacaaaaacctcttggaaacaatgtcgaagccgacttccttgaaactattggcaatgtgggatgatgcaaggctcgattattatct |
| | tcctgaaaaagtggtagaagttggaccaggaaaggttcgtgatgaagtgctcaaagatgtgactagttccagggaagacactctgtttggaa |
| | gatttttaaatgcgttttgcggtcctggcatggatgacagacaaaggttgaatagattgagtaaggttgaagaagagatcaaggaggttgatat |
| | ctcaaaagcgtcttcccctaccaaagaagtattgagtacactgaaactctggtttgagggaaaatgtgaggacggtaagcttaccaaaatctc |
| | ctttgagacatggcttggcaaaatggaaagaaaagcaaatgaaatagacactgaatcttttaatgcgcacttagagacactcttgaggggag |
| | tacctggattaacaaaatttcttgaagaagatgtagcgaaagaaccaatacttgaaggatctttgtgcactatagatttagatgatcagcaagc |
| | ctcaacgcaaaaggagcagattccaataaagcagaatccagtaggaataactttgagactcgacagggaggaaagcatatcgattatgcct |
| | gaccctgacaatatcagagcgtctgaagactacaaagaattatggaaatttattgataaggttgaagattggtactggggaggcacttgttttg |
| | ccgaagacaaaaaaaataatcttgtatttcctctaaaaaccatcacttacccattacttggtgagatagaagggagtaaaggttaccgatttgc |
| | agttatcggcttatcaaataacagtgaagatttagaaatatttgtgaataagttgaagagtgtatccattactgaaaagggtcaacgcacatatc |
| | ctgtagatgcatccggtagcaggatagcggaagaggtcagggattatacgcagaaaccactaaacgttgttgtgctgattattaaatatacat |
| | atgaagagtaa |
| SEQUENCE ID | atgattacaactgaattggaattttcacaaacttttgacttgctgctcaagtatggccagatcagtctggagaccgattttacagattatgtgac |
| NO: 50 | gaattggccactgagatcaggtcaacggtagcagaaaaaaaagataatgattctatcatacaggatatcagtaacctagaagatgccataaa |
| | tgctgcctgtgagttctggaagaaaatccatcacttggttttgtcgagggatgatgagtcatggtctgaagctcaggacactttcaggatttttg |
| | actggtccaattatttttcacaagcggtatttgaattgaagtctgatattcttaattttcatcctgagttatctttattaatcttaaaatcagacgaactt |
| | gttggcaaggtcaacgatatcttgattgatgacgataccatatctatgcttaggatggttccactgaacagttttagacagtggaaaatcagtct |
| | gcttcctgaaaactgccgatatatgtatccttggtatgaaacatacagcgaactgcctgatacgttcctggcatcattggctgaaaactggaaa |
| | aacattggcaatggcgatgtttcacaactggaaacacctgaaaacctttcaatcgaaatgatttttgatgaccttaaagccgacaaaatactctt |
| | tcagtccatagaatccaaccaccatgagacgatattactcaaaaatgcgctgaaatcaatgtatccccaccggctgtggaatctgagcgaag |
| | atgccagttttgataatccgcttctggaaggtgtgtatgaaaagggactgatccgtctggcaattcgtattcttgatgaaaaggttactccggat |
| | gaaataatcgggcagattttctgggtcgccttctgtggagccggtctctccgacaagcagcgattagaaaatttcaaatgggttatgccgcat |
| | atagagaaaaaatgttatgtgtccgaaaactccatttatctatctatactcggtaaactgcagaattgggtggctggtgaaagcaagggtttcg |
| | aatttgcaaatacagtctataagtcgtggaatgagcacctatttaaaatagcaaatcagcacctgctcgcagacgttgaaacagacgaatcgg |
| | aagaacaactgatcaacagaataaatcagataaaggccaaaattcagataaaggacgcaatagaggattttaaaaagatttgggaaagtata |
| | tgcaccaagttggcggatattaggagcaaatttaatccctttactgtacgatgtgcaggatatgctggggttaaagagaaagaatataccatca |
| | cctatggcaaaatacctgcccggttatcaatagaatctatcggtggcaaaattcagctgccaaatttgccggcggacttaaaagtattagaag |
| | aagttcttttagaagaagttcttaaagatattaccgaaccatattattcctttggtttttcatggaatgttgatggtgaaatgaagccaattgaagag |
| | cataatcatgatgaagactttaaccgtgagattgacgatcagtttaatgacaagaaaattcaggaaattctgcttgtgctggaattcgatgaaaa |
| | ggagttgaaagatacgatgaaaacctttacagaatggatagccaatcctgacgtgatcaagcctccagaattaaaaaacgtcattgtcctcta |
| | ttattccaccagctaa |
| SEQUENCE ID | atgaatcaagaagtaacaacgaataaaccaacgctaaaaccaggaacactgttttcccttcccttccctggtccgcaaatgccttcaacatac |
| NO: 51 | cttgctcttgcacagcaggatcatagatttgaatgcgctcgtgctagcggcggggaggaggactatactggagatcacgatatattctggtcc |
| | ggtctcataattgaggtttggaataccatttatctcacttccgaacttattaccgcgcaaggctgcgcccttcgccctattccttcggatattcttg |
| | aggactgccttgagataaggagtcagatcacagaagatcaaccggaatatccaaatgatccgaatagttcttttgatgagcatactttccagtt |
| | aattgaaaaaggcgctaatgacgcgcttgccaaagcaatcgcgggaaattctgttacccccttaagaattgtgccggtctggaaaaaactgg |
| | gtgatcttcatgaagtcattaccgataaaatgaaagcatcaatactgcggactaagttgaccttgatctatgaaaatgcattgcccccgccgca |
| | gtgcctgatctgggactctacagattccttccgcatagcagtattggctccagatagcaatacaaacactgtttttacggtctggaagaaacaa |
| | atgaacagcggatggaaggcagttgttcaagtcccctatgatataatgaagttagcgcttgttactcccgagggtagcgctattctggaaatc |
| | cttccatcttccaatatgcaaagtgcagcaagccttgcagaaaggctttttgttgcccggcgtgaatcgcttgccggcttggtctttaccagtga |
| | accgcagttaaaaaccccccatccttatggagtgcttgaggaacttgcgactgccaatcatagcgggttacgagctctcatgcttggcctgaa |
| | aaaccatattggttttgccgtggccgcaggcctctgctgccagaatctgcccggcatccttcagctagagctgtttcagctcgcggaagacat |
| | gactaaaaaatcagacagcggcaaggaaggacaagagatcggcgctctggctgaccacctcaaaatttggcagacaagtaaaagcgaa |
| | cctgactggatctgcaagagggcctttaaactatgggacaatgaacttgttaaaaattttgcacaagccaaggccatgaagccaagcgagg |
| | atattacccatgcatttgagcggtatgctgattggcttacgacttgggtgcctgtcctctgtgaaattgaaaagggaaaaacgtggacagcaat |
| | catggcagctactattaaggaaaatctttctaacgtaattaccggcaggcttcctcagccacaggcagcccatgcgaccaatgtcggcagac |
| | aacctcaagatccatcttcagctaacatcctcgaagcagccatcacatccataccagataactggaatccattcgacaccgcggggaccctt |
| | agacccctcgatgctagatcacgaactgcaatcaagctattgctggagaattggaagaagaaagatcaatggttcgctcttttgataattggc |
| | gaggacaatattcatatcgttggacccagtaaccattgtcagacctctcccacggcctgggagcagaactggatctccgttttcgttatccttg |
| | gccatttagaggcttcggtcaaaaaggcgctcgatgcggtcgaaaacattgcgtctggccagccaagtaatgaggactcgccagtattacc |
| | cacgagcccggcggaggtggcggttattcactggagcataaaccagtga |
| SEQUENCE ID | ATGCGCGAAGAATATTACAACTCTGAACTCACAATGACCCCCTTATTGAAGGAAAT |
| NO: 52 | GGGCAGGGCTTTACTGAAGACACCTAACTTTGTGGCGGGGATTCAGAGCATGGTAG |
| | CGGGATTCCAGGAATATTCGGACGGAGAAACTATTGTACTCGCAGAGATGAGTGAG |
| | CTTCTTGAGGCAGCAAGAGAAGCCAGAGTTTTTCTCGGCGGGATTCAATTAAATCCC |
| | CCTAAGAATAGAAAAGTGCAGGATGAAGATGAACTATTTCTTCAAGCTCAGGAAGC |
| | CTTTCAGGCGCTTGATAGCGTCTTGTTTTGCAAAACGGCACTGGAACACTTAAAGAA |
| | GCTAGATATTTGGAAAAACACGGAGGTATTGTCAAGACTTGAAGAGTGGCAGGAAG |
| | ATATAGCGGCCTGGATAGGGGGCGAAGGAGGTTTTTCTCCGTTAAGACTTATAGTCT |
| | TTCAACAGATCAGAAGGAGCATTTTGGAATCATTCCCCGAAGAAGTACATTATCTTT |
| | TCCCGTGGTATGAAGCTTTTTCCGAAGAGGAAGAGGGCGCTTTGTTTCTGCTAATTA |
| | CAAATTACGACAAAATAGCTGATCCTAAAAAACACAGCTTATTACCGGAAGAGTTA |
| | CGACTTAAAGCGAATGCATACTATTTAGAGTTAGCCATGGATAAAGAATTAAGGAG |
| | AGATGTTGCTAAAGCCTATAAAATACATCAAGCCCTAGCAAAGGCTATTAATGAAT |
| | CTTTCGCTATCCGGTTGTTTGGTATCCTTGAAGAGGCTTCAATGGCTACTCCCTACCC |
| | CGATGACTTGCTCGAGTATGGTTTTGGTAGCATTGCTGCCCAGATAATTAAAGGAGC |
| | CAAGAATATATACTGGAATCGTAGGGAACAAATAGGAATGGCATTGTTAATCGGAT |
| | TTTGCTGTGAGGGTCTGAGCGATGCCGTTAGGTTTAAACTCTTTTCGTGGGTTGAAG |
| | ACCAGATGAGTAAAGTCGATCTCGGTACATCAGTTAATGGCAGCACGGGGGAGAAG |
| | GCTCTTTCGCGTTTGAATACTTGGGTGACCAGCAAAGTAGATAAGTGGCTTTATAGT |
| | AGTGAGTATGAAAACGGTCTTTACGCCTTATGGTTTGACGAAATGAGAAAAAACAA |
| | GAACAAAGTTGCTACATCCGATGAGGTAAGTGAAGAAGAATTATGCCGGCTGGCCG |
| | AGGAATTAATAGAATCATACGATAAGTTCTGGGTTATCATCCAGTTGTTAGTTGGTA |
| | TACAAATTATTTACAAAGCGGCATCTCTAGCTCTTCTTTCGAAAGTTATGAAGCCTTT |
| | ATCTCTACCTGTTAGCATGGGTCCAGGAGCCGCTCCCCCTCCGAAGTTTCCCACTCC |
| | ATACAATCTCATCGGGGTCCCATGGGTCAGAAGGGAAGGGATCGAGACCTTTTTTG |
| | AAAAATCTTTGCGCGATATCGGCGACGATGCTGCAAAGGCGTATCATGCATACGAA |
| | TTCTCACAAGAGAGAAATCATAGCTACTGCGTACTGTACCTCGGTTCTGAAGGCGAG |
| | ATACTTGACAGAAGAGGGCCTGCTCCTCTTCCCCGAAGAGGTAAGTCTATAGCTTTG |
| | GAGCTTCCTGAAAATACGGCGGCCGTACTCTTTGGTATAGGACCGAGGACATCCTTA |
| | GAAGCAGAATGGAGAATACTGGAAACTCCTGACTTACCAGTTGGCGAGAAAAGCGA |
| | AGTATATTGGGTCTTATTTGCCCCCCCCTGCTGA |
| SEQUENCE ID | Atgaatgacaagatgatcgaaaaaatgattacaaacagagggaaaaaggtccgaccgttccatgaagcgtatgaaccggaacccggtgt |
| NO: 53 | gatcttccttccgcctcgtcagacagaagattggaagtcatcctacctgattattgaagatgttgaggatttttatatgtgcgtgcgcctgagtgt |
| | ccgagagccggccgcagccggaatatatgatatgattattgaagatatgattattgaaacatggaatgccctgagcattcccaaagtaatgca |
| | gatcaaaggttacagacaaatctctgacaaagtgacagaagcagtccttcgggtgcgccgggcatggtgggatgatgaacttccggatgta |
| | tcctatcctgtcggcggagatattttcgaggtgcctgaccggctgttattttatgaatccgaacttcaggcaaacgagcaccttcagagaaatc |
| | tgattgaaggaaccgaatcgctctcacgggtgaaattatggacccggatcgggcataaagtgggcctgacagattattttgaagaattgcag |
| | cccatccatttacatggaaatttttctgcctacattcaccatcgttatctcttttttttatgggaggcacgtcataaaacgccgccacctgaggcat |
| | ctgtcagagaaacgggcagcggactgtttgaaattctttcttggaaagaattgaagaaagagaatatatgggaagcgttggtgactattccgg |
| | aaggggttgggtctgccgtattaaaaatcggtgagacagaatgtttccttgaactgttgccgtccgaagatgaacccgtttcagcagattcgc |
| | catag |
| SEQUENCE ID | ATGGAATCCATGAGGCAATATACAACAATCTTTGATACACTTATAGAAATAGGTCG |
| NO: 54 | CATAAAAAGTATTAATAAAGGCTGGCTTCCAGAGATAGAAAAATATGAAAAGCATA |
| | TCTTAGAATCCGCTTTCAGAAAGAATGAAGAAAAAGCAAAGCAAGAAATGAATCAC |
| | GGGATAAATCTTTTAAAAGAAGAAGTGAATAAGGCAAAAAAAAGTTTATTTCAGGA |
| | TGACATTGTATTTTCTGATGTAGAATTAATGGAAGACACGGTTGAATACTGTCTGAA |
| | TATTTTTGATGAAGTTGTATTTGCAAAAATCGGCATAGATGAAGAAGCAAAGAAAC |
| | TAGAGAATCTTAAAAATATTTTCAATGTAGCATCCCAATCTATTGACAAAATTTTTG |
| | AGGGTCTTTTTGATTGGTTTTATGAGGAGTTTGAGCCTTATCGTTTGGTGTTATATAA |
| | TGAGCTTAGAAGAGAATATGTAAGAAAAATTCCTGAGGATCTTAAGCACTTTTTCCC |
| | ATGGTATAACCTTTATATAGAAGAAGATGAAAATGTATTTTTAAAACTGGCTTTAGC |
| | TGGAGTGGGTAAGCAAGGAAAAGTCAAAATTACTCCTGAATTTGCTCTTGCTTATGA |
| | GGCAATTAAAAGAGATAAAGTACTTAAAGCTTACGTTTTGCAAGAATATAAGGACT |
| | TTAAGCTCTTTTATGATGTTATTGAAGAAAATCCATATCTTATTCTTTTTCACGAAAT |
| | TTGTGAGATTGGTGATTTTATAGAACTGCCAGAAGTCATTGAACAAAAAGGATTTTT |
| | CTATGTAGTGGACAAAACGTTTAATCGCCTAAAAGAAAGAATACGTCCAATTGACG |
| | CCCTTGTCCTTGCTGTTTGTTGTGGACCTATGGAGGATGAAAGAAGGATTGAGTGGC |
| | TAAAAGGACTTTTGGATTTGCCTGAACAGATTTTGAAAGAAGGTAAACATGCAAGG |
| | GAAATTGCACAATTTAAAGAAACTAAAAGAGGGGTAGAACATTTTATTGAAAAACT |
| | CTTTGATGTATGGGTAAAAGAGTGTGAAAAAACGGCCAAAGAATTAGAGCTAGAAA |
| | AAAAAGAGGAAACTTATTACCAACTTTTTCTTAGCGATGTTGAAAATCTTTTTAGCT |
| | CCATTTCAGAAAAAATAAAACCTTATGAACCAAAACCCTTAGAACCAATTCTATGG |
| | CTATTTATTATAGCCATAACCAAAAAACTCCAACGTTTTTCTATCCGTAGCGATCTTA |
| | TGAAAGAAGAAGCAGAAGAAGCAGTTTCTTCCCATTTTGAGATAAAAACTTCTTTG |
| | ACTGTACCTCTAACTCTTGTTCCAGAGGCAAGGGCATTAAAGAAGAGAAATGTCATT |
| | AAAGGAGATAAGCGAGAGTTGGAAAAGGCATTGAAGAGACTTAGGAAATTTTATTA |
| | CTTGGTAGTAGCTGAAAAGCAAGGAGAAGCATTTTTGATAGAAGGGCCTAAGGAAA |
| | GGAGAAGTTTCCCTTTAATAATTGAAAAAATTTCTCTCCCAAAGGATGCTACTTTTC |
| | ACGTGTTTTTAGCTTATGAAAAAGAGTCTCTCAATTGCTTGTTAGATATATCTAAAC |
| | AAAAAGAAATAGAAACAGCTTTAAAAGATATTCTGTATATAAAAGTCCATTTGGTT |
| | GACTAA |
| SEQUENCE ID | atggacatgccccctttttacgaccttcaattttcgcccacgatggaacggcttattgggcacgccagggcggaaatcctggattatcccaat |
| NO: 55 | caccaaacaggtgggctgcccgacgttgaccaggctatactggccaacgacctggtcgggtttcagaaagtgctgcggcaggccttgag |
| | ctttcttcgcagcccggccaccttcgatcttgcccctggtatggggccaagcctggcgcccgtcttcaacctcctggataatgcggttattctg |
| | gaaaaatacctcatcgaccgcgcgtcgcttatgggcgacaaagctgtaatgcaggtacttgccatcggagagatggcaaccacgaaacaa |
| | gcggttccattccaccgcttcatccccgtcaacgcctggcgccggcaatttcgggatcatctcccggaggatgtatcatgttttttcccctggt |
| | atgatttctgggctaacgaagacgaattgcttctggaggaattcgtttatttgttacccatcctccagcaaggtgacttttcctcttttgcatcgca |
| | agacgccccccggcttcgtcgtctgttcgcggaaattcaaagagacaagcctttcatagctcatctgaacaaacgccaccttctccttcaaaa |
| | aaccgtacaagctgtcggtaaaaataccgccctgcgcctcttttttctggctgatgggtgcgaacccggggtgtacctgccggaactcgtcct |
| | cgaaaagggcctgcataccgtggcggttgccgccatggcacaggcccccacctccgaggagaaacggcacgagctgctgtttctggca |
| | gcttttgccggcccggaactggaagacgaagaacggctcgccattttccggaagacagtagcctttatcggcggtgcccacgctctgcaa |
| | gaagggggtctcatggataaactgcggcagtgggcaaaaaacaccctggatgaccaggaattgtcaacaagcgtttaccgccgttggcgt |
| | gatctgcttaacgggacagccccggacattgtcaccccctttgcctaccaggacgggcaattttcccaggtggttggtaatttgcataccgctt |
| | ccgtgcttgaggaaaaaacggttgttcccgcttcggatgtgcagccgggtttgtgggcgtccctggctgctcctgcaaagaaattttgggata |
| | attttacagagatcatcggtcagttcagagaatctctcgttctggaaccggaactgagccgcgcaatgagcgaagaagggggagccaggg |
| | aacgcaagcccgtcgagatacctggcctgcgtctgcggcaggagttaatgccagaagatgggcaattcctgaccacggaatcagctcgg |
| | gaaatccgggaactgcttggcgacaaaatcctgtactttatcactctggcggtggattccgccactggacaaatcagcgctacccctccggc |
| | caggcttacccgcagttcccgaatcaaagacgatatccacggcaagccgtttgttgtgggcataggggcggacaaagaaggccttgaggc |
| | cgccatcgatgagttgactagccctgaagggatcagggatcaatccaagcttgatcatatcctctggattgctattaccatcaatgaccagatt |
| | ttacgggaccgacatgcgccttaa |
| SEQUENCE ID | ATGTTAAACGATTATGAAGAAAGACAGACTGAAGGTTTTTTGTACTCCCCTGCGCTG |
| NO: 56 | GATCTCCTCCTGACCTGGGGAAGCGGTTCTCTGTTTCAGGGAACAGAAGATGAGGG |
| | GGAGTTTGTTCAGGGAATTGAGGAAGAATGGCATTCACTCATGGAGGAGTCGGATG |
| | TGAAATCCGAGTTGCACTCCGGTTTGGAGGCACTGACCCAGGATATCAATGACGCC |
| | CTGGATTTCCTTTGTCATGCAATTCCGGAGTCAGACAGGGAGACTGCACTGGCTCAG |
| | ACCGCTCTGGAGATACTGGATCACGGGGTACGCTTGTTTCAGACAACCCGAAATTTC |
| | AGTGAGCAGAACCATTTCCCGGAAGGCTTTCAGGTCAGGGCAGAGGCATTGTTCAG |
| | AAATATCACCGTTGATCGCGCACCCTCTTATATCCGCCTGATTCCTCTGAACTATTGG |
| | CGACAGTTTATGCGCGGGAATATTTCGGAACACAGTTTTCATCTGTTTCCCTGGTAT |
| | GATGAATGGGCCGATATGCCTTCGGAAACCATAGAGATACTGATCGAAAACTGGAA |
| | TGAGATCAGCCATGGAAACTTTGAATGTTCGGAAATGGACACTGAGACATTGAAGG |
| | TTCTGTTTGCTGAGTTGTCAAATGACCGGGAGCTTTTGAATTATATACAGGAGGAAG |
| | CCAGATTTGGACACATTCTGCCCCGGGCAATCGGAAAAAGCCTTGTCCTTCGTCTTT |
| | TGATGATAAGGAATGAGGAAGTGGGGAAACACGCTGCTCCTCAAAAGGTGAGAGA |
| | GAAAGGGTTTGTCGCGTGTGCGTGTCACATTATTCAAAAGATAAAGAAAATCTTTCG |
| | CTCTGAGGAAGAGAGATTGGAGGGGATTTTTCTAAGTGGGTTCTGCGGTCCTCACTT |
| | GCGTGAGAATCAGAGAACCGAACTTTTTACTCAAGTGGAGCATGAACTGAAAACTC |
| | TGGATGTATCACTGCGCCCTGGAAGTCTGCTTGAGGAGCTTTGTCAATGGTCCCAGG |
| | GAAAGGTGGCTGATGAGGTCTTTTCCCGGCGCGTGTTTGAACACTGGAATAATGAA |
| | CTGAGTCTGTCCGCCAAAGCGGTCACAGAAAGGGTTCCGGAGGATGCTGCTATTTTT |
| | CTTCGGGCGGTCAATGAGTTGGCGTTTGCGAAGTTAGAGACGGAAACGGTTGCTGA |
| | GAAAATTGAATGGGGAATCGGACGGCTGTTTGATATTAATAAGCTAGCGGTCAATG |
| | AGTTGGCGTTTGCGAAGTTAGAGACAGAAACCGTTGCTGAGAAAATTGGACGGGGG |
| | ATTGGATGCTTGCCTGATAAGGTAATTAATCTCATCCCTTCGCTAATTGTGCGATTG |
| | AAGAATCCAGGTTATGCGGGAAATGGCGATGATGATGACGGTAACGAAAATGGAA |
| | GTGTTACACTCCCCCGAATGATCAATGTCTGCGGCCATCGAGAAGGGAATAATGTG |
| | AATCTTCTTGAAGCCAGTGAGACGATACCGGATGAAATAGAAAAACTGCTCCGACA |
| | GGAAACCGGAGAAGTTCCTAAAAAGTTGATCCCTGTTATGAACAGCCGAAAGAAAT |
| | TATTTTATCAGGTTTTAGCAGAATCATCGGATGACAAATGGGTATTCTGCTTTGATA |
| | AGCCTAAGAAAACTCGGGCAGCCCGAATCGAAGTTGCAGAAACCGTATATGATCGT |
| | TTTGTGCTGCTGCTGGATCCGGGCATAAAAGGTTTGAAACATAGTTCAGAGGTGATG |
| | ATGTCAGTTCTTAACAGTGTCAAGGCTGAAGATCGGCAAATTTCTCCTTACACGGTT |
| | ATCATTGATCTCGAAATACGAGATTCCGAAGGAGGTTCCAAATGA |

### Apoptotic Proteins

Apoptosis can be initiated through one of two pathways. In the intrinsic pathway the cell kills itself because it senses cell stress, while in the extrinsic pathway the cell kills itself because of signals from other cells. Weak external signals may also activate the intrinsic pathway of apoptosis. Both pathways induce cell death by activating caspases, which are proteases, or enzymes that degrade proteins. The two pathways both activate initiator caspases, which then activate executioner caspases, which then kill the cell by degrading proteins indiscriminately.

Caspases play the central role in the transduction of ER apoptotic signals. Caspases are proteins that are highly conserved, cysteine-dependent aspartate-specific proteases. There are two types of caspases: initiator caspases, caspase 2, 8, 9, 10, 11, 12, and effector caspases, caspase 3, 6, 7. The activation of initiator caspases requires binding to specific oligomeric activator protein. Effector caspases are then activated by these active initiator caspases through proteolytic cleavage. The active effector caspases then proteolytically degrade a host of intracellular proteins to carry out the cell death program.

### Inhibitory Peptides

Inhibitory peptides are peptides that inhibits the activity of a protein when an inhibitory peptide is fused to said protein. In some embodiments, the inhibitory peptide inhibits the activity of the protein via steric hindrance. In some embodiments, the inhibitory peptide comprises a specific degradation signal, or a degron. In some embodiments, the specific degradation signal, or a degron is derived from dihydrofolate reductase (DHFR).

A degradation signal or ‘degron’, is usually defined as a minimal element within a protein that is sufficient for recognition and degradation by a proteolytic apparatus. An important property of degrons is that they are transferable. That is, genetically engineered attachment of such sequences confers metabolic instability (a short half-life) on otherwise long-lived proteins. Degrons can be defined for distinct proteolytic pathways.

A degron may be a portion of a protein that is important in regulation of protein degradation rates. Known degrons include short amino acid sequences, structural motifs and exposed amino acids (often Lysine or Arginine) located anywhere in the protein. In fact, some proteins can even contain multiple degrons. Degrons are present in a variety of organisms, from the N-degrons first characterized in yeast to the PEST sequence of mouse ornithine decarboxylase. Degrons have been identified in prokaryotes as well as eukaryotes. While there are many types of different degrons, and a high degree of variability even within these groups, degrons are all similar for their involvement in regulating the rate of a protein's degradation. Much like protein degradation mechanisms are categorized by their dependence or lack thereof on Ubiquitin, a small protein involved in proteasomal protein degradation, Degrons may also be referred to as “Ubiquitin-dependent” or “Ubiquitin-independent”.

Examples of degron are disclosed in Cho, Sungchan, et al., Genes & Development. 24 (5): 438-442; Fortmann, Karen T., et al., Journal of Molecular Biology. 427 (17): 2748-2756; Dohmen, R. J., et al., Science, 1994. 263(5151): p. 1273-1276; Varshavsky, A. Proceedings of the National Academy of Sciences. 93 (22): 12142-12149; Kanarek, Naama, et al., Cold Spring Harbor Perspectives in Biology. 2 (2): a000166; Bachmair, A., et al., Science. 234 (4773): 179-186, Loetscher, P., et al., The Journal of Biological Chemistry. 266 (17): 11213-11220; Burns, Kristin E., et al., Journal of Biological Chemistry. 284 (5): 3069-3075; and Ravid, Nature Reviews. Molecular Cell Biology. 9 (9): 679-690.

In some embodiments, the inhibitory peptide inhibits the activity of the protein via degrading the protein. In eukaryotic cells, an ATP-dependent protease called the proteasome is responsible for much of this proteolysis. Proteins are targeted for proteasomal degradation by a two-part degron, which consists of a proteasome binding signal and a degradation initiation site. Here we describe how both components contribute to the specificity of degradation. Only substrates that contain specific degradation signals, or degrons, are recognized by the proteasome, processively unfolded, threaded into the degradation chamber, and digested. One strategy involves fusing a degron, derived from dihydrofolate reductase, to the N-terminus of the target protein, which thereby confers degradation.

### Quenching

Quenching refers to any process which decreases the fluorescence intensity of a given substance. A variety of processes can result in quenching, such as excited state reactions, energy transfer, complex-formation and collisional quenching. Molecular oxygen, iodide ions and acrylamide are common chemical quenchers. The chloride ion is a well-known quencher for quinine fluorescence.

Quenching and dequenching upon interaction with a specific molecular biological target is the basis for activatable optical contrast agents for molecular imaging. Here, the fluorescence of a fluorophore is quenched by the fluorophore-quencher interaction, but is activated after cellular the fluorophore and its quencher are disassociated.

The fluorophore may be covalently attached to a quencher via a Csx30 linker. Several different fluorophores (e.g. 6-carboxyfluorescein, acronym: FAM, or tetrachlorofluorescein, acronym: TET) and quenchers (e.g., tetramethylrhodamine, acronym: TAMRA) may be used. As long as the fluorophore and the quencher are in proximity, quenching inhibits any fluorescence signals.

### EXAMPLES

The invention now being generally described, it will be more readily understood by reference to the following examples which are included merely for purposes of illustration of certain aspects and embodiments of the present invention, and are not intended to limit the invention.

### Example 1: Experimental Procedures

Plasmid Construction

For the bacterial expression of the D. ishimotonii Cas7-11-crRNA-Csx29 complex, the gene encoding Cas7-11 was amplified by PCR and cloned into the modified pACYCDuet-1 plasmid vector (Novagen), expressing Cas7-11 with an N-terminal maltose-binding protein (MBP) and a C-terminal His₆-tag (SEQ ID NO: 83) (MBP-Cas7-11-His₆ (“His₆” disclosed as SEQ ID NO: 83)). The gene encoding Csx29 was cloned into the His₆-Twin-Strep-SUMO-pET28a vector (“His₆” disclosed as SEQ ID NO: 83), expressing Csx29 with an N-terminal His₆-Twin-Strep-SUMO tag (“His₆” disclosed as SEQ ID NO: 83) (His₆-Twin-Strep-SUMO-Csx29 (“His₆” disclosed as SEQ ID NO: 83)). The CRISPR array containing two direct repeats interspaced by a spacer with the 5′ LacI-repressed T7 promoter and 3′ T7 terminator sequences was synthesized by Eurofins Genomics. For the bacterial expression of Csx30 and Csx30-Csx3l-RpoE, the gene encoding Csx30 or Csx30 and Csx31 was amplified from the type III-E D. ishimotonii CRISPR locus and cloned into the modified pE-SUMO vector (LifeSensors), in which the SUMO-coding region is replaced with the HRV3C protease recognition site. The gene encoding RpoE was cloned into the pACYCDuet-1 vector, expressing RpoE with an N-terminal His₆-tag (SEQ ID NO: 83). The mutants of Cas7-11, Csx29, and Csx30 were generated by a PCR-based method, and the sequences were confirmed by DNA sequencing.

Sample Preparation

Cas7-11, Csx29, and the CRISPR array were co-expressed in E. coli BL21 (DE3) (Novagen) by induction with 0.25 mM isopropyl β-D-thiogalactopyranoside (Nacalai Tesque) at 18° C. overnight. The E. coli cells were lysed by sonication in buffer A (20 mM Tris-HCl, pH 7.5, 20 mM imidazole, 150 mM NaCl, 10% glycerol, and 3 mM 2-mercaptoethanol), and the lysate was clarified by centrifugation at 40,000 g. The supernatant was applied to Ni-NTA Superflow resin (QIAGEN), and the bound protein was eluted with buffer A containing 300 mM imidazole. The eluted fraction was diluted 2-fold with buffer B (20 mM Tris-HCl, pH 7.5, 150 mM NaCl, 10% glycerol, and 3 mM 2-mercaptoethanol), and applied to Strep-Tactin XT high capacity (IBA), and the bound protein was eluted with buffer C (100 mM Tris-HCl, pH 8.0, 150 mM NaCl, 10% glycerol, 1 mM EDTA, 50 mM biotin, and 3 mM 2-mercaptoethanol). The eluted protein was applied to Amylose resin (NEB) equilibrated with buffer D (20 mM Tris-HCl, pH 7.5, 150 mM NaCl, 10% glycerol, and 1 mM DTT). The resin was washed with buffer D (20 mM Tris-HCl, pH 7.5, 150 mM NaCl, 10% glycerol, and 1 mM DTT), and the bound protein was eluted with buffer D containing 10 mM D-maltose. For biochemical experiments, the eluted protein was dialyzed against buffer D to remove D-maltose. The concentration of the Cas7-11-Csx29-crRNA complex was measured using Pierce 660-nm Protein Assay Reagent (Thermo Fisher Scientific). Csx30 was expressed in E. coli Rosetta2 (DE3), and purified by Ni-NTA Superflow resin and a Superdex 200 Increase column (GE Healthcare). Csx30, Csx31, and RpoE were co-expressed in E. coli BL21 (DE3), and the Csx30-Csx31-RpoE complex was purified by Ni-NTA Superflow resin and a HiLoad 16/600 Superdex 200 column (GE Healthcare). The target RNAs were transcribed in vitro with T7 RNA polymerase, and purified by 10% denaturing (7 M urea) polyacrylamide gel electrophoresis. The purified materials were stored at −80° C. until use.

Cryo-EM Grid Preparation and Data Collection

To prevent tgRNA cleavage, the catalytically inactive Cas7-11 (D429A/D654A) was used for cryo-EM studies. The Cas7-11-crRNA-Csx29-tgRNA complex was reconstituted by mixing the purified Cas7-11-crRNA-Csx29 complex and the target RNA, at a molar ratio of 1:4. The complex was purified by size-exclusion chromatography on a Superose 6 Increase 10/300 column (GE Healthcare), equilibrated with buffer E (20 mM HEPES-NaOH, pH 7.0, 150 mM NaCl, 1 mM MgCl₂, and 1 mM DTT). The peak fraction containing Cas7-11-crRNA-Csx29-tgRNA was analyzed by TBE-urea gel, and concentrated to an A₂₆₀ of 3.0, using an Amicon Ultra-4 Centrifugal Filter Unit (MWCO 50 kDa). Cryo-EM grids were glow-discharged using PIB-10 Ion Bombarder (JEOL, Japan) with 10 mA current for 3 min. The sample (3 μL) was applied to freshly glow-discharged Au 300 mesh R1.2/1.3 grids (Quantifoil) in a Vitrobot Mark IV (FEI) at 4° C. under 100% humidity conditions, and the excess solution of the sample was blotted with filter paper (Agar Scientific) with a waiting time of 10 s and a blotting time of 4 s. The grids were plunge-frozen into liquid ethane cooled at liquid nitrogen temperature. For the cryo-EM analysis of the Cas7-11-crRNA-Csx29 complex, the purified Cas7-11-crRNA-Csx29 complex was further polished by size-exclusion chromatography on a Superose 6 Increase 10/300 column, equilibrated with buffer E. The peak fraction containing Cas7-11-crRNA-Csx29 was concentrated to an A₂₆₀ of 2.5. The sample was applied onto freshly glow-discharged Au 300 mesh R0.6/1 grids (Quantifoil) in a Vitrobot Mark IV under similar conditions to those for the Cas7-11-crRNA-Csx29-tgRNA complex. The cryo-EM data were collected using a Titan Krios G3i microscope (Thermo Fisher Scientific), running at 300 kV and equipped with a Gatan Quantum-LS Energy Filter (GIF) and a Gatan K3 Summit direct electron detector. Micrographs were recorded at a nominal magnification of ×105,000 with a pixel size of 0.83 Å in a total exposure of 48 e⁻/Å² per 48 frames with exposure time of 2.5 s. The data were automatically acquired by the image shift method using the EPU software (Thermo Fisher Scientific), with a defocus range of −0.8 to −2.0 m, and 2,924 and 6,084 movies were acquired for Cas7-11-crRNA-Csx29 and Cas7-11-crRNA-Csx29-tgRNA, respectively.

Image Processing

The data processing was performed using cryoSPARC v3.3.1 software packages. The dose-fractionated movies were aligned using the Patch motion correction and the contrast transfer function (CTF) parameters were estimated using Patch-Based CTF estimation with the default settings. Particles were automatically picked using Blob picker and Topaz followed by reference free 2D classification to curate particle sets. The particles were further curated by Heterogeneous Refinement with the default parameters using the map derived from cryoSPARC Ab initio Reconstruction as a template. For the Cas7-11-crRNA-Csx29-tgRNA complex, to further distinguish the conformational heterogeneity of Csx29, mask was generated for the Csx29 region, and the selected 349,129 particles after Heterogeneous Refinement were classified for 24 classes using 3D classification without alignment in the Principal Component Analysis (PCA) initialization mode. The selected particles after Heterogeneous Refinement were refined using Non-uniform refinement. Local motion correction followed by Non-uniform refinement with optimization of CTF value yielded a map at 2.49 Å and 2.84 Å resolution for Cas7-11-crRNA-Csx29 and Cas7-11-crRNA-Csx29-tgRNA, respectively, according to the gold-standard Fourier shell correlation (FSC)=0.143 criterion. The local resolution was estimated by BlocRes in cryoSPARC. Histograms of directional FSC curve and sphericity value were calculated in the 3DFSC server.

Model Building and Validation

For model building for Cas7-11, the previously published Cas7-11 structure (PDB ID: 7WAH) was rigid-body fitted into the reconstructed density maps in UCSF ChimeraX. The initial model of Csx29 was predicted using AlphaFold2, and fitted into the density map using the Dock Predicted Model in PHENIX package. These models were manually modified using COOT against the density map sharpened using DeepEMhancer. The models were refined using Real-space refinement in PHENIX with the secondary structure and the Ramachandran restraints. Since the MBP, His₆ (SEQ ID NO: 83) and SUMO tags were not resolved in the density map, they were not included in the final models. The structures were validated using MolProbity from the PHENIX package and EMRinger. The curve representing model vs. full map was calculated using phenix.mtriage, based on the final model and the full, filtered and sharpened map. The cryo-EM density maps were calculated with UCSF ChimeraX, and molecular graphics figures were prepared with CueMol (world wide web at cuemol.org).

In Vitro Csx30 Cleavage Experiment

The purified Cas7-11-crRNA-Csx29 complex (5 nM) was incubated at 37° C. for 10 min with the purified Csx30 protein (15 μM) in the presence or absence of the tgRNA (20 nM) in reaction buffer (20 mM HEPES-NaOH, pH 7.5, 150 mM NaCl, 5 mM MgCl₂, and 2 mM DTT). The reaction was quenched by the addition of an SDS-PAGE sample buffer, and the mixture was then analyzed by SDS-PAGE. The gels were stained with Bullet CBB Stain One (Nacalai Tesque), and then imaged using a FUSION Solo S system (Vilber Bio Imaging).

In Vitro Target RNA Cleavage Experiment

The purified Cas7-11-crRNA-Csx29 complex (200 nM) was incubated at 37° C. for 10 min with a 5′-Cy5-labeled ssRNA target (600 nM) in reaction buffer (20 mM HEPES-NaOH, pH 7.5, 50 mM NaCl, 5 mM MgCl₂, and 2 mM DTT). The reaction was quenched by the addition of quenching solution (0.45 mg/mL proteinase K (Nacalai Tesque), 6 mM EDTA, and 200 μM urea), and then incubated at 50° C. for 15 min. The mixture was incubated at 100° C. for 2 min with 4.5 M urea denaturing buffer, and then analyzed using a 15% Novex PAGE Tris-borate-EDTA (TBE)-urea gel (Invitrogen). The gels were imaged using a FUSION Solo S system, using either Cy5 fluorescence or SYBR Gold fluorescence (Thermo Fisher Scientific).

N-Terminal Analysis

The purified Csx30 protein was cleaved by the purified dCas7-11-crRNA-Csx29 complex in the presence of the tgRNA in reaction buffer (20 mM HEPES-NaOH, pH 7.5, 150 mM NaCl, and 2 mM DTT). The proteins were then separated by SDS-PAGE, blotted onto a PVDF memrbrane, and stained with Bullet CBB Stain One. The protein band corresponding to a ˜15 kDa Csx30 fragment (Csx30-2) was cut out from the membrane, and its N-terminal amino-acid sequence was analyzed by Edman mircrosequencing on a Procise 494 cLC protein sequencer (Applied Biosystems), using the standard pulsed-liquid program for PVDF-blotted proteins.

Bacterial Cell Growth Experiment

E. coli DH5α Competent Cells (Thermo Fisher Scientific) were transformed with a plasmid expressing Csx30 alone or plasmids expressing both Csx30 and Csx31 (Table 4). Single colonies were picked into Terrific broth (Fisher Scientific) containing relevant antibiotics and 1% v/v glucose, and then cultured at 37° C. overnight. Following overnight culture, bacterial OD₆₀₀ values were measured and normalized to an initial OD₆₀₀ of 0.00046 in 200 μL final volume by dilution in Terrific broth containing relevant antibiotics as well as 1% glucose for non-induced conditions and 1% arabinose for induced conditions. Using BioCoat Cellware 96-well tissue culture plates (Corning), growth assays were performed using a BioTek Synergy Neo2 at 37° C. with continuous shaking, reading OD₆₀₀ values at 10 min intervals for up to 22 hours. For growth curves with different temperatures, the same culturing conditions were used and the BioTek Synergy Neo2 is set to either 30° C., 37° C., or 42° C. with continuous shaking for up to 22 hours during the measurement.

TABLE 4
Csx29 and 30 plasmid maps

| Name | Full Description | Benchling Link |
|---|---|---|
| Full-length | Expression of | At world wide web at benchling.com/s/seq- |
| wtCsx30 bacterial | Csx30 under | Q8N6zZrjkadwhqQWdWFI?m=slm- |
| expression | arabinose-inducible | UsJYgu75dA4PfEC2m68z |
| | promoter, | |
| | kanamycin | |
| | resistance | |
| Csx30-1 bacterial | Expression of N- | At world wide web at benchling.com/s/seq- |
| expression | term Csx30 under | dHVl08GZ3SPm5OM5K8F7?m=slm- |
| | arabinose-inducible | NyNp8JdLh5on8sbrh9De |
| | promoter, | |
| | kanamycin | |
| | resistance | |
| Csx30-2 bacterial | Expression of C- | At world wide web at benchling.com/s/seq- |
| expression | term Csx30 under | XoNvTE5ZUo8cMxdGirXr?m=slm- |
| | arabinose-inducible | vY9Hy9iGb63fP0EwJjZq |
| | promoter, | |
| | kanamycin | |
| | resistance | |
| Full-length | Expression of | At world wide web at benchling.com/s/seq- |
| wtCsx31-Csx30 cis | wtCsx31-Csx30 | k9hlylchBnXsjbewceKR?m=slm- |
| bacterial expression | with endogenous | SKDnnzCdvqn6co4ymX1h |
| | RBS under | |
| | arabinose-inducible | |
| | promoter, | |
| | kanamycin | |
| | resistance | |
| Full-length | Expression of | At world wide web at benchling.com/s/seq- |
| wtCsx31-Csx30- | wtCsx31-Csx30- | Shz0rSS3hWUXewSaPOFY?m=slm- |
| rpoE cis bacterial | rpoE with | SnkUthyZpjjiLotTvShW |
| expression | endogenous RBS | |
| | under arabinose- | |
| | inducible promoter, | |
| | kanamycin | |
| | resistance | |
| Csx30-1-Csx31 | Expression of | At world wide web at benchling.com/s/seq- |
| bacterial expression | wtCsx31-N-term | 8nUKhBl0m1sSfEbfwgXX?m=slm- |
| | Csx30 with | vvxiFmq3QD7zaLSnRy6F |
| | endogenous RBS | |
| | under arabinose- | |
| | inducible promoter, | |
| | kanamycin | |
| | resistance | |

In Vitro Binding Experiment

The purified Csx30-Csx31-RpoE complex (15 μM) was incubated at 37° C. for 15 min with the purified dCas7-11-crRNA-Csx29 complex (25 nM) in the presence of the tgRNA (100 nM) in reaction buffer (20 mM HEPES-NaOH, pH 7.5, 150 mM NaCl, 5 mM MgCl₂, and 2 mM DTT). The Csx30-Csx31-RpoE complex with or without the dCas7-11-crRNA-Csx29 treatment was analyzed on a Superdex 200 Increase 10/300 column equilibrated with buffer (20 mM HEPES-NaOH, pH 7.5, 150 mM NaCl, and 2 mM DTT). The peak fractions were analyzed by SDS-PAGE. The gels were stained with Bullet CBB Stain One, and then imaged using a FUSION Solo S system.

Confocal Imaging

E. coli DH5α Competent Cells were transformed with a EGFP-Csx30 fusion plasmid, a EGFP-Csx31 fusion plasmid together with a Csx30 plasmid, or a regular EGFP plasmid. Single colonies were picked into Terrific broth containing relevant antibiotics and 1% v/v arabinose, and then cultured at 37° C. overnight. Following overnight culture, bacteria was spinned down at 4000 g for 5 min and resuspended using diluted Agar solution (0.7% LB agar w/v). Then, 20 μL of bacterial solution was dropped onto a glass slide for confocal imaging. The bacterial cells were imaged with a Zeiss LSM 900 Airyscan 2 using a 63× oil immersion objective. Prior to imaging, a drop of immersol W solution is applied onto the coverslip of the slide.

Mammalian Cell Culture

HEK293FT cells (Thermofisher—R70007) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific). Adherent cells were maintained at confluency below 80-90% at 37° C. and 5% CO₂.

Transfection for Luciferase Sensors

HEK293FT cells were plated at 1×10⁴ cells/well the day prior to transfection in a 96-well plate coated with poly-D-lysine (BD Biocoat), and were transfected with Lipofectamine 3000 (Thermo Fisher Scientific) according to manufacturer's specifications. 35 ng of mammalian codon-optimized Cas7-11 and Csx29 expressing plasmid, 25 ng of targeting guide RNA or non-targeting guide RNA, 25 ng of targets (Gaussia luciferase), and 10 ng of the citrine-degron reporter were delivered to each well unless otherwise specified. 48 hours later, the medium is replaced with DMEM (without phenol red) for citrine signal measurement using a Biotek Synergy 4 plate reader with a gain of 100 for the citrine channel.

### Example 2: Structures of Cas7-11 in Complex with Csx29

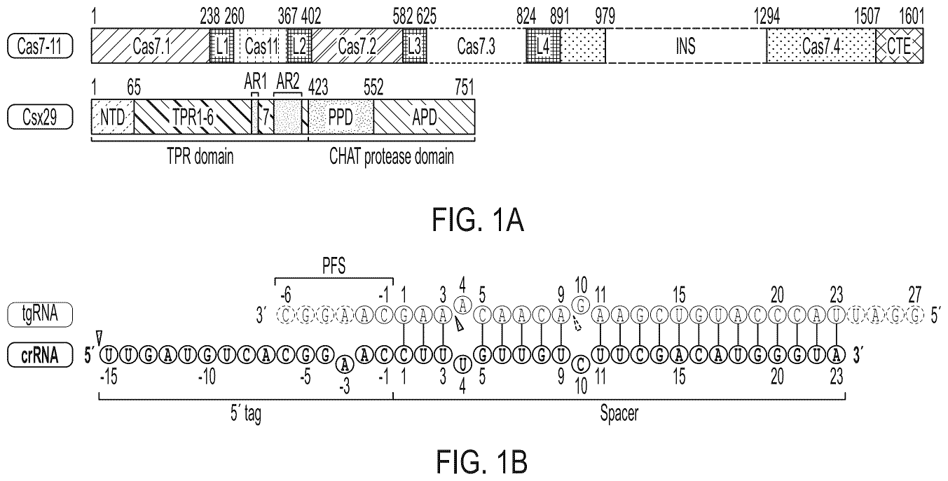

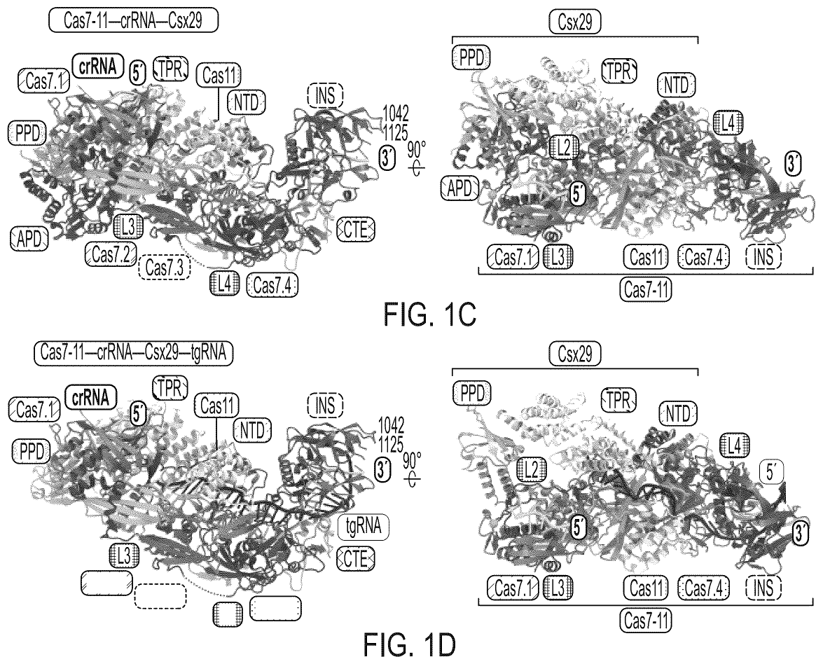

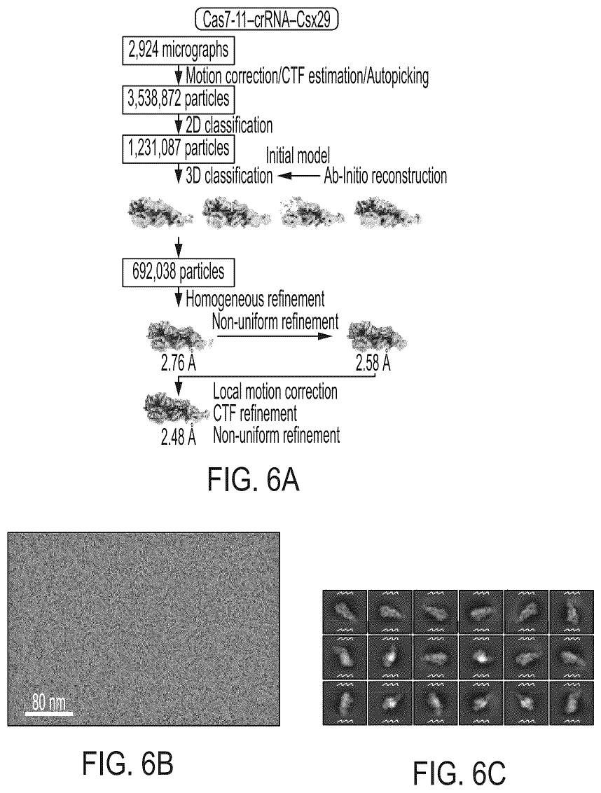

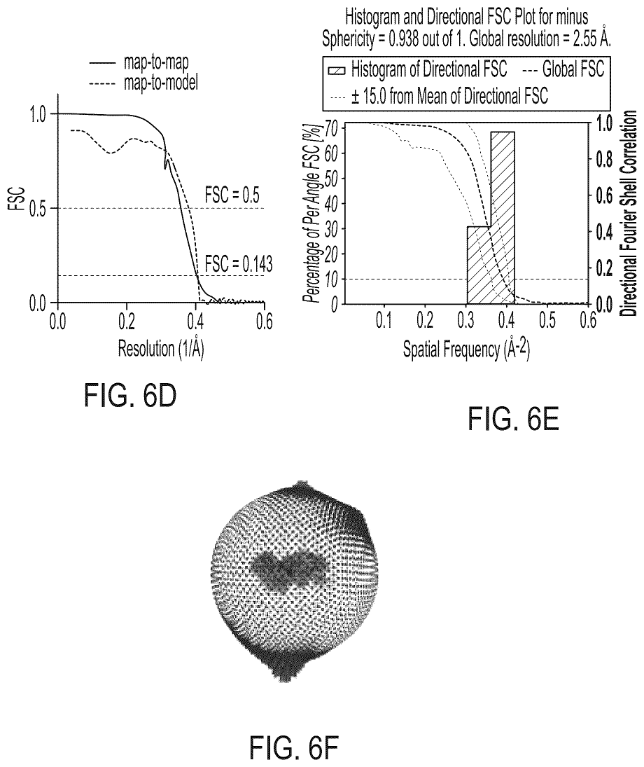

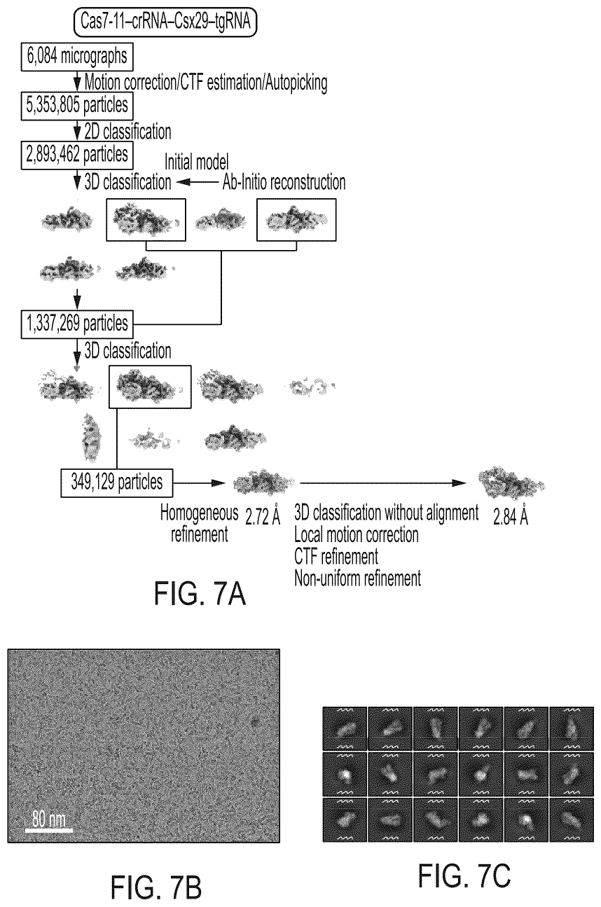

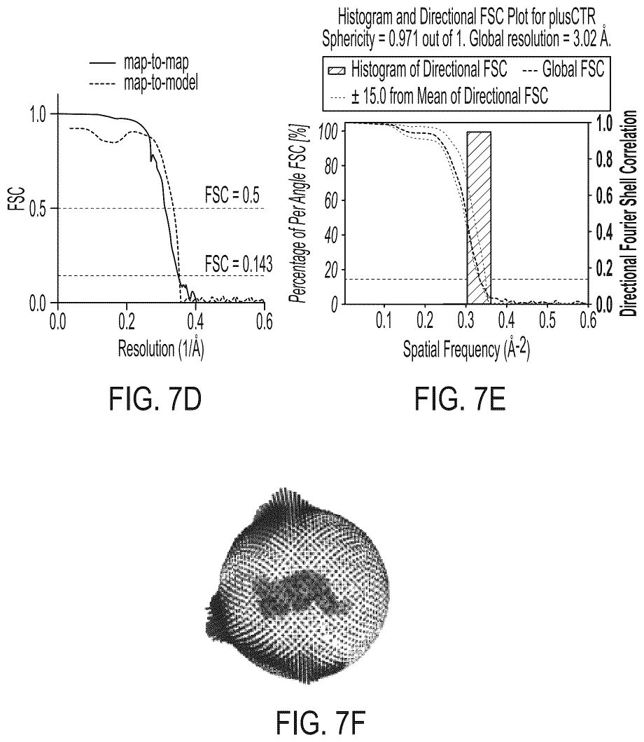

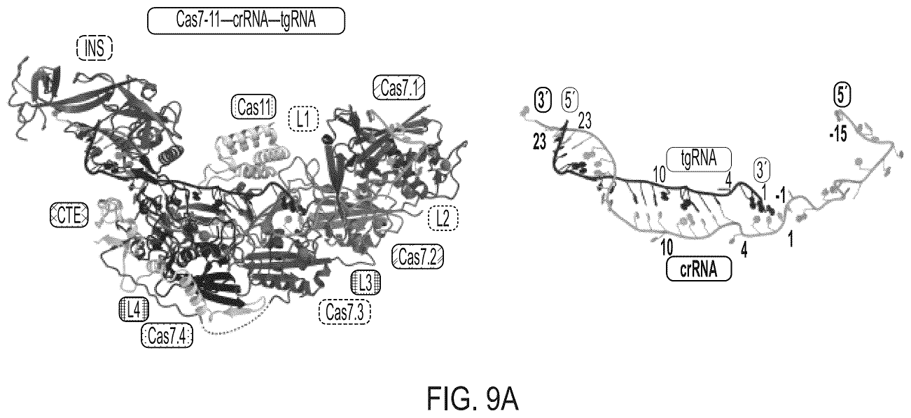

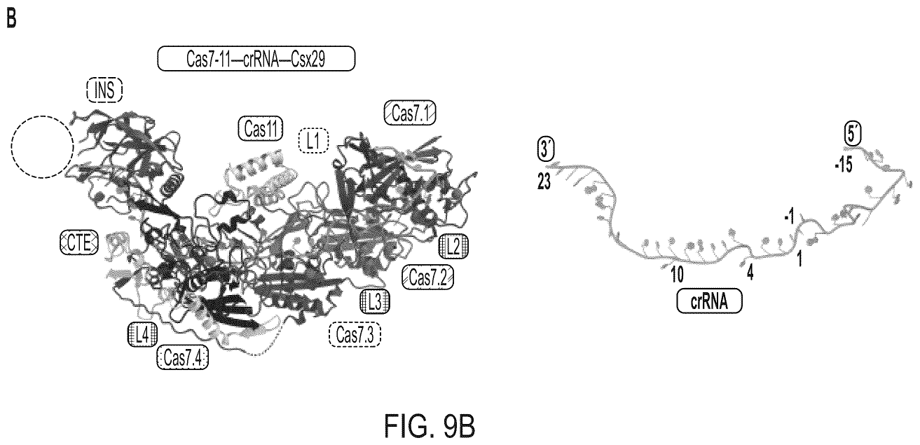

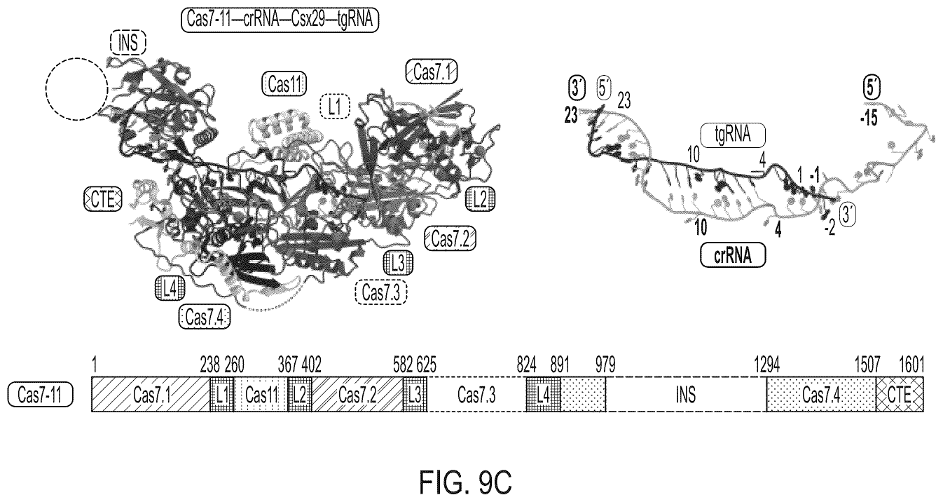

We reasoned that structural insights would allow for mechanistic understanding of the Cas7-11-Csx29 effector complex. To prepare the Cas7-11-crRNA-Csx29 complex for structural analysis, we co-expressed the catalytically inactive D. ishimotonii Cas7-11 mutant (referred to as Cas7-11 for simplicity), with D429A (Cas7.2) and D654A (Cas7.3) mutations introduced to prevent tgRNA cleavage by Cas7-11, together with Csx29 and a crRNA transcribed from a CRISPR array containing two repeat-spacer units. We determined the cryo-EM structures of the Cas7-11-crRNA-Csx29 complex with and without a tgRNA at 2.5- and 2.8-Å resolutions, respectively (FIGS. 1A-1D, FIGS. 6-8). In both structures, Cas7-11 adopts a modular architecture consisting of four Cas7 domains (Cas7.1-Cas7.4) with a zinc finger (ZF) motif, a Cas11 domain, an insertion (INS) domain inserted within the Cas7.4 domain, a C-terminal extension (CTE) domain, and four interdomain linkers (L1-L4) (FIGS. 1C and 1D), as in the Csx29-unbound Cas7-11-crRNA-tgRNA structure (FIG. 9).

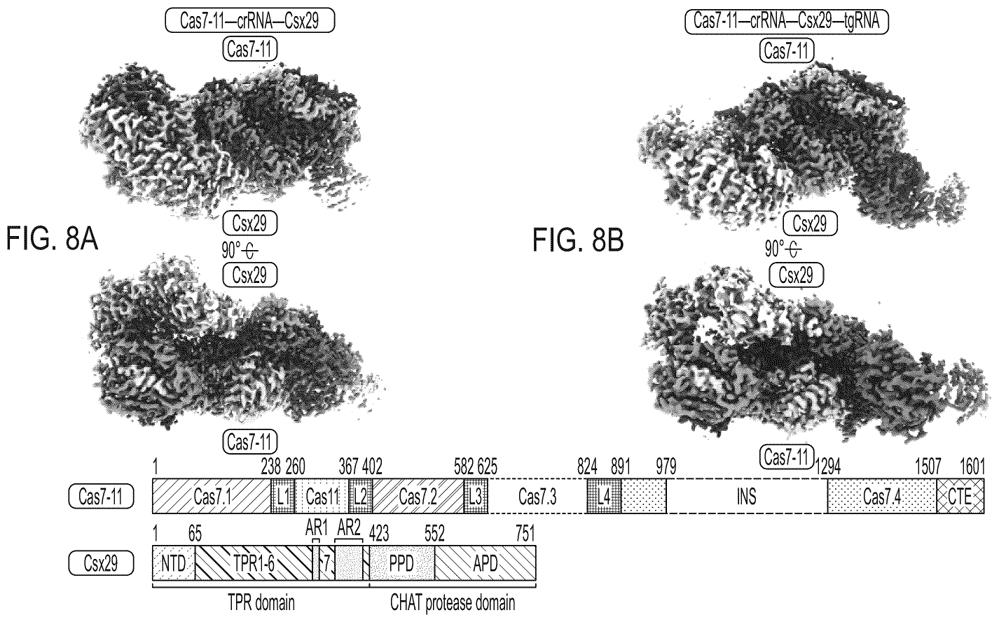

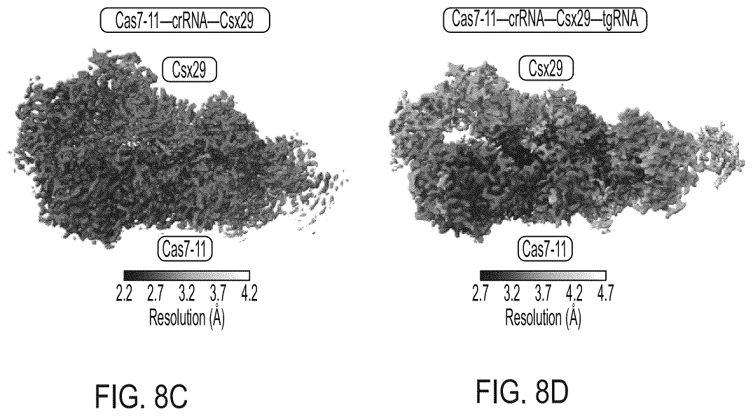

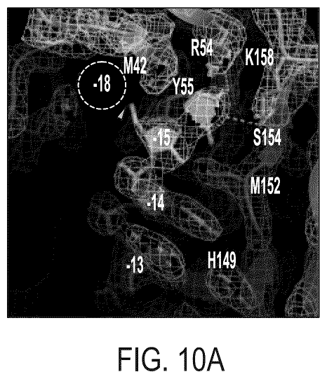

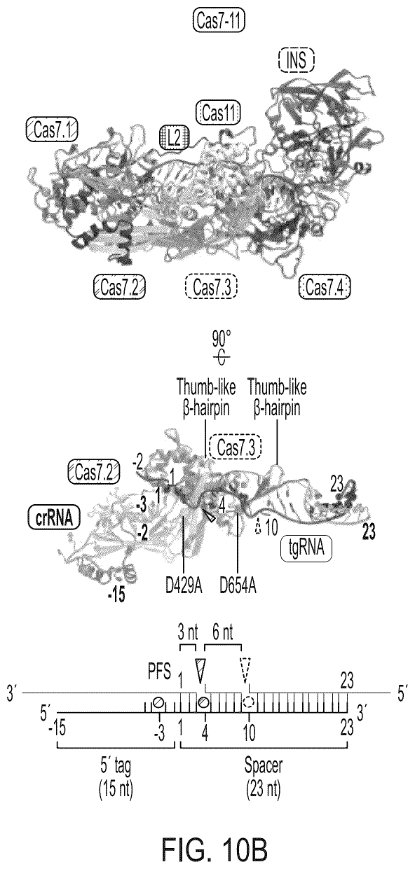

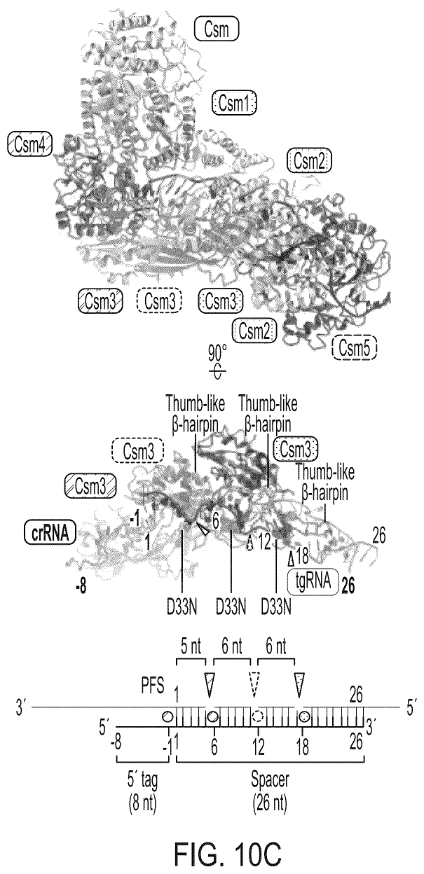

The 15-nt 5′ tag region (U(-15)-C(-1)) in the 38-nt crRNA (U(-15)-A23) is anchored by the Cas7.1 and Cas7.2 domains (FIGS. 1C and 1D). Nucleotide U(-16) was not resolved in the density map (FIG. 10A), suggesting that the co-expressed pre-crRNA was processed by Cas7-11 into the mature crRNA. U(-15) is surrounded by H43, R53, Y55, N152, and S154 in the Cas7.1 domain (FIG. 10A), consistent with the proposed pre-crRNA processing mechanism, in which H43 functions as a general base to deprotonate the 2′-hydroxy group of U(-16). In the tgRNA-free structure, the 23-nt crRNA spacer region (C1-A23) is recognized by the Cas7.2-Cas7.4 domains (FIG. 1C and FIG. 9B), while in the tgRNA-bound structure, the crRNA spacer region (C1-A23, except for U4 and C10) hybridizes with the tgRNA (G1-U23, except for A4 and G10) to form a guide-target duplex (FIG. 1D and FIG. 9C), as in the Csx29-unbound Cas7-11-crRNA-tgRNA structure. A(-3) in the 5′ tag (6-nt downstream of the first flipped-out spacer nucleotide) is flipped out due to the interaction with the thumb-like β-hairpin in the Cas7.1 domain (FIG. 10B), similar to the equivalent nucleotide C(-1) in the type III-A Csm effector complex (FIG. 10C). Nonetheless, unlike in the Csm complex, A(-2) and C(-1), which are located upstream of A(-3), cannot base pair with a target RNA, due to the presence of the L2 linker (FIG. 10B). These structural differences explain the distinct RNA cleavage patterns between the Cas7-11 and Csm effector complexes. In the present structures, the peripheral region (residues 1043-1124) of the INS domain was less resolved in the density map, probably due to its flexibility (FIG. 8). Thus, the peripheral region of the INS domain was not included in the final models of both structures.

### Example 3: Csx29 Structure

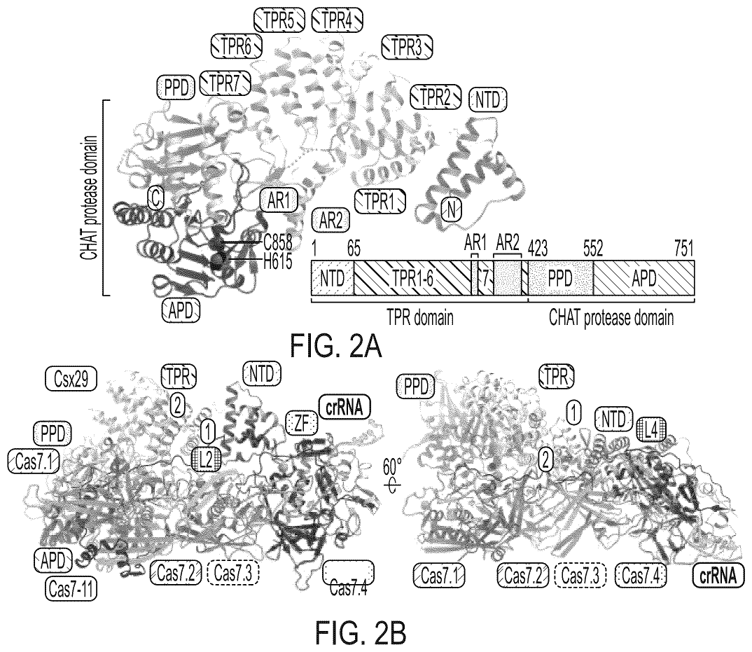

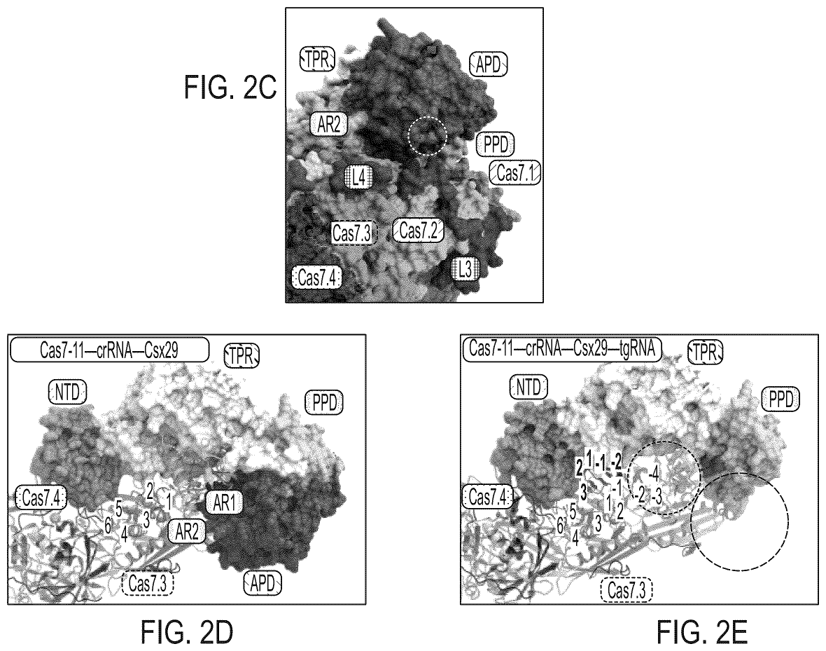

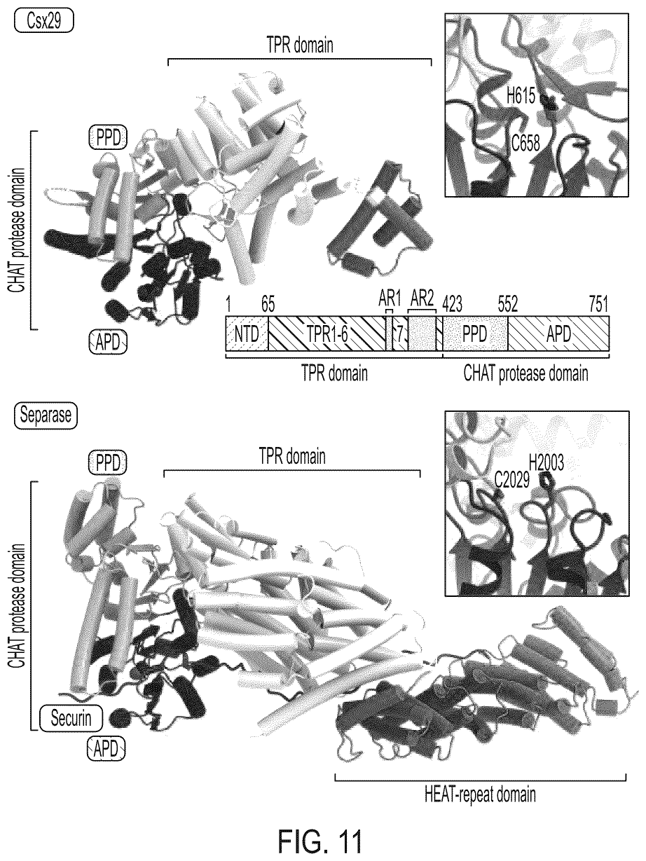

Csx29 consists of a TRP (tetratricopeptide repeat) domain (residues 1-422) and a CHAT (Caspase HetF Associated with TPRs) protease domain (residues 423-751) (FIG. 2A). The TRP domain can be divided into an N-terminal domain (NTD) (residues 1-64), seven TPR units (TPR1-TPR7), and a central region (referred to as an activation region (AR)). The NTD adopts a three-helix bundle and interacts with the Cas7.4 domain of Cas7-11 (FIG. 2B). In Csx29, each TPR unit contains two α helices, similar to canonical TPR-containing proteins where TPRs interact with their protein targets. TPR1 and TPR2 of Csx29 interact with the L2 linker of Cas7-11 (FIG. 2B). The CHAT domain of Csx29 consists of a central 11-stranded mixed β-sheet and flanking α-helices, and can be divided into a pseudo-protease domain (residues 423-551) and an active-protease domain (residues 552-751) with the conserved putative catalytic residues H615 and C658 (FIG. 2A). A Dali search confirmed that the CHAT domain of Csx29 structurally resembles caspase-like cysteine proteases, such as human separase (FIG. 11). In the Cas7-11-crRNA-Csx29 structure, the AR consists of two regions, AR1 (a β-hairpin between TPR6 and TPR7) and AR2 (a β-strand and a helix-loop-helix after TPR7), and interacts with the TPR1-TPR6 and APD (FIG. 2A).

### Example 4: Interactions between Cas7-11 and Csx29

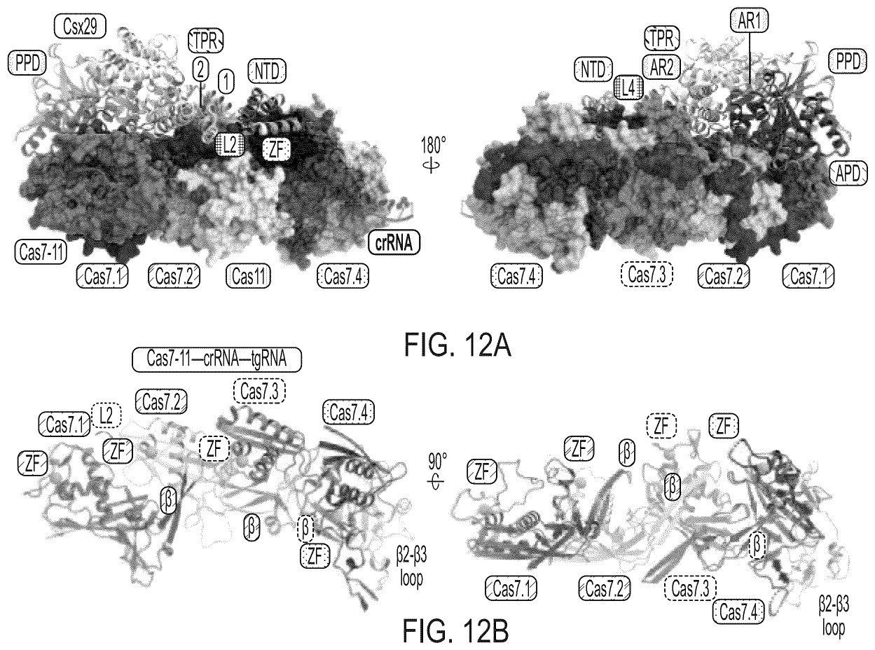

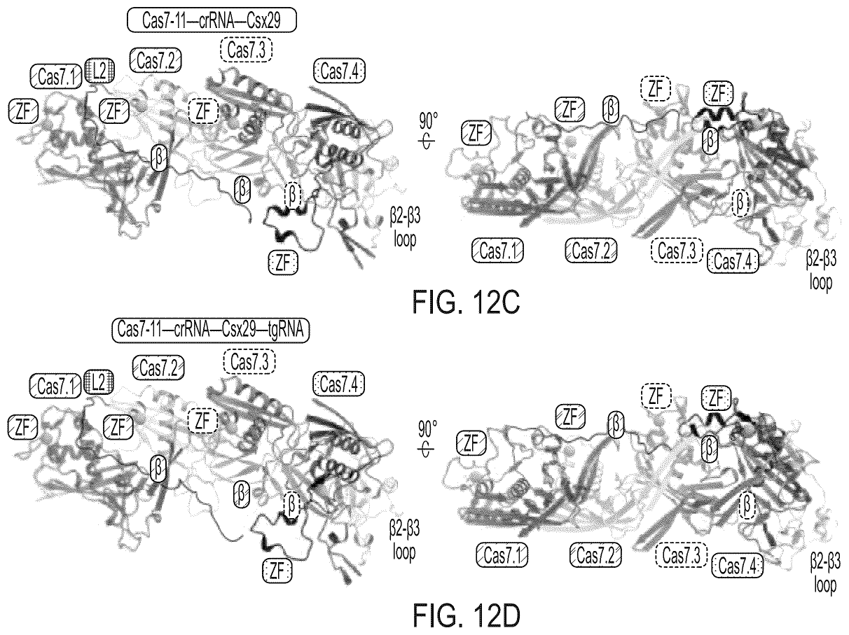

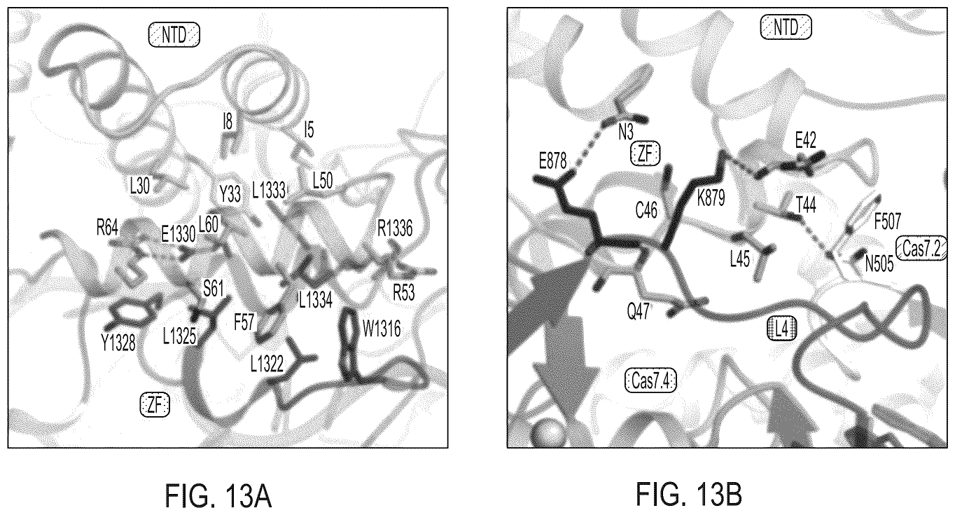

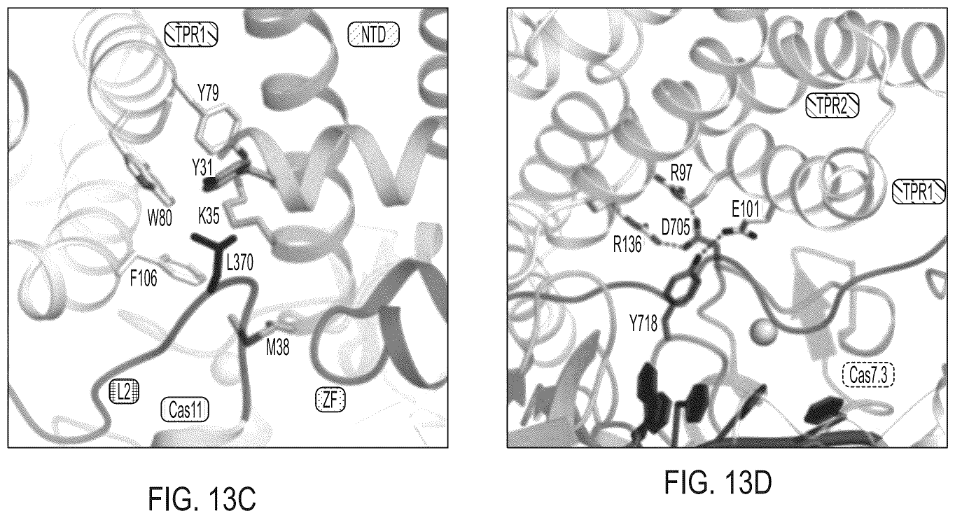

In the Cas7-11-crRNA-Csx29 structure, Csx29 interacts with Cas7-11 at multiple regions (FIG. 2B and FIG. 12A). The L2 linker (residues 367-401) and an α-helical insertion (residues 1313-1341) in the Cas7.4 ZF motif, which are disordered in the Csx29-unbound Cas7-11 structure (FIG. 12B), are ordered and form interactions with Csx29 in the Cas7-11-crRNA-Csx29 structure (FIG. 2B and FIGS. 12A and 12C). This α-helical insertion in the ZF motif is unique to Cas7.4 and absent in Cas7.1-Cas7.3 (FIG. 12C). The NTD of Csx29 mainly interacts with the Cas7.4 domain of Cas7-11 (FIG. 2B and FIG. 12A). I5, I8, L30, Y33, L50, R53, F57, L60, S61, and R64 of Csx29 hydrophobically interact with W1316, L1322, L1325, Y1328, L1333, and L1334 of the α-helical insertion region of Cas7-11, while R53 and R64 form hydrogen bonds with R1336 and E1330 of Cas7-11, respectively (FIG. 13A). In addition, the Cas7.2 thumb-like β-hairpin and the L2/L4 linkers contribute to the binding to the Csx29 NTD (FIG. 2B). N505 and F507 of Cas7-11 (Cas7.2) interact with T44 and E42/L45 of Csx29, respectively, and K879 and E878 of Cas7-11 (L4) hydrogen bond with E42 and N3/Q47 of Csx29, respectively (FIG. 13B). Furthermore, L370 of Cas7-11 (L2) is accommodated within a hydrophobic pocket at the NTD-TPR1 interface of Csx29 (FIG. 13C).

TPR1 and TPR2 interact with Cas7.3 (ZF) and L2 of Cas7-11, respectively (FIG. 2B and FIG. 12A). D705 and Y718 of Cas7-11 hydrogen bond with R97/R136 and E101 of Csx29, respectively (FIG. 13D). TPR2 also interacts with Cas7.1 (thumb-like β-hairpin) and Cas7.2 (ZF) (FIG. 2B). TPR1 and TPR2 are the only TPR domains that mediate the Cas7-11-Csx29 interaction, and TPR3-TPR7 do not contact Cas7-11. The CHAT protease domain of Csx29 interacts with Cas7.1 (thumb-like β-hairpin), Cas7.2 (ZF), Cas7.3 (ZF), and L2 of Cas7-11 (FIG. 2B and FIG. 12A). Notably, the protease active site of Csx29 is located in the vicinity of the Cas7.2 domain of Cas7-11 (FIG. 2C), suggesting limited accessibility for the peptide substrate in this conformation. Furthermore, unlike in the separase-securine structure, the side chain of the catalytic residue C658 is buried inside the CHAT domain in the present structure (FIG. 11), indicating that a structural rearrangement of C658 would be required for the substrate cleavage. These observations suggest that the Cas7-11-crRNA-Csx29 structure represents the inactive state of the Csx29 putative protease.

### Example 5: Target RNA Binding-Induced Structural Change in the Cas7-11-Csx29 Complex

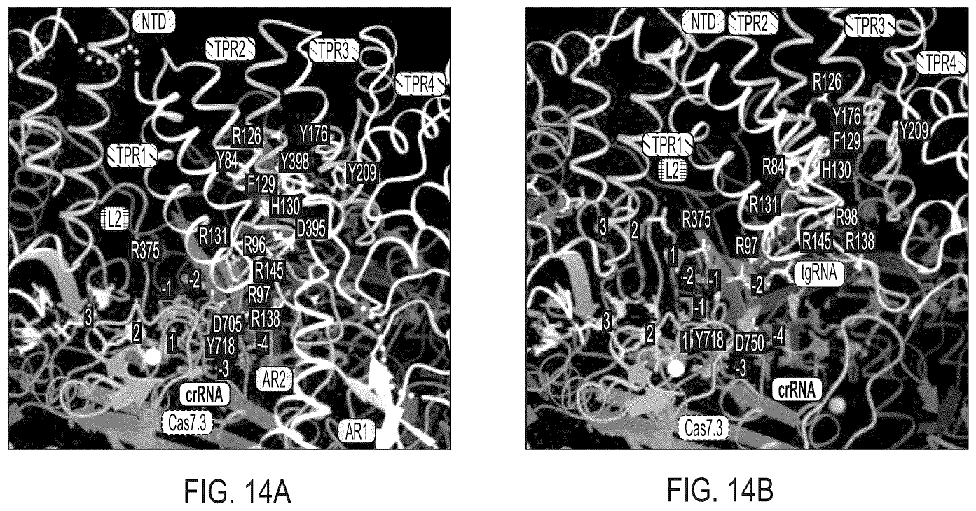

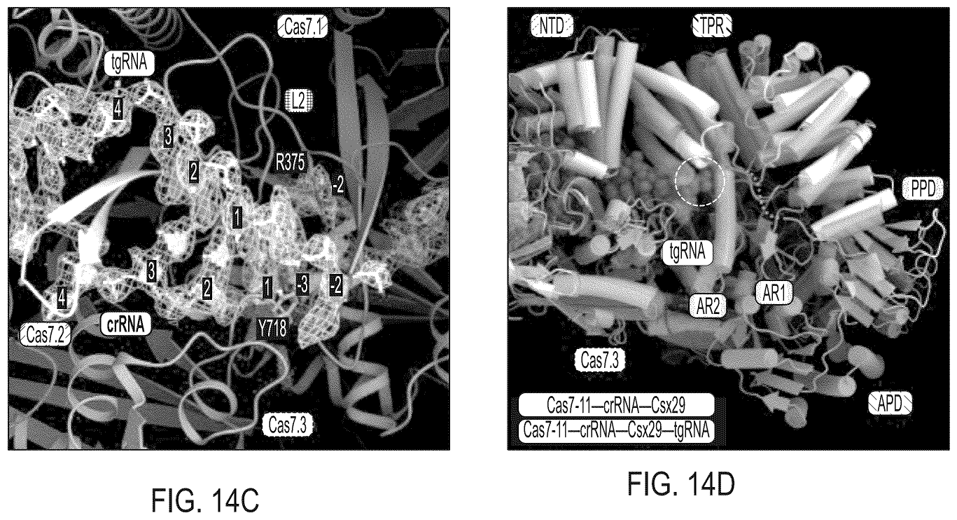

A comparison of the Cas7-11-crRNA-Csx29 structures with and without the tgRNA revealed a notable conformational difference in Csx29 (FIGS. 2D and 2E). In the tgRNA-free structure, TPR1 and TPR2 of Csx29 interact with Cas7.3 and Cas7.1/Cas7.2 of Cas7-11, respectively (FIG. 2D and FIG. 14A). In contrast, in the tgRNA-bound structure, TPR1 and TPR2 of Csx29 move away from Cas7-11 and do not interact with Cas7.1-Cas7.3 of Cas7-11, due to the binding of the tgRNA 3′ region between Cas7-11 and Csx29 (FIG. 2E and FIG. 14B). Among a 6-nt protospacer flanking sequence (PFS) in the tgRNA, only C(-1) and A(-2) are well resolved in the density map, and interact with Cas7-11 (L2/Cas7.3) and Csx29 (TPR1/TPR2) (FIGS. 14B and 14C). The nucleobases C(-1) and A(-2) stack with R375 (L2) and Y718 (Cas7.3), respectively (FIGS. 14B and 14C). In addition, the phosphate groups between A(-3) and A(-2) and between A(-2) and C(-1) interact with R131 (TPR2) and R145 (TPR2), respectively (FIG. 14B). These interactions induce a kink turn between A(-2) and C(-1) in the PFS, thereby projecting tgRNA nucleotides downstream of position −2 toward the AR of Csx29.

There are also structural differences in the AR-APD of Csx29 between the RNA-free and RNA-bound structures. In the tgRNA-free structure, the AR extensively interacts with the TPR1-TPR5 and APD (FIG. 2D). In particular, Y398 (AR2) is accommodated within a pocket formed by Y84 (TPR1), R126/F129/H130 (TPR2), Y176 (TPR3), and Y209 (TPR4), with its hydroxyl group forming hydrogen bonds with Y84 and R126 (FIG. 14A). In addition, D395 (AR2) forms a salt bridge with R96 (TPR1). In the tgRNA-bound structure, the AR-APD of Csx29 is not resolved in the density map (FIG. 2E and FIG. 8B). In addition, the PPD of Csx29 in the tgRNA-bound structure exhibits weaker density, as compared to that in the tgRNA-free structure (FIGS. 8A and 8B). These structural observations suggest that tgRNA binding increases the conformational flexibility of the CHAT protease domain of Csx29 and this conformational change releases the steric block on the Csx29 active site, allowing access to the substrate protein. A structural comparison of the two Cas7-11-Csx29 complexes suggests steric clash between the tgRNA PFS and the Csx29 AR (FIG. 14D), indicating the importance of the PFS for the tgRNA-induced conformational change in Csx29. Together, our structural data suggest that Csx29 is a target RNA-triggered protease.

### Example 6: Target RNA-Triggered Csx30 Cleavage by Csx29

Given that Csx30 and Csx31 are encoded together with Cas7-11 and Csx29 in the D. ishimotonii CRISPR locus and are highly conserved among the type III-E systems, we hypothesized that Csx29 could target either Csx30 or Csx31. To test this hypothesis, we attempted to prepare the recombinant Csx30 and Csx31 proteins and examine whether they are cleaved by Csx29 in a tgRNA-dependent manner. Csx30 could be purified as a soluble protein, whereas Csx31 was expressed in an insoluble fraction. We examined the in vitro cleavage of Csx30 by Cas7-11-crRNA-Csx29 in the absence and presence of the tgRNA, and found that Cas7-11-crRNA-Csx29 cleaves Csx30 into two fragments, Csx30-1 (-50 kDa) and Csx30-2 (-15 kDa), only in the presence of the tgRNA (FIGS. 3A and 3B).

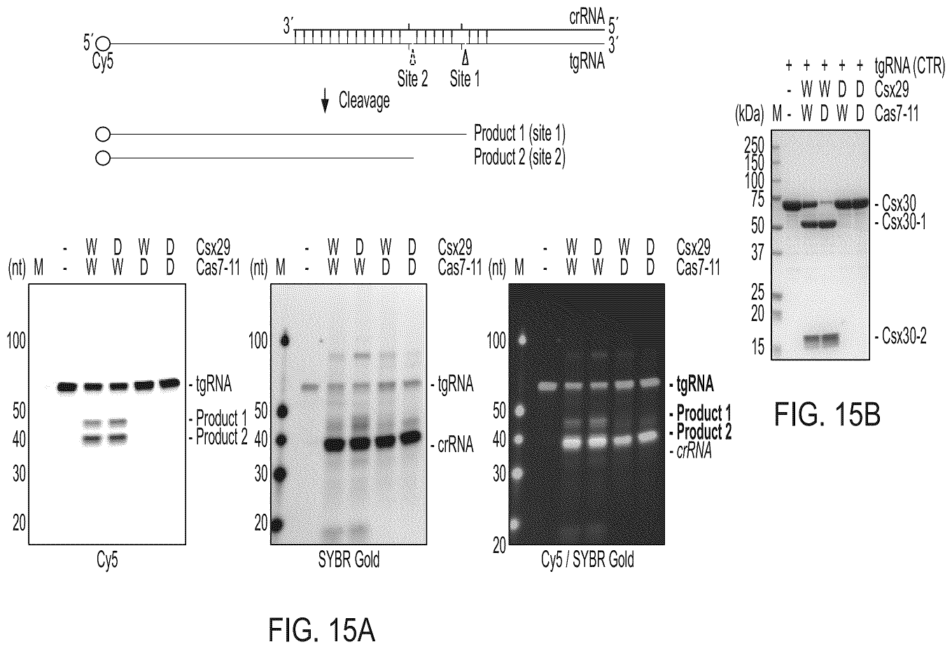

The H615A/C658A mutations in Csx29 abolished the Csx30 cleavage (FIG. 3B), but did not affect the tgRNA cleavage by Cas7-11 (FIG. 15A), indicating the separable nuclease and protease activities. Furthermore, the D429A/D654A catalytic mutations in Cas7-11 (Sequence of Cas7-11 is SEQ ID NO: 5 and sequence of mutant version is SEQ ID NO: 34) abolished tgRNA cleavage (FIG. 15A), as previously observed, and, unexpectedly, improved the Csx30 cleavage by Csx29 (FIG. 3B and FIG. 15B). This improvement in the proteolytic activity suggests that the tgRNA dissociates from the effector complex after the Cas7-11-mediated cleavage and that the Csx29 protease is only active as long as a target RNA is bound to the Cas7-11-Csx29 complex. These results demonstrated that Csx30 is cleaved by the CHAT protease domain of Csx29 in a target RNA-dependent manner.

Base complementarity between the crRNA 5′ tag and a tgRNA PFS regulates the activities of the type III-A Csm effector complex, to avoid autoimmune response in the type III-A system. Thus, we examined the effects of the PFS in the tgRNA on Csx30 cleavage, using either a tgRNA without a PFS (TR), a cognate tgRNA with a non-matching PFS (CTR), or a non-cognate tgRNA with a matching PFS (NTR) (FIG. 3A). Csx30 was cleaved by the Cas7-11-Csx29 complex efficiently in the presence of CTR, but not TR and NTR (FIG. 3C), consistent with our structural observation that a non-matching PFS plays a role in structural changes and protease activation in Csx29.

N-terminal analysis of the Csx30-2 fragment showed that it begins with K428 (FIG. 16), indicating that Csx30 is cleaved by Csx29 between M427 and K428 (FIG. 3D). A structural prediction using AlphaFold2 indicated that Csx30 consists of an N-terminal domain (NTD) and a C-terminal domain (CTD), which are connected by a linker region. The NTD (residues 1-377) contains two α-helical subdomains, whereas the CTD (residues 418-565) comprises a core β-barrel with flanking α helices (FIG. 3D). The cleavage site between M427 and K428 is located at a 0-hairpin in the Csx30 CTD (FIG. 3D). We examined the in vitro Csx29-mediated cleavage of eight Csx30 mutants, in which residues V425-K431 were individually replaced with an alanine. G416A and M427A mutations slightly and substantially reduced the Csx30 cleavage, respectively, whereas the other mutations had almost no effect (FIG. 3E). Thus, Csx29 seems to primarily recognize M427 at the P1 site within the AVGMIKKDK (SEQ ID NO: 37) sequence in Csx30 and cleaves Csx30 between M427 (P1) and K428 (P1′). Together, these results demonstrated that the Cas7-11-Csx29 complex catalyzes target RNA-triggered Csx30 proteolytic cleavage.

### Example 7: Effects of Csx30 and Csx31 on Bacterial Cell Growth

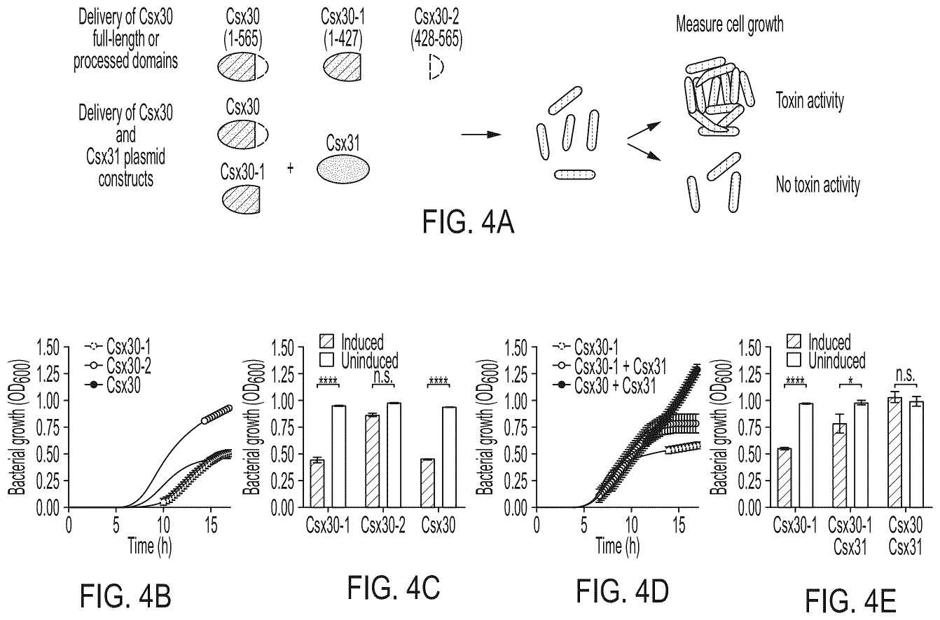

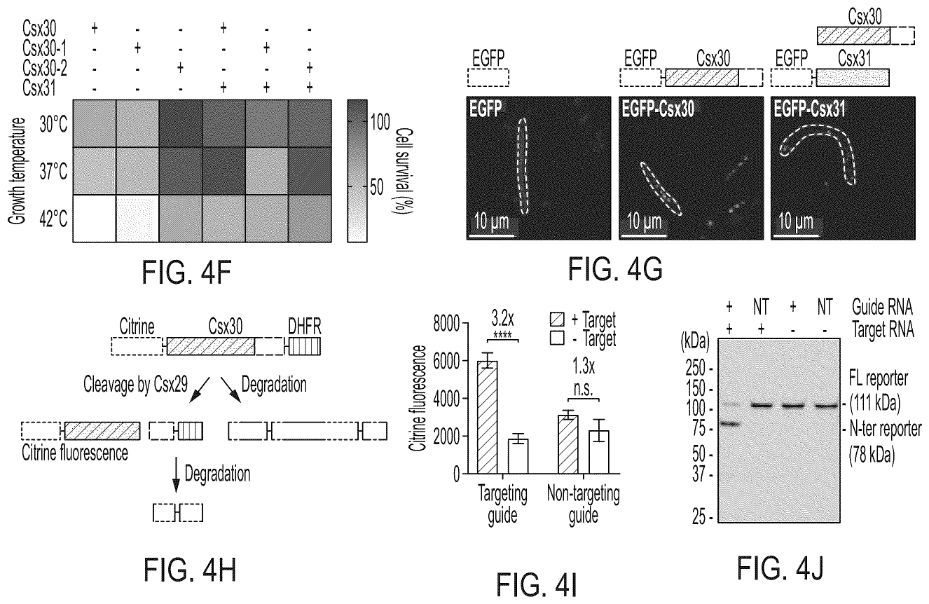

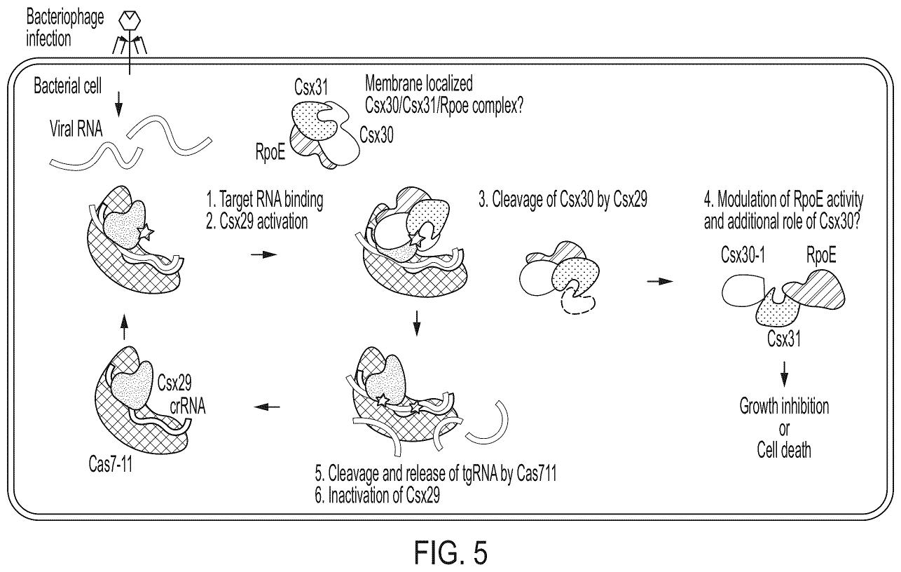

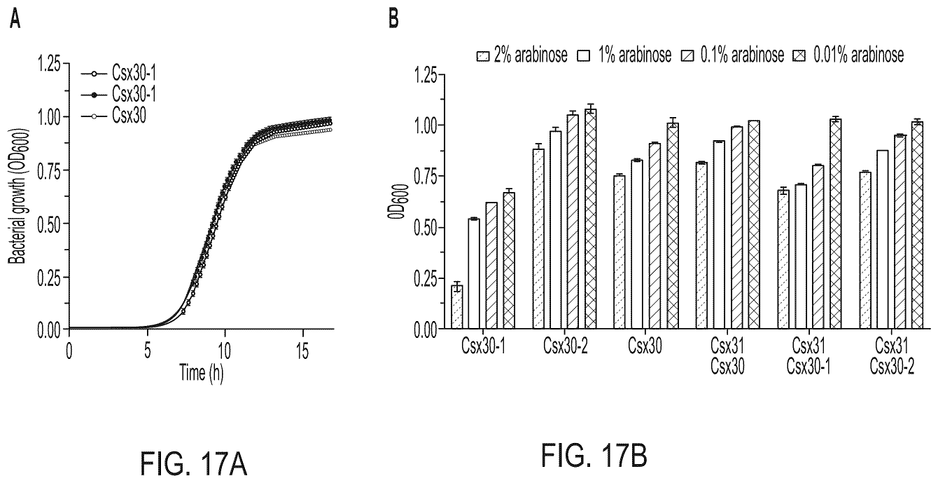

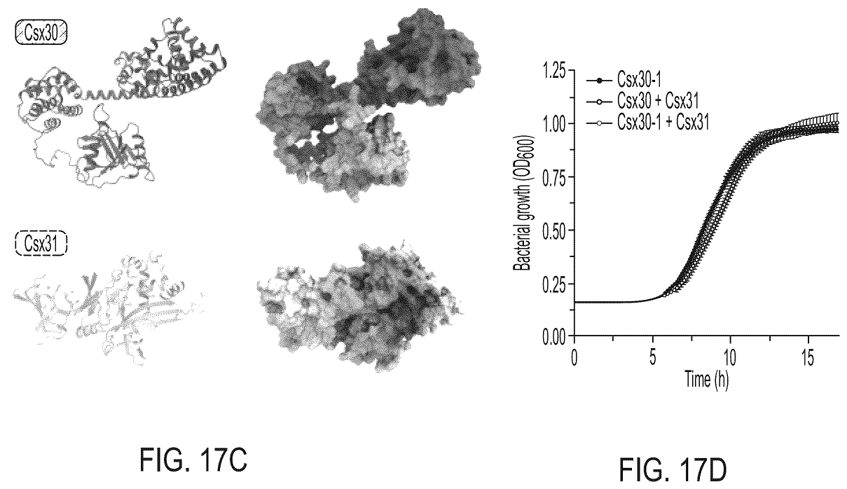

To explore the physiological relevance of the Csx29-mediated Csx30 cleavage, we overexpressed in Escherichia coli the full-length Csx30 (referred to as Csx30 for simplicity), the N-terminal fragment of Csx30 (residues 1-427, Csx30-1), or the C-terminal fragment of Csx30 (residues 428-565, Csx30-2), and monitored the cell growth (FIG. 4A). Overexpression of Csx30 substantially inhibited the cell growth compared to uninduced controls (FIG. 4B, 4C, and FIG. 17A, 17B). Overexpression of Csx30-1 similarly caused pronounced growth suppression, whereas Csx30-2 displayed only mild inhibition (FIG. 4B, 4C, and FIG. 17A, 17B), indicating that Csx30-1 is necessary and sufficient for the observed growth effects of the full-length Csx30. Because the AlphaFold2 structural prediction suggested that Csx30 and Csx31 have oppositely charged surfaces and could electrostatically interact with each other (FIG. 17C), we also explored the effect of Csx31 on bacterial growth. Overexpression of Csx31 rescued the Csx30-mediated growth defect, but could not completely eliminate the Csx30-1-induced growth suppression (FIG. 4D, 4E, 4E, and FIG. 17B, 17D). These data indicate that Csx31 interacts with Csx30 and regulates Csx30-induced growth suppression, whereas the generation of the Csx30-1 and Csx30-2 fragments by the Cas7-11-Csx29 protease interferes with this regulation.

### Example 8: Interactions between Csx30, Csx31, and RpoE

The common co-occurrence of Cas7-11, Csx30, Csx31, and the stress-associated sigma factor RpoE in type III-E CRISPR loci suggests interplay between the four proteins in the locus and that the observed Csx30-induced growth effects might be due to interactions with endogenous E. coli RpoE (EcRpoE). Given the involvement of EcRpoE in the cellular heat shock responses, we hypothesized that the growth defects might be more pronounced at higher temperatures due to inhibition of EcRpoE by Csx30 and Csx31, and tested for the effect of Csx30 and Csx31 in E. coli at different temperatures from 30° C. to 42° C. Corroborating our hypothesis, the growth suppression of Csx30 was more dramatic at higher temperatures across all the combinations tested (FIG. 4F), implicating the involvement of EcRpoE in the observed growth defects due to the overexpression of Csx30 and Csx31.

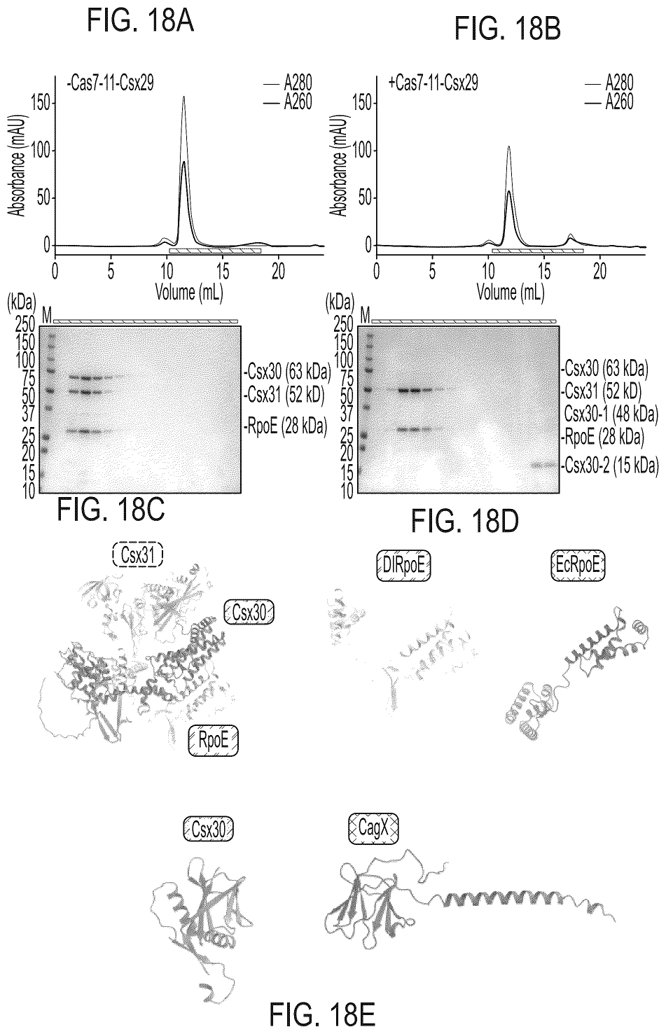

To examine direct interactions between Csx30, Csx31, and D. ishimotonii RpoE (DiRpoE), we co-expressed the three proteins in E. coli and analyzed complex formation using gel-filtration. Csx30, Csx31, and DiRpoE eluted as a single peak from the column (FIG. 18A), indicating that they form a stable complex. Like isolated Csx30, Csx30 in the Csx30-Csx31-DiRpoE complex was cleaved by the Cas7-11-Csx29 complex, and Csx30-1, Csx31, and DiRpoE co-eluted from the column (FIG. 18B), indicating that Csx30-1, Csx31, and RpoE maintain a complex formation after Csx29 cleavage, with separation from Csx30-2. Consistently, structural prediction using AlphaFold2 implied that Csx30, Csx31, and DiRpoE form a ternary complex, in which the Csx30 NTD extensively interacts with DiRpoE (FIG. 18C). DiRpoE shares structural similarity with EcRpoE (FIG. 18D), implying that observed cell growth inhibition in our assays could be mediated via Csx30-EcRpoE interactions, similar to the mechanism of the anti-sigma factor RseA. While EcRpoE is involved in extracytoplasmic stress response in E. coli, associated regulatory proteins like RseA are not present in Desulfonema strains and there are different paralogs of RpoE in E. coli with varied functions, such as FecI, suggesting that DiRpoE could mediate an unknown transcriptional response in its natural role.

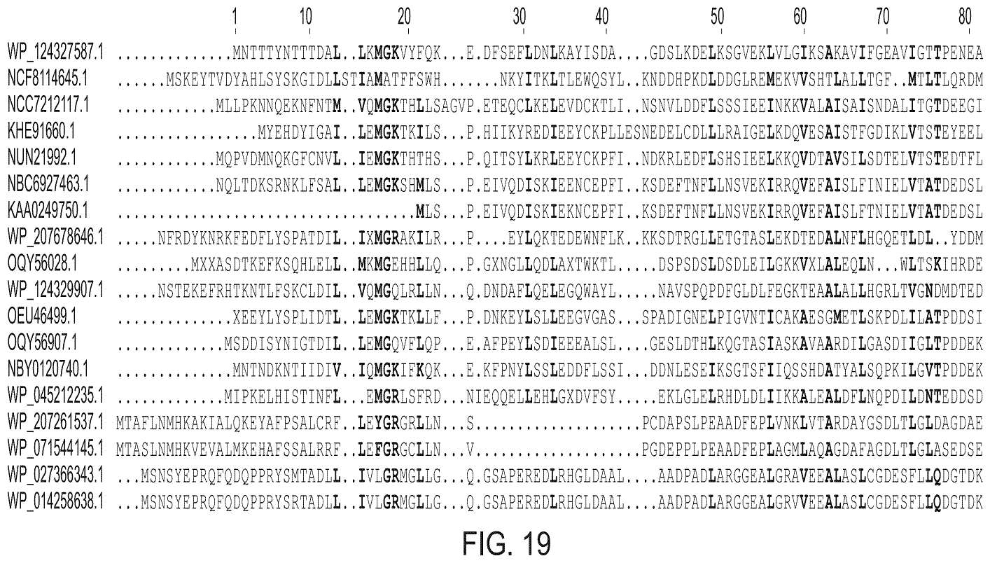

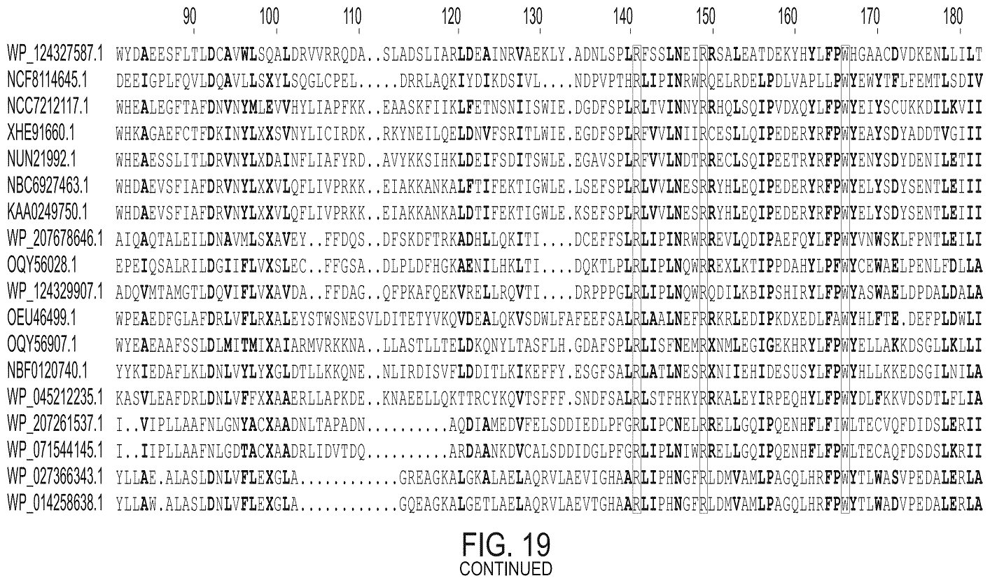

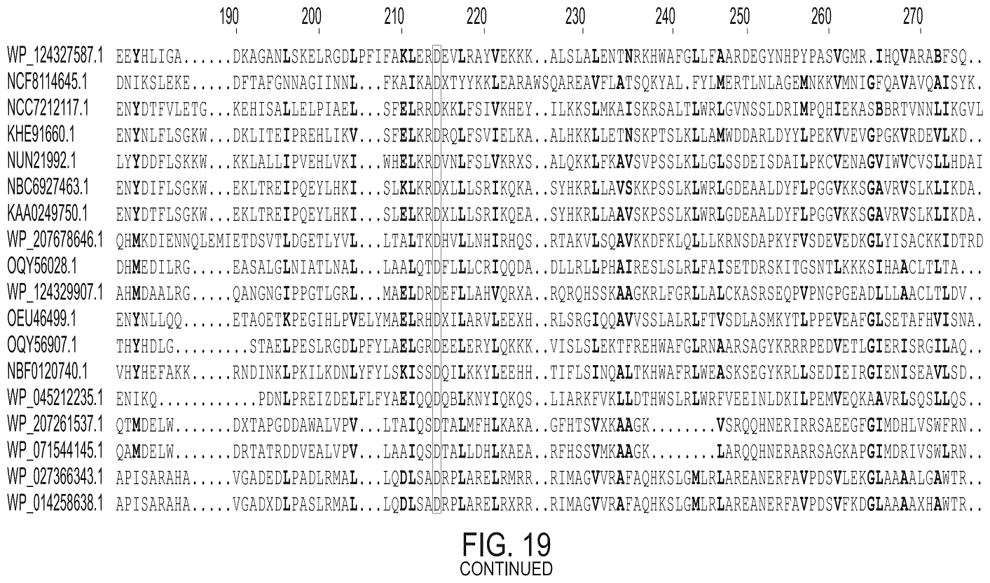

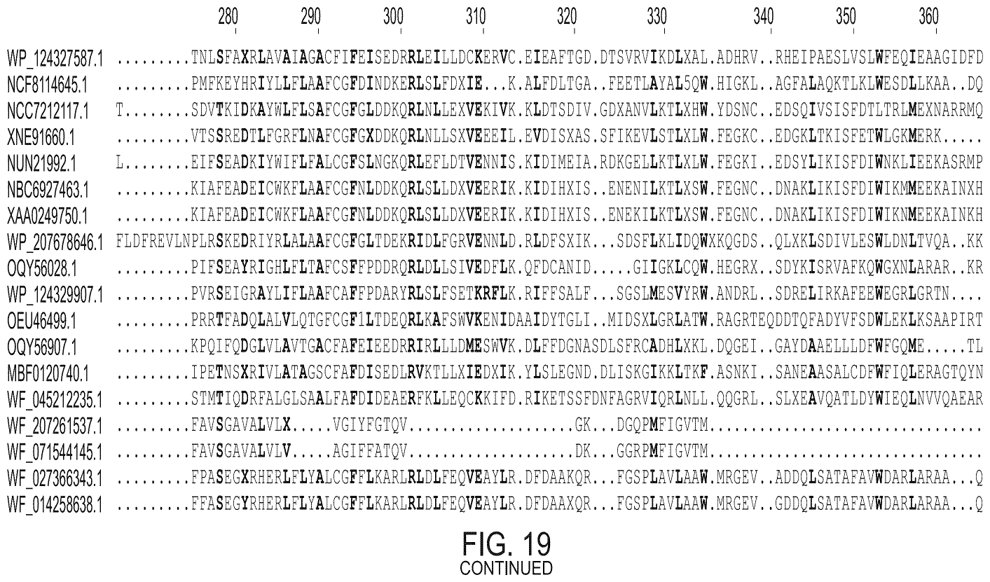

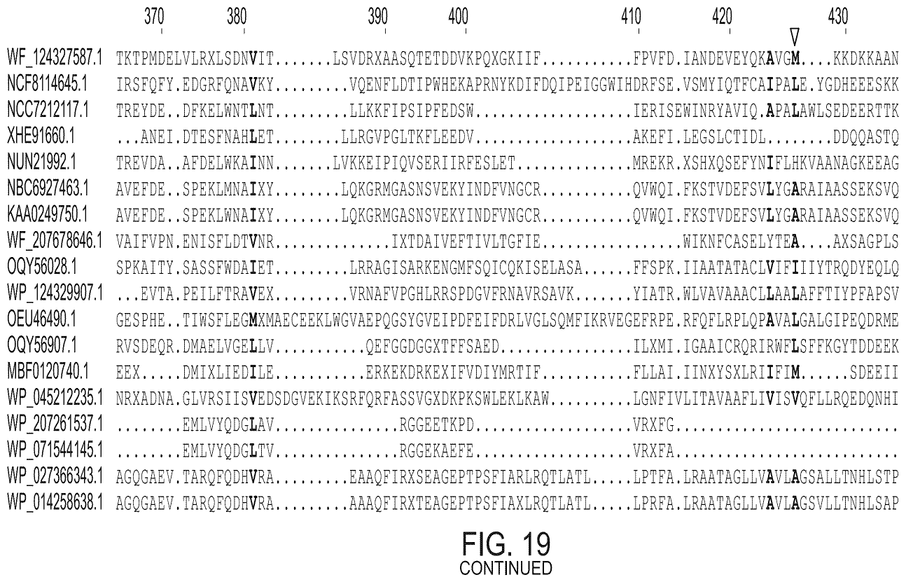

A Dali search revealed structural similarity between the Csx30 CTD and pore-forming proteins in type IV secretion systems, such as CagX (FIG. 18E). Given reduced growth effects of full-length Csx30 in E. coli, compared to Csx30-1, the Csx30 CTD might function as a membrane anchor, rather than a pore-forming protein, consistent with the role of membrane-localized RseA. The CTD and NTD of Csx30 are connected via a flexible linker, suggesting that the Csx29-mediated cleavage releases the N-terminal fragment of Csx30 (Csx30-1) into the cytoplasm, thereby modulating gene expression via RpoE suppression. Sequence analysis revealed that Csx30 NTDs are highly conserved (FIG. 19), whereas Csx30 CTDs are divergent and can be divided into seven distinct groups (FIG. 20), two of which belong to unrelated protein domains found in other contexts. One is an uncharacterized DUF4384 family, which is often fused to different protease domains (see domain architectures for DUF4384 in the CDD database). Another group is similar to pilus assembly protein PilP, which forms a periplasmic ring of bacterial type IV pili. These observations highlight the mechanistic diversity of Csx30-mediated RpoE interaction and programmed gene expression modulation.

### Example 9: Localization of Csx30 and Csx31 in Bacterial Cells

To explore the growth suppression associated with the expression of Csx30, the putative membrane localization of the Csx30 CTD, and the corresponding regulatory function of Csx31, we imaged Csx30 and Csx31 by fusing bacterial codon-optimized enhanced green fluorescent protein (EGFP) at the N termini of both proteins. We imaged protein localization in E. coli with either a plasmid expressing EGFP-Csx30, plasmids expressing EGFP-Csx31 and unlabeled Csx30, or a plasmid expressing EGFP alone. We found that both labeled Csx30 alone and labeled Csx31 co-expressed with Csx30 localized to individual foci, whereas EGFP diffuses throughout the cells (FIG. 4G). These results support a direct interaction between Csx30 and Csx31 via co-localization at foci in bacterial cells prior to Csx29-mediated Csx30 cleavage.

### Example 10: Engineering Csx29 and Csx30 for Programmable RNA Sensing in Mammalian Cells

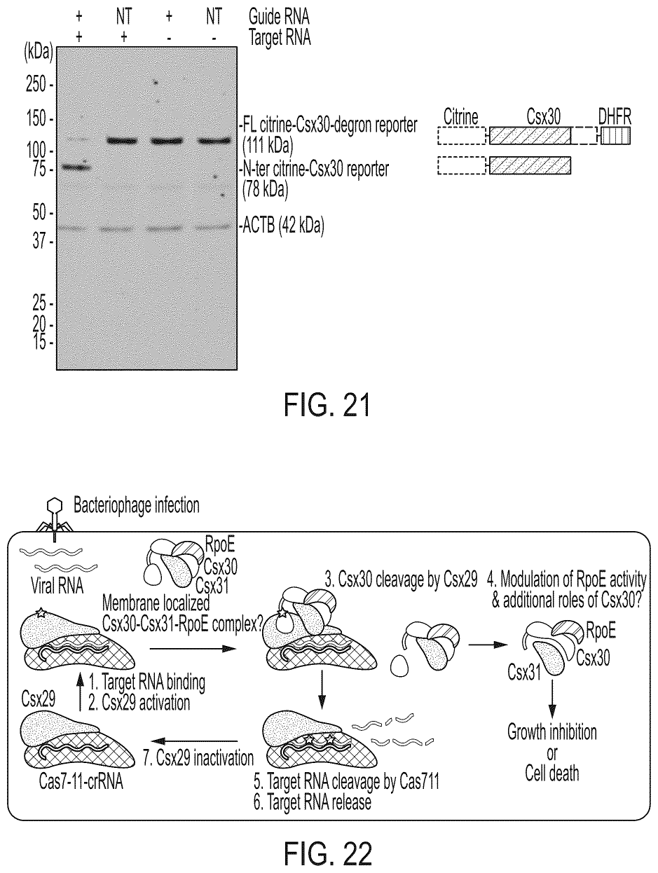

The programmable transcript-activated protease activity of the Cas7-11-Csx29-Csx30 system could enable multiple applications in mammalian cells, including for transcript sensing. To engineer and reprogram the system for mammalian applications, we codon-optimized Cas7-11, Csx29, and Csx30 for mammalian cells, and placed the Csx30 protein sequence between a citrine protein and a dihydrofolate reductase (DHFR) degron, which would eliminate citrine fluorescence unless Csx30 was cleaved by Cas7-11-Csx29 due to sequence specific recognition of a target sequence (FIG. 4H). We transfected HEK293FT cells with either targeting or non-targeting guide RNAs toward a Gaussia luciferase (Gluc) target to test activation of the Cas7-11-Csx29-Csx30 system. In the presence of Gluc mRNA target, we observed 3-fold higher citrine fluorescence in the presence of the targeting, but not non-targeting, guide RNA (FIG. 4I), indicating that Csx29 is activated and cleaving off the DHFR degron from the C-terminal end of the citrine reporter. To validate that the increase in citrine fluorescence is due to the cleavage of Csx30 in the reporter, we analyzed the total protein from the HEK293FT cells by western blot using an anti-FLAG antibody, and visualized the N-terminally FLAG-tagged reporter. The molecular mass of the reporter protein decreased from −110 kDa to 78 kDa only in the presence of the target RNA and targeting guide, indicating the Csx29-mediated cleavage of Csx30 in the reporter (FIG. 4J and FIG. 21). These results demonstrate that the Cas7-11-Csx29-Csx30 system is reprogrammable in mammalian cells and can be used as a protease-based RNA-guided post-translational modification system in a variety of diagnostic and therapeutic settings.

### INCORPORATION BY REFERENCE

All US and PCT patent application publications and US patents mentioned herein are hereby incorporated by reference in their entirety as if each individual patent application publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating cancer, comprising administering to a subject in need thereof an effective amount of:
a) a Cas7-11:Csx29 complex or a first nucleic acid encoding the Cas7-11:Csx29 complex,
b) a guide RNA that specifically hybridizes to a RNA target, and
c) an apoptotic protein fused to an inhibitory peptide via a Csx30 linker or a second nucleic acid encoding the apoptotic protein fused to the inhibitory peptide via the Csx30 linker, the apoptotic activity of the apoptotic protein is inhibited by the inhibitory peptide and the apoptotic activity of the apoptotic protein is activated upon the cleavage of Csx30,
wherein the cancer comprises cells comprising the target RNA; and Csx29 cleaves Csx30 when Cas7-11:Csx29 complex binds to the target RNA.

2. The method of claim 1, wherein the Cas7-11 is a polypeptide comprising an amino acid sequence selected from the group consisting of SEQ ID NOs: 1-34.

3. The method of claim 1, wherein the Csx29 is a polypeptide comprising an amino acid sequence selected from the group consisting of SEQ ID NO: 35 and 57-69.

4. The method of claim 1, wherein the guide RNA is a pre-crRNA.

5. The method of claim 1, wherein the guide RNA is a mature crRNA.

6. The method of claim 1, wherein the RNA target is a single-strand RNA (ssRNA).

7. The method of claim 1, wherein the apoptotic protein is caspase 2, caspase 8, caspase 9, caspase 10, caspase 11, caspase 12, caspase 3, caspase 6, or caspase 7.

8. The method of claim 1, wherein the apoptotic protein is an immune activating cytokine.

9. The method of claim 8, wherein the immune activating cytokine is a cytokine or a chemokine.

10. The method of claim 8, wherein the immune activating cytokine is interleukin 12 (IL-12), interleukin 7 (IL-7), interleukin 15 (IL-15), interleukin 2 (IL-2), interleukin 18 (IL-18), interleukin 21 (IL-21), interleukin 23 (IL-23), interleukin 1 beta (IL-1β), interleukin 6 (IL-6), interleukin 8 (IL-8), CD40L, macrophage inflammatory protein 1 alpha (CCL3) (M1β-1α), macrophage inflammatory protein 1 beta (CCL4) (M1β-1β), interferon gamma (IFNγ), Interferon beta (IFNβ), tumor necrosis factor alpha (TNFα), interleukin-1 receptor antagonist (IL-1ra), or interleukin 10 (IL-10).

11. The method of claim 1, wherein the inhibitory peptide inhibits the activity of the protein via steric hindrance.

12. The method of claim 1, wherein the inhibitory peptide inhibits the activity of the protein via degrading the protein.

13. The method of claim 12, wherein the inhibitory peptide comprises a specific degradation signal, or a degron.

14. The method of claim 13, wherein the specific degradation signal, or a degron is derived from dihydrofolate reductase (DHFR).

15. The method of claim 1, wherein the Csx30 linker is a polypeptide comprising an amino acid sequence selected from the group consisting of SEQ ID NOs: 36-46.

16. The method of claim 1, wherein the cancer is hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, or superficial spreading melanoma.

17. A method of identifying a cell type of a cell based on the presence of a RNA target in the cell, comprising delivering into the cell:
a) a Cas7-11:Csx29 complex or a first nucleic acid encoding the Cas7-11:Csx29 complex,
b) a guide RNA that specifically hybridizes to the RNA target, and
c) a fluorescent protein fused to an inhibitory peptide via a Csx30 linker or a second nucleic acid encoding the fluorescent protein fused to the inhibitory peptide via the Csx30 linker, the fluorescence of the fluorescent protein is inhibited by the inhibitory protein and the fluorescence of the fluorescent protein is activated upon the cleavage of Csx30 or a fluorophore attached to a quencher via a Csx30 linker, the fluorescence of the fluorophore is inhibited by the quencher and the fluorescence of the fluorophore is activated upon the cleavage of Csx30,
wherein the cell type is identified as comprising the target RNA, if Csx29 cleaves Csx30 when Cas7-11:Csx29 complex binds to the target RNA and fluorescence is detected.

18. The method of claim 17, wherein the Cas7-11 is a polypeptide comprising an amino acid sequence selected from the group consisting of SEQ ID NOs: 1-34.

19. The method of claim 17, wherein the Csx29 is a polypeptide comprising an amino acid sequence selected from the group consisting of SEQ ID NO: 35 and 57-69.

20-38. (canceled)

39. A method of modifying a genomic sequence in a target cell based on the presence of a RNA target in the cell, comprising delivering into the cell effective amounts of:
a) a Cas7-11:Csx29 complex or a first nucleic acid encoding the Cas7-11:Csx29 complex,
b) a guide RNA that specifically hybridizes to the RNA target, and
c) a gene editing enzyme attached to an inhibitory peptide via a Csx30 linker or a second nucleic acid encoding the gene editing enzyme fused to the inhibitory peptide via the Csx30 linker, the gene editing activity of the gene editing enzyme is inhibited by the inhibitory peptide and the gene editing activity of the gene editing enzyme is activated upon the cleavage of Csx30.

40-96. (canceled)


SEQUENCE LISTING (122 sequences)

SEQ ID NO: 1 - protein, 1812 aa, synthetic construct
MHTILPIHLTFLEPYRLAEWHAKADRKKNKRYLRGMSFAQWHKDKDGIGKPYITGTLLRSAVLNAAEELISLNQGMWAKEPCCNGKFETEKDKPAVLRKRPTIQWKTGRPAICDPEKQEKKDACPLCMLLGRFDKAGKRHRDNKYDKHDYDIHFDNLNLITDKKFSHPDDIASERILNRVDYTTGKAHDYFKVWEVDDDQWWQFTGTITMHDDCSKAKGLLLASLCFVDKLCGALCRIEVTGNNSQDENKEYAHPDTGIITSLNLKYQNNSTIHQDAVPLSGSAHDNDEPPVHDNDSSLDNDTITLLSMKAKEIVGAFRESGKIEKARTLADVIRAMRLQKPDIWEKLPKGINDKHHLWDREVNGKKLRNILEELWRLMNKRNAWRTFCEVLGNELYRCYKEKTGGIVLRFRTLGETEYYPEPEKTEPCLISDNSIPITPLGGVKEWIIIGRLKAETPFYFGVQSSFDSTQDDLDLVPDIVNTDEKLEANEQTSFRILMDKKGRYRIPRSLIRGVLRRDLRTAFGGSGCIVELGRMIPCDCKVCAIMRKITVMDSRSENIELPDIRYRIRLNPYTATVDEGALFDMEIGPEGITFPFVFRYRGEDALPRELWSVIRYWMDGMAWLGGSGSTGKGRFALIDIKVFEWDLCNEEGLKAYICSRGLRGIEKEVLLENKTIAEITNLFKTEEVKFFESYSKHIKQLCHECIINQISFLWGLRSYYEYLGPLWTEVKYEIKIASPLLSSDTISALLNKDNIDCIAYEKRKWENGGIKFVPTIKGETIRGIVRMAVGKRSGDLGMDDHEDCSCTLCTIFGNEHEAGKLRFEDLEVVEEKLPSEQNSDSNKIPFGPVQDGDGNREKECVTAVKSYKKKLIDHVAIDRFHGGAEDKMKFNTLPLAGSFEKPIILKGRFWIKKDIVKDYKKKIEDAMVDIRDGLYPIGGKTGIGYGWVTDLTILNPQSGFQIPVKKDISPEPGTYSTYPSHSTPSLNKGHIYYPHYFLAPANTVHREQEMIGHEQFHKEQKGELLVSGKIVCTLKTVTPLIIPDTENEDAFGLQNTYSGHKNYQFFHINDEIMVPGSEIRGMISSVYEAITNSCFRVYDETKYITRRLSPEKKDESNDKNKSQDDASQKIRKGLVKKTDEGFSIIEVERYSMKTKGGTKLVDKVYRLPLYDSEAVIASIQFEQYGEKNEKRNAKIRAAIKRNEVIAEVARKNLIFLRSLTPEELKKVLQGEILVKFSLKSGKNPNDYLAELHENGTERGLIKFTGLNMVNIKNVNEEDKDFNDTWDWEKLNIFHNAHEKRNSLKQGYPRPVLKFIKDRVEYTIPKRCERIFCIPVKNTIEYKVSSKVCKQYKDVLSDYEKNFGHINKIFTTKIQKRELTDGDLVYFIPNEGADKTVQAIMPVPLSRITDSRTLGERLPHKNLLPCVHEVNEGLLSGILDSLDKKLLSIHPEGLCPTCRLFGTTYYKGRVRFGFANLMNKPKWLTERENGCGGYVTLPLLERPRLTWSVPSDKCDVPGRKFYIHHNGWQEVLRNNDITPKTENNRTVEPLAADNRFTFDVYFENLREWELGLLCYCLELEPGMGHKLGMGKPMGFGSVKIAIERLQTFTVHQDGINWKPSENEIGVYVQKGREKLVEWFTPSAPHKNMEWNGVKHIKDLRSLLSIPGDKPTVKYPTLNKDAEGAISDYTYERLSDTKLLPHDKRVEYLRTPWSPWNAFVKEAEYSPSEKSDEKGRETIRTKPKSLPSVKSIGKVKWFDEGKGFGILIMDDGKEVSISKNSIRGNILLKKGQKVTFHIVQGLIPKAEDIEIAK

SEQ ID NO: 2 - protein, 1717 aa, synthetic construct
MNITVELTFFEPYRLVEWFDWDARKKSHSAMRGQAFAQWTWKGKGRTAGKSFITGTLVRSAVIKAVEELLSLNNGKWEGVPCCNGSFQTDESKGKKPSFLRKRHTLQWQANNKNICDKEEACPFCILLGRFDNAGKVHERNKDYDIHFSNFDLDHKQEKNDLRLVDIASGRILNRVDFDTGKAKDYFRTWEADYETYGTYTGRITLRNEHAKKLLLASLGFVDKLCGALCRIEVIKKSESPLPSDTKEQSYTKDDTVEVLSEDHNDELRKQAEVIVEAFKQNDKLEKIRILADAIRTLRLHGEGVIEKDELPDGKEERDKGHHLWDIKVQGTALRTKLKELWQSNKDIGWRKFTEMLGSNLYLIYKKETGGVSTRFRILGDTEYYSKAHDSEGSDLFIPVTPPEGIETKEWIIVGRLKAATPFYFGVQQPSDSIPGKEKKSEDSLVINEHTSFNILLDKENRYRIPRSALRGALRRDLRTAFGSGCNVSLGGQILCNCKVCIEMRRITLKDSVSDFSEPPEIRYRIAKNPGTATVEDGSLFDIEVGPEGLTFPFVLRYRGHKFPEQLSSVIRYWEENDGKNGMAWLGGLDSTGKGRFALKDIKIFEWDLNQKINEYIKERGMRGKEKELLEMGESSLPDGLIPYKFFEERECLFPYKENLKPQWSEVQYTIEVGSPLLTADTISALTEPGNRDAIAYKKRVYNDGNNAIEPEPRFAVKSETHRGIFRTAVGRRTGDLGKEDHEDCTCDMCIIFGNEHESSKIRFEDLELINGNEFEKLEKHIDHVAIDRFTGGALDKAKFDTYPLAGSPKKPLKLKGRFWIKKGFSGDHKLLITTALSDIRDGLYPLGSKGGVGYGWVAGISIDDNVPDDFKEMINKTEMPLPEEVEESNNGPINNDYVHPGHQSPKQDHKNKNIYYPHYFLDSGSKVYREKDIITHEEFTEELLSGKINCKLETLTPLIIPDTSDENGLKLQGNKPGHKNYKFFNINGELMIPGSELRGMLRTHFEALTKSCFAIFGEDSTLSWRMNADEKDYKIDSNSIRKMESQRNPKYRIPDELQKELRNSGNGLFNRLYTSERRFWSDVSNKFENSIDYKREILRCAGRPKNYKGGIIRQRKDSLMAEELKVHRLPLYDNFDIPDSAYKANDHCRKSATCSTSRGCRERFTCGIKVRDKNRVFLNAANNNRQYLNNIKKSNHDLYLQYLKGEKKIRFNSKVITGSERSPIDVIAELNERGRQTGFIKLSGLNNSNKSQGNTGTTFNSGWDRFELNILLDDLETRPSKSDYPRPRLLFTKDQYEYNITKRCERVFEIDKGNKTGYPVDDQIKKNYEDILDSYDGIKDQEVAERFDTFTRGSKLKVGDLVYFHIDGDNKIDSLIPVRISRKCASKTLGGKLDKALHPCTGLSDGLCPGCHLFGTTDYKGRVKFGFAKYENGPEWLITRGNNPERSLTLGVLESPRPAFSIPDDESEIPGRKFYLHHNGWRIIRQKQLEIRETVQPERNVTTEVMDKGNVFSFDVRFENLREWELGLLLQSLDPGKNIAHKLGKGKPYGFGSVKIKIDSLHTFKINSNNDKIKRVPQSDIREYINKGYQKLIEWSGNNSIQKGNVLPQWHVIPHIDKLYKLLWVPFLNDSKLEPDVRYPVLNEESKGYIEGSDYTYKKLGDKDNLPYKTRVKGLTTPWSPWNPFQVIAEHEEQEVNVTGSRPSVTDKIERDGKMV

SEQ ID NO: 3 - protein, 1659 aa, synthetic construct
MKITLRFLEPFRMLDWIRPEERISGNKAFQRGLTFARWHKSKADDKGKPFITGTLLRSAVIRAAEHLLVLSKGKVGEKACCPGKFLTETDTETNKAPTMFLRKRPTLKWTDRKGCDPDFPCPLCELLGPGAVGKKEGEAGINSYVNFGNLSFPGDTGYSNAREIAVRRVVNRVDYASGKAHDFFRIFEVDHIAFPCFHGEIAFGENVSSQARNLLQDSLRFTDRLCGALCVIRYDGDIPKCGKTAPLPETESIQNAAEETARAIVRVFHGGRKDPEQAQIDKAEQIQLLSAAVRELGRDKKKVSALPLNHEGKEDHYLWDKKAGGETIRTILKAAAEKEAVANQWRQFCIELSEELYKEAKKAHGGLEPARRIMGDAEFSDKSVPDTVSHSIGISVEKETIIMGTLKAETPFFFGIESKEKKQTDLMLLLDGQNHYRIPRSALRGILRRDIRSVLGTGCNAEVGGRPCLCPVCRIMKNITVMDTRSSTDTLPEVRPRIRLNPFTGSVQEKALFNMEMGTEGIEFPFVLSYRGKKTLPKELRNVLNWWTEGKAFLGGAASTGKSIFQLSDIHAFSSDLSDETARESYLSNHGWRGIMENSIVHESPLEGGAGGCSFGLSDLPKLGWHAEDLKLSDIEKYKPFHRQKISVKITLNSPFLNGDPVRALTEDVADIVSFKKYTQGGEKIIYAYKSESFRGVVRTALGLRNQGNDDITGKKNVPLIALTHQDCECMLCRFFGSEYEAGRLYFEDLTFESEPEPRRFDHVAIDRFTGGAVNQKKFDDRSLVPGKEGFMTLIGCFWMRKDKELSRNEIEELGKAFADIRDGLYPLGAKGSMGYGQVAELSIVDDEDSDDENNPAKLLAESMKNASPSLGTPTSLKKKDAGLSLRFDENADYYPYYFLEPEKSVHRDPVPPGHEEAFRGGLLTGRITCRLTVRTPLIVPNTETDDAFNMKEKAGKKKDAYHKSYRFFTLNRVPMIPGSEIRGMISSVFEALSNSCFRIFDEKYRLSWRMDADVKELEQFKPGRVADDGKRIEEMKEIRYPFYDRTYPERNAQNGYFRWDARISLTDNSMRKMEKDGVPRNVIYKLNTLKNKAYKSEKSFLFDLKNKAGGVGRYKKLVLKHAEVRGGEIPYYSHPTPTDCKLLSLVGPNRQLCRQDTLVQYRIIKHRRGAKPEEDFMFVGTPSENQKGHKENNDHGGGYLKISGPNKIEKENVLTSGVPSVPENMGAVVHNCPPRLVEVTVRCGRKQEEECKRKRLVPEYVCADPEKKVTYTMTKRCERIFLEKSRRIIPFTNDAVDKFEILVKEYRRNAEQQDTPEAFQTILPENGTVNPGDLLYFREEKGKAAEIVPVRISRKVDDRHIGKRIDPELRPCHGEWIEDGDLSKLDAYPAEKKLLTRHPKGLCPACRVFGTGSYKSRVRFGFAALKGTPKWLKEDPAEPSQGKGITLPLLERPRPTWAVLHNDKENSEIPGRKFYVHHNGWKGISEGIHPISGENIEPDENNRTVEVLDKGNRFVFELSFENLEPRELGLLIHSLQLEKGLAHKLGMAKSMGFGSVEIDVESVRVKHRSGEWDYKDGETVDGWIEEGKRGVAAKGKANDLRKLLYLPGEKQNPHVHYPTLKKEKKGDPPGYEDLKKSFREKKLNRRKMLTTLWEPWHK

SEQ ID NO: 4 - protein, 1657 aa, synthetic construct
MLKLKVKITYFQPFRVIPWIKEDDRNSDRNYLRGGTFARWHKDKKDDIHGKPYITGTLLRSALFTEIEKIKIHHSDFIHCCNAIDRTEGKHQPSFLRKRPVYTENKNIQACNKCPLCLIMGRGDDRGEDLKKKKHYNGKHYQNWTVHFSNFDTQATFYWKDIVQKRILNRVDQTCGKAKDFFKVCEVDHIACPTLNGIIRINDEKLSQEEISKIKQLIAVGLAQIESLAGGICRIDITNQNHDDLIKSFFETKPSKILQPNLKESGEERFELAKLELLAEYLTQSFDANQKEQQLRRLADAIRDLRKYSPDYLKDLPKGKKGGRTSIWNKKVADDFTLRDCLKNQKIPNELWRQFCEGLGREVYKISKNISNRSDAKPRLLGETEYAGLPLRKEDEKEYSPTYQNQESLPKTKWIISGELQAITPFYIGHVNKTSHTRSTIFLNMNGQFCIPRSTLRGALRRDLRLVFGDSCNTPVGSRVCYCQVCQIMRCIKFEDALSDVDSPPEVRHRIRLNCHTGVVEEGALFDMETGFQGMIFPFRLYYESKNEIMSQHLYEVLNNWTNGQAFFGGEAGTGFGRFKLLNNEVFLWEIDGEEEDYLQYLFSRGYKGIETDEIKKVADPIKWKTLFTKLEIPPEKIPLTQLNYTLTIDSPLISRDPIAAMLDNRNPDAVMVKKTILVYEQDSSTHKNVPKEVPKYFIKSETIRGLLRSIISRTEIKLEDGKKERIFNLDHEDCDCLQCRLFGNVHQQGILRFEDAEITNKNVSDCCIDHVAIDRFTGGGVEKMKFNDYPLSASPKNCLNLKGSIWITSALKDSEKEALSKALSELKYGYASLGGLSAIGYGRVKELTLEENDIIQLTEITESNLNSQSRLSLKPDVKKELSNNHFYYPHYFIKPAPKEVVRESRLISHVQGHDTEGEFLLTGKIKCRLQTLGPLFIANNDKGDDYFELQHNNPGHLNYAFFRINDHIAIPGASIRGMISSVFETLTHSCFRVMDDKKYLTRRVIPESETTQKRKSGRYQVEESDPDLFPGRVQKKGNKYKIEKMDEIVRLPIYDNFSLVERIREYHYSEECASYVPSVKKAIDYNRMLAQAADSNREFLYNHPEAKSILQGKKEVYYILHKQESKNRGKTKEINPNARYACLTDENTPGSRKGFIKFTGPDMVTVNKELKSKIAPIYDPEWEKDIPDWERSNQESNHKYSFILHNEIEMRSSQKKKYPRPVFICKKNGVEYRMQKRCERIFDFTKEEEKDKEIVIPQKVVSQYNAILKDNKENTETIPGLFNSKMVNKELEDGDLVYFKYKEGKVTELTPVAISRKTDNKPMGKRFPKISINGKMKPNDSLRSCSHTCTEDCDDCPNLCESVKDYFKPHPDGLCPACHLFGTTFYKSRLSFGLAWLENNAKWYISNDFQQKDSKKEKGGKLTLPLLERPRPTWSMPNNNAEVPGRKFYVHHPWSVENIKNNQGNQKDISLKPDSDAIKIKENNRTIEPLGKDNVFNFEISFNNLRDWELGLLLYAIELEDHLAHKLGMAKAFGMGSVKIEIKNLLIKGSINDISKAELIKKGFKKLGIDSLEKDDLSEYLHIKQLREILWFSDKPVGTIEYPKLENKTNSRIPSYTDFVQEKDHETGFKNPKYQNLKSRLHILQNPWNAWWKNEE

SEQ ID NO: 5 - protein, 1601 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 6 - protein, 1573 aa, synthetic construct
MSKTDDKIDIKLTFLEPYRMVNWLENGLRMTDPRYLRGLSFARWHRNKNGKAGRPYITGTLLRSAVIRAAEELLSLNLGKWGKQLCCPGQFETEREMRKNKTFLRRRPTPAWSAETKKEICTTHGSACAFCLLLGRRLHGGKEDVNEDAPGSCRKPVGFGNLSLPFQPTKRQIQDVCKERVLNRVDFRTGKAQDYFRVFEIDHEDWGVYTGEITITEPRVQEMLEASLKFVDTLCGALCRIEIVGSADETKRTTSSKEGCPASTTTRDCSSSENDDTSPEDPVREDLKKIAHVIANAFQNSGNREKVHALADAIRAMRLEESSIINTLPKGKSEKTTEQIEVNKHYLWDEIPVNDTSVRHILIEQWRRWQSKKDDPEWWKFCDFLGECLYKEYKKLTSGIQSRARVMGETEYYGALGMPDKVIPLLKSDKTKEWILVGSLKAETPFFFGLETEQTEEVEHTSLRLVMDKKGRFRIPRSVLRGALRRDMRIAFDSGCDVKLGSPLPCDCSVCQVMRSITIKDSRSEAGKLPQIRHRIRLNPFSGTVDEGALFDIEVAPEGVIFPFVMRYRGEEFPPALLSVIRYWQDGKAWLGGEGATGKGRFALAKDLKMYEWKLEDKSLHAYIDTYGHRGNEHAIGTGQGIDGFRSGSLSDLLSDISKESFRDPLASYHNYLDKRWIKVGYQITIGAPLLSADPIGALLDPNNVDAIVFEKMKLDGDQVKYLPAIKGETIRGIVRTALGKRNNLLAKNDHDDCTCSLCAIFGNENETGKIRFEDLEVYDKDIAKKIDHVAIDRFTGGARDQMKFDTLPLIGSPERPLRLKGLFWMRRDVSPDEKARILLAFLEIREGLYPIGGKTGSGYGWVSDLEFDGDAPEAFKEMNSKRGKQASFKEKISFRYPSGAPKHIQNLKATSFYYPHYFLEPGSKVIREQKMIGHEQYYESYPSGASGEKLLSGRIICSMTTHTPLIVPDTGVIKDPENKHATYDFFQMNNAIMIPGSEIRGMISAVYEAMTNSCFRIFHEKQYLTRRISPEDKELREFIPGIVRIINGDVYIEKAEREYRLPLYDDVHIITNYEELEYEKYIKKNPGREQKIKNAHRFNKNIARIAESNRNYLCSLDRAVRREILSGRKKVNFRLVKVNDNKNPDKEAVELCKTGPLEGLVKFSGLNAVNISNLRPGTAEEGFDAKWDMWSLNIILNRMDVRNSQKKEYPRPALHFNHDGKEYTIPKRCERVFVRAEAGKRAETEGSYKVPRKVQEQYQNILRDYESNIGHIDNTFRTLIENCGLNNGSLVYFKPDNSRKEVVAITPVKISRKTDRLPQGDRFPHTSSDLRPCVRDCLDTEGDIRMLENSPFKRLFHIHPEGLCPACQLFGTTNYRGRVRFGFASLSDGPKWFRKDEGNETCHITLPLLERPRPTWSMPDDTSTIPGRKFYVHHMGYETVKKNQRTLVKTENNRTVKALDKENEFTFEVFFENLREWELGLLLHCLELEPEMGHKLGMGKPLGFGSVKIRIDKLQKCVVNVKDGCVLWEPEEDKIQHYIAKGLGKLTTWFGKEWDRLEHIQGLRSLQRLLPL

SEQ ID NO: 7 - protein, 1526 aa, synthetic construct
FESYARWCKSNSGLWKPYIPGTLLRSAVLESVEYLLALIGSKNKVEICPGLYTQSENNPDTKYLRRRPWYELHAQKEICKTRDTACPLCLLMRTKLDNDGDGETEKNVKFGNLYPTSPLEPLQKIRPRILNRMDPGTSKARDYFRVFEIENQLCSQFRGWIWLSGDLPNMELVKSLLAAGLSNVATLAGAVCRIRIVSTDNPSMKQDLTTQDLIDDFTNYYLKGDTPPANLAASGKGDAFPRFSPGSGDHPDTTGVSHADMASSHEGTALAKDIAEKCKDILSQISASEQLRRLADIMRDLRQDSNREIMYRQVAEENHEKASLLYKKTKKGDSIAALIAGKTEGMDAETWRRLCEFLGQTFYGEAKEAGLVETPVPRILGESERYSLQKKPTVRTDLAAELVPDIEFIIKGNLIAETPFFFGTDIATETHTDLPILLTSDRHFRIPRSVLRGILRRDLRLVTGSGCSVKLGRSEPCACDVCQIMRSLTMRDCVSSCKVPPEIRHRIRLNPVTETVEEGALFDMEIGPQGISFPFVLRSRGVNSSFSTRLKNVLTWWSEGKIFMGGDKGTGKGRFTLAELEAYYFRLTTKRIGKNVWVIGNYLKSQGWRGAELETHFDSLKEWKSLSFSDSDVKVFTWHKITWKVSFEGPVLTNDPIAADIRNESDAVFYQKSVAGEKGPVYALKGEGLRGIVSSSLCKKKNLSSNLHEDCECLRCKIFGSKHQEGNIRFEDMTVSQESEVREKLFDHVSIDRFTGGAANKLKFDDKPLVGNPLVFQGVFWVHQSIGNNEKTQEALSDAFKDVRDGLYPVGAKGSIGYGWIKGIEVVEGPDWLKDALSAEKTVEAGIASEESEYKLPDLPWISLLPKGRAIYNPHYFLGIPKVTPEREREPVGHDRFQTDLHTGRIICTLKTITPLIIPDTENDKAFEVENASADHERFKFMRMGSQAAIPGSAIRSMTSSVFEALTNSCFRVLDQKSHLSWRMEADDAGDYKPGRFEKKDDKAVIRKFKKKARFPFYAGPDTREAFTSDQIMGKEKVTLWVKDFEASLTVPDEIGWKKKRGYLKVTGPNKVEIDTENISENNPSPPDSWQDVRINDDGTIPDKKNRKFICQYGTTTYTVDKWCEAFFCDEEKDPYELAPDVERKYRLLMDSYHNNPQAPPQIFRSLPLFSETGPKKTLEHGDLVYFRLSEVNKQSQSKKQVRERVTDIVPVSISRIANNQPIGKHIAAAFRPCAYVCIEECEPCDAKTCPIPVYREGYPIKGLCRACHLFGTTGYKGRVRFSFAKLNGDAVWAKGAGGKDYFTLPLLEKPRPTWTMPNEGAKIPGRKFYVHHNEWKTVQEGKNPIDQKAIRPNPNNSSVEVLNLGNEFQFEVSFENLEEWELGLLLYCLELEPGLAHKLGRGKAFGFGSIEAEVSKIEMRIKSGTWKNETSGKEKFIQSGLSQVPSFFKQDEKQWNKVEQVKNIRKLLQLSWNKGNAVEPEVRYPALREKDDENKRPGYVELKDNGYDAGKKLVSPWAPWHPIKK

SEQ ID NO: 8 - protein, 1403 aa, synthetic construct
MTKKPGTEDKATLWGKESASKSVKTILEESIQGFTVEQKRSFFANLADQLVSRAGEQGAKSVRSQGLIIGRKENYAKPSAQEPTRHHLYRQPSNASAFLATGWLIAETPFFIGSGTEGQKQTDDQAESLHLRTLRDGHGRFRIPFTTIRGVMDKELRDILQAGCAKGRSLRAPCPCQVCTLMRRIQVRDAIAADILPPDLRMRTRIDPSHGTVAHLFSLEMAPQGLKLPFFLKLKGVETIDPDKELLEILNDWSAGQCFLGGLWGTGKGRFRLDDLQWHRLELDNADYYTPLLQDRFFAGETISDLRQGLQSINIQPERIPAQTPSRNMPYCRVDCILEFKSPVLSGDPVAALFESDAPDNVAYKKPVVQYDETGRLRTTDPGPVEMLTCLKGEGVRGVVAYLAGKAYDQHDLSHDSCNCTFCQAFGNGQKAGSLRFDDFMPVQFESDQAGNFSWSPHTPHAMRSDRVALDVFGGAMPEAKFDDRPLAASPGKPLNFKSTIWYREDMGKEAGKALKRALIDLQNNMAAIGSGGGIGRGWVSRVCFEGDIPDFLEDFPEPITVTEPEQDSQLLKNQAVADETAVSACDTADAPHPLAVTLEPGARYFPRVIIPRAPTVKRDECVTGQRYHTGRLSGKIFCELNTLGPLFVPDTDYSAGVPVPISDEQLAECQLQAVFENTSKFNEFFATYPEETVTKLKDLLCAADDKWILAVKDITADLRQEIGEDTFQRIIRKAGHKTQRFHQINDEIGLPGASLRGMVLSNYQILTNSCYRNLKATEEITRRMPADEAKYRKAGRVTVSGDGAQKKYSIQEMEVLRLPIYDNMNTPDNMPDVAKQATTAKRCNNLMNEAAKTSRVELKARWREGQSKIKYQIIDALNKVDPIIQVISSSKQINPNNGKTGWGYVKYTGANVFAKSLVAPIDCLRKKDAGHVCCQVNLNPAWEASNFDILINEKCPVERQSGPRPTLRCKGQDSAWYTLTKRSERIFTDKKPVPDPINIPPREVKRYNELRDSYKKNTAHVPKPLQTFFNQESLANGDLVYFEVNQFGEASQLTPVSISRTTDLFPIGGRLPQGHKDLFPCTAMCLSECKNCVPASFCEFHSRSHEKLCPACSLAGTTGNRGRIKFSEAWLSGLPKWHSVSQDNVGRGLGVTMPRLERSRRTWHLPTKDAYLLGQSIYLNHPVPAILPSDQVPSENNQTVEPLGPKNIFSFQLAFDNLSIEELGLLLYSLELESGMAHRLGRGRALGMGSVQISVKDIQIRDNKSFLFSSNISKKSEWIQCGKDEFAQEAWFGESWDNIDHIQRLRQALTIPVKGDVGCIRYPKLEAEGGMPDYIKLRKRLTPLCDREEPVRYRINPVQLARMILPFVPWHGACPALLNEQVMIEAKRLTELXXXDRANWPC

SEQ ID NO: 9 - protein, 1367 aa, synthetic construct
ASEDDDTPTLRKVLKDEINGQEDMWRKFCEALGNSLYDLSKKAKERKRTEALPRLLGETEIYGLPMRENKEDEPLPSSLTYKFKWLIAGELRAETPFFFGTEVQEGQTSATILLNRDGYFRLPRSVIRGALRRDLRLVMGNDGCNMPIGGQMCECGVCRVMRHIVIEDGLSDCKIPPEVRHRIRLNCHTGTVEEGALFDMETGYQGMTFPFRLYCETENSDLDSYLWEVLNNWQNGQSLFGGDTGTGFGRFELTEPKVFLWNFSKKEKHEAYLLNRGFKGQMPVQDVKTKSFKTKTWFQIHRELDISPKKLPWYSTDYRFNVTSPLISRDPIGAMLDPRNTDAIMVRKTVFCPDPNAKNRPAPATVYMIKGESIRGILRSIVVRNEELYDTDHEDCDCILCRLFGSIHQQGSLRFEDAEVQNSVSDKKMDHVAIDRFTGGGVDQMKFDDYPLPGCPAQPLILEGKFWVKDDIDDESKSALEKAFADFRDGLVSLGGLGAIGYGQIGDFELIGGSADWLNLPKPEENRTDVPCGDRSAQGPEIKISLDADKIYHPHFFLKPSDKNVYRERELVSHAKKKGPDGKSLFTGKITCRLSTEGPVFIPDTDLGEDYFEMQASHKKHKNYGFFRINGNVAIPGSSIRGMISSVFEALTNSCFRVFDQERYLSRSEKPDPTELTKYYPGKVKRDGNKFFILKMKDFFRLPLYDFDFEGEAESLRPNYDEDRNEEENKGKNKNTQKVKNAVEFNIKMAGFAKHNRDFLKKYKEQEIKDIFMGKKKVYFTAGKHKPNEAHDNDKIALLTKGSNKKAEKGYFKFTGPGMVNVKAGVEGEECDFHIDESDPDVYWNMSSILPHNQIKWRPSQKKEYPRPVLKCVKDGTEYVMLKRSEHVFAEASSEDSYPVPGKVRKQFNSISRDNVQNTDHLSSMFQSRRLHDELSHGDLVYFRHDEKRKVTDIAYVRVSRTVDDRPMGKRFKNESLRPCNHVCVEGCDECPDRCKELEDYFSPHPEGLCPACHLFGTTDYKGRVSFGLGWHESNTPKWYMPEDNSQKGSHLTLPLLERPRPTWSMPNKKSEIPGRKFYVHHPWSVDKIRNRQFDPAKEKQPDDVIKPNENNRTVEPLGKGNEFTFEVRFNNLREWELGLLLYSLELEDNMAHKLGMGKALGMGSARIKAEAIELRCESAGQNAELKDKAAFVRKGFEFLEIDKPGENDPMNFDHIRQLRELLWFLPENVSANVRYPMLEKEDDGTPGYTDFIKQEEPSTGKRNPSYLSSEKRRNILQTPWKHWYLIPPFQASAQSETVFEGTVKWFDDKKGFGFIKINDGGKDVFVHHSSIVGTGFKSLNEGDSVAFKMGVGPKGPCAEKVKKIGN

SEQ ID NO: 10 - protein, 1322 aa, synthetic construct
MRRQRLLGDAEYYGGTGREQPASIVISTDSDPDHKVYEWIITGQLKAETGFFFGTKAGAGGHTDLSILLGKDGHYRVPRSVFRGALRRDLRVAFGAGCRVEVGRERPCECPVCKVMRQITVMDTISSYREAPEIRQRIRLNPYTGTVDKGALFDMEVGPEGIEFPFVLRFRGSKSFPSELAAVIGSWTKGTAWLGGAAATGKGRFSLLGLSIHKWNLSTAEGRKSYLAAYGLRDAADKTVKRLSIDKGGKGDVGLPAGLERDALPSSVREPLWKKLVCTVDFSSPLLLADPIAALLGVEGDERIGFDNIAYEKRRYNGETNTTESIPAVKGETFRGIVRTALGKRHGNLTRDHEDCRCRLCAVFGKEQEAGKIRFEDLMPVGAWTRKHLDHVAIDRFHGGAEENMKFDTYALAASPTNPLRMKGLIWVRSDLFETGHDGPTPPYVKDIIDALADVKRGLYPVGGKTGSGYGWIKDVTIDGLPQGLSLPPAEERVDGVNEVPPYNYSAPPDLPSAAEGEYFFPHVFIKPYDKVDRVSRLTGHDRFRQGRITGRITCTLKTLTPLIIPDSEGIQTDATGHKMCKFFSVAGKPMIPGSEIRGMISSVYEALTNSCFRVFDEEKYLTRRVQPKKGAKSSELVPGIIVWGQNGGLAVQQVKNAYRVPLYDDPAVTSAIPTEAQKNKERWESVPSVNLQGALDWNLTTANIARDNRTFLNSRPEEKDAILSGTKPISFELEGTNPNDMLVRLVPDGVDGAHSGYLKFTGLNMVLKANKKTSRKLAPSEEDVRTLAILHNDFDSRRDWRRPPNSQRYFPRSVLRFSLERSTYTIPKRCERVFEGTCGEPYSVPSDVERQYNSIIDDISKNYGRISETYLTKTANRKLTVGDLVYFIADLDKNMATHILPVFISRISDEKPLGELLPFSGKLIPCEGEPPTILKKMAPSLLTEAWRTLISTHLEGFCPACRLFGTTSYKGRIRFGFAEHTGTPKWLREELDWARPFLTLPIQERPRPTWSVPDDKSEVPGRKFYLHHHGGNRIVESNLRNRPEVNQTKNNSSVEPISAGNTFTFDVCFENLEAWELGLLLYCLELSPKLAHKLGRAKAFGFGSVKIHVERIEERTTDGAYQDVTAVKKNGWITTGHDKLREWFHRDDWEDVDHIRNLRTVLRFPDADQEHDVRYPELKANNGVSGYVELRDKMTASERQESLRTPWYRWFPQNGTGGSGRHEQAATSQEQDTAKDESVLSATQRRQAVIDVSDPDERLSGTVESFDRQKGDGYIGCGVRQFYVRLEDIRSRTALCEGQVVTFRARKEWEGHEAYDVEIDQ

SEQ ID NO: 11 - protein, 476 aa, synthetic construct
MLEKALADFRDGVVSLGGLGAIGYGRIGDFEVAEESGTWLKIPEKKLPEDSVQCGERYRFSSDPATRFEKEKIYYPHYFLKPSDEDVRRETRLVSHVYQEDTDGKTRLLTGTIRCRLTTEGPIFIPDTDDPKEDYFQMEIEGHKSYGFFRINEQVAIPGSSIRGMVSSVFEALTNSCFRVFDQKRYLSRSTKPDPRELEKYLPGKVKRIDNKWVLLELEDIFRLPLYDLKDVGPKSLDSAYGLEKFKNEKRFRLKKIENAVAFNKKMAGYAKHTREFLKNNYTETELGKILRGEMKVWFTIGHKPNSAHDNDKIALLTKKTNKRAKSGFIKFTGPSMVNIKADASNSDCEECRFDMKSEDKDGLIFHNAIECRPSQKKEYPRPVLKCVKEAVEYTMIKRCEQVFSEGKKPPRSYSIPDKSRRQYNGILKDNRDNTEHIPSFFRNRMKNKELSDEDLVYFRYKGKKVTNIAPVRVSR

SEQ ID NO: 12 - protein, 437 aa, synthetic construct
QYNLPLNPDAFPKFRWIVTGHLRAETPFFFGRGEIKDRTIEEETEQTSKTILLNKDNFFRLPRSVIRGALRRDLRLVIGNGCNTPVGGKFCECDVCRIMRHVVVEDTISSCRKPPEIRYNIRLNGHTRTVEEGALFDTETGYQGMRFPFRLCFETRASEFDPDTSEPIPKFDPYLSEVMKHWKAGQAVFGGDTGAGFGRFRLEGDIRFFSMDVAKKEEYDPYLLARGFKGMSSQEILEKIGSGRTYDWNSVPKIALNIPPNKLPWKEICYTIEVISPLISRDPIRAMMDPRNTDTIMIRKRVFVPDGKGGTLPEPESRYFIKSETLRGILRSLVGGNKTADGEYLCDLDHEDCDCVQCRLFGSIHQQGCLRFEDAEVWNSVRDKKMDHVAIDRFTGGALDQMKFDDYPLLGCPEYPVILGGRFWIRDDISDKEKEIA

SEQ ID NO: 13 - protein, 350 aa, synthetic construct
MESIPVTLTFLEPYRVVEWYANEDRRSAERYLRGQSFARWHRKKNDKKGRPYITGTLLRSAAIRAAEELLSLSGGVWDGQHCCKGQFLSGGVKPEYMRKRPTYIWAEKEGACSAPDYCPFCIFLGDRDQAEKKAESQNGYPDKSYHIRFGNLSLPDPPPLLDLKEVAVERTLNRVDFQTAKAHDYFKVWEISHEDLGVYTGQIVIHYTGPWQEKVKSLLEGSLRFVDRLCGALCKIEMAPKPARPLPKSLSVDMTEHAKIIVTAFDDAKKAEKVRGLADAMRSMGSKGPTILDKLPAGHDDRDHHTWDVTIVDKTPLRTYLKGVLRADDAASWPALCKALGNALYDVSQG

SEQ ID NO: 14 - protein, 348 aa, synthetic construct
EKQGFRDKGFNIVGSLKDAIGKEIGLREISLRPAKEETMPRWQCVEYTIIVNSPLHTADPIEALLHSGNYDSVVYKKTVVRNGNIKQIPVFKGETIRGIVRTAFARILRTENVEFDEEHEDCTCPLCQVFGNEHRAGRVRFEDLVIEGYTSEKKFDHVSIDRFTGGAAEKRKFDDLSLKGSPRRPIVLRGKVWIRNDMDSKGIEKLKQAFMDIRDGLYPLGSRGGIGYGWVTDLKIENTEVEEFRLDKVSTTEGSGPATEEFNFPSLPEIQLNKDAVYHPHYFIRPHEKVNREIRPVGHERFHDDLLTGRIKCTLKTLTPLIIPDTEDPDAFGLQAEHKGHQNFRFFR

SEQ ID NO: 15 - protein, 1528 aa, synthetic construct
MKSIPITLTFLEPYRILPWAEKGKRDKKEYLRGANYVRLHKDKNGKFKPYITGTLIRSAVLSAIEMLLDITNGEWNGKECCLAKFHTEGEKPSFLRKKPIYIRAEKDEICTSRETACPLCLILGRFDKAEKKEKDKEKFDVHFSNLNLYSSKEFSTIEELAPKRALNRIEQYTGKAQDYFTVYEALNKEFWTFKGRIRIKEDIYDKVTDLLFSALRCVEKIAGALCRIEIDKEPSQQKGFVKRQLSKQAKEDIEKIFQVVKDAQKLRLLSDCFRELTRMANKDELALPLGPEDDGHYLWDKIKVEGKTLRIFLRNCFSQYKDNWLCFCDEASKKGYQKYREKRHKLTDRELPTATPKHFAEKKDPQISPIYIDKDDKVYEWIIVGRLIAQTPFHFGDEEKAEGAILLTPDNRFRLPRTALRGILRRDLKLAGASACEVEVGRSEPCPCDVCKIMRRVTLLDTVSEDLRDFLPELRKRIRINPQSGTVAEGALFDTEVGPEGLSFPFVLRYKCEKLPDSLTTVLCWWQEGLAFLSGESATGKGRFRLEINGAFVWDLQKGLFNYIKNHGFRGEERLFLEGNEAELEKMGIQINTELLQPEMIKKEKNFTDFPYDLIKYQLNISSPLLLNDPIRAIALYEGEGKAPDAVFFKKYVFENGKIEEKPCFKAESIRGIFRTAVGRIKNVLTKNHEDCICVLCHLFGNVHETGRLKFEDLKIVSGQEEKFFDHVAIDRFLGGAKEKYKFDDKPIIGAPDTPIVLEGKIWVKKDINDEAKETLSQAFSDINTGIYYLGANGSIGYGWIEEVKALKAPSWLKIKEKPNFEKDTSLNISAIMNEFKKDIQTLNLDKTYLPYGFLKLLEKVKRTSSPITHERFYENHLTGFIECSLKVLSPLIIPDTETPEKEENGHKYYHFLKIDNKPIIPGAEIRGAVSSIYEALTNSCFRVFGEKKVLSWRMEGKDAKEFMPGRVSKKKGKLYMVKMQALRLPVYDNPALANEIRSGSIYEKYKNSKVEIIFFQTVEGIRKFLRGNFNNVEWKKVLVTGIDPLAILPSQKIPGNDKWVKNLQSKISPVRGYFKFTGPNKIETKRREEEKDEKLRTKANKVSCLQKDKWYEAMHNHVEYKQDYTPPNSPKTEPLERPRNIPCFVCSDKEKIYRMTKRCERVFVSLGENAPKYEIPISAIKRYEVILSAYRENWERNKTPELFRTRLPGDGRTLNEDDLVYFRADENEKVKDIIPVCISRIVDEVPLIKRLSQELWPCVLAECPLLGFECKKCELEGLPEKIWFRINKDGLCPACRLFGTQIYKSRVRFSFAYAKNWKFYDGYITLPRLESPRATWLILKEKDKHYIKYKVCGRKFYLHNSTYEDIINNSKKEKEKKTENNASFEVLKEGEFTFKVYFENLENWELGLLLLSLTGLGEAIKIGHAKPLGFGSVKIEAKKIYFREEAGKFHPCEKADEYLKKGLNKLTSWFGKNEINEHMRNLLLFMTYYQNLPKVKYPDFDGYAKWRCSYVEQDKVEYFQNRWIVAS

SEQ ID NO: 16 - protein, 1652 aa, synthetic construct
MIINITVKFLGPFRMLEWTDPDNRNRKNREFMRGQAFARWHNSNPQKGSQPYITGTLVRSAVIRSAENLLMLSEGKVGKEKCCPGEFRTENRKKRDAMLHLRQRSTLQWKTDKPLCNGKSLCPICELLGRRIGKTDEVKKKGDFRIHFGNLTPLNRYDDPSDIGTQRTLNRVDYATGKAHDFFKVWEIDHSLLSVFQGKISIADNIGDGATKLLEDSLRFTDRLCGAICVISYDCIENSDGKENGKTGEAAHIMGESDAGKTDAENIANAIADMMGTAGEPEKLRILADAVRALRIGKNTVSQLPLDHEGKENHHLWDIGEGKSIRELLLEKAESLPSDQWRKFCEDVGEILYLKSKDPTGGLTVSQRILGDEAFWSKADRQLNPSAVSIPVTTETLICGKLISETPFFFGTEIEDAKHTNLKVLLDRQNRYRLPRSAIRGVLRRDLRTAFGGKGCNVELGGRPCLCDVCRIMRGITIMDARSEYAEPPEIRHRIRLNPYTGTVAEGALFDMELGPQGLSFDFILRYRGKGKSIPKALRNVLKWWTKGQAFLSGAASTGKGIFRLDDLKYISFDLSDKDKRKDYLDNYGWRNRIEALSLEKMPLDRMNDYAEPLWQKVSVEIEIGSPFLNGDPIRALIEKDGSDIVSFRKYADDSGKEVYAYKAESFRGVVRAALARQHFDKEGKPLDKEGKPLLTLIHQDCECLICRLFGSEHETGRLRFEDLLFDPQPEPMIFDHVAIDRFTGGAVDKKKFDDCSLPGTPGHPLTLKGCFWIRKELEKPDEDKSEREALSKALADIHNGLYPLGGKGAIGYGQVMNLKIKGAGDVIKAALQSESSRMSASEPEHKKPDSGLKLSFDDKKAVYYPHYFLKPAAEEVNRKPIPTGHETLNSGLLTGKIRCRLTTRTPLIVPDTSNDDFFQTGVEGHESYAFFSVNGDIMLPGSEIRGMLSSVYEALTNSCFRVFDEGYRLSWRMEADRNVLMQFKPGRVTDNGLRIEEMKEYRYPFYDRDCSDKKSQEAYFDEWERSITLTDDSLEKMAERKGDISPKDLKVLKSLKGKNYKSTEGLLAAFKDKGGDTGGNILGLIFKYAERIGDVPRYEHPTDTDRMMLSLSEYNRNQKSDGKRAYKIIKPASKLGKGAYFMFAGTSVENKRICNPACTDKANKSVKGYLKISGPNKLEKYNISEPELDGVPEDRNCQIIHNRIYLRKIFVANAKKRKERDRLVGEFACYDPEKKVTYSMTKRCERIFIKDRGRTLPITHEASELFEILVQEYRENAKRQDTPEVFQTLLPDNGRLNPGDLVYFREEKGKTVEIIPVRISRKIDDSPIGKRLREDLRPCHGEWIEGDDLSQLSEYPEKKLFTRNTEGLCPACRLFGTGAYKGRLRFGFAKLENDPKWLMKNSDGPSHGGPLTLPLLERPRPTWSMPDDTLNRLKKDGKQEPKKQKGKKGPQVPGRKFYVHHDGWKEINCGCHPTTKENIVQNQNNRTVEPLDKGNTFSFEICFENLEPYELGLLLYTLELEKGLAHKLGMAKPMGFGSIDIEVENVSLRTDSGQWKDANEQISEWTDKGKKDAGKWFKTDWEAAEHIKNLKKLLFLPGEEQNPRVIYPALKQKDIPNSRLPGYEELKKNLNMEKRKEMLTTPWAPWHPIKK

SEQ ID NO: 17 - protein, 1723 aa, synthetic construct
MTQITIQVTFFHPFRVVPWNHRDHRKTDRKYLRGGTFAKWHCTASEGKSGRPYITGTLLRSALFAEIEKLIAFHDPFKCCRGKDKTENGNAKPLFLRRRPRADCDPCGTCPLCLLMGRSDTVRRDAKKQKKDWSVHFCNLREATERSFNWKETAIERIVNRVDPSSGKAKDYMRIWEIDPLVCSQFNGIITINLDTDNAGKVKLLMAAGLAQINILAGSICRADIISEDHDALIKQFMAIDVREPEVSTSFPLQDDELNNAPAGCGDDEISTDQPVGHNLVDRVRISKIAESIEDVFSQEQKAQQLRRMADAIRDLRRSKPDETTLDALPKGKTDKDNSVWDKPLKKDILPSPRMPASEDDDTPTLRKVLKDEINGQEDMWRKFCEALGNSLYDLSKKAKERKRTEALPRLLGETEIYGLPMRENKEDEPLPSSLTYKFKWLIAGELRAETPFFFGTEVQEGQTSATILLNRDGYFRLPRSVIRGALRRDLRLVMGNDGCNMPIGGQMCECGVCRVMRHIVIEDGLSDCKIPPEVRHRIRLNCHTGTVEEGALFDMETGYQGMTFPFRLYCETENSDLDSYLWEVLNNWQNGQSLFGGDTGTGFGRFELTEPKVFLWNFSKKEKHEAYLLNRGFKGQMPVQDVKTKSFKTKTWFQIHRELDISPKKLPWYSTDYRFNVTSPLISRDPIGAMLDPRNTDAIMVRKTVFCPDPNAKNRPAPATVYMIKGESIRGILRSIVVRNEELYDTDHEDCDCILCRLFGSIHQQGSLRFEDAEVQNSVSDKKMDHVAIDRFTGGGVDQMKFDDYPLPGCPAQPLILEGKFWVKDDIDDESKSALEKAFADFRDGLVSLGGLGAIGYGQIGDFELIGGSADWLNLPKPEENRTDVPCGDRSAQGPEIKISLDADKIYHPHFFLKPSDKNVYRERELVSHAKKKGPDGKSLFTGKITCRLSTEGPVFIPDTDLGEDYFEMQASHKKHKNYGFFRINGNVAIPGSSIRGMISSVFEALTNSCFRVFDQERYLSRSEKPDPTELTKYYPGKVKRDGNKFFILKMKDFFRLPLYDFDFEGEAESLRPNYDEDRNEEENKGKNKNTQKVKNAVEFNIKMAGFAKHNRDFLKKYKEQEIKDIFMGKKKVYFTAGKHKPNEAHDNDKIALLTKGSNKKAEKGYFKFTGPGMVNVKAGVEGEECDFHIDESDPDVYWNMSSILPHNQIKWRPSQKKEYPRPVLKCVKDGTEYVMLKRSEHVFAEASSEDSYPVPGKVRKQFNSISRDNVQNTDHLSSMFQSRRLHDELSHGDLVYFRHDEKRKVTDIAYVRVSRTVDDRPMGKRFKNESLRPCNHVCVEGCDECPDRCKELEDYFSPHPEGLCPACHLFGTTDYKGRVSFGLGWHESNTPKWYMPEDNSQKGSHLTLPLLERPRPTWSMPNKKSEIPGRKFYVHHPWSVDKIRNRQFDPAKEKQPDDVIKPNENNRTVEPLGKGNEFTFEVRFNNLREWELGLLLYSLELEDNMAHKLGMGKALGMGSARIKAEAIELRCESAGQNAELKDKAAFVRKGFEFLEIDKPGENDPMNFDHIRQLRELLWFLPENVSANVRYPMLEKEDDGTPGYTDFIKQEEPSTGKRNPSYLSSEKRRNILQTPWKHWYLIPPFQASAQSETVFEGTVKWFDDKKGFGFIKINDGGKDVFVHHSSIVGTGFKSLNEGDSVAFKMGVGPKGPCAEKVKKIGN

SEQ ID NO: 18 - protein, 1548 aa, synthetic construct
MTKIPISLTFLEPFRLVDWVSESERDKSEFLRGLSFARWHRIKNQREDENQGRPYITGTLLRSAVIKAAEELIFLNGGKWQSEECCNGQFKGSKAKYRKVECPRRRHRATLKWTDNTCSDYHNACPFCLLLGCLKPNSKENSDIHFSNLSLPNKQIFKNPPEIGIRRILNRVDFTTGKAQDYFYVWEVEHSMCPKFQGTVKINEDMPKYNVVKDLLISSIQFVDKLCGALCVIEIGKTKNYICQSFSSNIPEEEIKKLAQEIRDILKGEDALDKMRVLADTVLQMRTKGPEIVNELPRGIEKKGGHWLWDKLRLRKKFKEIANNYKDSWQELCEKLGNELYISYKELTGGIAVKKRIIGETEYRKIPEQEISFLPSKAGYSYEWIILGKLISENPFFFGKETKTEEQIDMQILLTKDGRYRLPRSVLRGALRRDLRLVIGSGCDVELGSKRPCPCPVCRIMRRVTLKDARSDYCKPPEVRKRIRINPLTGTVQKGALFTMEVAPEGISFPFQLRFRGEDKFHDALQNVLVWWKEGKLFLGGGASTGKGRFKLEIEHVLKWDLKNNFHSYLQYKGLRDKGDFNSIKEIEGLKVETEEFKVKKPFPWSCVEYTIFIESPFVSGDPVEAVLDSSNTDLVTFKKYKLEESKEVFAIKGESIRGVFRTAVGKNEGKLTTENEHEDCTCILCRLFGNEHETGKVRFEDLELINDSAPKRLDHVAIDRFTGGAKEQAKFDDSPLIGSPDSPLEFTGIVWVRDDIDEEEKKALKSAFLDIKSGYYPLGGKKGVGYGWVSNLKIESGPEWLRLEVQEKSSQENVLSPVILSEVMDIEFNPPKIDENGVYFPYAFLRPLNEVKRTREPIGHNEWKKSLISGYLTCRLELLTPLIIPDTSEEVIKEKVNNGEHPVYKFFRLGGHLCIPAAEIRGMISSVYEALTNSCFRVFDEKRLISWRMTAEEAKRPDPKKSEEQNRMRFRPGRIIKKDKKFYAQEMLELRIPVYDNKDKRNEISQNDPTRPSEYNHPTEPERIFFSNAEKIRNFLKRNSNYLHGSTPLLFRQWSISNRYDKIALIGNKSQGHLKFTGPNKIEVSEGTKCPKYETIPGRDEWDKAVHNYVEPGKFVTVISRKKGQKPKAVQRRRNVPAFCCYDYNTNRCFVMNKRCERVFKVSRDKPKYEIPPDAIRRYEHVLRKYRENWERYDIPEVFRTRLPGDGETLNEGDLVYFRLDENNRVLDIIPVSISRISDTQYLGRRLPDHLRSCVRECLYEGWGDCKPCKLSLFPEKMWIRINPEGLCPACHLFGTQVYKGRVRFGFARAGSNWKFREEQLTLPRFETPRPTWVIPKRKDEYQIPGRKFYLHHNGWEEIYKKNKKNEIKKEKNNATFEVLKQGTFYFKVFFENLELWELGLLIFSAELGGEEFAHKLGHGKALGFGSVKISVDKIILRRDPGQFEQRGQKFKRDAVDKGFCVLENRFGKTNFKIYLNNFLQLLYWPNNKKVKVRYPYLRQEDDPEKLPGYVELKKHQMLKDDNRYSLFARPRAVWLKWTEMVQRDKS

SEQ ID NO: 19 - protein, 1720 aa, synthetic construct
MSVEEFYVRLTFLEPFRVVPWVRNGDERKGDRIYQRGGTYARWHKINDSHGQPYITGTMLRSAVLREIENTLTLHNTYGCCPGGTRTTEGKLEKPLYLRRRDGFEFENHAEKPCSEEDPCPLCLIQGRFDKLRRDEKKQFVRQGNISFCSVNFSNLNISSGIKSFSWEEIAVSRVVNRVDPNSGKAKDFFRVWEIDHKLCPNFLGKMSISLSEKLEDVKALLAVGLAQVNVLSGALCRVDIIDPETQKDTVHQHLIQQFVTRIQDKEKGDAADIPAFTLPPAGLSPSSNEWNDTIKSLAEKIRKIKELEQGQKLRQMADVIRELRRKTPAYLDQLPAGKPEGRESIWEKTPTGETLTLRQLLKSANVPGESWRAFCEELGEQLYRLEKNLYSHARPLPRLLGETEFYGQPARKSDDPPMIRASYRAFPSYVWVLDGILRAETPFYFGTETSEGQTSQAIILCPDGSYRLPRSLLRGVIRRDLRAILGTGCNVSLGKVRPCSCPVCEIMRRITVQQGVSSYREPAEVRQRIRSNPHTGTVEEGALFDLETGPQGMTFPFRLYFRTRSPYIDRALWLTINHWQEGKAIFGGDIGVGMGRFRLENLQIRSADLVSRRDFSLYLRARGLKGLSREEVTRIGLNEEQWEAVMADDPGTHYNPFPWEKISYTLLIHSPLISNDPIAAMLDHDNKDAVMVQKTVLFVDESGNYSQMPHHFLKGSGIRGACRFLLGRKDAPNENGLTYFEADHEECDCLLCSLFGSKHYQGKLRFEDAELQDEVEAIKCDHVAIDRFHGGTVHRMKYDDYPLPGSPNRPLRIKGNIWVKRDLSDTEKEAVKDVLTELRDGLIPLGANGGAGYGRIQRLMIDDGPGWLALPERKEDERPQPSFSPVSLGPVHVNLKSGSDTADVYYYHPHYFLEPPSQTVSRELDIISHARTRDSGGEALLTGRILCRLITRGPIFIPDTNNDNAFGLEGGIGHKNYRFFRINDELAIPGSELRGMVSSVYEALTNSCFRIMEEGRYLSRRMGADEFKDFHPGIVVDGAKIREMKRYRLPLYDTPDKTSRTKEMTCPELFTRKDGRPERAKKFNEEIAKVAVQNRAYLLSLDEKERREVLLGNREVTFDECPDDEYSDDEYSELKYAQKYKDFIAVLKKNGQKRGYIKFTGPNTANKKNEDAPDKNYRSDWDPFKLNILLESDPECRVSNIHCYPRPLLVCIKDKAEYRIHKRCEAIFCSIGSPSDLYDIPQKVSNQYRTILQDYNDNTGKIVEIFRTQIKHDQLTTGDLVYFKPAANGQVNAVIPVSISRKTDENPLAKRFKNDSLRPCAGLCVEDCNECPARCKKVADYFNPHPRGLCPACHLFGTTFYKGRVRFGFAWLTGEDGAPRWYKGPDPCDSGKGRPMTIPLLERPRPTWSIPDNSFDIPGRKFYVHHPYSVDGIDGETRTPNNRTIEPLAEGNEFVFDIDFENLRDWELGLLLYSLELEDSLAHKLGLGKPLGFGTVQINIRGISLKNGSKGWDTKTGDDKNQWIKKGFAHLGIDIKEANERPYIKQLRELLWVPTGDNLPHVRYPELESKTKDVPGYTSLLKEKDLADRVSLLKAPWKPWKPWSGTAPHPDKGTNRLRASIVERDRIQRKTDTAKPEKKEETKVGKSSSSDIEKRYVGTVKWFNDKKGYGFILYGTDEEIFVHRSGVADNSIPKEGQKVGFRIERGARGSHAVEVKAIE

SEQ ID NO: 20 - protein, 1610 aa, synthetic construct
MPRFQLSLTFFDEPFRLIEWTDKSNRNSANTQWMRGQGFARWHKITLEKGFPFVTGTAVRSKIIREVEALLSRNKGTWNGIPCCSGFFDTKGPSPTHLRYRPTLEWEYGKTVCTSEADVCPLCLLLGRFDQAGKKSDTPCQSTDYHVHWENLSAGVAQYRLEDIAQKRTSNRVDFFSKKAHDHYGVWEVTAVKNLLGYIYISDAITESHQKTVISLLKAALSFTDTLCGANCKLELSDEPVDSIHSNQSASNFNPHSGAAPSQCSQSMPPFNMDQETKELANTLCKAFTGNMRHLRTLADAVREMRRMSPGISSLPRGRLNKEGEITAHYLWDERIDEKTIRQVLEDTIELSPARSIIYKNWISFCNQLGQKLYERAKDNDPILERKRPLGEAAFSKVPTSSHAPRHDMNSRVKGGFTREWIIVGTLRALTPFYMGTGSQAGKQTSMPTLQDSNDHFRLPRTALRGALRRDINQASDGMGCVVELGPHNLCSCPVCQVLRQIRLLDTKSKFSMPPAIRQKICKNPVLSIVNEGSLFDVELGIEGETFPFVMRYRGGAKIPDTIITVLSWWKNERLFIGGESGTGRGRFVLECPRIFCWDVEKGQNDYIQYHGFRNKEDELLSVYSTVSGLAEKNDVNLNNARDFSFDKICWEVQFDGPVLTGDPLAALFHGNTDSVFYKKPILKSGEKEPSYQWAIKSDTVRGLIRSAFGKRDALLIKSHEDCDCLLCEAFGSKHHEGKLRFEDLTPKSDEIKTYRMDHVAIDRISGGAVDQCKYDDEPLVGTSKHPLVFKGMFWINRDSSVEMQRALIAAFKEIRDGLYPLGSNGGTGYGWISHLAITNGPDWLNLEEVPLPQPTADIPVEECTAEPYPKFQKPDLDQNAVYYPHYFLQPGKPAERERHPVSHDHIDDKLLTGRLVCTLTTKTPLIIPDTQTNTMLPPNDAPEGHKSFRFFRIDDEVLIPGSEIRGMVSTVFEALTGSCFRVINQKAHLSWRINADMAKHYRPGRIIQNNEKMFIQPYKMFRLPFYAGFDPRNCLSEKQLLGIEPVKLWVKDFVASLVKPQTDIDIEWKEKIGFVRVTGPNKVEVDSSNTPDPSLPECESDWKDIHITEDGSTPSKNDRVYRCQLKGVTYTVAKWCEAFWVKDEGKKPITVNAEAINRYHLIMKSYQDNPQSPPIIFRSLPVLNYKQDQKIIGSMIFYRESAKSDKIVNEIIPVKISRTADTELLAKHLPNNDFLPCAATCLNECDTCNAKTCKFLPLYREGYPVNGLCPSCHLFGTTGYQGRVRFGFAKMNGNAKFCQGGERPEDRAVTLPLQERPKLTWVMPNENSTIPGRKFFLHHQGWKKIVDEGKNPINGDVIEPDANNRTVEPLAAGNDFSFEVFFENLREWELGLLRYTLELESELAHKLGMGKAFGFGSVKIKIKSVDLRKQGEWEKATNTLVSEDKKSSWYNIHTVNNLRTALYYVEDDKIQVNYPKLKKDNESDNRPGYVEMKKTAFPVRDILTTPWWPWWPPTPPPMNQSGNQSYARSEEPARITESQPEVYKTGTVKFYKHDKKFGFITMDGRENIHFAGNQICRPETSLQSGDKVKFIEGENYKGPTALKVERLKG

SEQ ID NO: 21 - protein, 1575 aa, synthetic construct
MRLKINIHFLEPFRLIEWHEQDRRNKGNSRWQRGQSFARWHRRKDNDQGRPYITGTLLRSVVIRAVEEELARPDTAWQSCGGLFITPDGQTKPQHLRHRATVRARQTAKDKCADRQSACPFCLLLGRFDQVGKDGDKKGEGLRFDVRFSNLDLPKDFSPRDFDGPQEIGSRRTINRVDDETGKAHDFFSIWEVDAVREFQGEIVLAADLPSRDQVESLLHHALGFVDRLCGARCVISIADQKPAEREERTVAAGDEKATIADYDQVKGLPYTRLRPLADAVRNLRQLDLAELNKPDGKFLPPGRVNKDGRRVPHYVWDIPLGKGDTLRKRLEFLAASCEGDQAKWRNICESEGQALYEKSKKLKDSPAAPGRHLGAAEQVRPPQPPVSYSEESINSDLPLAEWIITGTLRAETPFAIGMDAPIDDDQTSSRTLVDRDGRYRLPRSTLRGILRRDLSLASGDQGCQVRLGPERPCTCPVCLILRQVVIADTVSETTVPADIRQRIRRNPITGTAADGGLFDTERGPKGAGFPFSLRYRGHAPMPKALRTVLQWWSAGKCFAGSDGGVGCGRFALDNLEVYRWDLGTFAFRQAYSENNGLRSPEEEFDLAVIHELAEGLAKEDGQKILKGTEPFTCWQERSWQFSFTGPLLQGDPLAALNSDTADIISFRRTVVDNGEVLREPVLRGEGLRGLLRTAVGRVAGDDLLTRSHQDCKCEICQLFGSEHRAGILRFEDLPPVSPTTVADKRLDHVAIDRFDQSVVEKYDDRPLVGSPKQPLVFKGCFWVQTSGMTHQLTELLAQAWRDIAAGHYPVGGKGGIGYGWINSLVVDGEKITCRPDGDSISLTTVTGDIPPRPALTPPAGAIYYPHYFLPPNPEHKPKRSDKIIGHHTFATDPDSFTGRITCKLEVVTPLIVPDTEGEQPKDQHKNFPFFKINDEIMLPGAPLWAAVSQVYEALTNSCFRVMKQKRFLSWRMEAEDYKDFYPGRVLDGGKQIKKMGDKAIRMPLYDDSTATGSIKDDQLISDCCPKSDEKLQKALATNQKIALAAKHNQEYLAQLSPDEREEALQGLKKVSFWTESLANNEAPPFLIAKLGEERGKPKRAGYLKITGPNNANIANTNNPDDGGYIPSWKDQFDYSFRLLGPPRCLPNTKGNREYPRPGFTCVIDGKEYSLTKRCERIFEDISGGENQVVRAVTERVREQYREILASYRANAAGIAEGFRTRMYDTEELRENDLVYFKTAKQADGKERVVAISPVCISREADDRPLGKRLPAGFQPCSHVCLEDCNTCSAKNCPVPLYREGWPVNGLCPACRLFGAQMYKGRVNFGFARLPDDKQPETKTLTLPLLERPRPTWVLPKSVKGSNTEDATIPGRKFYLRHDGWRIVMAGTNPITGESIEKTANNATVEAIMPGATFTFDIVCENLDQQELGLLLYSLELEEGMSHTLGRGKPLGFGNVRIKVEKIEKRLSDGSRREMIPPKGAGLFMTDKVQDALRGLTEGGDWHQRPHISGLRRLLTRYPEIKARYPKLSQGEDKEPGYIELKSQKDENGVPIYNPNRELRVSENGPLPWFLLAKK

SEQ ID NO: 22 - protein, 1237 aa, synthetic construct
MSNQTRWIIEGTLELITPLHIGTGLDEKERDENKETRWLEAVALDHKGQPYIPGASLKGALRALAKRHDFRNLFDNKEVDGDFVRQAEFLSAWCVPDTDKGRLIQPRVAIDRVTGTAQDKKLFQTRLITPGTRFAMKIVVQNAVENEIADLLGLLNLLPDDPQFSLGAYANQGQGRVQWFGKIQTRCFGINEAKAWYEEIRKDESKCWTAFAKPKNVSTPPTPAKEAQLTLPLNLAFHTPFLVKQAGIKADDADAVPRRTHDDKIVLPASSLRGRLRTQSERILRTLGCETPQGHTAPAYRKGQPHDDLAVLLFGAAGWRGIVQTSDCIVEDKSIKTRRHEMLAIDRFTGGGKDGAKFNVDYVECPTLAGKLSLDLARLKNAKLKGGKDALLPALGLMTLMLRDLAEGDIPFGYGISKGYGQCRASSALGDWAELLKQHLGADSADTTVQALREYLGNPKGQELKLDPPSADATQAGVPAQQNAAKTQAQGAQEKFHNPYHFIPLSKPDISQWPEPQKLTEKGHSHDRYASLSGRIVCRLTTQTPLFIGSEQTTPTNPQAPKSLHPFKLNNGLAIPATSLRGMISSLFESVSNSNFRVLDEKTYSMRKTMQQSLSAMGRIVRHDQKLYLLPLTLPTLPQGPHGVYDLGEKWSAVFDWQPPPLRIYFDPPPRRTYQSQQPCYMKLSTVKYSESNPNQIIAGENLGALRFPRGNQNTQFLIGQSNQDECPITQAEYAQKSEDERNEYTSGWVRTLVKPGRDLPRSVKHHVFLPDVFIDAPPPVNDLYPIPDSVIQRFHDLADQVLASMNLKPEEIVDSTNLLPYTPVGRRSDSDCRDTRLQAGDLIFFDIDPPLHPGEKSQITEISFSSIWRSGIGKDHLLTTPDLLTNFDVNLQPHGMPGRTQSLSPAELLFGLVGTQNDQATTAYAGKVRIGFGLPEEGHNPRLDARITLKELSSPKPPSPALYFRKKSGKDEYVSKANLADKPEDYILRGRKMYLHAWRKNEQVVELSDTGHDGGVRPPWVSKFDESADEGNKRRVSIEPIAKDESFYFEVDFHNLSRTELAQLCATLYPNEKFEHRLGMGKPLGLGSIKITPLSLFLVNRSQRYATDGLDKPRYHAVWHTGTASEPRWPDHLQREQQGIAFEGVSTAPTVMSLAAEAKVSDDVKRALELLGNPDEITVPVHYPQLHNGLMESKQFDWFVQNDKSGRDQPANNRQHLSSFTKDTEKLEPLIRIMRR

SEQ ID NO: 23 - protein, 1291 aa, synthetic construct
MTTPSAPKSSLPALHWLIRAELEVLTPLHLGTGTDQRITPDAPDADPYWQADIALDADGRPYLPGASLKGALKALARRRQVDAPCLPLFGDLNRGGAPHPDCIPPRRTRAGLAEFRDALQSHATQADGPDTAQPRIAIDRITGSVVDKKLFHTQTVPVGTRFSVEIILRRADQNLAAQLVALLQHGPTDPDFRLGAHANLGFGRVGLYGNIDTRRFGPQQAQHWFAAAQTQADARWTDFAEAVTLTAPAPAPAQPAPHRLALPLSLTFHTPFLVKQPEHKHRKPQDNAPDGTPRQRGDRALLPGASLRGRLRSQAERILRTLGCKVAQGHAVPPVKNNTCPDPATLLFGTAGWRGLLRTDDCTGTAPATLVDHDMLAIDRFTGGGKDGAKFKLRYAECPTLEGQLSLDLSRLRSARLDGANAADTPWIALGLLTLVLRDLAEGDIPFGHGSAKGYGRCRAQGLPDRWRQALEAHFGPNADARALAALRAWCRTHATAALDAPCSLAGSAPTPAAAAPSGQAAPADAFHNPYHFIPFSQPDIDRWLSPDAHRKTGGHSRYRGLSGRLVCALTTVTPLFVGAAARTPASDQHPKPVAGFALQNQPAIPATSLRGLLSSLFESISGSNLRVLHPTPYSIRKTTKEALSAIGRIVERNGELKLYPLTLPTIHQNADNAYPVPARWRKVFYWESPVPLRVYFGSRKQTYDSRQPHYLPIQELSYLPNDSDCIAPDQGDLRFPSRDRDRKFLIGQCPISRYDCPIPETDLPKLSPQERPRYTRGWVRSLWTSNREKELPHTVKHQLFIPDPVETPAADDLLPIPQGVLDTFHALADLALAGQHWGKDETPADDQLLPFTPAGRQRHDADRPPRDADRQTRLQPGDLVCFDLGDDGAVSEISFSSIWREGLRLAGKPNLATTADLLAQVSPHLLPLGMPGRSARLSPVEQLFGVVEYRPPQTAKGTRKPTDAPAAYALAGKLQVGFGRPARPFEREPAVTLKELSTPKPPSPALYFRPKAGDGYVSKAKLASQPQDYAPAGRKHYLHALRRQGQVARLDNSGHVPSDGSGRPPWQSRFDGQEDSGNKRRVRVEPIPAGETFHFEIDFDNLSPTELEQLCATLLPHPAFEHRLGMGKPIGLGSVKLAVEGLLLVDRPRRYAEDEPNAPRHHRGWRANADAGWPDHLQGDSPAAPLEATEQPAALAERAMARVPADVRRALQLLGNPGAVAAPVHYPQVKDAQIEEKHYLWFVANDDEKTAGGNRHLPRLHANSPGLPTLPRLVKREKDHSSNTGKPRRK

SEQ ID NO: 24 - protein, 1801 aa, synthetic construct
MIPDLRSLVVHISFLTPYRQAPWFPPEKRRNNNRDWLRMQSYARWHKVAPEEGHPFITGTLLRSRVIRAVEEELCLANGIWRGVACCPGEFNSQAKKKPKHLRRRTTLQWYPEGAKSCSKQDGRENACPFCLLLDRFGGEKSEEGRKKNNDYDVHFSNLNPFYPGSSPKVWSGPEEIGRLRTLNRIDRLTTKAQDFFRIYEVDQVRDFFGTITLAGDLPRKVDVEFLLRRGLGFVSTLCGAQCEIKVVDLKKKQNNKEDSILPVSEVPFFLEPEVLAKMCQDVFPSGKLRMLADVILRLREEGPDNLTLPMGSQGLGGRLPHHLWDVPLVSKDRETQTLRSCLEKIAAQCKSEQTQFRLFCQKLGSSLFRINKGVYLAPNSKISPEPCLDPSKTIRTKGPVPGKQKHRFSLLPPFEWIITGTLKAQTPFFIPDEQGSHDHTSRKILLTRDFYYRLPRSLLRGIIRRDLHEATDKGGCRVELAPDVPCTCQVCRLLGRMLLADTTSTTKVAPDMRHRVGVDRSCGIVRDGALFDTEYGIEGVCFPLEIRYRGNKDLEGPIRQLLSWWQQGLLFLGGDFGIGKGRFRLENMKIHRWDLRDESARADYVQKCGLRRGVGDDTAINLEKDLSLNLPESGYPWKKHAWKLSFQVPLLTADPIMAQTRHEEDSVYFQKRIFTSDGRVVLVPALRGEGLRGLLRTAVSRAYGISLINDEHEDCDCPLCKIFGNEHHAGMLRFDDMVPVGTWNDKKIDHVSCSRFDASVVNKFDDRSLVGSPDSPLHFEGTFWLHRDFQNDVEIKTALQDFADGLYSIGGKGGIGYGWLFDMEIPRSLRKLNSGFREASSIQDALLDSAKEIPLSAPLTFTPVKGAVYNPYYYLPFPAEKPERCLVPPSHARLQSDRYTGCLTCELETVSPLLLPDTCREKDGNYKEYPSFRLNNTPMIPGAGLRAAVSQVYEVLTNSCIRIMDQGQTLSWRMSTSEHKDYQPGKITDNGRKIQPMGKQAIRLPLYDEVIHHVSTPGDTDDLEKLKAIVLELTRPWKELPEEQKKKRFEKCKNILDGRMLQQKELRALENSGFAYWRDKTSLTFDSFLKDAIEQEYPRYSGDYQRIKALVVNITLPWKLLKKEERHKRFDKCRRILKGQQPLTKDERKALEESGFANWHGRELLFDRFLKDENSCLIKAETTDRVIASVAKNNRDYLFEIKQQDFARYKRIIQGLERVPFSLRSLAKSKETSFQIACLGLRRGRFLRKGYLKISGPNNANVEISGGSHSNSGYSDIWDDPLDFSFRLSGKSELRPNTQKTREYPRPSFTCTVDGKQYTVNKRCERVFEDSAAPAIELPRMVREGYKGILTDYEQNAKHIPQGFQTRFSSYRELNDGDLVYYKTDSQGRVTDLAPVCLSRLADDRPLGKRLPEEYRPCAHVCLEECDPCTGKDCPVPIYREGYPARGFCPACQLFGTQMYKGRVRFSFGVPVNSTRSPQLKYVTLPSQERPRPTWVLPESCKGKEKDVPGRKFYLRHDGWREMWGDDDKPDSRPSSEECQDIIEGIGPGEKFHFRVAFENLDKNELGRLLYSLELDAGMNHHLGRGKAFGFGQVKIRVTKLERRLEPGQWRSEKICTDLPVTSSELVISSLKKVEERRKLLRLVMTPYKGLTACYPGLERENGRPGYTDLKMLATYDPYRELVVQIGSNQPLRPWYEPGKSFKPSPGNDCTGRGGSVSKSLISEPKVVPAIAPFCEGVVKWFNSVKGFGFIETKEQRDIFVHFSAIRGEGYKILEPGEKVRFEIGEGRKGPQAINVIRIR

SEQ ID NO: 25 - protein, 1322 aa, synthetic construct
MKMNKTWPFREHWEISGYLRTVTPLHIGSGRTVTRPELTVADRDELVDINAVVTDYTGKPYLPGSTIKGALHAWLQKRLKEESRTCLIQLFGQEEEAEEKKKNNHGGKAEFFDARVIFPHTGPGSLPYWDDCRQTYVAATVAIDNITRTARHRHLIHAEMVPPGVTFALTLAGPLDEEDIGLLLAALQGFNESPPALVIGAHTANGLGRFSWELSTVRRFGKEHLQGWLEAETRAMRTEAMQPLSREGVEDLLQDGIAQIDHNEDQVRLGLELCFDGPFLVNDPPTKKEQDDKKKRRSNTPNLRPLRDAIGRPCLPESSVRGALRAQAERIIRTMEGTCSEDNPAYKKEIHTDAEIEELSAVCRVFGAPGWKSLLEISDFEFVDGEDCDNIQEFVAIDRFTGGAKDKAKFNAEYIGSPRFTGTIALDKRRDLPDWGKGLLYLVLRDLAEGDITLGFGRSKGYGVCRAKIKNLDLLLPEKSVAALHKKFAISPADDKPATDQIAEDTTSGNLGISAGGPAQKTTESYEPPNPSGPGTFHNPYHFVPVVKPSAADRQHWLDKGILPSASENKTQHTHACYLDTTNGKKIYHGRIVCRLQAETPMFIGGRHRENTEPTEILPFTLGGKPAIPATSLRGMLSSIAEAASNSSLRVLEDKTLSYRKSMRANRNEDKPLSALGMIKKIETGDKVEYRLLPLTLPTLVKRGQYYILPEEYQTMFPDGRAKLKVYLNSNYTASDGTNQDFLKGKKSWRLPHGEIYYMKLCQDFSLQNGQLTFDSQNQNMLHFPKNRNNFVVGQRSIDNTPPMTKAEWRTNHQTGVPGMLRIMQASGRNFPTGRYHEIFIPVPTKKDCKQLYPVDEKAVERFLDLAGEQTKSQQNEKNLKQYQILPYHPVGTKRNTDPETNDRYMDLNSGDIVYFRPDATGTKVEEISFSSIWRDRVEDDNHNRAGVHAFFGNIDKELLPFNPKRAEISPAELLFGFVEERERGKVDDGQAPAFAGKVRVSFGRLSSEKKPDTIFQDQVTLKALSSPKPPSPALYFTGNGNGSIAKPDLTLSRHSPQGRKFYLHAWQEENEIIKFLSNGKKTSPTVINGLYPWESKSNLSRQLPDKHAKLKSAITPIKKGTVFYFHVDFDNLSEWELGLLCYALQPGKEFRHKLGMGKPIGLGSIHIEPAGLYLVHRGNRYSLDGVPDNSRYNGGIWQSEDKRLQEWQELYPRESTAAASSAAASPADFALKFSGTMTPSVQQALKRLGDPDNVVAPVHYPQVEGADLEEETYKWFVANDVGSQTKVGNRTTCTEEAARKSMLPLAGRGPLPRLKRYKWCP

SEQ ID NO: 26 - protein, 1225 aa, synthetic construct
MAAVQDRWTLMDQQGNELKRFRITAELETASSLHIGASETVEHDLIKNDDGTPVQINALITGAGGLPIIPGSTIKGRFLARLRERGVDSALLETLFGKGHDRETEDQGRGGRAEFHDAPLCHRLSGARHFPYWRPERQIWVKAQTAVDRHRGTALRRSLRYTEMVPPGVRFRLTITGCMTDAEADVLFALLEDLGDPRQACSFGGAGADGNGTMRLFGRPEVYCLDRSGILGWLASFEKGGNGGMAMTAAALLQADTVQRRADKVRQAWQPPDVGPRLHVELRFSGPFLVNDPSRNTPDITQAPDMVPLVDEDGNPMLPASSFRGALRAQAERIIRTLGGRCCDTSSPCRPLGSSDKVGELCLACQVFGAPGWGTTLHIQGFTCTSVFRREQEQTFVAIDRFHGGCKEGALYTIRHAESPRFEGHLVIDPRMPAWGRGLLALVFRDLREGDITFGLGAGKGYGVVDAAVVQDMAELEPYVEAFREQCRQHQGMADCHSAPSPQPLRDHDLAEIPPAEEAPGETFLNPYHFVPIREPDTGSWLARDELDSSCCHSHGFYRQQVDDRPLYHGRLTCLLETETPLFIGATGDSSVPSRIENYRLGNRIAIPAASLRGMLSSLAEAASNSAMRVLHQGILSYRKKAKNALREIGMIVLRDGKRFILPLVPLMEVTKLRHAYTDPAMKHFLDDKNSWSPRCNRVYYLGRDGNQIPAETRGAGMRPGILRLLGREGRHDALQNKKHEYFIEIPERYVDQDHCFDYRMFIRDRARNGTLVPISPVAWERYHCLAEERTLSQKNDPELREDKACASLKWLPFHPKGRVRERDPENDVCHLSLRHGDLVFYAEQNRVVSEISFSAIWRSRVETSDSYQAVTVDCFVPKELRPFNRDRRAISPAELLFGFVELDESEHSTEKSRYEQMAFAGKVRLSAGLPVEDVEDSALLEPKPIVLKALSSPKPPSPPFYFVMRDGSGAYIAKKDLSPDRHRIKGRKHYLHGLRQRGNPDRVQSLDRYGHATETAANPPWETCHPEERPQIKVRVQPVRRKTKFFFHLDFSNLSRWELGLLCYVLRPTACFRHKLGMGKPLGLGSVRIDIASLQLIDRVRRYGTDDLTAGRYNMGGHFNASCLDLLPQQDSPAPDDSGAAPDPGTLRQDFVKTMDETVFRALDLLGNPAHVQRPVHYPQVREMDIEDQTFLWFVDNDKQWKDALQPLTSSSTQLPPLTRRNKR

SEQ ID NO: 27 - protein, 1234 aa, synthetic construct
MTTVKEKSWAFTGLKRWKIITTLETQTPLHIGSGEVAEIEINDSQGDRRQVQANAIIRGKDDKPIIPGSTLKGKLRSHFESCLDHSKALERVFGKEYQSDEEQGRGGLAEFHDAVCSYVAPGNSYYPNWNEARNTYIEASTTIDCHTGTAADATLHYNECVPPGTRFLVTVTGAMSDKDAALIVAALQAFGDETNPIHLGAEEANGKGRMGLFGNVEVSCLDHDDIIAWISQGSDARMATDKFKPLGKEKVNDLAKNITTPTATGGAQRQHFGIELKFDGPFLVNDPSKYSKGDGDQPAVHQPLTDRSNNPILPARSFRGAIRAQAERIIRTMGGACCDTQSPCQNSGQLCIACQMFGTTGWKTTLSISDFTYDGEYRPAKTQQFVAIDRFHGGGKDGALFSIKYFERPVLKGGISLKLRNQNADELSWRKGLLALLFRDLQEGDITFGFGANKGYGGVEEACITNADVISTADIEAFRAKCHANHADSWCSPVSKPTNRDDKSSLPSINPATGAGHAFHNPYHFIPIKAPDTSTWLDKHKLATPGSPHSHAYYRSCSDDDKPLHHGRITCKLTAETPLFVGSGDAENQLTDSEAKLKEHYQLNNKLAIPATSLRGLISSLAEAASNSALRVLDNGVLSYRKPASRALRKIGILFKREEQWRLVQMEGNLANAIKLKSAYTNQKMMDFLANKQSWSPEHNVVYYLSADFRPGDVPQETYLAGRICGILRILGGKDGDRKNELENKKHELFIRVDEQYVDTEINRFDYEEYVRQGGIPVSPSAVERYTELADQRSLSQKNSRDLKGDNNCCSDKWLPFHLKGAARYKKEKACLLPLREYDLVYFDSDGTQVTEISFSAIWRDRVADKVHAFFPEELRPFNQKRKWISPAELLFGFVELNDNKDERDHAQAFTGKVRVAAGVLSPDDSIRQGDLQEHEPIMLKALSSPKLPSPALYFKQKSGDHRYIAKPDLKKASHQAQGRKIYLHALRDQKDDVQKLNTKGQPANGNGAHLPWKTADEDERPQLKVRIRPLKPGTSFYFHLDYNNLTEWELGLLCYVLQPSETFRHKLGMGKPIGLGTVKIEIATLQTIDRQKRYREAGANEHRHNGSNWVNESLRDELERLPGTVELSPDRQPEAKLRPDELRQSFIATMDNDIYRAIELLGDPHNIKYPVHYPQVRNKSIEQENFKWFVANDSGSGDQRKGTGIDAKEEPMRSIDQISTTIPTLNRYEWNGD

SEQ ID NO: 28 - protein, 1258 aa, synthetic construct
MARNNKQYHFIPRWEIKVNLTTRSFLHIGCDEFTDRPGLEIEQKDGSKVKAEINAFIKDSNGKPYLPGSTIKGNIRKWLETNKKADEETCKLFNTLLGFTVKMQDEGCGGSAEFHNAVISSPLEDGNNFPYWDVDLQTSVETSTVIDRVTGTVVDGRLFSTEVVPPEVSFTLIITGAMTEQQVSLLTAVIKDGFAEDCPTPITIGADSGNGFGRFRFDSIHMKCLGTGEVLNWLEDGSQDMAATAMRSLSPDDIEQHIIKGRNYLKSPSVSDTVTIEFGFAGPFLVNDPSRKKRKEDIDHQPLRDSAGNARLPAKSIRGAMRSQAEKIIRTLGGWCCDPVNPCPSVFSVVEINDRLCLACRVFGATGWKSRISIQKVEYKGTAESTRQETVQDFVAIDRFHGGGKETAKFDASFSWRPQYSILMHIPSDLEGWAKGLLALTFRDFKEGDIFLGYGRSKGYGRVDSDSVKPGIDTMLTESNLELFRRKCDDNPGEYPCKTRQPPNLVQPVERNNLTEAADEGSFHNPYHFIPTPKPMIESWLAKEDFDETMHDSHALYRDVDENEEPLYHGKISCTLTTETPVFVGGKHDPRNDTEPQQVDHYTENGEIAIPATTLRGLLSSLSEAASNSSMRVLDDGMMSYRQPVGSGSLSAIGMVVIRDGKKFIYPLALPIFGERDKLPQEYHIMFPYTQKAPLKVYLERAYLAGNMKSFLDKQNSWNLLNEKIFYLPVPEFSFSRVHTMGAENRDVLKISRRGNLILGARLPVNLCPRSKEKALPGDIPGILRILGKEGRDGEVPVGKKHELFIPVSDGFASNPRSFIDNLTSKELFKIPDEVVDRFEELADDRTTQQIKHPGNVKNNNQWLPFHLKGCTRNDGLTGKDEKRLRVQEGDLMYFRPSPQSPQVAEISFSAVWRGRVNKTVHNYFPPELVQFNKNREKISPAELLFGFVQQDKHEKSLSFAGKVVLSSGKQLRETESVSRENEVTLKILASPKLPSPSLYFKRENYIEGGNYIAKNEMNNSSNIKPRGRKQYLHALSNSEDPKGVQKISRTGSVDDGGNYPWQSMNNDNIKQKVCIRPVSKDGCFTFEMEFENCTEWELGMLLYALRPSQQYRHKIGMGKSIGLGTVRIDINNLQFIHRKNRYNAGIIDVPRYNYEAGHDMDYFHNKFADTIMPEIKNSIELLGDPRNVRFPVHYPQVHGADIEDKTYQWFVANDSGTNNGQNGAAYKKNKAEESSLTELDEISNTIPGLERHEWLGR

SEQ ID NO: 29 - protein, 1235 aa, synthetic construct
MALKTWTLNGEERWHISVVLETVTPLHIGSGEFCYRPELTNADQKPVDINACIKGANNLPIIPGSTVKGKFNAWLTARQVDTPLLEAIFGKGHNPDDDDQGSGGKVEFHDAWISTKIKDTSTWPYWQVATQTFIDAATAIDRHSRTALDASLHYTECVPPGVQFTLNITGVMQEHEAALIIAALDRFDQHDDQPYFGAGDANGQGQLILVGHLAVKVMGKTEITEWLAHFNNKASDMAMSHARSLGAEDIAGLIKLGQTLLKPVPPTVSLGIQLQFAGPFLVNDPYAVKKLEADPKTKIDHYPLLDNHKKPRLPSASIRGVLRSQAERIIRSLGVHCCDTRDPCPSLYKHQDLSQLCLACQIFGAAGWKSVINISDFTCVDANELKTQEFIAIDRFHGGGKDGAKFNAKHSERPYFQGRITLSPRMANHQLDWGKGLLALVIRDLQEGDLSFGFGANKGYGALESVLITGIDQLQTDAIEAFRRLCVTQAAPQAFITPTSAVVIGDKAPLVVTDKKLPDNSFHNPYHFIPINSPDTRHWLPTETDLAESHHSHAYYRQQPELFHGQLICRLYTETPTFIGASKKDDTLPAELDNYRLNGQLAIPATSLRGMISSLAEAASNSAMRVLDNGLLSYRKDASLALSKIGITFINRQGQWQLIPMEKIKLKNAYSAENMRLFVEQSHSWSPDYNTVYYFSEKAGAFDVPQRTPKPGWQPGILRLLGKEGRSQELENKKHEWFIPVPENYIDKQLNAFKYQEYLKDNSSKAIDIPAPVLNRYNELAYQRTLSQKKDTELVADGDSPAWLPFHLKGQQRQPQMVGKHLVYTLPMTEYSLVYYAATNKVATEISYSSIWRGRVQDDADQAATVNHFIPDDLLPFNPKRTSLSPAELLFGFTELDPDKHSNDPTRSFAGKVRIGAATLAAYPSNDSDLLAPEHITLKALSSPKLPSPALYFRTLQGNNSNVYIPKHELNPNHHTAKGRKYYLHATRTPDQKRILKLSDQGHPPQNNAVKLPWLSHQETKNLQLKVKIKPIKPKQSFYFQVDFNNLTAWELGLLCYALRPTIDFRHRIGMGKPLGLGSVKIDILALQTLDRQKRYAQDSQDSARYNQHRWVNSSVTDMLAQAGYDVIEPTANPLVPKDLKTLFSQTMAANIDRALTLLGEPQHVKQPVHYPQVRDTAIQVRDTAIEEESYQWFVANDNLSDNSSAAKQTLHDITETSEGLPTLIRHQKKKETQP

SEQ ID NO: 30 - protein, 1276 aa, synthetic construct
MSDTQKQAIHENKWHFRGIKRWEISAYLKTLSPLHIGDGGTIPVTIKDTQGKNREVEVNSVITGKAALPIIPGSTIKGRLRHYFSKHFSDKALLNKVFGEESDATDDDQGRGGLAEFHDAKWNPEKNRNLQGRYPYWNNTRKTYIEVSTAINRHTGAAKDKSLHHTECVPPGTVFEIKITGSMDDRCAALVVAALEAIQTTGSRIFLGAEDANGNGRIGLTGKITVKQMDQAHIIQWLQKDSTTCVASFSNVKAENETQVKQMVQRHIAPKLNSVVSAAGPSYDITLHFDGPFVVNDSDKCKAEDTPDIYPLEEKNGVPAFPVRSFRGAIRSQAERIIRTIGGQCCDGSINNTCKNPKNLCIACEMFGSTGWKTSIEMDPFLCVDRELKPFIIQEFVAIDRFHGGGKDEAKFNAAHYQAPVFKGKVRVSQRVGNDISWRKGLLALIFRDLKEGDIYFGFGTNKGYGAVKKAEINPDGNASDFSESDIEAFINKCREKKGLYNCNPIKKPGKTKVSKNLPPAIVPLDRTDSKFYNPYHFIPVKKPNTSSWAEKTAFGTADSPHSHGFYRKQTNEQQPLYSGRLICMLTSETPFFIGAQAESDPTENENQASLRHPYQLDGEPAIPSTSLRGLISTMTEAAANCAMRVLDSEIISYRKPMNPSHILSALGMVTKRGEDFWLIPLAMPALSLNDEEHNYKLDKRYRTMFPDGLAKLKVYLEKAYSNNVMKTFLNNENTWTLAQSKIHYLPLTPIQMQNGGINSYYNNLRTPSRSNNFLIGQTVAHGNGIPASGPGAGMVPGILRILGKEHRQNDLPQNKKHELFIPVPDAFVADPKTFLDTATAFLIPRNVIDAFEKIAEKQTQSQKQDKLKHDEERLPFHLKGTRREQNHTLQIKTGDLVYFRPNAKGDEVEEIAFSSIWRGKTSGTTADFFPDKELLPFNRNRSRVSPAELLFGFTENNPKEMKIDRGLAFAGKIRISAGTLSDKFSDTTESDLFEPETTLKALSSPKPPSPALYFKEKKSGTQYIKKQDLNPGKHEIQGRKIYLHALRNENNQNVQRITSQGKFDNAANRTQPWVSQNEERNHLKTKCKPLKSGLNFFFHIDFNNLTQWELGLLCYALRPCETFRHKIGMGKPIGLGTVKIDIAVLQTIDRYARYTDTTQDSERYNQGAWISQELQNEIPNQYKGKGISNKKGMLSPEDCRKVFMETMDADIQRAIELLGDPGNVTSPVHYPQLDRKNIETKNYEWFKQNEIEQQVLKPITKNTTHLTPFARWEQG

SEQ ID NO: 31 - protein, 1221 aa, synthetic construct
MNLPTWKLNNEKRWHISIVLTTATPLHIGSGEFCEHDDVKNNDGEPVKINACIKGSKGRPIIPGSTIKGKLYEWLKTRNTEENLLEKLFGKGHNSVSQDQGRGGKAEFHDAEIIEPLTGSQPWPYWREEHQAFIAASTAIDRHKQVALQQSLHYMETVPAGIRFKFTFTGVMRDEEAALLIAALDSFDKNQNQPCFGVDRANAYGRMELHGHLHVKVMGATEISSWLNSFSENDKKMAMESARNLEQQEINTLIKQGNALFKASCDEVKLGLTLKFKGPFLVNDPYAVKILSSNENAKTDHYPLLDKNRNPYLPVSSFRGVLRSQAERIIRTLGGKCCSTDDPCKPIFDKGDLSKLCLACQIFGASGWKTVINIHDFKAINKSKKTKQDFVAIDRFHGGGKDGAKFDATHFERPEFEGAISFSPRMANNDLDWGKGLLALVLRDMQEGDMTFGYGANKGYGGLESASITGIEQITSDIQAFRDKCVASPQTWLCDEAVKPANQQDKIPPAGIQVANSGFHNPYQFIPSKDPDTGHWLPVLGLNADSHHSHAFYRDQTDNGEKLYHGRLICCLNTETPIFIGADKKKDTEPAEINNYRLNGELAIPATSLRGMISSLAEAASNSAMRVLDNGLLSYRKTADDALRKVGMVIYVDNKSFIIKLNDAIKLKQTYTPGNMKDFIEKSNSWSPEHNTVYYLDNNQIPQESYMNGMKPGILRILGKEGREQELENKLHELFIPVPLEYVDTENNKFDYQAYKKAFLYRAIEIPEPVLKRYSELADQRTMSQKSNKELKKDDTCQSVGWLPFHLKGTKRQLDDKHKVGKLQIDEYDLIYYEASGKEVTEVAFSSIWRGRVETNSSQANKVYSFIPGELLPFNESRKKVSPAELLFGFTQINKDGSKADDKAQAFAGKVRISAGTISEYPESEANLLEQEVTLKALSTPKLPSPALYFRTINGNGSAYISKQELEPSKHLAKGRKYYLHALRTGDNKVQKLGSQGETANGGDSKLPWVTHNPDERPQLKVKIKPIKAEFIFSLDFNNLTEWELGLLCYALRPTDSFRHRIGMGKPLGLGSVKIDIMALQTINRQQRYAQDGLEENRFNRHNWVNPPHQPRLDKAGYSISLSSTPLNPEILRATFTKTMNADIYRTLELLGNPQNVKRPVHYPQVENHNIEQENYKWFVANDQGSGKGRNKIDPAEKALKILTENSDCLPTLSRLDWRDE

SEQ ID NO: 32 - protein, 1444 aa, synthetic construct
MNNKGSNMTDTVKSGRWIITGQFQLVTPMHIGTGLDEEMDKQSGESVDKKQNNSWIQAIALDLNKKPYIPGASIKGALKALARRYYCASNLNIFGDTIDTKDGDNKRKSVTVAGQAEFLNAWYAADQEDKPFDTITRVAIDRVTGTAEDRKLFNTRRVNPGVCFNYKIIIQNACETEIQYLLDLLRKAAKDPSFSLGAGANQSQGKVRCLSSCVRYFGKQEMHDWFRAIQNGKQEHWQIFAKPSNIKYADLERPDIIANSLNLPLTLDFHTPFLVKASKKKDEAKNEADAKPRTNHQGQVILPASSLRGRLRAQAEKILRTMGQDIPQGHAAPAYDGIAHRDLISLLFGTAGWKGIVCASDLIHSIPEYALQFNGVRETISDLSDTVKSCIIVDLVKTSTAAEKTEEQLHIRIVDSAGSLIVHKSENSSWANDTFRDASVKDNFKARLKEIADPQDLSDALRADIKKRAFQLATLTRHEMVAIDRFTGGGKEGAKFNVDYIECPTLTGAIYLDLHRLKQAQLKNDEDALKPALGLISLLLRDLAEGDIAFGFGANKGYGQCREHAVLDNWEERLKKIGAGLTIDGALQALRDTVALEPPAEFPPEIEKTTDDNQPEAPDFNLKPASNGFHNPYHFIPLNNPKIGDWPEAKAETLKANREGHDQYHTGKFSGRIVCSLTTQTPLFIGAETKPSTSDREPSEARPFKLNGKHAIPATSLRGMLSSLFESVSNSNFRVLHPEHYSVRKSLDDYVALSAMGRIVDDQGELKLQPLTLPTLFGNRNNVPAKWEKIFGTPSEDDFLRIYFDDIPSKFSSNKRYFYNCKATELKDFIKSDKYFIGKRTPTVFPKSSTEKSHLESLEFIDVEKFKKAVENLEITPGNNPYIHGWVRNLKDEFREDIPDNVKHHVFLPDTTKRVSPLEIPPHVKKRFHELADLALAGLHLKQGETIASPYKILPYTPIGRNKLENHIHRVPNDLTCYMTRLKKGDLVFFDVDNDGQITEISFSSIWRAGIGTKNKLQTTADLLSQRDPNLVQLGMGVRTKNTDRFKLSPAERLFGVVEHRDDDNTTVENVNQPNDKAQAFAFAGKVRIGFGLPDKKTTVNGVSPVTLKELSSPKPPSPAFYLKRKNNDDFVSKKVAAECSETMTLRGRKCYLHAWREQNGNVMKLDAIGVNSGGSTCKPPWKTHKPAANDQKEFEEDKNKFITSRQVKIAPISENTPFYFEIDFNNLDATELAQLCATLQPAPKFEHRLGMGKPLGLGSVKIEPVGLFLINRHQRYTTDSTNCDRYHYAWLKGEHAAWDWPEYFRQNVVTADCTQTFNDTFDKLVQNGLAGTDADIKHALQLLGDPQYIGVPVHYPIAGNSTLENKHFEWFGNNDKASVLRQKAQANSKNHHYQPKQQATPEEPQYLHTITKDSKQISLLKKNKIEDIENRDQQKHRYSNHRR

SEQ ID NO: 33 - protein, 1329 aa, synthetic construct
MFPKGRQMRRQRLLGDAEYYGGTGREQPASIVISTDSDPDHKVYEWIITGQLKAETGFFFGTKAGAGGHTDLSILLGKDGHYRVPRSVFRGALRRDLRVAFGAGCRVEVGRERPCECPVCKVMRQITVMDTISSYREAPEIRQRIRLNPYTGTVDKGALFDMEVGPEGIEFPFVLRFRGSKSFPSELAAVIGSWTKGTAWLGGAAATGKGRFSLLGLSIHKWNLSTAEGRKSYLAAYGLRDAADKTVKRLSIDKGGKGDVGLPAGLERDALPSSVREPLWKKLVCTVDFSSPLLLADPIAALLGVEGDERIGFDNIAYEKRRYNGETNTTESIPAVKGETFRGIVRTALGKRHGNLTRDHEDCRCRLCAVFGKEQEAGKIRFEDLMPVGAWTRKHLDHVAIDRFHGGAEENMKFDTYALAASPTNPLRMKGLIWVRSDLFETGHDGPTPPYVKDIIDALADVKRGLYPVGGKTGSGYGWIKDVTIDGLPQGLSLPPAEERVDGVNEVPPYNYSAPPDLPSAAEGEYFFPHVFIKPYDKVDRVSRLTGHDRFRQGRITGRITCTLKTLTPLIIPDSEGIQTDATGHKMCKFFSVAGKPMIPGSEIRGMISSVYEALTNSCFRVFDEEKYLTRRVQPKKGAKSSELVPGIIVWGQNGGLAVQQVKNAYRVPLYDDPAVTSAIPTEAQKNKERWESVPSVNLQGALDWNLTTANIARDNRTFLNSRPEEKDAILSGTKPISFELEGTNPNDMLVRLVPDGVDGAHSGYLKFTGLNMVLKANKKTSRKLAPSEEDVRTLAILHNDFDSRRDWRRPPNSQRYFPRSVLRFSLERSTYTIPKRCERVFEGTCGEPYSVPSDVERQYNSIIDDISKNYGRISETYLTKTANRKLTVGDLVYFIADLDKNMATHILPVFISRISDEKPLGELLPFSGKLIPCEGEPPTILKKMAPSLLTEAWRTLISTHLEGFCPACRLFGTTSYKGRIRFGFAEHTGTPKWLREELDWARPFLTLPIQERPRPTWSVPDDKSEVPGRKFYLHHHGGNRIVESNLRNRPEVNQTKNNSSVEPISAGNTFTFDVCFENLEAWELGLLLYCLELSPKLAHKLGRAKAFGFGSVKIHVERIEERTTDGAYQDVTAVKKNGWITTGHDKLREWFHRDDWEDVDHIRNLRTVLRFPDADQEHDVRYPELKANNGVSGYVELRDKMTASERQESLRTPWYRWFPQNGTGGSGRHEQAATSQEQDTAKDESVLSATQRRQAVIDVSDPDERLSGTVESFDRQKGDGYIGCGVRQFYVRLEDIRSRTALCEGQVVTFRARKEWEGHEAYDVEIDQ

SEQ ID NO: 34 - protein, 1601 aa, synthetic construct
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTALQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTAVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 35 - protein, 751 aa, synthetic construct
MSNPIRDIQDRLKTAKFDNKDDMMNLASSLYKYEKQLMDSSEATLCQQGLSNRPNSFSQLSQFRDSDIQSKAGGQTGKFWQNEYEACKNFQTHKERRETLEQIIRFLQNGAEEKDADDLLLKTLARAYFHRGLLYRPKGFSVPARKVEAMKKAIAYCEIILDKNEEESEALRIWLYAAMELRRCGEEYPENFAEKLFYLANDGFISELYDIRLFLEYTEREEDNNFLDMILQENQDRERLFELCLYKARACFHLNQLNDVRIYGESAIDNAPGAFADPFWDELVEFIRMLRNKKSELWKEIAIKAWDKCREKEMKVGNNIYLSWYWARQRELYDLAFMAQDGIEKKTRIADSLKSRTTLRIQELNELRKDAHRKQNRRLEDKLDRIIEQENEARDGAYLRRNPPCFTGGKREEIPFARLPQNWIAVHFYLNELESHEGGKGGHALIYDPQKAEKDQWQDKSFDYKELHRKFLEWQENYILNEEGSADFLVTLCREIEKAMPFLFKSEVIPEDRPVLWIPHGFLHRLPLHAAMKSGNNSNIEIFWERHASRYLPAWHLFDPAPYSREESSTLLKNFEEYDFQNLENGEIEVYAPSSPKKVKEAIRENPAILLLLCHGEADMTNPFRSCLKLKNKDMTIFDLLTVEDVRLSGSRILLGACESDMVPPLEFSVDEHLSVSGAFLSHKAGEIVAGLWTVDSEKVDECYSYLVEEKDFLRNLQEWQMAETENFRSENDSSLFYKIAPFRIIGFPAE

SEQ ID NO: 36 - protein, 565 aa, synthetic construct
MNTTTYNTTTDALLEWGKVYFQKEDFSEFLDNLEAYISDAGDSLKDELESGVEKLVLGIKSAEAVIFGEAVIGTTPENEAWYDAEESFLTLDCAVWLSQALDRVVRRQDASLADSLIARLDEAINRVAEKLYADNLSPLRFSSLNEIRRSALEATDEKYHYLFPWHGAACDVDENILLILTEEYHLIGADKAGANLSEELRGDLPFIFAELERDEVLRAYVEKENALSLALENTMREHWAFGLLEAARDEGYNHPYPADVGMRIHQVARAVFSQTNLSPAERLAVAIAGACFTPEISEDRRLEILLDCEERVCEIEAPTGDDTSVRVIKDLKALADHRVRHEIPAESLVSLWFEQIEAAGTDFDTKTPMDELVLRMLSDNVITLSVDRKAASQTETDDVKPQKGKIIPFPVPDIANDEVEYQKAVGMKKDKKAANDSKVKFPGLLEIQGCRDGDKAILLEDTDDAAANHRKLFSILKAGKLNSAFFIQSDDGEWVESESKPTMEDNRIILHDSHHSSFVWILDTGSMQLRQSVKCVKDALNKKTGSAKKLKPKTMIVWVTIPQEG

SEQ ID NO: 37 - protein, 8 aa, synthetic construct
AVGMKKDK

SEQ ID NO: 38 - protein, 578 aa, synthetic construct
MQLTDKSRNELFSALLEWGKSHMLSPEIVQDISEIEENCEPFIKSDEFTNFLLNSVEKIRRQVEFAISLFTNIELVTATDEDSLWHDAEVSFIAFDRVNYLMEVLQFLIVPRKKEIAEKAMKALDTIFEKTIGWLEESEFSPLRLVVLNESRRYHLEQIPEDERYRFPWYELYSDYSENTLEIIIENFDTFLSGKWEKLTREIPQEYLHEISLELKRDKLLLSRIKQEASYHKRLLAAVSKPSSLKLWRLGDEAALDYFLPGGVKKSGAVRVSLKLIEDAKIAFEADEICWKFLAAFCGPNLDDKQRLSLLDKVEERIKKIDIHKISENENILKTLKSWFEGNCDNAKLIKISFDTWIKMMEEKAINMHAVEFDESPEKLWNAIMELQKGRMGASNSVEKYINDFVNGCRQVWQIFKSTVDEPSVLYGARAIAASSEKSVQPRKLELNNNPILLSLKPNPKGEYIILSSLALKRVVLGEGVEDYEKIWNYLEHTKNDYWCGCFITNDDKPDVASVQQIENRILAKKTRDYKKAIIGVSPEKIVLEEFIQELPAVIFEGKGPLKDSLVKKVIILVISLE

SEQ ID NO: 39 - protein, 570 aa, synthetic construct
MYEHDYIGAILEWGKTKILSPHIIKYREDIEEYCKPLLESNEDELCDLLLRAIGELKDQVESAISTFGDIKLVTSTEYEELWHKAGAEFCTFDKINYLMESVHYLICIRDKRKYNEILQELDNVFSRITLWIEEGDFSPLRFVVLNEIRCESLLQIPEDERYRFPWYEAYSDYADDTVGIIIENFNLFLSGNWDKLITEIPREHLIEVSFELKRDRQLFSVIELKAALHKNLLETMSKPTSLKLLAMWDDARLDYYLPEKVVEVGPGKVRDEVLKDVTSSREDTLFGRFLNAFCGPGMDDRQRLNRLSKVEEEIKEVDISKASSPTKEVLSTLKLWFEGKCEDGKLTKISFETWLGKMERKANEIDTESFNAHLETLLRGVPGLTKFLEEDVAKEPILEGSLCTIDLDDQQASTQKEQIPIKQNPVGITLRLDREESISIMPDPDNIRASEDYKELWKFIDKVEDWYWGGTCFAEDKKNNLVFPLKTITYPLLGEIEGSKGYRFAVIGLSNNSEDLEIFVNKLKSVSITEKGQRTYPVDASGSRIAEEVRDYTQKPLNVVVLIIKYTYEE

SEQ ID NO: 40 - protein, 571 aa, synthetic construct
MITTELEFSQTFDLLLKYGQISLETDFTDYRDELATEIRSTVAEKKDNDSIIQDISNLEDAINAACEFWKKIHHLVLSRDDESWSEAQDTFRIFDWSNYFSQAVFELKSDILNFHPELSLLILKSDELVGKVNDILIDDDTISMLRMVPLNSFRQWKISLLPENCRYMYPWYETYSELPDTFLASLAENWKNIGNGDVSQLETPENLSIEMIFDDLKADKILFQSIESNHHETILLKNALKSMYPHRLWNLSEDASFDNPLLEGVYEKGLIRLAIRILDEKVTPDEIIGQIFWVAFCGAGLSDKQRLENFKWVMPHIEKKCYVSENSIYLSILGKLQNWVAGESKGFEFANTVYKSWNEHLFKIANQHLLADVETDESEEQLINRINQIKAKIQIKDAIEDFKKIWESICTKLADIRSKFNPFTVRCAGYAGVKEKEYTITYGKIPARLSIESIGGKIQLPNLPADLKVLEEVLLEEVLKDITEPYYSFGFSWNVDGEMKPIEEHNHDEDFNREIDDQFNDKKIQEILLVLEFDEKELKDTMKTFTEWIANPDVIKPPELKNVIVLYYSTS

SEQ ID NO: 41 - protein, 604 aa, synthetic construct
MNQEVTTNKPTLKPGTLFSLPFPGPQMPSTYLALAQQDHRFECARASGGEEDYTGDHDIFWSGLIIEVWNTIYLTSELITAQGCALRPIPSDILEDCLEIRSQITEDQPEYPNDPNSSFDEHTFQLIEKGANDALAKAIAGNSVTPLRIVPVWKKLGDLHEVITDKMKASILRTKLTLIYENALPPPQCLIWDSTDSFRIAVLAPDSNTNTVFTVWKKQMNSGWKAVVQVPYDIMKLALVTPEGSAILEILPSSNMQSAASLAERLFVARRESLAGLVFTSEPQLKTPHPYGVLEELATANHSGLRALMLGLKNHIGFAVAAGLCCQNLPGILQLELFQLAEDMTKKSDSGKEGQEIGALADHLKIWQTSKSEPDWICKRAFKLWDNELVKNFAQAKAMKPSEDITHAFERYADWLTTWVPVLCEIEKGKTWTAIMAATIKENLSNVITGRLPQPQAAHATNVGRQPQDPSSANILEAAITSIPDNWNPFDTAGTLRPLDARSRTAIKLLLENWKKKDQWFALLIIGEDNIHIVGPSNHCQTSPTAWEQNWISVFVILGHLEASVKKALDAVENIASGQPSNEDSPVLPTSPAEVAVIHWSINQ

SEQ ID NO: 42 - protein, 576 aa, synthetic construct
MREEYYNSELTMTPLLKEMGRALLKTPNFVAGIQSMVAGFQEYSDGETIVLAEMSELLEAAREARVFLGGIQLNPPKNRKVQDEDELFLQAQEAFQALDSVLFCKTALEHLKKLDIWKNTEVLSRLEEWQEDIAAWIGGEGGFSPLRLIVFQQIRRSILESFPEEVHYLFPWYEAFSEEEEGALFLLITNYDKIADPKKHSLLPEELRLKANAYYLELAMDKELRRDVAKAYKIHQALAKAINESFAIRLFGILEEASMATPYPDDLLEYGFGSIAAQIIKGAKNIYWNRREQIGMALLIGFCCEGLSDAVRFKLFSWVEDQMSKVDLGTSVNGSTGEKALSRLNTWVTSKVDKWLYSSEYENGLYALWFDEMRKNKNKVATSDEVSEEELCRLAEELIESYDKFWVIIQLLVGIQIIYKAASLALLSKVMKPLSLPVSMGPGAAPPPKFPTPYNLIGVPWVRREGIETFFEKSLRDIGDDAAKAYHAYEFSQERNHSYCVLYLGSEGEILDRRGPAPLPRRGKSIALELPENTAAVLFGIGPRTSLEAEWRILETPDLPVGEKSEVYWVLFAPPC

SEQ ID NO: 43 - protein, 280 aa, synthetic construct
MNDKMIEKMITNRGKKVRPFHEAYEPEPGVIFLPPRQTEDWKSSYLIIEDVEDFYMCVRLSVREPAAAGIYDMIIEDMIIETWNALSIPKVMQIKGYRQISDKVTEAVLRVRRAWWDDELPDVSYPVGGDIFEVPDRLLFYESELQANEHLQRNLIEGTESLSRVKLWTRIGHKVGLTDYFEELQPIHLHGNFSAYIHHRYLFFLWEARHKTPPPEASVRETGSGLFEILSWKELKKENIWEALVTIPEGVGSAVLKIGETECFLELLPSEDEPVSADSP

SEQ ID NO: 44 - protein, 548 aa, synthetic construct
MESMRQYTTIFDTLIEIGRIKSINKGWLPEIEKYEKHILESAFRKNEEKAKQEMNHGINLLKEEVNKAKKSLFQDDIVFSDVELMEDTVEYCLNIFDEVVFAKIGIDEEAKKLENLKNIFNVASQSIDKIFEGLFDWFYEEFEPYRLVLYNELRREYVRKIPEDLKHFFPWYNLYIEEDENVFLKLALAGVGKQGKVKITPEFALAYEAIKRDKVLKAYVLQEYKDFKLFYDVIEENPYLILFHEICEIGDFIELPEVIEQKGFFYVVDKTFNRLKERIRPIDALVLAVCCGPMEDERRIEWLKGLLDLPEQILKEGKHAREIAQFKETKRGVEHFIEKLFDVWVKECEKTAKELELEKKEETYYQLFLSDVENLFSSISEKIKPYEPKPLEPILWLFIIAITKKLQRFSIRSDLMKEEAEEAVSSHFEIKTSLTVPLTLVPEARALKKRNVIKGDKRELEKALKRLRKFYYLVVAEKQGEAFLIEGPKERRSFPLIIEKISLPKDATFHVFLAYEKESLNCLLDISKQKEIETALKDILYIKVHLVD

SEQ ID NO: 45 - protein, 558 aa, synthetic construct
MDMPPFYDLQFSPTMERLIGHARAEILDYPNHQTGGLPDVDQAILANDLVGFQKVLRQALSFLRSPATFDLAPGMGPSLAPVFNLLDNAVILEKYLIDRASLMGDKAVMQVLAIGEMATTKQAVPFHRFIPVNAWRRQFRDHLPEDVSCFFPWYDFWANEDELLLEEFVYLLPILQQGDFSSFASQDAPRLRRLFAEIQRDKPFIAHLNKRHLLLQKTVQAVGKNTALRLFFLADGCEPGVYLPELVLEKGLHTVAVAAMAQAPTSEEKRHELLFLAAFAGPELEDEERLAIFRKTVAFIGGAHALQEGGLMDKLRQWAKNTLDDQELSTSVYRRWRDLLNGTAPDIVTPFAYQDGQFSQVVGNLHTASVLEEKTVVPASDVQPGLWASLAAPAKKFWDNFTEIIGQFRESLVLEPELSRAMSEEGGARERKPVEIPGLRLRQELMPEDGQFLTTESAREIRELLGDKILYFITLAVDSATGQISATPPARLTRSSRIKDDIHGKPFVVGIGADKEGLEAAIDELTSPEGIRDQSKLDHILWIAITINDQILRDRHAP

SEQ ID NO: 46 - protein, 616 aa, synthetic construct
MLNDYEERQTEGFLYSPALDLLLTWGSGSLFQGTEDEGEFVQGIEEEWHSLMEESDVKSELHSGLEALTQDINDALDFLCHAIPESDRETALAQTALEILDHGVRLFQTTRNFSEQNHFPEGFQVRAEALFRNITVDRAPSYIRLIPLNYWRQFMRGNISEHSFHLFPWYDEWADMPSETIEILIENWNEISHGNFECSEMDTETLKVLFAELSNDRELLNYIQEEARFGHILPRAIGKSLVLRLLMIRNEEVGKHAAPQKVREKGFVACACHIIQKIKKIFRSEEERLEGIFLSGFCGPHLRENQRTELFTQVEHELKTLDVSLRPGSLLEELCQWSQGKVADEVFSRRVFEHWNNELSLSAKAVTERVPEDAAIFLRAVNELAFAKLETETVAEKIEWGIGRLFDINKLAVNELAFAKLETETVAEKIGRGIGCLPDKVINLIPSLIVRLKNPGYAGNGDDDDGNENGSVTLPRMINVCGHREGNNVNLLEASETIPDEIEKLLRQETGEVPKKLIPVMNSRKKLFYQVLAESSDDKWVFCFDKPKKTRAARIEVAETVYDRFVLLLDPGIKGLKHSSEVMMSVLNSVKAEDRQISPYTVIIDLEIRDSEGGSK

SEQ ID NO: 47 - DNA, 1698 bp, synthetic construct
  source 1..1698: mol_type = other DNA
atgaatacaacaacatacaacaccacaacagacgcgcttttggaatggggcaaggtatattttcagaaagaggatttttctgaattccttgataatctcgaagcgtatatttcagatgccggggacagtctgaaagatgaacttgagagcggggtggaaaagcttgttttgggcataaaatcggcggaagcagttattttcggagaggctgtgatcgggactacgccggaaaatgaagcatggtacgatgccgaagaatcctttctgactttggattgtgccgtatggctgtcccaagcgcttgatcgcgttgttcgcaggcaggatgcgtcattggccgacagcctgatcgcccgcttggacgaagccataaaccgcgtcgccgaaaaactgtatgcggacaatttatctccgcttcgtttttcatcgctcaatgaaatccgtcgaagcgctctggaggcaacggatgagaaatatcattatctgtttccctggcacggggccgcatgtgatgttgatgaaaacattctgctgattctgacggaggaatatcatctgatcggggcagataaagccggagccaatctttcagaggaactgcggggcgacctgccttttatttttgccgagttggagcgggatgaggtgctgcgggcatatgttgaaaaggagaacgccctttctcttgctttggaaaataccatgcgggaacactgggcattcggcctgctggaagcggcccgtgatgaagggtacaaccatccctatcccgcagatgtcgggatgaggattcatcaggttgcccgtgccgtattttcccagacaaacctctctccggcggaacgccttgctgttgccatagccggggcctgttttacgcctgagatcagcgaagaccggcggcttgagattctgctggactgtgaggagcgggtgtgtgaaatcgaagccccgaccggagacgatacatccgtccgcgttattaaagacttgaaagcgctggccgatcaccgtgtccgccatgaaatcccggcagaaagtcttgtcagtctgtggtttgagcagattgaggcggcggggacggattttgatacaaagacaccgatggatgaattggtgttgcgaatgctttccgataacgtcatcactctgtctgttgaccgaaaagcggcttctcagacagaaacagatgatgtgaaaccgcagaaaggaaaaatcataccctttcctgttcctgatattgccaacgatgaagttgaataccaaaaagctgtgggaatgaagaaggataaaaaggcggctaatgacagcaaagtcaaatttcccggcctgcttgaaatccagggttgtcgtgacggggacaaggctattttgctggaagacacagacgatgcggcagctaatcaccggaaactgttttccattctgaaagcaggtaaattgaattcagcttttttcattcaaagcgatgatggagaatgggttgagtcggaatccaaaccgacgatggaagataaccgtatcatattacatgacagccatcattccagctttgtatggatattggataccggttccatgcagctcagacagagtgtaaagtgtgtcaaagacgcattgaataagaagacagggagtgcgaagaaactgaaaccgaaaacaatgatcgtttgggtcacgattccgcaggagggatga

SEQ ID NO: 48 - DNA, 1737 bp, synthetic construct
  source 1..1737: mol_type = other DNA
atgcaattaacagacaaaagtagaaacgaattgtttagcgctctccttgaatggggtaagtcacacatgctttcgccagaaattgttcaagatatctctgagatcgaggagaactgcgagccttttataaaaagtgatgaatttacaaactttttattaaattcagtggaaaaaatcaggcggcaagtagaatttgcaatttccttgtttaccaatatagagctagttactgcaactgatgaagattctctatggcatgatgcagaggtgtcttttattgccttcgatcgagtaaactatttgatggaggtattacagtttttaattgtaccccgcaaaaaggaaattgctgaaaaagctatgaaggcgttagataccatatttgaaaaaacaataggctggcttgaagaaagtgagttttcaccattaaggttagtagtacttaatgaatcccgccgttatcatcttgaacaaataccagaagatgaacgatacaggttcccttggtatgaattatattcggattatagtgaaaataccctggaaataattattgaaaacttcgacacttttttgtcgggaaaatgggaaaaattaacgagggagataccccaagagtacctgcatgaaatatcgcttgaattaaaaagagataaactattactttctcgaataaaacaagaggcatcttatcataaaagattactcgctgcggtatcaaaaccctcatctcttaagttatggcgattgggagatgaagcggcattggattactttcttcctggtggggtaaaaaaatctggtgcagtgcgagttagtttaaaattgatagaagatgcaaagattgcttttgaggcagatgagatttgttggaaattccttgctgctttttgtgggccaaatttggatgataaacaacgcctctccctgcttgataaagtggaggaaaggataaagaaaattgatatacataaaatttcagaaaatgaaaatatcctgaaaacactaaaatcttggtttgagggaaattgcgataatgcaaaacttattaagatttcttttgacacttggattaaaatgatggaagaaaaagcaatcaacatgcatgctgtagagtttgacgaatcgccagaaaagttgtggaatgccataatggaacttcaaaaagggagaatgggcgcttcaaattctgttgaaaaatatataaacgattttgttaatgggtgccgacaagtatggcaaattttcaaatcgaccgttgatgaaccatcggtattgtatggtgctcgggctattgctgcatcatctgaaaagtctgtgcaaccaagaaagttagaactcaataataatccaattcttctttcgttgaagccgaaccctaaaggagaatatattattttatcatcgctggcattaaaaagggtagttcttggtgaaggtgttgaggattatgagaagatatggaactatcttgagcataccaaaaatgattactggtgcggctgttttattacaaatgatgacaaacctgatgtagcttctgtacaacaaatagagaatcgaattttggcaaaaaaaacgagagattataaaaaagcaattattggtgtatctcctgaaaaaatcgttttagaagaattcattcaggaattgccggctgtgatatttgaaggaaaaggcccgttaaaggattctttagttaagaaggttatcatattggttatttcacttgaatga

SEQ ID NO: 49 - DNA, 1713 bp, synthetic construct
  source 1..1713: mol_type = other DNA
atgtatgaacacgattatataggagcaatacttgaatggggcaagacgaagattttatccccacacattattaaatatcgcgaagatatagaagaatattgcaaaccattattagaatcaaacgaagatgaattatgtgatcttctcttaagagcaattggtgaacttaaagaccaggtggaatctgcaatctctacatttggcgatattaagttggtgacatctactgaatacgaagagttatggcataaagcgggagcagagttttgtacttttgataagattaattatctcatggaatctgttcattacttgatatgtattcgtgataagagaaagtataatgaaattttgcaagaactggataatgttttttcacgtatcactttatggatagaagaaggtgatttttctcctctcaggtttgtcgtcttaaatgaaatacgttgcgaatctttgttacaaataccggaagacgaacggtatcggtttccatggtatgaagcttattctgattatgctgatgatacagttggaataattatcgagaattttaatctttttctatcaggcaactgggataagttaataacagaaatacccagggaacatcttattgaggtatcttttgagctaaaacgtgaccgtcagcttttttctgtaatagaacttaaagcggccttgcacaaaaacctcttggaaacaatgtcgaagccgacttccttgaaactattggcaatgtgggatgatgcaaggctcgattattatcttcctgaaaaagtggtagaagttggaccaggaaaggttcgtgatgaagtgctcaaagatgtgactagttccagggaagacactctgtttggaagatttttaaatgcgttttgcggtcctggcatggatgacagacaaaggttgaatagattgagtaaggttgaagaagagatcaaggaggttgatatctcaaaagcgtcttcccctaccaaagaagtattgagtacactgaaactctggtttgagggaaaatgtgaggacggtaagcttaccaaaatctcctttgagacatggcttggcaaaatggaaagaaaagcaaatgaaatagacactgaatcttttaatgcgcacttagagacactcttgaggggagtacctggattaacaaaatttcttgaagaagatgtagcgaaagaaccaatacttgaaggatctttgtgcactatagatttagatgatcagcaagcctcaacgcaaaaggagcagattccaataaagcagaatccagtaggaataactttgagactcgacagggaggaaagcatatcgattatgcctgaccctgacaatatcagagcgtctgaagactacaaagaattatggaaatttattgataaggttgaagattggtactggggaggcacttgttttgccgaagacaaaaaaaataatcttgtatttcctctaaaaaccatcacttacccattacttggtgagatagaagggagtaaaggttaccgatttgcagttatcggcttatcaaataacagtgaagatttagaaatatttgtgaataagttgaagagtgtatccattactgaaaagggtcaacgcacatatcctgtagatgcatccggtagcaggatagcggaagaggtcagggattatacgcagaaaccactaaacgttgttgtgctgattattaaatatacatatgaagagtaa

SEQ ID NO: 50 - DNA, 1716 bp, synthetic construct
  source 1..1716: mol_type = other DNA
atgattacaactgaattggaattttcacaaacttttgacttgctgctcaagtatggccagatcagtctggagaccgattttacagattatcgtgacgaattggccactgagatcaggtcaacggtagcagaaaaaaaagataatgattctatcatacaggatatcagtaacctagaagatgccataaatgctgcctgtgagttctggaagaaaatccatcacttggttttgtcgagggatgatgagtcatggtctgaagctcaggacactttcaggatttttgactggtccaattatttttcacaagcggtatttgaattgaagtctgatattcttaattttcatcctgagttatctttattaatcttaaaatcagacgaacttgttggcaaggtcaacgatatcttgattgatgacgataccatatctatgcttaggatggttccactgaacagttttagacagtggaaaatcagtctgcttcctgaaaactgccgatatatgtatccttggtatgaaacatacagcgaactgcctgatacgttcctggcatcattggctgaaaactggaaaaacattggcaatggcgatgtttcacaactggaaacacctgaaaacctttcaatcgaaatgatttttgatgaccttaaagccgacaaaatactctttcagtccatagaatccaaccaccatgagacgatattactcaaaaatgcgctgaaatcaatgtatccccaccggctgtggaatctgagcgaagatgccagttttgataatccgcttctggaaggtgtgtatgaaaagggactgatccgtctggcaattcgtattcttgatgaaaaggttactccggatgaaataatcgggcagattttctgggtcgccttctgtggagccggtctctccgacaagcagcgattagaaaatttcaaatgggttatgccgcatatagagaaaaaatgttatgtgtccgaaaactccatttatctatctatactcggtaaactgcagaattgggtggctggtgaaagcaagggtttcgaatttgcaaatacagtctataagtcgtggaatgagcacctatttaaaatagcaaatcagcacctgctcgcagacgttgaaacagacgaatcggaagaacaactgatcaacagaataaatcagataaaggccaaaattcagataaaggacgcaatagaggattttaaaaagatttgggaaagtatatgcaccaagttggcggatattaggagcaaatttaatccctttactgtacgatgtgcaggatatgctggggttaaagagaaagaatataccatcacctatggcaaaatacctgcccggttatcaatagaatctatcggtggcaaaattcagctgccaaatttgccggcggacttaaaagtattagaagaagttcttttagaagaagttcttaaagatattaccgaaccatattattcctttggtttttcatggaatgttgatggtgaaatgaagccaattgaagagcataatcatgatgaagactttaaccgtgagattgacgatcagtttaatgacaagaaaattcaggaaattctgcttgtgctggaattcgatgaaaaggagttgaaagatacgatgaaaacctttacagaatggatagccaatcctgacgtgatcaagcctccagaattaaaaaacgtcattgtcctctattattccaccagctaa

SEQ ID NO: 51 - DNA, 1815 bp, synthetic construct
  source 1..1815: mol_type = other DNA
atgaatcaagaagtaacaacgaataaaccaacgctaaaaccaggaacactgttttcccttcccttccctggtccgcaaatgccttcaacataccttgctcttgcacagcaggatcatagatttgaatgcgctcgtgctagcggcggggaggaggactatactggagatcacgatatattctggtccggtctcataattgaggtttggaataccatttatctcacttccgaacttattaccgcgcaaggctgcgcccttcgccctattccttcggatattcttgaggactgccttgagataaggagtcagatcacagaagatcaaccggaatatccaaatgatccgaatagttcttttgatgagcatactttccagttaattgaaaaaggcgctaatgacgcgcttgccaaagcaatcgcgggaaattctgttacccccttaagaattgtgccggtctggaaaaaactgggtgatcttcatgaagtcattaccgataaaatgaaagcatcaatactgcggactaagttgaccttgatctatgaaaatgcattgcccccgccgcagtgcctgatctgggactctacagattccttccgcatagcagtattggctccagatagcaatacaaacactgtttttacggtctggaagaaacaaatgaacagcggatggaaggcagttgttcaagtcccctatgatataatgaagttagcgcttgttactcccgagggtagcgctattctggaaatccttccatcttccaatatgcaaagtgcagcaagccttgcagaaaggctttttgttgcccggcgtgaatcgcttgccggcttggtctttaccagtgaaccgcagttaaaaaccccccatccttatggagtgcttgaggaacttgcgactgccaatcatagcgggttacgagctctcatgcttggcctgaaaaaccatattggttttgccgtggccgcaggcctctgctgccagaatctgcccggcatccttcagctagagctgtttcagctcgcggaagacatgactaaaaaatcagacagcggcaaggaaggacaagagatcggcgctctggctgaccacctcaaaatttggcagacaagtaaaagcgaacctgactggatctgcaagagggcctttaaactatgggacaatgaacttgttaaaaattttgcacaagccaaggccatgaagccaagcgaggatattacccatgcatttgagcggtatgctgattggcttacgacttgggtgcctgtcctctgtgaaattgaaaagggaaaaacgtggacagcaatcatggcagctactattaaggaaaatctttctaacgtaattaccggcaggcttcctcagccacaggcagcccatgcgaccaatgtcggcagacaacctcaagatccatcttcagctaacatcctcgaagcagccatcacatccataccagataactggaatccattcgacaccgcggggacccttagacccctcgatgctagatcacgaactgcaatcaagctattgctggagaattggaagaagaaagatcaatggttcgctcttttgataattggcgaggacaatattcatatcgttggacccagtaaccattgtcagacctctcccacggcctgggagcagaactggatctccgttttcgttatccttggccatttagaggcttcggtcaaaaaggcgctcgatgcggtcgaaaacattgcgtctggccagccaagtaatgaggactcgccagtattacccacgagcccggcggaggtggcggttattcactggagcataaaccagtga

SEQ ID NO: 52 - DNA, 1731 bp, synthetic construct
  source 1..1731: mol_type = other DNA
atgcgcgaagaatattacaactctgaactcacaatgacccccttattgaaggaaatgggcagggctttactgaagacacctaactttgtggcggggattcagagcatggtagcgggattccaggaatattcggacggagaaactattgtactcgcagagatgagtgagcttcttgaggcagcaagagaagccagagtttttctcggcgggattcaattaaatccccctaagaatagaaaagtgcaggatgaagatgaactatttcttcaagctcaggaagcctttcaggcgcttgatagcgtcttgttttgcaaaacggcactggaacacttaaagaagctagatatttggaaaaacacggaggtattgtcaagacttgaagagtggcaggaagatatagcggcctggatagggggcgaaggaggtttttctccgttaagacttatagtctttcaacagatcagaaggagcattttggaatcattccccgaagaagtacattatcttttcccgtggtatgaagctttttccgaagaggaagagggcgctttgtttctgctaattacaaattacgacaaaatagctgatcctaaaaaacacagcttattaccggaagagttacgacttaaagcgaatgcatactatttagagttagccatggataaagaattaaggagagatgttgctaaagcctataaaatacatcaagccctagcaaaggctattaatgaatctttcgctatccggttgtttggtatccttgaagaggcttcaatggctactccctaccccgatgacttgctcgagtatggttttggtagcattgctgcccagataattaaaggagccaagaatatatactggaatcgtagggaacaaataggaatggcattgttaatcggattttgctgtgagggtctgagcgatgccgttaggtttaaactcttttcgtgggttgaagaccagatgagtaaagtcgatctcggtacatcagttaatggcagcacgggggagaaggctctttcgcgtttgaatacttgggtgaccagcaaagtagataagtggctttatagtagtgagtatgaaaacggtctttacgccttatggtttgacgaaatgagaaaaaacaagaacaaagttgctacatccgatgaggtaagtgaagaagaattatgccggctggccgaggaattaatagaatcatacgataagttctgggttatcatccagttgttagttggtatacaaattatttacaaagcggcatctctagctcttctttcgaaagttatgaagcctttatctctacctgttagcatgggtccaggagccgctccccctccgaagtttcccactccatacaatctcatcggggtcccatgggtcagaagggaagggatcgagaccttttttgaaaaatctttgcgcgatatcggcgacgatgctgcaaaggcgtatcatgcatacgaattctcacaagagagaaatcatagctactgcgtactgtacctcggttctgaaggcgagatacttgacagaagagggcctgctcctcttccccgaagaggtaagtctatagctttggagcttcctgaaaatacggcggccgtactctttggtataggaccgaggacatccttagaagcagaatggagaatactggaaactcctgacttaccagttggcgagaaaagcgaagtatattgggtcttatttgcccccccctgctga

SEQ ID NO: 53 - DNA, 843 bp, synthetic construct
  source 1..843: mol_type = other DNA
atgaatgacaagatgatcgaaaaaatgattacaaacagagggaaaaaggtccgaccgttccatgaagcgtatgaaccggaacccggtgtgatcttccttccgcctcgtcagacagaagattggaagtcatcctacctgattattgaagatgttgaggatttttatatgtgcgtgcgcctgagtgtccgagagccggccgcagccggaatatatgatatgattattgaagatatgattattgaaacatggaatgccctgagcattcccaaagtaatgcagatcaaaggttacagacaaatctctgacaaagtgacagaagcagtccttcgggtgcgccgggcatggtgggatgatgaacttccggatgtatcctatcctgtcggcggagatattttcgaggtgcctgaccggctgttattttatgaatccgaacttcaggcaaacgagcaccttcagagaaatctgattgaaggaaccgaatcgctctcacgggtgaaattatggacccggatcgggcataaagtgggcctgacagattattttgaagaattgcagcccatccatttacatggaaatttttctgcctacattcaccatcgttatctcttttttttatgggaggcacgtcataaaacgccgccacctgaggcatctgtcagagaaacgggcagcggactgtttgaaattctttcttggaaagaattgaagaaagagaatatatgggaagcgttggtgactattccggaaggggttgggtctgccgtattaaaaatcggtgagacagaatgtttccttgaactgttgccgtccgaagatgaacccgtttcagcagattcgccatag

SEQ ID NO: 54 - DNA, 1647 bp, synthetic construct
  source 1..1647: mol_type = other DNA
atggaatccatgaggcaatatacaacaatctttgatacacttatagaaataggtcgcataaaaagtattaataaaggctggcttccagagatagaaaaatatgaaaagcatatcttagaatccgctttcagaaagaatgaagaaaaagcaaagcaagaaatgaatcacgggataaatcttttaaaagaagaagtgaataaggcaaaaaaaagtttatttcaggatgacattgtattttctgatgtagaattaatggaagacacggttgaatactgtctgaatatttttgatgaagttgtatttgcaaaaatcggcatagatgaagaagcaaagaaactagagaatcttaaaaatattttcaatgtagcatcccaatctattgacaaaatttttgagggtctttttgattggttttatgaggagtttgagccttatcgtttggtgttatataatgagcttagaagagaatatgtaagaaaaattcctgaggatcttaagcactttttcccatggtataacctttatatagaagaagatgaaaatgtatttttaaaactggctttagctggagtgggtaagcaaggaaaagtcaaaattactcctgaatttgctcttgcttatgaggcaattaaaagagataaagtacttaaagcttacgttttgcaagaatataaggactttaagctcttttatgatgttattgaagaaaatccatatcttattctttttcacgaaatttgtgagattggtgattttatagaactgccagaagtcattgaacaaaaaggatttttctatgtagtggacaaaacgtttaatcgcctaaaagaaagaatacgtccaattgacgcccttgtccttgctgtttgttgtggacctatggaggatgaaagaaggattgagtggctaaaaggacttttggatttgcctgaacagattttgaaagaaggtaaacatgcaagggaaattgcacaatttaaagaaactaaaagaggggtagaacattttattgaaaaactctttgatgtatgggtaaaagagtgtgaaaaaacggccaaagaattagagctagaaaaaaaagaggaaacttattaccaactttttcttagcgatgttgaaaatctttttagctccatttcagaaaaaataaaaccttatgaaccaaaacccttagaaccaattctatggctatttattatagccataaccaaaaaactccaacgtttttctatccgtagcgatcttatgaaagaagaagcagaagaagcagtttcttcccattttgagataaaaacttctttgactgtacctctaactcttgttccagaggcaagggcattaaagaagagaaatgtcattaaaggagataagcgagagttggaaaaggcattgaagagacttaggaaattttattacttggtagtagctgaaaagcaaggagaagcatttttgatagaagggcctaaggaaaggagaagtttccctttaataattgaaaaaatttctctcccaaaggatgctacttttcacgtgtttttagcttatgaaaaagagtctctcaattgcttgttagatatatctaaacaaaaagaaatagaaacagctttaaaagatattctgtatataaaagtccatttggttgactaa

SEQ ID NO: 55 - DNA, 1677 bp, synthetic construct
  source 1..1677: mol_type = other DNA
atggacatgccccctttttacgaccttcaattttcgcccacgatggaacggcttattgggcacgccagggcggaaatcctggattatcccaatcaccaaacaggtgggctgcccgacgttgaccaggctatactggccaacgacctggtcgggtttcagaaagtgctgcggcaggccttgagctttcttcgcagcccggccaccttcgatcttgcccctggtatggggccaagcctggcgcccgtcttcaacctcctggataatgcggttattctggaaaaatacctcatcgaccgcgcgtcgcttatgggcgacaaagctgtaatgcaggtacttgccatcggagagatggcaaccacgaaacaagcggttccattccaccgcttcatccccgtcaacgcctggcgccggcaatttcgggatcatctcccggaggatgtatcatgttttttcccctggtatgatttctgggctaacgaagacgaattgcttctggaggaattcgtttatttgttacccatcctccagcaaggtgacttttcctcttttgcatcgcaagacgccccccggcttcgtcgtctgttcgcggaaattcaaagagacaagcctttcatagctcatctgaacaaacgccaccttctccttcaaaaaaccgtacaagctgtcggtaaaaataccgccctgcgcctcttttttctggctgatgggtgcgaacccggggtgtacctgccggaactcgtcctcgaaaagggcctgcataccgtggcggttgccgccatggcacaggcccccacctccgaggagaaacggcacgagctgctgtttctggcagcttttgccggcccggaactggaagacgaagaacggctcgccattttccggaagacagtagcctttatcggcggtgcccacgctctgcaagaagggggtctcatggataaactgcggcagtgggcaaaaaacaccctggatgaccaggaattgtcaacaagcgtttaccgccgttggcgtgatctgcttaacgggacagccccggacattgtcaccccctttgcctaccaggacgggcaattttcccaggtggttggtaatttgcataccgcttccgtgcttgaggaaaaaacggttgttcccgcttcggatgtgcagccgggtttgtgggcgtccctggctgctcctgcaaagaaattttgggataattttacagagatcatcggtcagttcagagaatctctcgttctggaaccggaactgagccgcgcaatgagcgaagaagggggagccagggaacgcaagcccgtcgagatacctggcctgcgtctgcggcaggagttaatgccagaagatgggcaattcctgaccacggaatcagctcgggaaatccgggaactgcttggcgacaaaatcctgtactttatcactctggcggtggattccgccactggacaaatcagcgctacccctccggccaggcttacccgcagttcccgaatcaaagacgatatccacggcaagccgtttgttgtgggcataggggcggacaaagaaggccttgaggccgccatcgatgagttgactagccctgaagggatcagggatcaatccaagcttgatcatatcctctggattgctattaccatcaatgaccagattttacgggaccgacatgcgccttaa

SEQ ID NO: 56 - DNA, 1851 bp, synthetic construct
  source 1..1851: mol_type = other DNA
atgttaaacgattatgaagaaagacagactgaaggttttttgtactcccctgcgctggatctcctcctgacctggggaagcggttctctgtttcagggaacagaagatgagggggagtttgttcagggaattgaggaagaatggcattcactcatggaggagtcggatgtgaaatccgagttgcactccggtttggaggcactgacccaggatatcaatgacgccctggatttcctttgtcatgcaattccggagtcagacagggagactgcactggctcagaccgctctggagatactggatcacggggtacgcttgtttcagacaacccgaaatttcagtgagcagaaccatttcccggaaggctttcaggtcagggcagaggcattgttcagaaatatcaccgttgatcgcgcaccctcttatatccgcctgattcctctgaactattggcgacagtttatgcgcgggaatatttcggaacacagttttcatctgtttccctggtatgatgaatgggccgatatgccttcggaaaccatagagatactgatcgaaaactggaatgagatcagccatggaaactttgaatgttcggaaatggacactgagacattgaaggttctgtttgctgagttgtcaaatgaccgggagcttttgaattatatacaggaggaagccagatttggacacattctgccccgggcaatcggaaaaagccttgtccttcgtcttttgatgataaggaatgaggaagtggggaaacacgctgctcctcaaaaggtgagagagaaagggtttgtcgcgtgtgcgtgtcacattattcaaaagataaagaaaatctttcgctctgaggaagagagattggaggggatttttctaagtgggttctgcggtcctcacttgcgtgagaatcagagaaccgaactttttactcaagtggagcatgaactgaaaactctggatgtatcactgcgccctggaagtctgcttgaggagctttgtcaatggtcccagggaaaggtggctgatgaggtcttttcccggcgcgtgtttgaacactggaataatgaactgagtctgtccgccaaagcggtcacagaaagggttccggaggatgctgctatttttcttcgggcggtcaatgagttggcgtttgcgaagttagagacggaaacggttgctgagaaaattgaatggggaatcggacggctgtttgatattaataagctagcggtcaatgagttggcgtttgcgaagttagagacagaaaccgttgctgagaaaattggacgggggattggatgcttgcctgataaggtaattaatctcatcccttcgctaattgtgcgattgaagaatccaggttatgcgggaaatggcgatgatgatgacggtaacgaaaatggaagtgttacactcccccgaatgatcaatgtctgcggccatcgagaagggaataatgtgaatcttcttgaagccagtgagacgataccggatgaaatagaaaaactgctccgacaggaaaccggagaagttcctaaaaagttgatccctgttatgaacagccgaaagaaattattttatcaggttttagcagaatcatcggatgacaaatgggtattctgctttgataagcctaagaaaactcgggcagcccgaatcgaagttgcagaaaccgtatatgatcgttttgtgctgctgctggatccgggcataaaaggtttgaaacatagttcagaggtgatgatgtcagttcttaacagtgtcaaggctgaagatcggcaaatttctccttacacggttatcattgatctcgaaatacgagattccgaaggaggttccaaatga

SEQ ID NO: 57 - protein, 778 aa, synthetic construct
MRYSSRTNCEAIDNLAEALQDQENMPEIARRVLEFEAENAKPENALCQHGLPHTKKAASQIAGVRDKHSEFYDNALLDLVEEWLKTYEEAKKLTHRERRQEMEDKIRVLQPVLQAKGKDADPRFLSLLARIYLYRGMLFRPKGFTTPARKIEALKKAVQLSEKAVEKEKDNPNFLRTWAQAALELEAIPETSFKVSSGLLKDAAVCINRDGIHSLNDLQVILEYAESEGKTSFLQHVLVEKRYWKRPFDLFLLKARAAFALNRMDDVRYFLKSAMDKTPKALSSPFWDHLVDFLKKLRTKEGSDLWKEMAVAAHRLCREKEVKIANNIYLYRHWARQKSLYNMAFLAQNDLKEKAKIADSLKSRPVLRYQALREMKEHQNIAKLLEQDDQERDGGYHKQQVEMDERTGKRLSEKMEKAGVSYENLPVPWISVHFYLNESENSEDEGSKGYALIFDALTQSWKERRFDYAKLHRKFMTWQEAYISAKKSSFAKDSLVELCREIGNTMPFLFDTACIRDGAPVLWIPHGFLHRLPLHAAIRDEATNEIFLENHASRYLPAWSILNSASARRGKDSYMIKRFRAEDYEKEPFSELEDMEWDNEEHEKLATPDDLKHFMAKNPGVFAVLCHGHGDILNPLKSWLELEGGGVSVLDILRYEKANLSGTRVLLGACEADMAPPVEYAIDEHVSLSAAFLSHKAQEVIAGLWEINIGEADECYAEILDCSDLSTELKDWQCDWVEKWRDDVEASGDNSTFYHITPFRIMGFPLKLKENNESEAKQ

SEQ ID NO: 58 - protein, 816 aa, synthetic construct
MEHKTMTEPAGQNPSATDNDFEKFIIDTGCVFFATPQEDPKYQNNKVEWHQGLCRFAQNDSPPTVIGSAIFFLQKLQEPGLFSGLPVSPELCSKISKDKNEIVAYHQQCILRLCEELLVKGREAKEHRERRQAFDQAIKFLLVLKKGTSSDTPSPNGHIHFQDQVSILLAEAYYLRGKIIRPKGFSVPAKKIETLEVAEKILVDLVARDTTGKARRLRAMVHIDLAALRDPADDSGNLQDYRQALEQAVSSIGDTKTCGRDEIVIILARAEDNAGWTGSDGLSARLEELVNNGAAGPLDQARAYLLLGQNNLAVTQTEKAITRMAATDNPTPFSHEDWRLLVRLLRDLKHQNTAGIDKLILDTWRKVHQIERQTKNGMHVRWYWSRQRDLYDLAFHAAGNDARLKAQIADSLKARPALHLGQAADLGLAVEQMEAGLLDRYMPGKMLEQTTDMAAPAAPGSAGWPELPRPWIAVHFYLSNGFGHPEGKQQGHALIQDSSKGDGKDTWSERTFDYFPIWAAFMTWQENYQRLKKEAAPDLERLCQVMGRQMPFLFAPEDLPLERPVVFVPHDFLHRLPLHAALIDNGEESGIPAQSHPITYLPGWWMVTSQAANPNETASKNTPSPVAPVALVHWDNSEDIHDIIKQANGTVVVNASRSDWLKLKHNAVGLKVLYCHGQAGYTNPFASSLKLDGGGLYLKDVVKGPPLVGRFILAACESDLVLPASTTLDEYFSFSTGLLQKGAAEILGTLWEVNETDALSLIETVLRAPASGNLSFVLRDWLRDNLRSLTTELFYDIAAFRALGGPYPVDTKEEHR

SEQ ID NO: 59 - protein, 769 aa, synthetic construct
MNTVELLQEEERLTLDLVFLPPGSKNKEQKKNALVDLLLKIVEHGELTRKYSALLTLSRGALRGEVHFGEKLLPSPEACANLAKPEEIKKMIRQHFQYRLDLLEAIVKKAADNTYSHARRRKALRIAIKELEQICEEALDELCFKARLLLAEALFERGRIVRPKGFSEPGKKKELFQKAINCIEGNCSEEALRLRARIYLQWYRFFHDEPPCDLDDIFTKALAVTDDKMLKTELLLLCGERKEPDPYTDDLRALLNDQNVSPLSRARAAVLLEDWERCNVEIYEAIEDLGKTDFFQQDWELVVTLLKKNYNQFHGWSRACTRLWEITVEKESKDAGHGCVLRWYWSRQRDVYNLAFAAFEECEDKARVVDSLKNRPAHHFSQLEQLAQSSDIIKQWIESEEIINQDSFAHSLRRHEKGAKSHSGGSLRIFPCLPKGWIAVHFFLASWPEPKGYALIHNADTNTWEQRDFKYEQLWATYIAWQEVSLHNKIRESALLLKSLCETLGKEMRWLFDEFLFPKERRRVLFVPHDFLHRLPLHMAIDIESQTVFAAKQPVCYLPAYHLQNNITENKKTSIYALVNLRENKQQKKDEEIFAEKVEKMGAIVRRPALESDLLNLNPVPEKLVLYCHGIGHSANPFASKLCLGDTGVSYRDILALNRSLAGCRVLLFACETDLVPAQTSSIDEHLSISNALLQKGAFEVLGSLWALPGKTIYGITKTFIDNDDTSAVLHSSLKRLFEHYEKKNEKTRAQLLYNWASLRVLAPAREFS

SEQ ID NO: 60 - protein, 703 aa, synthetic construct
MEEKHFLYNLYEKVKNYGDKAVFSGIKPSPGVCSKFKGLLNKPEATLTETFIKEVFKDELRLEPNKAKARARTYDNLIRLLSYWQETPLLALLLARLCYERALLIMQKAYGKSKKKESLLKQALNILDKILKHSDYPEALELKALVYLELKYMDVSPSDFSDILRPAFEKKKDCDAKITLALAEAGNGEALTCLSSKTIPSNYNYLDRTRIAILENHRSLAKKWLNEALSKEVPFVFSSPWWDELIEVLNKLPPNLKFTFSTKAFEKIYTLERSFKIHNLHLLWYWSKLKDIYEMAFIESISQKEYLKALFIADALKGRVMIKWHLMEKVLGEEFSDILEKEILGRLGYFVKSLEKKQKPSSTTWSWPNLNEYFFDFIPPDFAVVHLFFTEKQFENQGYAFILQKDNNVELKSFNIEKIWNYFLQFKNAYLFADKYPVSTASFSSAVKSLLEILGEELSFLFDKIVCKYVLFIPYGFLHQLPLHAMKHEEKGYFFEKYLTAYFPAWSFVYTISPEENASKQIVMLKYFDRHKFSKLKNAFRSFSIKDPASKEDFLNLTSPLNTLVIVSHGEANLVNSFESTLKLNPPLTLKEILEHKNNAFRGSKVLLIGCETDLEVPPKKIVDEYISLSTIFLLKGAKEVIGTLWEVYADTAEEAFLKLLHTNGKESLEKYQQYLLQVLSEGEIQIEEYIPLRIHLHPKSYL

SEQ ID NO: 61 - protein, 809 aa, synthetic construct
MNEEQTNRWPDLFDKFEDIIMEVDKKCDIEERTQFLRERKEFNAETLTSLEKADDWIRFGVILKVLSRESENSPVPVFTFRPSQNHCENLKKNSDKIDKAIAELNCKRAEKLLGHARTGKVRKYTDRHRLVESAITLVWEQFEKRDNGQFKWLHIHVKTEKQACNFIAKCYLLRSKLALPKGSSIPEKKLEALDNAWEWAKRGSPETDDLKMEVALQKHRWDPNLGKRWFQKQLNAFLDSNKLDLSNPLHWAVNDIVGDKALVSEEYDLEMLNDASVRNLTKNWEWKDKSGIPLYQARAAFRTHASDLDRRLINAVKKLKWLPLSHHLWEDTVALIKNVSEDDSFNGKWEMAAILAWAICQNAEARIKLSVQLRWYWSRARELYDLAFQAALKRKRPFLLVRITDSEKSRPTIKMQAAEKSFANAAAFQTYLEAETLFATGNFNAGLKELNSVPIEKMRTRSVRAVPEGWAAVHFNIIDKNESHALIVENRECHSIRIDLPDVWDAFQKWNTERRDLKLIKKSETSLEILCEKSGIMLEPILNQIKSENILFIPYGFLHLVPLHASKIKKPDETYTYLFQEKQCLFLPSWSLAPVEKENIHTGEHDLLLLAKMRGKDIQNIMDREDWCNEKNAENIENTADDFFNCLFDTLERFRKPPHLLVLYCHGQGDFVNPYCSKFIMEGRPLTHQDIVQDLQDLPVLQGTKVILTACETDLVSRHFGLIDEHLSLATAFLCKGASQVIASLFTCTTDISCEIIVHAKDNPEKSLGQILQEKQNQWAANEALYRLSVFRVMGFPGSARAMEEEISP

SEQ ID NO: 62 - protein, 731 aa, synthetic construct
MLLDSNALKEFLKKFKTFSQQSRKEQAKLLAKWEFFCNDTKFQAHPFGIEPEEELCKKVVKYSKDISLKSEAYLFLAREFIDNCKSQPNLLHREKRKYLEEAIRVLLEVIPEAETQKINLLNEIYFTIAKAYLLRSQIFRPKGMTVPEKKKEALKKALEWVKRIDFNNLEEAYLLKSEIYLELERIDERLADDAKETFEHGLNCKDCKAEPHIIAQIAVRWAELKNDINTKNILQTKVLEQSDVSHLEKAKAAFLLGQQNKVEQYLKRLSNELRNRYCLFSNPLWDGTVKFLKQLKDSNMDIWKDVSIKIWEVCEEKVRKASALHMRWYWSRQRDLYDLAFLAEEDPYKKAKIADSLKSRPSQKYKVWEKEARKYLEQEEAALGKRYIKEIEYDETPLPKLVPFTSLPAPWIAIHFYLNHLEKKGYALIYDAKKQKWEQPLSFEFYPIFEAFEVWQTNYFERKIGAAKFLEGLCKEIGNQMSFLFELPSERPVLFITHDFIHRLPLHAAIKNRKLFLDNNPSMYLPAWGFITRNDAENPRGRILLQNFKKYDFPKLKGMFGDQNPPPATRDHLKAMNEPPELLVILCHGTSDIVNPFSAKLHLAEGGITHREILRSELYINNSIVVLGACDTDLVPPITTSLDEHLSLNTAFLTKGARAVVGTLWKIKAEEMETFILRLDNSSARSSNIVYIIQELQKEASKKWKQNKKPEILYESMCFKAIGYPLLRNTP

SEQ ID NO: 63 - protein, 775 aa, synthetic construct
MENKAYSDALNLILEVDSKPDLIEGRQLLSQKRHVFEEALKTLKKAHSWSQLGALLCFLHEAYDYSLAPIEMPAREKALCANLDKYSDQIEEECTRINWQRAQGLRKTATDFSKEIKHTSRERLLERAIRLAWSRFSPEGEWPYPAVAAKAEVCEFISRCYLERSKLALPKGSSIPEKKLEALNKAWHWAKKATATLNFCQMEIALERDRWEEDLPESWLETLLKEFLSSQKLDFKNPSHWVIAHRARSLRLGDSTYDKELLAIDPSKFEERKELLWLPLFQSYAALRLNNSEVPRFLERAINKLSRVPFSDPLWDATVSLVEEVAKEGKDKWEPVATKLWEICKEKEEQVRLSIQLRWYWAQHQKLYALAFRAAIRQENCRLAAEIADSLKSRPTIKMMAIEKSMRSDEDREMSALQVEVDAVFAAGGFSHHYDNLLERVRELSKHKTPNQRRPIEDIPAGWAAVHFFLLSDEEGYALICKNGEFEKSPPLSLSRLWITYQAWEKARQTDPPDSYSLVEATERVCEALGDAFPFLFEIQENIIFIPHGFLHLLPLHAAKDDGRYLFLDKTCLYLPAWSLAPMGNKDSVSAQDMLFVNWEDTALREQLLKHKWFVEIDSATGRDLIDNLNKSACPPGLLVIICHGQGDLTNPYNSRLLLAEGGITHRELLKSLPSVGGSRVILAACETDFAPSGSGILDEHLSVSAAFLQKAAGEVGGTLFAAMAEECSEFVLAAKANPEKPLYEVLQKKQNEWANKININRLVPFRIMGFPQNK

SEQ ID NO: 64 - protein, 849 aa, synthetic construct
MNQNIDRAVGAILAIETATPLTESSTLAQRERHQKLLHDETKKIEQAFIALAQPPQCRAVEIAALSRFLQMTPLAVGPLRKRVICRAEPLKDDAHEQEIASHFNGLLLRLAKGLLASALNPAGIPWRRRVLWLEKAAHIAHRFDKEPLADDKERTEAAGVLARCCLHLALAHLPKGKDKSAMAERQEDLLQSLMWAQKAIVLAGQDKLSGEEYKLLKALVLIELDNLSPGRFQQQLNYVLYDLAVIWLERDTATKPFHPQELFVLWRYLATDFEPDLNMLLFKGSNTSERTAAVQQASPEAERFRPLLPLIHAWSAWKLDPPNNKIAEVILQAVNNLDEHQVYEQVWKWTVDFLQELRNTGAVDWQLPAIAAWELCNKKEKELPFGFQIRQYWSRLDSLYRLAFDGALELKDCMTAARIVDSLKSRTPLTWRDMDTLFAKLPKEKADQLREAFYSMEVQARMGFYAEAKEDANKLKKLLAAQVRKIRDIESVPAGWTVVHFHLREDQDLGYALACRLTADGMSYWTNHIFPVAGIRRAYDCWLEAYHGMEPGAREKSGYQLVELSEIMGKDLDFLFELAGEDGARGLLFVPHGFSHLLPLHAAKKDGSYLFEKIPSLTLPAWEFAPDVDQIPVSDGQDFCFISQRANEQDLVGNIERSHTWNGVCNKNAAWTNVLNTNKEWSKAPPRWLVFWCHGQADPHVAFRSKLLLGTLGVSLFEIQEAALSLTGTKVVLAVCESDLAPPEEYEKTDDHLSLAAPFLLKGARQVLAAIWEGAQLDLLKAMKEMLSNQDKHSWEILRELQSCWMRQPGAIFNDEYIRLYYAASFRILGFPEVATTNMATATAQEEIA

SEQ ID NO: 65 - protein, 808 aa, synthetic construct
MTSLELIKKSYERGLSHSQVLSITLRDDNKWQQRLKNRNSAFREVLTSYVKSVQETAEIINYVGSALLFLNDDQDEYASSFDGVGFSAEKCSALAGAEDVILRFHLDQQIKLNNQILYNIQEKGRLSHTIRRSALDQTIKNLLLMRQPPLCDLVDRKMQAQALSLSYLMRSRIIRQKGFSVPAKKIEGFQQALEALAFGYSKYPGDIEYLRIKSLILLEQDKIKATDSSGLEQCLKSYFGKLGIAGPDKADYPLILWYARKTNCHAYLDHILADGEPIEKLESAILLNCSSPEITGYANETITDLSEKPFSHDDWKTIVAILKAHPGLALKDITIALWDAARQRESITTSNCHLRWYWSQQQDIYEMAFHAADEASKKAEIADSLKGRPVLKRQAVEELARNDKSLKKYIDDQDAGWMGYIPKFKPAPSTSPPKKLKKTDNISQKKIFIATPQPWIAVHFFITTDAIGKKKGYAMVHDSQKNDQNNCWITHGPFNLDIVWSQYMIWQESYHRLGACGGDKNDSAPYMKKLCESIGKELSFLFVLPREQPVVFIPNGFLHLVPIHMAIDVANSEKNPHQIWAYKRKFTYLPAWSLIGSDHGSASPYAASVQILKYFEEGEYNYNKLRRRNLLMNDQASSSDILALQNTSSHLFLLCHGTANPNRPFDSGLKLKNGRLTIREILSMPRIPGMAILLGACETDMVGATASPLDEHISVSTSFLERGANEVIGGLFELRKKYTEDVALAIHDEMNNKYLYQIFTLFLSKKIDQYIKDKKCVSFYEMAAFRPLGLQSTVKSTPLENSVLKTQS

SEQ ID NO: 66 - protein, 769 aa, synthetic construct
MTSSRTNCSFIDRIEKALQKEDLESTLPELALRLIEFETANAEPENALCQRGISNANNAAVRIAKALGEKSALADMAEVRIKDYEVRKPRLTHRQRRQYLEDTIRILQPEEEKSKESGMLASLARVYLYRGVLYRPKGRITPARKTEAVRKAVRLSEKAIQNLSDKSGKAVFVWRTWAEAALELERAGDYSAPLETLEAAALQINADGITSLTDILILLRYAERSKKNAFKGKLTDLLDKKEHWWGHTSDIYLLKARIAFLFGHSDKEVWKYLKNALDHVPDAFSNPFWDDLVDFVKKLRDEESDMWKKTAIRAHGECRKKEAEIASGVVLRWYWSRQKDLYDLAFLAADHAEKKAEIADSLKSRPVLRYQTLRELKDIGTIGEILDREDEARDGRYLKTKPEPKEKEIVKEIKKKQAVPFKDMPEPWIAIHFYLNDFEEKGYALIFDATSRDDDGWKECRFDYRELHRKFMAWQELYFSGSEDSAADALVLLCREIGRAMPFLFDGTLPENSRVLWIPHGFLHRLPLHAAIRADENDTLFLEKHISRYLPAWNMLTSDSVKDNEASEDKGGFHMIKRLRPEDSDNYFKLNKRKWKNKEDEGIYRAREEDLKASMEKNPQALTLICHGHGDILNPLKSWLELEDSGMTVLDILKSEAKLSGTRVLLGACESDMAPPTEHTIDEHLSLCTVFLSHNAREIVAGLWEIQTNMVDGCYNQILDSNDISEALKQWQEDQMKKRWKKKQDHTIFYLIAPFRVMGFPKRVSSEAN

SEQ ID NO: 67 - protein, 716 aa, synthetic construct
MNNTEENIDRIQEPTREDIDRKEAERLLDEAFNPRTKPVDRKKIINSALKILIGLYKEKKDDLTSASFISIARAYYLVSITILPKGTTIPEKKKEALRKGIEFIDRAINKFNGSILDSQRAFRIKSVLSIEFNRIDREKCDNIKLKNLLNEAVDKGCTDFDTYEWDIQIAIRLCELGVDMEGHFDNLIKSNKANDLQKAKAYYFIKKDDHKAKEHMDKCTASLKYTPCSHRLWDETVGFIERLKGDSSTLWRDFAIKTYRSCRVQEKETGTLRLRWYWSRHRVLYDMAFLAVKEQADDEEPDVNVKQAKIKKLAEISDSLKSRFSLRLSDMEKMPKSDDESNHEFKKFLDKCVTAYQDGYVINRSEDKEGQGENKSTTSKQPEPRPQAKLLELTQVPEGWVVVHFYLNKLEGMGNAIVFDKCANSWQYKEFQYKELFEVFLTWQANYNLYKENAAEHLVTLCKKIGETMPFLFCDNFIPNGKDVLFVPHDFLHRLPLHGSIENKTNGKLFLENHSCCYLPAWSFASEKEASTSDEYVLLKNFDQGHFETLQNNQIWGTQSVKDGASSDDLENIRNNPRLLTILCHGEANMSNPFRSMLKLANGGITYLEILNSVKGLKGSQVILGACETDLVPPLSDVMDEHYSVATALLLIGAAGVVGTMWKVRSNKTKSLIEWKLENIEYKLNEWQKETGGAAYKDHPPTFYRSIAFRSIGFPL

SEQ ID NO: 68 - protein, 760 aa, synthetic construct
MKNRVQIEAIIRNLQGAARDSKTNKLSENIIAYDEYRKIHKSASLYQFGIIPAKESSSVLAENETNHVACENAIFEMAEKNIENFSSEDIHKKRKETIESALRLLMGLYKDRHEKLQPRTFVLIAKAYLLRSLITRPKGITIPEKKKEALKKGIGFVESAIKKIQSSENILSHSSDIDLLEKAWRIKSQLYLEYYRVNKDECDKNTLKEVLENSLISGCDKFDKNIEDVQIAIRYCELESSREYLEQIISSHLEGIEFEKARAYKLLELENENEDEIRKSMKVVIEEYLSGFSDPLWEDAVEFINKLKSDNKNCWKELSLDMYKVCREQEAETASLHLRWYWSRQRRLYDLAFIAADKEEEKAKIADSLKSRLSLRWSALEETGKKSKNKREKEEISRILEAEAVAMLGGYIKGARKILKKRRRPLPDEQRSIPKDWIVIHFYVNQLENKCYALIYNKDENTWKCEFVKEYQRLFHVFLTWQTNYNRCKERAADSLVQLCKEIGNAMPFLFDECIIPQDKNVLFIPHDFLHRLPLHGAIHEKNNGVFLENHPCCYLPAWSFAAKENNAVVQGSILLKNFPEYSYEELVSNSTLWTSPVKDPASPDDLKTIIASPEMLVILCHGEADAVNPFNARLKLTGNGISHLEILQSTKMILKGSKIILGACETDLVPPLSDIMDEHLSITTAFLTNDAREILGTMYEALDVRISSIIQKIYRQEHYSSMMKQLWEWQKVGVENYRENGDTPAFYNTVVFRVIGLSI

SEQ ID NO: 69 - protein, 773 aa, synthetic construct
MNDTLLRHLGLDIEKIAEEMQLLSADIEGNKEALVKTLVRYDEAKRIAKNAALWQFGLRPNQILFSVIDQTRQNQTMKEQAVRAVATQYLETFKQSREDGRDKCLTHNDQRELLESALKILVNFEKEMDGKIEPATCALIARTYLLRSAIMLPKGFTVPEKKKEALRKGSEYIRTIDDLTEEALRVRGSLLLEQRHIDILEKNRESNGDNQTLIKELREALENGCDKFNNTIEDVRIALCYIELTDDKTDLLQKIIDSQLDFPGIELYRLKAYFLKGDYAAISDEALKEELSGIRFNHPVWNEAMIFIKQLKDAQADCWRKLALAAYQVCRTRESETSSLHLRWYWSGYRLLYDLAFIAEDDLHRKAEIADSLKSRVSLHAKALDEIIKNDKEREEYYNAHAVAYAGGYVKGAGRIHTGRKEKDCDTNNVFKALPKDVAIVAFYLNYCEKNKDSRGRGYALIAENGTWNIKEFPFDSLYKAYLTWQTNYARHKESASPSLVELCEEIGRAMPFLFEITKKRIVFVPHDFLHRLPLHGAIKREWPKVLLEEYSCLYLPAWSLLHADTTKSSQTARKRMLIECFHEYDYHELQTKINAQIKESKGVVWEKREKAKPKDLLQIPEAPEILMILSHGRADMTNPYYARLKLEGGDVSALEIMKAKTGTMSIKGSNVIMGCCETDLLPVLSTPIDEHVSPATALYTRGANFVVGTMWEINPIDIERHFIELLTKNDNSMLEGVGNWQREGLSDDKWKKHKESRFFYAIIGFRVLGIFT

SEQ ID NO: 70 - DNA, 2337 bp, synthetic construct
  source 1..2337: mol_type = other DNA
atgagatattcatcccggacaaattgcgaagcaattgacaacttggcagaagctcttcaggatcaggagaatatgccggagattgccagacgggttcttgaatttgaggctgagaatgccaaaccggagaatgccctttgtcagcacggactgcctcatacaaagaaagcggcaagtcagatcgccggtgttcgtgacaaacactcagaattctatgacaacgcgttgcttgatctggtcgaagagtggctgaaaacctatgaggaagcgaaaaagctgacccacagggaacgacgtcaggagatggaggataaaatacgcgtccttcagccagttctccaggcaaaggggaaagatgctgacccgcgcttcttgtccctgcttgcccgcatctatctttacagaggaatgcttttcaggcccaagggatttaccacacctgcaagaaagattgaggctttgaaaaaagcggtgcagttgtctgaaaaagccgtggaaaaagaaaaagataatccgaactttctgagaacatgggcgcaggccgcgttggagcttgaagcaattccggaaacatctttcaaagtctcctctggtcttttgaaagacgcggctgtctgtataaacagggacggaattcatagtctgaatgaccttcaggttatccttgaatatgctgaaagtgaaggaaagacttcttttcttcaacatgttctggttgaaaaacgttattggaaacgtccctttgatctgtttcttcttaaagccagggcggcttttgccctgaaccggatggatgatgtcagatattttctcaaatcggcaatggacaaaacgccgaaagcactgtccagtcctttctgggatcatctcgttgattttctgaaaaagctcaggacaaaggaaggctcggatttgtggaaagagatggctgtggccgcgcatcgtctatgtcgggaaaaagaggtgaagatcgccaacaacatctatctgtaccggcactgggccagacaaaagtcgctgtataatatggcttttctcgctcagaacgatctaaaggaaaaagcaaaaatagcggattcgctcaaatccaggccggtcctcagatatcaggcattgcgtgagatgaaggagcatcaaaacatcgccaagcttcttgagcaggatgaccaggaaagggacggaggctatcataagcaacaggtggaaatggatgagcgaaccgggaaaagactatctgaaaagatggaaaaagccggggtgtcttatgagaatctgccggttccttggatttcagtccatttctatctcaatgaatcagaaaactctgaggatgaaggtagcaaaggatatgcgctgatctttgacgcattaacccaatcgtggaaggagcggcgtttcgattatgccaaacttcaccggaaatttatgacttggcaggaggcttatatttctgcaaaaaaatcgtcttttgcgaaggattctctggtggaactttgccgggagatcggcaatacgatgccgtttctctttgacacggcgtgtatccgggatggtgctccggtgctttggatacctcatggttttttacaccggcttccgctccatgcggccattcgtgatgaagctaccaacgaaatttttttggaaaaccatgcttccagatatctgccggcatggagcattctgaactcagcctccgccagaagaggaaaggattcttatatgatcaaaaggtttcgtgcggaagactatgaaaaggagcctttttcagaactggaggacatggaatgggataatgaagagcatgaaaagctcgcaacccctgatgatttaaaacattttatggctaaaaaccctggggtgttcgcagttctctgtcacggtcacggtgacattctgaatcctctcaagtcatggttggaacttgaaggaggcggtgtcagtgtacttgatattctcagatatgaaaaagcgaacctttcaggaacccgagtcctgttaggcgcatgcgaggcggacatggccccgccggtggaatatgcgatagatgagcatgtttcattgagcgctgcattcctgtcacataaggctcaggaagtaattgcaggattatgggagataaatatcggtgaggcagacgagtgttacgccgagatacttgattgcagtgatctttcgacagaattgaaagactggcagtgtgactgggttgaaaaatggagagatgatgttgaagccagtggagataattctacattctatcatattacccccttccgcatcatgggcttccccctcaaactgaaggaaaacaacgaaagcgaggcaaaacaatga

SEQ ID NO: 71 - DNA, 2451 bp, synthetic construct
  source 1..2451: mol_type = other DNA
atggagcataaaaccatgaccgagcctgccgggcaaaacccatcggccaccgataatgatttcgaaaaattcattatcgataccggctgcgtttttttcgccacccctcaagaagaccctaaatatcagaataataaggttgagtggcaccaggggctttgccgttttgctcaaaatgactccccgccaacagtaattggctcagctatattcttccttcaaaagctccaagagccggggctcttttccggtttgcccgtatcaccagaattatgttcgaaaatttcgaaggataagaacgaaatcgttgcctaccaccagcaatgcattttgaggctttgcgaagagttgcttgtaaagggcagggaagctaaagagcaccgcgaaagaagacaggcattcgaccaagcaataaaatttttgcttgtccttaaaaagggtacctcaagcgataccccttccccaaacggccatattcattttcaggaccaggtttcgatccttctggcagaggcatattacctgcgaggcaaaatcatccgacccaagggtttctccgtaccggccaaaaagatcgaaacccttgaggtggcagaaaaaattcttgttgatctcgtcgctcgtgacaccaccggcaaggctagacgcttgagggcaatggttcacattgacctggcagctttgcgcgaccccgctgatgacagcggtaacttgcaggactatcggcaggcactcgaacaggccgtttcctccatcggtgacacgaaaacgtgcggtcgggatgaaatcgtgattatcctggcaagggccgaggataatgccgggtggacaggaagcgatgggctgagcgcccggcttgaagaacttgtgaacaacggagcagccggaccattggaccaggcccgcgcttaccttttgctgggacagaacaacctggcggtgacgcagacggaaaaagccataacccgaatggctgccaccgacaacccaacgcccttcagccatgaggactggcggttgctggttcggctgttgcgtgacctgaaacaccaaaatacagcgggtattgacaaactcattctcgacacctggagaaaagtccatcagatcgaacgacagaccaaaaacggtatgcatgtgcgctggtactggtcccgccagcgggatttgtacgacctggcctttcacgccgccgggaatgacgccaggctgaaagcgcaaatcgccgactcgctcaaggcccggcccgccctgcacctgggccaggccgccgatcttggtcttgccgtggaacagatggaagcggggcttcttgatcgctacatgccgggaaaaatgctcgaacaaaccaccgacatggccgcgccggccgcgcccggttcggctggctggcccgaactgccaaggccatggattgcggtacatttttatctgagcaacggcttcggccaccccgaaggaaagcagcagggccacgccctgattcaggacagcagtaaaggggacgggaaagatacctggtcggaaagaacctttgactatttccccatctgggctgcctttatgacctggcaggaaaattatcagcggctgaaaaaggaggcggccccggatctggaaaggctgtgccaggttatgggccggcagatgccattcctcttcgccccggaagacttgccacttgaacgaccggtagtgtttgttccccacgatttcctgcatcgcctgcccctgcatgctgccctcatcgacaatggcgaagaaagcgggattccagcgcaatctcatccgatcacctacctgcccggatggtggatggtaacgagtcaagcggcaaaccccaacgaaacagcatctaaaaatacaccgtcgcccgtggccccagtggccttggttcattgggataattcagaagacatccatgatattattaaacaggctaacggcactgtggtcgtcaatgccagtcgatccgattggctgaagctgaaacataatgcagtgggacttaaggtgctctattgccatggccaggctggttataccaaccctttcgcctccagcctcaaattggacggaggcggtctgtacttgaaggatgttgttaaagggccgcctctggtcggccgctttatccttgctgcctgcgagagcgatctggttctgcctgcctctaccaccctggatgaatatttttccttttctaccggtttattgcaaaaaggggccgccgaaattctcggcactttatgggaagtaaacgagaccgatgccctcagcctgatcgagacagtcttgcgggcacctgcttcaggcaacttgtcttttgtgctcagggactggctccgggacaacctccgctctctaacaacagaactattctacgatattgccgcttttcgcgcgttaggcggtccatatccagttgatacaaaggaagagcaccgatga

SEQ ID NO: 72 - DNA, 2310 bp, synthetic construct
  source 1..2310: mol_type = other DNA
atgaatacagtcgaattacttcaggaggaagaacgcttgaccctggatttggtctttttgccaccaggtagtaagaataaagagcaaaaaaagaatgctttggtagaccttttgttgaaaatagtggagcatggggaattaacccgtaaatactcggcactgctgaccctctctagaggggctttacggggagaagtgcatttcggagaaaagcttcttccatctccagaggcatgtgcaaacctggccaagccggaagagataaagaagatgataagacagcatttccagtataggctggatttgttagaagctattgtaaaaaaggctgctgataatacctactctcacgctcgccgaagaaaagcgttgcgaattgcgataaaagagctggagcaaatatgtgaagaagctctggatgaactgtgtttcaaggctagattattgttggccgaggcgttgtttgaaagggggcggattgtcagacctaaagggttctctgaacctggaaaaaagaaagagctatttcaaaaagctattaactgtatagaaggaaactgttctgaagaggccttgagactaagagcccggatctatcttcaatggtaccgcttttttcatgatgaaccaccttgtgacctggatgatattttcacaaaagctcttgctgtaactgatgataaaatgctgaaaactgagcttttgttattgtgcggggagcgcaaggagcctgatccatatacagatgacttaagagccttgttgaacgaccaaaatgttagtcctctctctagggcaagagctgcggttcttcttgaagattgggaacgatgtaatgtagagatatatgaggccattgaagatcttggtaaaaccgatttctttcaacaagactgggaattggtcgtgaccttgctgaagaaaaattacaatcagtttcatgggtggtcccgggcatgtacaaggttgtgggaaattaccgttgaaaaggagagcaaagatgccggtcatggctgtgttcttcgttggtattggtcgaggcaaagagacgtgtataatcttgcgtttgcagctttcgaagaatgcgaagataaggcccgcgttgttgactctctgaaaaacagaccggcccatcatttttctcagttggaacaattggctcaaagtagtgatataatcaaacaatggatcgaaagtgaagaaataattaatcaggatagttttgctcattctttaaggcgccatgaaaagggtgcgaaaagtcacagcggtggttctttgcgtatattcccctgcctcccaaaaggatggattgcggtccatttttttcttgcttcctggcctgagcctaaaggctatgcactgattcacaacgccgataccaatacatgggagcaaagggattttaaatacgaacaattgtgggcgacctatatcgcttggcaggaggtctccctgcataataaaatcagggaatctgcactcctgttaaagagcctttgtgaaacattgggtaaggagatgcggtggttatttgatgagtttttatttccaaaggagcgaaggagagtgctttttgtcccccatgattttctccatcgtctccctctgcacatggcgattgatattgaatctcaaacagtttttgccgcaaaacagcctgtttgctatctccctgcctatcatctgcaaaataatattacagagaataaaaagacaagtatttatgcgcttgttaatcttagagaaaataagcagcaaaaaaaagatgaagaaatatttgctgaaaaagtagagaagatgggcgctatagtgagacgacccgcactggagagtgatcttttaaatctgaacccggtaccagaaaaacttgttctgtattgtcatggaattggacattcagccaatccttttgcatctaaactatgccttggtgacactggggtgtcatatcgggatattcttgctttgaaccgttctcttgcggggtgcagggttttgctttttgcctgtgaaacagatcttgttcctgctcagacatccagtattgacgaacatctttccatttcaaatgccttgttgcaaaaaggagcttttgaagtactggggagcctttgggcccttccaggtaaaacgatttatggaattaccaaaacctttatcgacaatgatgatacctccgctgtgctccatagttcattaaaaagattatttgagcattacgagaagaaaaatgaaaaaactcgtgcacagcttctctataattgggcgtctttacgtgttctcgctcctgccagggaatttagttga

SEQ ID NO: 73 - DNA, 2112 bp, synthetic construct
  source 1..2112: mol_type = other DNA
atggaagagaaacattttctttataatctttatgaaaaggtaaaaaattatggtgataaagctgttttttcaggtattaaaccttctcctggagtttgctctaagtttaaagggcttctcaataagccagaagcaacgttaacggaaacatttataaaagaagtatttaaagatgaacttcgtttagaaccaaataaagcaaaagcaagagctaggacttatgataaccttatccgcttattatcctactggcaggaaactcctttacttgcacttttactcgcaaggctatgttatgaaagagcacttcttattatgcaaaaggcctatggtaaatcaaaaaagaaagaaagtcttttaaaacaggctctgaatatccttgataaaattttgaagcactcagattatccagaagcattagagcttaaagcccttgtatatcttgagctaaagtatatggatgtttctccatccgatttttcagacatattacgacctgcttttgagaaaaagaaggactgcgatgctaaaataacgcttgctctagcagaagccggaaatggagaagcgctaacgtgtttaagtagtaaaaccatcccttctaattacaattatctagacagaacacgcattgctatactcgaaaatcaccgttcattagcaaagaaatggttaaatgaagctctttctaaagaagtcccttttgttttttcctctccttggtgggatgaacttatagaggtgttaaataaacttccaccaaacctaaaatttactttctctacaaaagcatttgaaaaaatctacaccttagaaagaagtttcaaaatacacaatcttcacctcttgtggtattggtcaaagttaaaagacatttatgagatggcatttattgaatcaattagccaaaaagaatacttaaaggcactctttattgctgatgctctaaaaggaagggtaatgataaagtggcaccttatggaaaaagtgttaggagaagaattctctgatatcctagaaaaggagatcctaggtaggcttggatattttgttaagagtttagaaaaaaaacagaagccatcttctacaacatggagttggcctaatttaaacgagtacttttttgactttattcctccagactttgcggtagttcatctcttcttcactgaaaaacaatttgaaaatcaaggttatgcctttattttgcaaaaagataataatgttgagttaaaatcttttaatatagaaaaaatttggaattactttttgcaatttaaaaatgcatatctttttgctgataaataccctgtgtcaactgctagcttttcaagcgctgttaaatctcttttagaaattttgggcgaagaattatcttttctttttgataaaatcgtttgcaaatatgtcttgtttataccatatggttttttgcatcaacttccattacatgcaatgaaacatgaagaaaaaggctatttctttgaaaagtatcttacagcttactttcctgcttggagttttgtttatactatttcacctgaagagaatgcatcaaaacaaattgtaatgctgaaatattttgataggcataaattttctaaactaaagaacgcttttcgttctttttctattaaagaccctgcttcaaaagaagactttttaaatctaacgtctcctttaaatactcttgttattgtttctcatggtgaggcaaacttggttaattcctttgaatcaactttaaaattgaaccctcccttaactttaaaagaaatcttagaacataaaaacaatgcctttaggggctcaaaggttcttttaatcggatgtgaaacagatctggaggtgccacctaaaaaaatagtagatgaatatatttcactatcaacgatattcttgcttaaaggagcaaaagaagtaatcggaacgctttgggaggtttacgccgatacagcagaagaagctttcttaaagcttcttcatacaaatggtaaagaaagcctagagaaatatcaacaataccttttgcaagttctttctgaaggagaaattcaaattgaagaatatatacctcttcgcattcatttacacccaaaaagttatttgtag

SEQ ID NO: 74 - DNA, 2430 bp, synthetic construct
  source 1..2430: mol_type = other DNA
atgaatgaagaacaaacaaacagatggccggaccttttcgacaaatttgaagatattatcatggaggtcgataaaaaatgtgatattgaagaacggacccaatttctcagagagcgcaaggagttcaacgctgaaacccttaccagcttggagaaagccgatgactggatccgttttggcgtgattctcaaagtcctgtcaagagaatctgagaactcccctgtgccggtgtttacattccggccctcacagaaccattgcgagaatctgaaaaagaatagcgataagattgataaagccattgcggaacttaactgtaagcgtgctgagaaattattgggtcatgcccggaccggaaaggtcagaaaatatacggacaggcatcgcttggtggaatcagccatcacgctggtatgggaacagtttgaaaaacgtgataatggccagttcaaatggcttcatatccatgttaaaactgaaaaacaggcctgtaattttatcgcaaaatgttatctcctcagatccaagcttgcgcttcccaaaggctcaagtattcctgagaaaaaactggaagccttggacaatgcgtgggaatgggccaaaagaggatcacctgaaacagatgatctgaagatggaagttgctctccaaaagcaccggtgggatcccaatcttgggaagaggtggtttcaaaaacaactcaatgcttttcttgatagcaataaacttgatctttccaatcccctgcactgggcagtgaacgacattgtgggggacaaggccctggtttccgaagaatacgatcttgagatgttgaatgacgcgagcgtccgtaatcttacgaagaattgggaatggaaagacaaatccggcatcccattgtatcaggcccgggccgcatttcgcacccacgcatcagatttggacaggagactcataaatgctgtaaaaaaactgaaatggcttccgctttcccatcatctttgggaagatactgtcgccctgatcaaaaacgtctcggaagatgacagcttcaatggaaaatgggaaatggcagcgatcctggcatgggcaatatgtcagaatgccgaagctcgcatcaaactgagtgtgcagttgcgatggtactggtccagagccagggaactttacgacttggcttttcaggccgcgctgaaaaggaaaagaccttttttgctggtcaggatcaccgattctgagaaaagccggcccaccatcaagatgcaggccgcggaaaaatcgtttgcgaatgctgccgcgtttcagacataccttgaagcggaaaccctgtttgcgacgggcaattttaacgcgggcttgaaagaactaaattctgtccccattgaaaaaatgagaacccgaagtgttcgggcggtgccggaaggatgggcagcggttcactttaacatcattgataaaaatgagagtcacgcattaattgttgaaaacagagaatgccattcgattcgcattgatctcccggatgtgtgggatgcttttcagaaatggaacacggagcgtcgtgatcttaaactgataaaaaaatccgaaacgtcattggaaatcctttgtgagaaatccggcatcatgcttgagccgatactgaatcagatcaaatcggagaatatcctgttcatcccttatggttttcttcacctcgtgccgctccatgcgtcaaagataaagaaaccggatgaaacatacacatatctgtttcaggaaaaacaatgcctgtttttgccttcctggtcattggctccggtggaaaaggagaatattcatacaggtgaacatgatcttctgctgctggcaaagatgagaggaaaggacatccagaatatcatggatcgagaagattggtgtaatgagaagaacgcagagaacattgagaacaccgcggatgacttcttcaactgtctttttgatacactggagagattcaggaaaccgcctcatctccttgttctttattgtcacgggcagggagactttgtgaatccctattgctcaaaattcataatggaggggagacccctgactcatcaggatattgttcaggatctgcaagacctgccggttttacaaggcacgaaggtcatcctcactgcctgcgaaacagaccttgtcagtcgtcattttggtctgatagatgaacatctctcactggccactgcttttctgtgcaaaggggccagccaagtcattgcctcactcttcacatgtacgacggatatctcatgtgagatcattgtccatgcaaaagataacccggaaaagtctctgggacagatccttcaggaaaaacagaaccagtgggcggccaacgaagcattatacagactgtcggtcttccgggtgatgggcttccccggttcggcccgggcgatggaagaggagatatcgccatga

SEQ ID NO: 75 - DNA, 2196 bp, synthetic construct
  source 1..2196: mol_type = other DNA
atgttgcttgactcaaatgcgctaaaagaatttttaaaaaagttcaaaacattttctcagcaaagtcgaaaagagcaagccaaactgctagctaagtgggaattcttttgcaatgataccaagttccaggcccacccttttggaattgagccagaagaagagctatgtaagaaggtagtgaagtattccaaagacatttccctaaagagtgaggcttatctctttttggccagggaatttatagataattgtaaatctcaaccaaacctccttcacagggaaaaacgtaagtatcttgaagaagcaattcgtgttttactggaagtcattccagaagctgaaacacaaaaaattaatttattaaatgagatttacttcaccatagccaaggcctatctgcttcgcagtcagattttccgtccaaaaggaatgaccgttccagaaaagaaaaaagaggccttaaaaaaggctttggaatgggtcaaaaggattgactttaataatttagaagaagcatatctgcttaagtctgagatatacttggaactagagagaatcgatgaaaggttggcagacgatgctaaagaaacatttgaacatggtcttaattgtaaggattgtaaggcagagccacatataattgcccaaattgctgtgcgctgggcagaattaaagaatgatattaacactaaaaatatcctacaaacaaaagttctggagcaaagcgatgtgagccaccttgaaaaggcaaaggcagcttttcttttgggtcagcaaaataaggttgagcaatatctaaaaaggctttctaatgaacttagaaatcgttattgtctttttagtaaccctttatgggatggtactgtaaaatttttaaaacagctaaaagacagtaatatggatatctggaaagacgtttcaattaaaatctgggaagtctgtgaagaaaaagtgcgtaaggcaagtgcgcttcatatgcgctggtattggtctcgccaaagggacttatatgaccttgcttttctagcagaagaagacccatacaaaaaagcaaaaattgcagattccctaaaaagtcgcccttctcaaaagtataaagtatgggaaaaggaagcaagaaagtatctagaacaagaagaagccgcattgggcaaaagatatatcaaagaaattgaatatgatgaaactcctttaccaaaactagtcccgtttacatcccttcctgctccatggatagccatccacttttatctaaatcacttggagaaaaaaggatatgctttaatttatgatgcaaaaaaacaaaagtgggaacagccgctttcatttgaattttaccctatttttgaggcatttgaggtttggcaaacaaattattttgaaaggaagataggagctgccaaatttttggaaggcttgtgcaaagaaattgggaaccagatgtcctttctttttgaattaccctcagagaggccagtactgtttatcacccatgactttattcatcgattaccactgcatgcggccataaagaataggaaattatttttagacaacaatccaagcatgtatttacccgcgtggggatttataacaaggaatgacgctgaaaacccaagaggaaggattcttttacagaattttaaaaaatatgatttcccaaagttaaaaggcatgtttggggatcagaacccaccgccagccacccgtgaccatctaaaagccatgaacgagccgcctgaattattggtaattctttgtcatgggacatcagatatagtgaatccctttagtgcaaaacttcatttagcagaaggaggaataacacatcgtgagattttgcgctcagaactttacatcaacaatagcatcgttgttttaggggcctgtgatacagatttagtccctcccattacaacgagtttagatgagcatctttctttaaatacagcatttttaacaaaaggtgcccgcgcagtggtcggaacattatggaaaataaaggctgaggaaatggaaacattcattttacgactggataattcatctgccagaagctcaaatatagtatatataattcaggaactgcaaaaagaagcaagtaaaaaatggaaacaaaacaaaaagccagaaatcttatatgagagtatgtgcttcaaagcaataggatatcctttattaaggaatactccatga

SEQ ID NO: 76 - DNA, 2328 bp, synthetic construct
  source 1..2328: mol_type = other DNA
atggagaataaagcttacagtgatgcactaaatctgatcctggaggttgattcaaaacctgatttgatagaaggccgccaactgctgtcacaaaaaaggcatgtctttgaagaagccttgaaaaccctaaagaaggctcattcctggtctcagctaggtgccttactctgctttttacatgaagcgtatgattattcccttgctcctattgagatgccggcacgagaaaaggctttatgtgccaatttagataaatacagcgaccaaatagaggaagaatgtacaaggatcaattggcaacgggcccaggggctacggaagaccgcgacagatttctccaaagaaataaagcatacgagccgggagcgacttcttgaacgggctattcgccttgcctggagccgtttttctcccgaaggagaatggccttatccggcggttgccgcaaaggctgaggtgtgtgaattcatctcccgttgctacctggaacgttccaagctggcgctaccaaagggaagttccattccggagaagaagcttgaggctctgaataaggcttggcattgggctaaaaaggctaccgccaccttgaatttttgtcagatggaaatagccttagaacgtgatcgctgggaagaagacctgcctgaaagctggttagaaacgctcttgaaagagtttttaagctcgcaaaagcttgattttaagaacccctcacattgggtaattgcccaccgagcacgttccctaagattgggcgactccacctatgataaagagttacttgctattgacccttctaaatttgaagaacgcaaagaattattatggcttccccttttccaatcttatgccgccctgcgtttaaataactctgaagtgccgcgcttccttgaaagggcaatcaataagctgagcagggtgcctttctctgatcccctctgggatgctacggtatcgctggtagaggaggttgcaaaagagggaaaggacaaatgggagcccgtagctaccaaactctgggagatctgtaaagaaaaggaggagcaagtcaggttaagcattcaacttcgctggtattgggcacagcaccagaaactctatgccctggccttccgcgctgccattcgccaagaaaattgccggctcgctgctgaaatagcagattctctcaagagcagaccaaccatcaagatgatggcaatagaaaagtccatgcgcagcgatgaggatcgggaaatgtccgccctgcaggtcgaagtcgatgctgtctttgccgcaggggggttcagtcatcattacgataacttacttgaaagggtgcgagaactttccaagcataagactccaaaccagaggcggccgatagaggatatcccggcaggctgggctgccgtccattttttcctcttaagtgatgaagaaggctatgccctcatttgtaaaaacggtgaatttgagaaaagtcctcccttaagtctctcaagattgtggattacctaccaagcctgggaaaaggccagacagactgatccgcccgatagttactcattagtagaagcgacggaaagggtgtgtgaggcacttggagacgcctttcctttcttatttgaaatccaggaaaatattatttttatccctcacggctttttacatctgctccccctccatgcggcgaaagatgatggaagatacctctttttggataaaacatgtctctatctgcctgcgtggtcactcgcccctatgggaaacaaagattctgtatccgcccaagatatgctcttcgtaaactgggaagatactgctcttcgggagcagctattgaagcataagtggtttgtcgagatagatagtgcgacaggaagggatctgattgataacttaaataagtctgcatgcccaccgggattgttggtgatcatctgccatggccagggggatcttactaatccttacaattcccgactgttgctggccgagggaggaataacccaccgggaacttcttaaatctcttccatcagttggcggtagcagggtcatcttagccgcctgtgaaacagacttcgccccgtccggctcaggcatactcgatgagcatctctctgtatctgcggcttttttgcaaaaggcagcaggtgaggtaggaggtacattatttgctgcaatggccgaggaatgtagcgaattcgtcctggcggctaaagctaacccagagaaacctctttacgaagtgttgcaaaagaagcaaaacgaatgggccaataagataaatattaaccggctagtacccttcaggatcatgggatttccccagaataagtag

SEQ ID NO: 77 - DNA, 2550 bp, synthetic construct
  source 1..2550: mol_type = other DNA
atgaatcaaaatatcgatcgtgcggttggtgcaattctagcgattgaaacagcgacaccccttaccgaatcttcaacactcgcgcaacgtgaaaggcatcagaagctgctgcatgatgaaaccaaaaagattgagcaagccttcatagccctggcgcagcctccccaatgccgcgcggttgagatagcagccctcagccgctttctccagatgacccccctagcggttggcccgctccgcaaacgggttatctgccgggccgagcctctgaaggacgatgcgcacgaacaagagatcgccagccattttaatggacttttgctcaggctggccaaggggctcttggccagcgcactaaatcctgcgggcattccttggcggcgaagggttctgtggcttgagaaggctgcccatatcgcccacaggttcgacaaggagcccttagccgatgacaaggaaagaaccgaggcagctggcgtcctggcccgttgctgcctgcatctggcccttgcccatttgcccaaggggaaagataaatccgccatggccgaacggcaggaagaccttttgcagtccctgatgtgggcgcaaaaagcaatcgtcctggcaggccaggacaagctttccggcgaagagtataaactgctcaaagcccttgtccttatcgagctcgacaatctttctccaggcaggttccaacaacaactcaattatgttctttatgacctggctgtaatttggcttgaacgcgatactgcaaccaaaccttttcatccacaggaactctttgtcttatggcgatatttagcaactgatttcgaaccagatttaaatatgttgcttttcaaaggatccaatacttccgagaggacggcagccgtgcaacaggcctcaccggaagcggagcgtttccggccgctgctccccttgattcacgcctggtcagcctggaaacttgaccctccgaacaacaagattgcggaagtaatactgcaagcagtcaacaatcttgacgaacatcaggtctatgaacaggtatggaaatggaccgtggattttctccaggaactccgcaataccggcgcggttgattggcaattaccggcgatagcggcctgggagctttgcaacaaaaaagaaaaggaacttcctttcggttttcaaatccgccagtattggtcgcggcttgattccctgtatcgtctcgcttttgatggagctctggaacttaaagattgcatgaccgctgcgcggatcgtcgattccctcaagtcccgaaccccccttacctggcgcgacatggatacccttttcgccaaactgccgaaagaaaaggccgatcaactccgagaggccttttactccatggaggtccaggcccggatgggtttctatgcggaagccaaggaagacgcgaataagctcaaaaaactgctggccgcccaggtccgcaaaattcgggatatcgaatccgtgccggccggctggactgtcgtacacttccacctgcgcgaggaccaggaccttggttatgccctggcatgccgtttgacggcagacggcatgtcttactggactaatcacattttcccggttgccggaatccgccgagcttatgactgttggcttgaggcgtaccacggcatggagcctggagcaagggagaaaagcggatatcagcttgtcgaactgagtgaaatcatgggcaaagacctggattttctctttgagcttgccggggaagatggggccagagggctcctctttgttcctcatggtttttcccatctcctgccgctgcatgccgcgaaaaaggacggcagctacctttttgaaaaaataccatctcttaccttgccggcctgggagtttgcccccgatgttgaccagatcccggtatcggatggccaagatttttgttttatctcgcaaagggcaaatgaacaggatttggtcggaaatatagaacgttcccatacttggaacggagtgtgtaataaaaacgccgcatggacaaatgtgcttaacacaaataaagaatggagtaaggcaccgccgcgttggctggtgttctggtgccatggccaggctgacccccatgtcgcgtttcgttcaaaacttctgctcggcacccttggcgtcagcctcttcgagatccaggaggctgccttgagtctcaccggcaccaaggtagtcctggctgtttgcgagagcgatcttgcgcccccggaagaatatgaaaaaaccgatgaccatctctctctggctgccccttttctgctcaagggagcccgccaggtcttggccgcaatctgggaaggcgctcagcttgatctgctgaaagccatgaaagaaatgctcagcaaccaagacaaacattcctgggaaatcctccgagaactgcaaagctgttggatgcgccaacccggtgccatttttaatgatgagtacatccgcctttattatgccgcctctttccggatactgggtttcccggaagttgcgactacaaatatggcgactgcaaccgcccaggaggaaatagcatga

SEQ ID NO: 78 - DNA, 2427 bp, synthetic construct
  source 1..2427: mol_type = other DNA
atgacaagcctggaattgatcaaaaaatcgtatgaaagaggtttatcacatagtcaggttctctcgattaccctaagagatgataacaaatggcagcaacgcctgaaaaatcgaaattcggcattccgtgaagtcctgacttcgtatgtcaaatccgttcaagaaactgctgaaatcataaactatgtgggttctgctcttctttttttaaatgatgatcaggatgaatatgcaagttcatttgatggggttggattttctgctgaaaaatgtagcgctctggctggagcagaagatgtgatattgcgttttcatcttgatcagcaaatcaaattaaacaatcaaattctttataatatccaggaaaaaggtcggttgtctcatacaatccgtcgatcagccctggatcaaaccatcaaaaacctgttgttgatgcgtcaaccgccgttatgcgaccttgttgatcgtaagatgcaggcacaggcgctttcactatcatatctgatgcgcagtcgaatcattcgtcagaaagggtttagcgtaccggcaaaaaaaattgagggttttcagcaggcgcttgaggctttagcgtttgggtatagtaaatatcccggtgatatagaatacctccgcataaaaagtctgatattattggaacaggataaaataaaagccacagacagttctggactggaacaatgccttaaatcttatttcggtaaattgggaatcgctggacctgacaaggcggattatccattgattctatggtatgcccgaaagacaaattgtcacgcctatctggaccacattttggcagatggggaacccattgaaaagttggaatcggccattctcttaaattgttcttcacctgaaattacaggctatgcaaacgaaacaattaccgacttgtctgaaaagcctttttctcacgacgactggaaaacaatcgttgctattctcaaagcacaccccggtctcgctctgaaagatatcacgatagctctctgggatgctgcacgacagcgagaatccatcactaccagcaactgccatttgcgctggtactggtcccagcagcaggatatttatgaaatggcatttcatgccgctgacgaagcatcaaaaaaagctgaaatcgctgattcactgaaaggccgaccggtgttgaaacgtcaagctgttgaagaactggcccgaaatgataaatccctgaaaaaatatattgatgatcaagacgcaggctggatgggatacatccctaaattcaaacctgcaccttctaccagtccaccgaaaaaactcaaaaaaacagacaacataagccagaaaaaaatattcattgcaacgccgcaaccctggatagccgttcattttttcataacgactgacgcaataggtaaaaaaaaaggttatgcgatggttcatgattctcagaaaaatgatcagaataattgctggataacacatgggccatttaatctcgatatcgtctggagtcagtatatgatatggcaagagtcatatcaccgtctgggagcatgtggtggtgataaaaacgactcagcaccgtacatgaaaaaattgtgcgaatctataggaaaagaactatcttttctttttgttcttccgagagaacagccagtagtttttattccgaatggttttttgcatctggtaccaattcatatggcgatagatgttgcaaactctgaaaaaaatccgcaccagatatgggcttataagagaaaattcacatacctccctgcatggtcattgataggtagtgaccatggatcagcatcgccatacgccgcaagtgtacagattttaaaatatttcgaggaaggggaatacaactataataaactgagaagaagaaatttgcttatgaatgatcaggcttcttcatcagatattcttgcgttacagaatacatcatcccatctttttcttctctgtcatggaacagccaacccaaatcgtcctttcgattccggtctgaaactaaaaaatggcagactaaccatccgcgaaatactttccatgccgcgaatacctggtatggctattctgctgggagcatgtgaaaccgacatggttggtgcaactgcttcgcctttggacgaacacatatcggtttctacatcatttttggaaagaggcgccaatgaagttatcggtggattgtttgagcttcggaaaaagtatacagaagatgttgcccttgctatccatgatgagatgaataataaatatctttaccaaatttttacgctttttttaagcaaaaaaatcgatcagtacatcaaggataagaaatgtgtttctttttatgaaatggccgctttccgtccgcttggattacaatcaacagtaaaaagtacaccactggaaaatagcgtgttaaagacgcaatcgtaa

SEQ ID NO: 79 - DNA, 2310 bp, synthetic construct
  source 1..2310: mol_type = other DNA
atgacatcatcccggacgaattgcagtttcattgacaggattgagaaggctctgcaaaaagaggatttggaaagcactttgcccgaactggcgctgcgacttatcgaattcgagacagcaaacgccgaaccggaaaacgctctctgtcagagaggaatttccaatgcaaacaatgccgccgtccggatcgccaaagccctgggggaaaagtccgcactggcagacatggctgaggtgcggataaaagattacgaagtccgaaagccgcgattaacgcatcgtcagaggcgccagtatctcgaagacaccatccgcatccttcagcctgaggaggaaaagagcaaggaatctggcatgcttgccagtctggcccgtgtttatctttacaggggagtgctttacagaccaaagggacggatcacgccagcgagaaaaacagaggccgtcaggaaagcggtccgtctgtcggaaaaggccatccaaaacctgtcagacaaatccggaaaagctgttttcgtttggagaacatgggccgaggccgcgcttgaactggaacgggctggagactattcagcgcctttggaaacactcgaagcagcggccttgcagatcaatgccgacggaatcacgagtctgactgatatcctgatcctgctgagatatgcggaacgtagcaagaagaatgcttttaaaggcaagctcaccgacctgctggataaaaaagaacactggtgggggcatacctctgatatatacctcctgaaagccagaatcgcttttctgttcgggcatagcgacaaagaagtgtggaaatatctgaaaaacgcactcgaccatgtcccggatgctttttccaatcccttctgggacgatcttgtggattttgtgaagaagctcagagacgaggagtcggatatgtggaaaaaaacagcgattcgcgcacatggcgaatgtcggaaaaaggaggcggagatcgccagcggcgttgtcctgcgctggtactggtcaagacagaaagacctctatgatctggcctttcttgccgcggaccatgccgagaaaaaagcagaaatagcggattctctcaagtccagaccggtactccgataccagactctgagggaacttaaagacatcggaacgataggtgagattctcgaccgggaggacgaggcacgggacgggcgatatctgaagacaaagccggaacctaaggaaaaggagatagtgaaagaaataaagaagaaacaggcggtgcctttcaaagatatgcccgaaccatggattgccatccatttctacctgaatgatttcgaagaaaaaggatatgctctgatttttgatgccacatcccgggatgatgatggatggaaagaatgcagatttgactaccgcgagcttcatcggaagtttatggcgtggcaggaactgtatttttcgggcagtgaagattccgctgcggatgcgcttgtgcttctgtgccgcgagatcggcagggccatgccttttctttttgacggcacacttccggaaaacagcagggttctttggataccccacggctttctgcatcggcttcccctccatgccgctattcgcgccgacgagaatgatacgctctttctggaaaagcatatctccagatatcttcccgcgtggaatatgctgacatcggacagtgtcaaagacaatgaggcttccgaagacaagggcggtttccacatgataaaaagactccggcccgaagactcggacaattatttcaaactgaacaaaagaaaatggaagaataaggaagatgagggaatatatcgggccagagaagaagatctgaaggcatctatggaaaaaaatccccaagccctgacgcttatttgtcacggccacggcgatatcctgaatcctttgaaatcctggctggaactggaggattccgggatgacggttcttgatattctcaaatccgaagcaaaattatcaggaaccagagttttgctgggagcctgcgaatcggacatggccccgcccacggaacacaccattgacgagcatctgtctctctgcaccgtatttctctcccataatgcccgggagattgttgcggggctgtgggaaattcagaccaatatggttgacggatgttataatcagatactcgacagcaacgatatttcagaggctttgaaacaatggcaggaagatcagatgaagaaaagatggaagaaaaaacaggatcacaccattttttatcttattgcccccttccgtgtcatgggcttccctaaacgggtcagtagtgaagctaattga

SEQ ID NO: 80 - DNA, 2151 bp, synthetic construct
  source 1..2151: mol_type = other DNA
atgaacaatacagaagaaaacattgaccgtatccaggaaccgaccagagaagacattgatagaaaagaagcagaacggcttcttgatgaggcttttaatccaaggaccaaacccgtcgataggaagaagataattaattctgccctgaagatactcatcggtctttataaagagaaaaaagacgatttgacttccgcttcttttatctccattgcacgggcatattacctcgtaagcattacaatccttcccaaaggcactactatcccggagaagaagaaagaggcgctgagaaaaggaattgaatttattgatcgcgcaattaataaattcaatggatctatcctcgattcacaacgtgcattcaggatcaagagtgttctgtccatagaatttaatcgtattgacagggagaaatgtgacaatataaagctaaagaatctacttaatgaagcagttgataaaggatgtacagattttgatacatatgaatgggacatacaaattgccatccgcctatgtgaattgggagtagatatggaaggtcattttgataatcttattaaatcgaataaggcaaatgatctccaaaaggcaaaggcatactattttataaaaaaggatgatcacaaagcaaaagaacatatggataaatgtacagcatcactgaaatatacgccttgttctcatcgtctctgggatgaaacggtaggttttattgaaaggttaaaaggtgatagttctacattgtggagggattttgcaataaaaacttataggtcttgcagggtgcaggagaaagaaacgggtacccttcgcctgagatggtactggtcacgacaccgggtattgtatgatatggccttccttgccgttaaagaacaagctgatgatgaagaaccagatgtaaacgttaaacaggctaaaataaagaaactggcagaaataagtgattcactgaagagccgtttctcactccgtctgtctgatatggaaaaaatgccgaaatcagatgatgagtcaaatcatgagtttaagaagttccttgataaatgtgtaacagcatatcaggatggttacgtaattaatagatctgaagacaaagaaggtcaaggagagaataaaagcacaacttctaaacagccagagccgcgtccgcaggcaaaactgttggagttgacacaggtaccggaaggctgggtggtcgtccacttttacctgaataaacttgaaggaatgggaaacgccattgtttttgacaaatgtgcaaactcttggcaatacaaagagtttcagtataaggagctctttgaagtatttttgacctggcaggcaaattataacctttacaaggagaacgcggcagaacatcttgtaacgctgtgcaaaaaaattggcgagacaatgccttttcttttttgtgacaattttattcctaatggtaaagacgtactttttgtcccccacgatttcttacacaggctgcctctgcatggctcaatagaaaataagacgaacggaaaattattcctggagaaccattcttgttgctatctcccagcatggtcatttgcttcagaaaaagaggcttcaacttctgacgaatatgttttactgaagaattttgatcaaggtcattttgaaactttgcaaaacaaccaaatttgggggacgcagtcagtcaaggacggcgcgagttctgatgacttggagaatattaggaataatcctagattgttaaccatcctctgccatggcgaggcgaatatgtcaaacccgttcaggtccatgctcaaactggcaaacggcggtataacgtacctcgaaatactaaattctgttaaaggtttaaaaggcagccaggtaatcctgggcgcatgcgagacagaccttgttccaccactatctgatgtaatggatgagcattattctgttgcaacggcattacttttaataggtgctgctggagttgtcgggactatgtggaaagttcgttcaaataaaactaaaagcctcattgagtggaagctcgaaaatatagagtataagttaaacgaatggcaaaaagaaactggcggggcagcctataaagatcatcctcctacgttttatagatctattgcttttcgtagtataggattccctttatga

SEQ ID NO: 81 - DNA, 2283 bp, synthetic construct
  source 1..2283: mol_type = other DNA
atgaaaaatagagtacagattgaagcaatcataagaaatcttcaaggcgctgcaagagactctaagacgaacaaactatcagagaacatcattgcttatgacgaatacaggaagatccataagagcgcttctttgtaccaatttggcataatccccgcgaaagaatcatcatcggtgcttgcagagaatgaaactaatcatgtcgcttgtgaaaacgctattttcgaaatggcagaaaagaatatagagaatttttcctccgaagatatacataagaaacgcaaagaaacgattgaatctgccttgagactacttatgggtctttataaggatagacatgaaaaacttcagccgagaacctttgtcctcatcgcaaaggcataccttctgagaagccttattactcgtccaaaaggtataacgatacccgaaaagaaaaaagaggcgctgaaaaaagggattggctttgttgaaagtgccattaaaaaaatccaatcctccgaaaatattttatctcattcttctgatatagatttgcttgagaaggcatggaggatcaaaagtcagttgtatcttgagtattatcgggttaacaaggatgaatgtgacaaaaatacattaaaagaagttctcgaaaattccctaatatcgggatgtgataaatttgacaaaaatatcgaagacgtacagattgctatccgctactgtgaattagagagtagtagagaatatttggaacaaattatttcctctcacctggaaggtatagaatttgagaaggccagggcatacaaactccttgaacttgaaaatgaaaatgaagatgaaataagaaaaagcatgaaggttgttattgaagagtatttatcgggtttttctgacccattatgggaagatgcagttgagtttatcaataaacttaaatccgacaataagaattgctggaaggaactatcgttagatatgtataaggtttgccgagaacaagaggcggaaactgcgtctctccatttgcgttggtactggtcaagacagagaaggctttacgacctggcattcattgcagcagataaagaagaagaaaaggcaaaaattgccgattcattgaaaagtcgcctctcgcttcgttggtcagcattagaagagacgggtaaaaaatcaaaaaataaacgggaaaaagaagaaataagcagaatccttgaagccgaagcagtagcgatgcttggaggatatatcaagggtgcacggaagatcttaaaaaagaggagaagacctttacctgatgagcaacgttccataccaaaagactggatagtcatccatttttatgtaaatcaattagaaaacaagtgctatgcccttatttataacaaggatgaaaatacctggaagtgtgaatttgttaaagaataccaaagattgtttcatgtctttttgacttggcagacaaactataaccgatgtaaagagagggctgcggattcccttgtgcaactctgcaaagagattgggaatgccatgccctttctctttgatgaatgtattattccacaagataaaaatgttttattcattccccacgatttcctgcatcgattgccgcttcatggggcaatacatgaaaaaaacaacggtgtatttttagaaaatcatccatgctgttaccttcctgcgtggtcatttgctgcgaaagaaaataatgcggtagtacagggaagcatcttgctcaagaatttccctgaatattcatatgaggagttagtttccaattcaacactatggacttctccggtaaaagacccggcgagtcctgatgacctcaaaacaattatcgcttcaccggaaatgcttgttattctttgtcatggagaagcagatgctgtaaaccctttcaatgccaggcttaagttaacaggaaacggcatatcgcatctcgagatattacaaagtacaaagatgattttaaaaggcagtaaaataatcctgggcgcttgtgaaacagacctcgtaccgccactatcagatattatggatgaacatctgtctattactacagcatttcttacaaacgacgccagggagattttgggaacgatgtacgaggcacttgatgtacgcatatcaagcatcattcaaaagatatataggcaagaacattatagtagtatgatgaagcaattgtgggaatggcaaaaagttggggtagaaaattatcgtgaaaatggtgatacaccagcattttataacaccgttgtttttcgtgttattggattatcaatatga

SEQ ID NO: 82 - DNA, 2322 bp, synthetic construct
  source 1..2322: mol_type = other DNA
atgaatgataccttactaagacacctgggtttagacattgaaaaaattgccgaagagatgcagttgctttccgctgatattgaaggaaacaaagaggctttggttaagacgcttgtcagatatgacgaggcaaaaaggatcgccaaaaatgccgcgctctggcagtttggtttgaggccgaaccaaatactattcagtgtgatagaccaaacacgtcagaatcaaaccatgaaagagcaggcggtgcgggcggtcgcgacacagtatctcgagacatttaaacagtcaagagaagacggcagggataaatgccttacccacaatgaccagagagaactcctggaatcagcgctgaagattctcgttaactttgagaaagagatggacgggaaaattgaaccagcaacatgcgcgctcattgcgagaacatatctgctcagaagcgctattatgctgcctaaaggttttacggtgccggaaaagaaaaaggaggcattgcgaaagggtagcgaatatatacgcacgattgacgatttaacggaagaagcattgcgcgttcgcggcagtcttcttctggaacagaggcatattgatattctggaaaaaaatcgtgaatccaacggcgataatcaaactcttataaaagagctgcgcgaagcccttgaaaacggctgcgacaaatttaataacacaattgaagacgtgaggattgccctctgttacatcgaattaacggatgacaagacagatctcttgcaaaagataatagactctcaactcgatttcccggggatcgaactctaccggctcaaggcctactttttgaaaggtgattatgccgctatcagtgatgaggctttaaaagaagagcttagcggtatccgttttaaccatccggtctggaatgaggcaatgatctttataaaacagcttaaggatgcacaggcagattgctggagaaaattagcgttagctgcttatcaggtatgcagaacgcgagaaagtgagacgtcctcacttcaccttcgctggtactggtctggataccggctgctgtatgatcttgcttttattgcagaagacgacctccacagaaaggcggaaattgccgattcgctaaaaagccgggtttcccttcatgcaaaggcgctggatgaaattattaaaaacgacaaggagagagaagaatactataatgcccatgcagttgcttatgctggtgggtacgtgaaaggggcgggaagaattcatacgggaaggaaagaaaaggactgcgacacaaacaatgtcttcaaagcacttccaaaggatgttgcaattgttgccttttatctgaattactgtgaaaagaacaaggactcacggggaaggggctacgccctgattgcggaaaacggcacatggaatataaaggaatttccctttgacagcctttacaaggcatatttgacgtggcagacaaattatgcacggcacaaggagtctgcgtcaccgtctctggtcgaattgtgtgaagagataggcagggcaatgccctttctttttgaaataacgaagaaaagaattgtctttgtgccacatgattttttgcatcgattgccgctgcatggggcaataaaaagagaatggccgaaagtcttattggaggaatactcttgcttgtacttgccggcctggtcattgttacacgccgatacaacaaaatcctcacagacggccaggaaaaggatgctcatagaatgttttcatgaatatgattatcatgaattacagacaaagataaatgcacagataaaagagagtaagggtgttgtatgggaaaagcgagagaaggcaaagccaaaagaccttttgcagattcctgaagcgcctgaaatcctcatgatattatcgcatggaagggccgatatgacaaacccctattatgcaaggcttaagctggaaggtggagatgtatctgctttggaaatcatgaaagccaaaaccggaaccatgagtatcaagggcagcaacgtaatcatgggttgctgtgaaactgatctgttgccagtattatcaacacccattgatgaacatgtgtcgccagcgacagcattatacaccaggggggcaaattttgtagttggaaccatgtgggaaataaaccccatagatatagaaaggcatttcattgaactattaacgaaaaatgataatagtatgttggaaggtgttggaaattggcaaagagaagggttgtcagatgataaatggaagaagcacaaagaatcgagatttttctatgctattattggattcagagtattaggtatctttacatga

SEQ ID NO: 83 - protein, 6 aa, synthetic construct
HHHHHH

SEQ ID NO: 84 - RNA, 33 nt, unidentified
  source 1..33: mol_type = unassigned RNA; note = target RNA
ggattacccatgtcgaagacaacaaagcaaggc

SEQ ID NO: 85 - RNA, 38 nt, synthetic construct
  source 1..38: mol_type = other RNA
ttgatgtcacggaacctttgttgtcttcgacatgggta

SEQ ID NO: 86 - RNA, 15 nt, synthetic construct
  source 1..15: mol_type = other RNA
ttgatgtcacggaac

SEQ ID NO: 87 - protein, 435 aa, synthetic construct
MNTTTYNTTTDALLEWGKVYFQKEDFSEFLDNLEAYISDAGDSLKDELESGVEKLVLGIKSAEAVIFGEAVIGTTPENEAWYDAEESFLTLDCAVWLSQALDRVVRRQDASLADSLIARLDEAINRVAEKLYADNLSPLRFSSLNEIRRSALEATDEKYHYLFPWHGAACDVDENILLILTEEYHLIGADKAGANLSEELRGDLPFIFAELERDEVLRAYVEKENALSLALENTMREHWAFGLLEAARDEGYNHPYPADVGMRIHQVARAVFSQTNLSPAERLAVAIAGACFTPEISEDRRLEILLDCEERVCEIEAPTGDDTSVRVIKDLKALADHRVRHEIPAESLVSLWFEQIEAAGTDFDTKTPMDELVLRMLSDNVITLSVDRKAASQTETDDVKPQKGKIIPFPVPDIANDEVEYQKAVGMKKDKKAAN

SEQ ID NO: 88 - protein, 442 aa, synthetic construct
MSKEYTVDYAHLSYSKGIDLLSTIAWATFFSWHNKYITKLTLEWQSYLKNDDHPKDLDDGLREMEKVVSHTLALLTGPMTLTLQRDMDEEIGPLFQVLDQAVLLSEYLSQGLCPELDRRLAQKIYDIKDSIVLNDPVPYHRLIPINEWRQELRDELPDLVAPLLPWYEWFTDLDEMTLSDIVDNIKSLEKEDFTAFGNNAGIINNLFKAIKADKTFYKELEARAWSQAREAVFLATSQKYALFYLMERTLNLAGEMNEKVMNIGFQAVAVQAISYKPMFKEYHRIELLFLAAFCGPDLNDKERLSLFDKIEKALPDLTGAPEETLAYALSQWHTGKLAGPALAQKTLKLWESDLLKAADQIRSPQPYEDGRFRNAVKYVQEMPLDTIPVHEKAPFNYKDIFDQIPEIGGWIHDRFSEVSMYIQTFCAIPALEYGDHEEESKK

SEQ ID NO: 89 - protein, 438 aa, synthetic construct
MLLPKNNQEKNFNTMVQWGKTHLLSAGVPETEQCLKELEVDCKTLINSNVLDDFLSSSIEEINKKVALAISAISNDALITGTDEEGIWHEALEGFTAFDMVNYMLEVVHYLIAPFKKEAASKFIIKLDETMSNIISWIEDGDFSPLRLTVINNYRRHQLSQIPVDKQYLFPWYEIYSCYNKDILEVIIENFDTFVLETGKEHISALLELPIAELSFELRRDKKLFSIVKHEYILHKSLMKAISKRSALTLWRLGVNSSLDRIMPQHIEKADVVRTVNNLIKGVLTSDVTNTDKAYWLFLSAFCGPGLDDKQRLNLLEKVEKIVKKIDTSDIVGDMANVLKTLKHWYDSNCEDSQIVSISFDTLTRLMEENARRMQTREYDEDPKELWNTLNTLLKKPIPSIPFEDSWIERISEWINRYAVIQAPALAWLSEDEERTTE

SEQ ID NO: 90 - protein, 415 aa, synthetic construct
MYEHDYIGAILEWGKTKILSPHIIKYREDIEEYCKPLLESNEDELCDLLLRAIGELKDQVESAISTFGDIKLVTSTEYEELWHKAGAEFCTFDKINYLMESVHYLICIRDKRKYNEILQELDNVFSRITLWIEEGDFSPLRFVVLNEIRCESLLQIPEDERYRFPWYEAYSDYADDTVGIIIENFNLFLSGNWDKLITEIPREHLIEVSFELKRDRQLFSVIELKAALHKNLLETMSKPTSLKLLAMWDDARLDYYLPEKVVEVGPGKVRDEVLKDVTSSREDTLFGRFLNAFCGPGMDDRQRLNRLSKVEEEIKEVDISKASSPTKEVLSTLKLWFEGKCEDGKLTKISFETWLGKMERKANEIDTESFNAHLETLLRGVPGLTKFLEEDVAKEPILEGSLCTIDLDDQQASTQ

SEQ ID NO: 91 - protein, 442 aa, synthetic construct
MQPVDMNQKGFCNVLIEWGKTHTHSPQITSYLKRLEEYCKPFIHDNRLEDFLSHSIEELKKQVDTAVSILSDTELVTSTEDTPLWHEAESSLITLDRVNYLMDAIHFLIAPYRDAVYKKSIHKLDEIFSFITSWLEEGAVSPLRFVVLNDTRRECLSQIPEETRYRFPWYENYSDYDENILETIILYFDDFLSKKWKKLAKKIPVEHLVEIWHELKRDVNLFSLVKRESALQKKLFKAVSVPSSLKLLGLSSDETSDAILPECVENAGVIWVCVSLLHDAILEIFSEAEKIYWTFLPALCGPSLNGKQRLEFLDTVENMISKIDIMEIARDKGELLKTLKLWFEGKIEDSYLIKISFDTWNKLIEEKASRMPTREVDAAPDELWKAINNLVKKEIPIQVSERIIRFESLETMREKRKSHKQSEFYNIFLHKVAANAGKEEAG

SEQ ID NO: 92 - protein, 441 aa, synthetic construct
MQLTDKSRNELFSALLEWGKSHMLSPEIVQDISEIEENCEPFIKSDEFTNFLLNSVEKIRRQVEFAISLFTNIELVTATDEDSLWHDAEVSFIAFDRVNYLMEVLQFLIVPRKKEIAEKAMKALDTIFEKTIGWLEESEFSPLRLVVLNESRRYHLEQIPEDERYRFPWYELYSDYSENTLEIIIENFDTFLSGKWEKLTREIPQEYLHEISLELKRDKLLLSRIKQEASYHKRLLAAVSKPSSLKLWRLGDEAALDYFLPGGVKKSGAVRVSLKLIEDAKIAFEADEICWKFLAAFCGPNLDDKQRLSLLDKVEERIKKIDIHKISENENILKTLKSWFEGNCDNAKLIKISFDTWIKMMEEKAINMHAVEFDESPEKLWNAIMELQKGRMGASNSVEKYINDFVNGCRQVWQIFKSTVDEPSVLYGARAIAASSEKSVQ

SEQ ID NO: 93 - protein, 419 aa, synthetic construct
MLSPEIVQDISEIEENCEPFIKSDEFTNFLLNSVEKIRRQVEFAISLFTNIELVTATDEDSLWHDAEVSFIAFDRVNYLMEVLQFLIVPRKKEIAEKAMKALDTIFEKTIGWLEESEFSPLRLVVLNESRRYHLEQIPEDERYRFPWYELYSDYSENTLEIIIENFDTFLSGKWEKLTREIPQEYLHEISLELKRDKLLLSRIKQEASYHKRLLAAVSKPSSLKLWRLGDEAALDYFLPGGVKKSGAVRVSLKLIEDAKIAFEADEICWKFLAAFCGPNLDDKQRLSLLDKVEERIKKIDIHKISENENILKTLKSWFEGNCDNAKLIKISFDTWIKMMEEKAINMHAVEFDESPEKLWNAIMELQKGRMGASNSVEKYINDFVNGCRQVWQIFKSTVDEPSVLYGARAIAASSEKSVQ

SEQ ID NO: 94 - protein, 435 aa, synthetic construct
MFRDYKNRKFEDFLYSPATDILINWGRAHILRPEYLQETEDEWNFLKKKSDTRGLLETGTASLEKDTEDALNFLHGQETLDLYDDMAIQAQTALEILDNAVMLSEAVEFFPDQSDFSEDFTRKADHLLQEITIDCEPPSLRLIPINRWRREVLQDIPAEFQYLFPWYVNWSELPPETLEILIQHWKDIENNQLEMIETDSVTLDGETLYVLLTALTKDHVLLNHIRHQSRTAEVLSQAVKKDFKLQLLLKRNSDAPKYFVSDEVEDKGLYISACKNIDTRDPLDPREVLNPLRSKEDRTERLFLAAFCGPGLTDEKRIDLFGRVENNLDRLDFSKIKSDSPLKLIDQWKKQGDSQLKKLSDIVLESWLDNLTVQAKKVAEPVPNENISFLDTVNRIMTDAIVEPTTVLTGFIEWIKNFCASELYTEAAKSAGPLS

SEQ ID NO: 95 - protein, 429 aa, synthetic construct
MMMASDTKEFHSQHLELLMKWGEHHLLQPGKNGLLQDLAKTWKTLDSPSDSLDSDLEILGKKVMLALEQLNWLTSNIHRDEEPEIQSALRILDGIIFLVESLECFPGSADLPEDFHGKAENILHKLTIDQETLPLRLIPLNQWRREMLKTIPPDAHYLFPWYCEWAELPENLFDLLADHWEDILRGEASALGLNIATLNALLAALQTDPLLLCRIQQDADLLRLLPHAIRESLSLRLFAISETDRSNITGSNTLEKKDIHAACLTLTAPIFSEAERIGHLFLTAFCSPFPDDRQRLDLLSIVEDFLKQPDCAHIDGIIGKLCQWHEGRMSDEKISRVAFKQWGKNLARARKRSPKAITYSASSFWDAIETLRRAGISARKENGMFSQICQKISELASAFFSPKIIAATATACLVIFIIIYTRQDYEQLQ

SEQ ID NO: 96 - protein, 433 aa, synthetic construct
MSTENEFRHTENTLFSECLDILVQWGQLRLLMQDNDAFLQELEGQWAYLNAVSPQPDFGLDLFEGKTEAALALLHGRLTVGMDMDTEDADQVMTAMGTLDQVIFLVEAVDAFPDAGQFPKAFQEKVRELLRQVTIDRPPPGLRLIPLNQWRQDILKVIPSHIRYLFPWYASWAELDPDALDALAAHWDAALRGQANGMGIPPGTLGRLMAELGRDAPLLAHVQRKARQRQHSSKAAGKRLPGRLLALCEASRSEQPVPNGPGEADLLLAACLTLDVPVRSETGRAELIFLAAFCAPFPDARYRISLFSETERFLKRIPPSALPSGSLMESVYRWAHDRLSDRELIRKAFEEWEGRLGRTNEVTAPEILFTRAVEKVRNAPVPGHLRRSPDGVFRNAVRSAVKYIATRWLVAVAAACLLAALAFFTIYPPAPSV

SEQ ID NO: 97 - protein, 462 aa, synthetic construct
MEEYLYSPLIDTLLEWGKTKLLFPDHKEYLSLLEEGVGASSPADIGNELPIGVNTICAEAESGMETLSKPDLILATPDDSIWPEAEDGFLAFDRLVFLREALEYSTWSNESVLDITETYVKQVDEALQKVSDWLFAGEEFSALRLAALNEFRRKRLEDIPKDKEDLFAWYHLFTEDEFPLDWLIENFNLLQQETAPETLPEGIHLPVELYMAELRHDKILARVLEEEHRLSRGIQQAVVSSLALRLFTVSDLASMKYTLPPEVEAPGLSATAFHVISNAPRRTPADQLALVLQTGFCGPQLTDEQRLEAFSWVEEHIDAAIDYTGLIMTDSMLGRLATWRAGRTEQDDTQFADYVFSDWLEKLKSAAPIRTGESPHETIWSFLEGMMMAECEEKLWGVAEPRGSYGVEIPDPEIFDRLVGLSQNFIKRVEGFFRPERFQFLRPLQPAVALGALGTPEQDRME

SEQ ID NO: 98 - protein, 424 aa, synthetic construct
MSDDISYNIGTDILLEWGQVFLQPEAFPEFLSDIEEEALSLGESLDTHLKQGTARIAAEAVSARDILGASDIIGLTPDDEKWYEAEAAFSSLDLMIYMTEAIARMVRKKNALLADTLLTELDEQMYLTADFLHGDAFSPLRLISFNEMRKNMLEGIGEKHRYLFPWYELLSEEDSGLLELLITHYHDLGSTAELPESLRGDLPFYLAELGRDEELERYLQKENVISLSLEKTFREHWAFGLRNAARSAGYKRRRPEDVETLGIERISRGILAQKPQIPQDGLVLAVTGACFAPEIEEDRRIRLLLDMESWVKDLPPDGNASDLSFRCADHLKKLDQGEIGAEDAAELLLDFWFGQMETLRVSDEQRDMAELVGELLVQEPGGDGGKTPPSAEDILKMIIGAAICRQRIRWPLSFPMGETDDEEK

SEQ ID NO: 99 - protein, 427 aa, synthetic construct
MNTNDKNTIIDIVIQWGKIFMQKEKFPNYLSSLEDDFLSSIDDNLESEIKSGTSFIIQSSHDATYALSQPEILGVTPDDEEYYKIEDAFLKLDNLVYLYEGLDTLLKKQNENLIRDISVFLDDTLTKINEFFYESGFSALRLATLNESRKNIIEGIDESYSYLFPWYHLLNKEDSGILNILAVHYHEFAKKRNDINKLPEILKDNLYFYLSEISSDQILKKYLEEHNTIFLSINQALTKHWAFRLWEASKSEGYKRLLSEDIEIRGIENISRAVLSDIPETNSERIVLATAGSCFAPDISEDLRVKTLLKIEDKIKYLSLEGNDDLISNGIKKLTNFASNKISANEAASALCDFWFTQLERAGTQYNEEKDMIMLIEDILEERKEKDRKEKTPVDTYNRTIFFLLAIIINKYSKLRIIPTMSDEEII

SEQ ID NO: 100 - protein, 443 aa, synthetic construct
MIPNELHISTINFLEWGRLSFRDHIEQQELLEHLGKDVFSYEKLGLELRHDLDLIIKKALEALDFLNQPDILDMTEDDSDKASVLEAFDRLDNLVFFMEAAERLLAPKDEKMAEELLQKTTRCYKQVTSFFFSHDFSALRLSTFHKYRRKALEYIRPEQHYLFPWYDLFNKVDSDTLFLIAENINQPDNLPREIKDELPLFYAEIQQDQVLKNYIQKQSLIARKFVKLLDTHWSLRLWRFVEEINLDKILPEMVEQKAAVRLSQSLLQSSTMTIQDRFALGLSAALFAPDIDEAERFKLLEQCEKIFDRIKETSSPDNPAGRVIQRLNLLQQGRLSLEEAVQATLDYWIEQLNVVQAEARNRKADMAGLVRSIISVEDSDGVEKIKSRPQRPASSVGKDKPKSWLEKLKAWLGNPIVLITAVAAFLIVISVQFLLRQEDQMHI

SEQ ID NO: 101 - protein, 312 aa, synthetic construct
MTAFLNMHKAEIALQKEYAFPSALCRFLEYGRGRLLNSPCDAPSLPEAADFEPLVKMLVTARDAYGSDLTLGLDAGDAEIVIPLLAAFNLGNYACEAADHLTAPADHAQDIAMEDVFELSDDIEDLPFGRLIPCNELRRELLGQIPQENHFLFTWLTECVQFDIDSLERIIQTWDELWDKTAPGDDAEALVPVLTAIQSDTALMDHLKAEAGFHTSVMKAAGEVSRQQHMERIRRSAEEGPGIMDHLVSWFRMPAVSGAVALVLMVGIYFGTQVGKDGQPMPIGVTMEMLVYQDGLAVRGGEETKPDVRKPG

SEQ ID NO: 102 - protein, 312 aa, synthetic construct
MTASLNMHKVEVALMKEHAFSSALRRFLEFGRGCLLNVPGDEPPLPEAADFEPLAGMLAQAGDAFAGDLTLGLASEDSEIIIPLLAAFNLGDYACEAADRLIDVTDQARDAAMKDVCALSDDIDGLPFGRLIPLNEWRRELLGQIPQENHFLFPWLTECAQFDSDSLERIIQAWDELWDRTATRDDVEALVPVLAAIQSDTALLDHLKAEARFHSSVMKAAGELARQQHMERARRSAGKAPGIMDRIVSWLRMPAVSGAVALVLVAGIFFATQVDKGGRPMPIGVTMEMLVYQDGLTVRGGEEAEPEVRKPA

SEQ ID NO: 103 - protein, 437 aa, synthetic construct
MSMSYEPRQFQDQPPRYSMTADLLIVLGRMGLLGQGSAPEREDLRHGLDAALAADPADLARGGEALGRAVEEALASLCGDESFLLQDGTDEYLLAEALASLDHLVFLEEGLAGREAGKALGEALAELAQRVLAEVTGHAARLIPHNGFRLDMVAMLPAGQLHRFPWYTLWADVPEDALERLAAPESARAHAVGADEDLPADLRMALLQDLSADRPLARELRMRRRIMAGVVRAFAQHKSLGMLRLAREANERFAVPDSVLENGLAAAALGAWTRPPASEGERHERLFLYALCGPFLEARLRLDLFEQVEAFLRDPDAAEQRPGSPLAVLAAWMRGEVADDQLSATAFAVWDARLARAAQAGQGAEVTARQFQDHVRAEAAQPIRKSEAGEPTPSFIARLRQTLATLLPTPALRAATAGLLVAVLAGSALLTMHLSTP

SEQ ID NO: 104 - protein, 437 aa, synthetic construct
MSMSYEPRQFQDQPPRYSRTADLLIVLGRMGLLGQGSAPEREDLRHGLDAALAADPADLARGGEVLGRAVEEALASLCGDESFLLQDGTDEYLLAEALASLDHLVFLEEGLAGQEAGKALGETLAELAQRVLAEVTGHAARLIPHNGFRLDMVAMLPAGQLHRFPWYTLWADVPEDALERLAAPESARAHAVGADEDLPADLRMALLQDLSADRPLARELRMRRRIMAGVVRAFAQHKSLGMLRLAREANERFAVPDSVPEDGLAAAAMHAWTRPPASEGERHERLFLYALCGPFLEARLRLDLFEQVEAFLRDPDAAEQRPGSPLAVLAAWMRGEVGDDQLSATAFAVWDARLARAAQAGQGAEVTARQFQDHVRAAAAQPIRKTEAGEPTPSFIAKLRQTLATLLPRPALRAATAGLLVAVLAGSVLLTMHLSAP

SEQ ID NO: 105 - protein, 145 aa, synthetic construct
NFEMRVYQVLPLTRSDEAETVITAESGAVLSSKDCFQLLFTPTRDVYAYLLHQDAGGQTTKYLEGPLHKDSTQIFPDKVSRSCFTADSGSEVFYLVYFDNAVDGFAELTSQMSNTDDVLDPVRFEQEIRTVFPNAQIEIFSFSYK

SEQ ID NO: 106 - protein, 139 aa, synthetic construct
TAPIGTLRLLAAGEEGQFRGGAPRIIEQSGEVMLSGERFQVRFTLEEPAHAYVFFRDAQGEVSTLFAGRVEAGEHVLPSPGESYVLDEETGSERVVLVLAAKEQKKLEDIEALLHGGEVEAIAERHRGVSVREIAFSHK

SEQ ID NO: 107 - protein, 139 aa, synthetic construct
TAPMGTLRLLAAGEAGQFRGGAPRIIAQSGETMLSGERFQVRFTLEEPAYAYVFFRDARGRVSTLFSGRAKAGEHVLPSEGESYVLDEETGSERVVLVLAAKEQKKLEDIEVLLHGGEVEAVAERHRGVSVREIAFSHK

SEQ ID NO: 108 - protein, 100 aa, synthetic construct
DIPPEEGDAIQLRFTLDEEAYIYLYHQESSDKRELLFSGKLEKGTHVFPDKSKRMRIGKPGEDEYIILMASERALNKIDQTNAMVNKTNQPGLIIIELEL

SEQ ID NO: 109 - protein, 102 aa, synthetic construct
DAPLEEGDAIQIRFMLEEAAYVYLFHEQKGAPHEPLFSGRLGKGEHIFPEEGKRIRVIAPSEEEGVALVASREPLSPADLEKALADEADGGAAKATIIHFQF

SEQ ID NO: 110 - protein, 155 aa, synthetic construct
KEQIPIKQNPVGITLRLDREESISIMPDPDNIRASEDYKELWKFIDKVEDWYWGGTCFAEDKKNNLVFPLKTITYPLLGEIEGSKGYRFAVIGLSNNSEDLEIFVNKLKSVSITEKGQRTYPVDASGSRIAEEVRDYTQKPLNVVVLIIKYTYEE

SEQ ID NO: 111 - protein, 139 aa, synthetic construct
TEELKVEKNHIYFSLLPYDRGAYPIIPPPEEVSGCTAVKEYKKLWDVLGNIKGDSCYAGGICLKEDWQIKEIPIQPIKRRIFERIESKEGFRNAVIVISNERARIEEFIKIFSKSFDWTEREINEFNVIVLDIWLEKSS

SEQ ID NO: 112 - protein, 137 aa, synthetic construct
PRKLELNNNPILLSLKPNPKGEYIILSSLALKRVVLGEGVEDYEKIWNYLEHTKNDYWCGCFITNDDKPDVASVQQIENRILAKKTRDYKKAIIGVSPEKIVLEEFIQELPAVIFEGKGPLKDSLVKKVIILVISLE

SEQ ID NO: 113 - protein, 137 aa, synthetic construct; identical to SEQ ID NO: 112

SEQ ID NO: 114 - protein, 140 aa, synthetic construct
QLLLDIGNKLIKLSLRPNAEGKYPIIPSPNMVLSCNAIEDYKNIWNYVGNTTGNYWGGCLIKEDGKVEEFTVKEIKKRIFHRIEGNIYKTAIIGISSKKGDIEEFVCRLQECSTLPEKSFEKLLNIVVLIISLETDNKNA

SEQ ID NO: 115 - protein, 84 aa, synthetic construct
DSKVKFPGLLEIQGCRDGDKAILLEDTDDAAANHRKLFSILKAGKLNSAFFIQSDDGEWVESESKPTMEDNRIILHDSHHSSFV

SEQ ID NO: 116 - protein, 94 aa, synthetic construct
TVTLPGVIRVSGKQEADVICLDIASVSVPPEMEQISHEILTKLLERLNSGRETFYYQVLLKNPEGRWSTFPDKPEEIYDLEIPVPDIVPDEGFI

SEQ ID NO: 117 - protein, 88 aa, synthetic construct
EETFEFPTHIKGTGYKKENSVILLDNSIQNKSDDFERFIQFWNNKKDLKLYYNVISMSKDKQWIILKGTCKTYKPSIEIPCDDSDIFF

SEQ ID NO: 118 - protein, 101 aa, synthetic construct
EDDSLKIRDDSPLQQLDNIEYYWNGWYEPLDGDMENIYSDAEEGDITPSFEIGKDKEYKTVVVCIATDEEIVNTVVSKINSKEEPLPEEKKNTIWLIYTKK

SEQ ID NO: 119 - protein, 99 aa, synthetic construct
GNETGELNELRSNYDLSFGAIAINGSGEINITEPKFWTGPVKRKKIDIDKDAIVLIIAVSGDKEQLKAALEQFRNISSKSDPLPVIENVFWIIYYDKKE

SEQ ID NO: 120 - protein, 229 aa, synthetic construct
QVPHVPMKSEDAVEKAAVPESQSLRREVKKKDAMSLPGKAERMERFSPAPVSEQTEPEPALAPAPAVPEKQKSLPSETKRIQRKVPNIKSPTVSEGKEEDADQAELRIPDEKADQLAPLPATEFKPKTQFHDPDSVARESLHFKTWLDKIDLDHLKLVAILGSDAGRSAVLEDAYGNEYAVVRGMALGRNSGHVKEILSDRIIVEEKIETASGEIMTSRRELWLQKFSD

SEQ ID NO: 121 - protein, 151 aa, synthetic construct
PPPPKPPVKKDVRPATSPAPQAPAPAEEKMRRNRPDPFQSPFDGTEGDGAVTTETGAVPLPGTLPQTPSDRATAEEFRLMGILLTPDGHKALMKAPDGRGYLLGKGLSAGFHKIRVTEIFRDRVMIEERQQDGQGSPPVRKRVLTLQGEAE

SEQ ID NO: 122 - protein, 108 aa, synthetic construct
PQHEIKSDESDKLEQYLKCFKTFGKYWQAGIIFGANLPQTWKPDSLSRTPEPITQYVPKQTAAILIGLCSDEELLEKAIALVCEGKECTEESILWILYRPARKHAELE